(19) United States
(12) Patent Application Publication
(10) Pub. No.: US 2023/0399327 A1
(43) Pub. Date: Dec. 14, 2023

(54) HIGH ACTIVITY HPK1 KINASE INHIBITOR

(71) Applicant: ADLAI NORTYE BIOPHARMA CO., LTD., Hangzhou, Zhejiang (CN)

(72) Inventors: Yufeng CHEN, Hangzhou, Zhejiang (CN); Canfeng LIU, Hangzhou, Zhejiang (CN); Meng LV, Hangzhou, Zhejiang (CN); Qiaodong WEN, Hangzhou, Zhejiang (CN); Yongqiang SHI, Hangzhou, Zhejiang (CN); Peng WU, Hangzhou, Zhejiang (CN); Kaixuan CHEN, Hangzhou, Zhejiang (CN); Han YANG, Hangzhou, Zhejiang (CN); Wanli CHENG, Hangzhou, Zhejiang (CN); Youping WANG, Hangzhou, Zhejiang (CN); Pingping LU, Hangzhou, Zhejiang (CN); Nanhai HE, Hangzhou, Zhejiang (CN)

(73) Assignee: ADLAI NORTYE BIOPHARMA CO., LTD., Hangzhou, Zhejiang (CN)

(21) Appl. No.: 18/034,367

(22) PCT Filed: Oct. 26, 2021
(86) PCT No.: PCT/CN2021/126307

§ 371 Date: Apr. 27, 2023

(30) Foreign Application Priority Data
Oct. 28, 2020 (CN) 202011168944.1

(51) Int. Cl. C07D 471/04; C07D 519/00

(52) U.S. Cl. CPC C07D 471/04; C07D 519/00

(57) ABSTRACT

A compound of formula (I) having the activity of inhibiting HPK1 kinase and a pharmaceutical composition comprising the compound. Also provided are the use of the compound in the prevention and/or treatment of cancers, tumors, inflammatory diseases, autoimmune diseases or immune-mediated diseases.

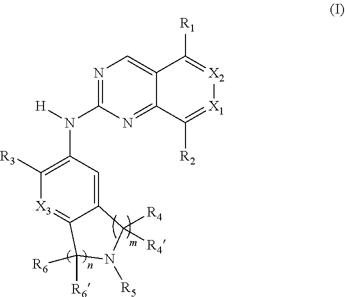

### CROSS REFERENCE TO RELATED APPLICATIONS

This application claims the priority of Chinese Patent Application No. 202011168944.1, filed with the China National Intellectual Property Administration on Oct. 28, 2020, and titled with “HIGH ACTIVITY HPK1 KINASE INHIBITOR”, which is hereby incorporated by reference in its entirety.

### TECHNICAL FIELD

The present invention relates to a heterocyclic compound, in particular to a highly active HPK1 kinase inhibitor and its application.

### BACKGROUND

HPK1, one of the members of the MAP4K family, is mainly expressed in cells of the hematopoietic system and acts as an intracellular negative regulator of T cell proliferation and signaling. After antigen stimulation of T cells, the adapter protein SLP-76 in the cytoplasm is recruited to the lipid membrane TCR complex, providing binding sites for signal transduction-related kinases to achieve TCR-mediated signal transmission and induce T cell activation. In this process, HPK1 is activated by phosphorylation of tyrosine kinases Lck and Zap70, and participates in the regulation of T cell receptor protein interaction. HPK1 phosphorylates the Ser376 site of the adapter protein SLP-76, allowing SLP-76 to bind to the scaffold protein 14-3-3ε and then be degraded by the proteasome, and this effect reduces the binding of SLP-76 to signal transduction-related kinases and blocks TCR signal transduction, and then inhibits T cell activation and proliferation. On the other hand, HPK1 is also involved in regulating the maturation and activation of dendritic cells (DCs), especially inhibiting the expression of proteins related to T cell activation in DCs cells such as CD80, CD86 and MHC complexes, thereby affecting the role of DCs in regulating T cell activation; and the presentation of activated DCs to tumor antigens and the mutual cooperation between DCs and T cells are one of the most important links in the anti-tumor immune system. In addition, there are a large number of immunosuppressive molecules such as PGE2 and TGF-β in the tumor microenvironment, and the immunosuppressive effect mediated by these factors is also closely related to HPK1. In general, small molecular compounds that specifically target and inhibit HPK1 can play a role in inhibiting tumor growth by enhancing the anti-tumor immune effect by improving T cell function, enhancing DCs cell function, and reversing the tumor immunosuppressive microenvironment at the same time.

### Contents of the Invention

The invention provides a compound with the activity of inhibiting HPK1 kinase as well as pharmaceutically acceptable salts, isotope derivatives and stereoisomers.

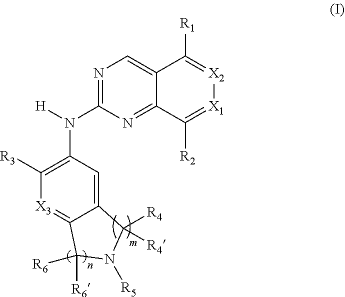

wherein R₁ represents hydrogen, halogen, (C₁-C₆) alkyl, (C₂-C₆) alkenyl, (C1-C6) alkoxy; wherein R₂ represents hydrogen, halogen, hydroxyl, (C₁-C₆) alkyl, (C₂-C₆) alkenyl, —(C₀-C₆ alkylene) (C₁-C₆) alkoxy, —(C₀-C₆ alkylene) (C₆-C₁₀) aryl, —(C₀-C₆ alkylene) (5-10) membered heteroaryl, —(C₀-C₆ alkylene) (4-10 membered) heterocycloalkyl, —(C₀-C₆ alkylene) (C₃-C₈) cycloalkyl, —NR^{L}R^{L′}, —OR^{L′}, —SR^{L};
wherein R₃ represents hydrogen, halogen, hydroxyl, (C₁-C₆) alkyl, (C₂-C₆) alkenyl, —(C₀-C₆ alkylene) (C₃-C₈) cycloalkyl, —(C₀-C₆ alkylene) (4-8 membered) heterocycloalkyl, (C₁-C₆) alkoxy, —(C₀-C₆ alkylene) (C₃-C₈) cycloalkyloxy, —(C₀-C₆ alkylene) (4-8 membered) heterocycloalkyloxy;
wherein R₄ and R_{4′} independently represent hydrogen, C₁-C₆ alkyl, (C₂-C₆) alkenyl, halogen;
alternatively, R₄ and R_{4′} form a 3-6-membered ring together with the carbon atom connected to it, and the ring can also optionally contain 0, 1, or 2 heteroatoms selected from N, O, and S;
wherein R₅ represents hydrogen, C₁-C₆ alkyl, (C₃-C₆) alkenyl, (C₃-C₈) cycloalkyl, (4-8 membered) heterocycloalkyl;
wherein R₆ and R_{6′} independently represent hydrogen, C₁-C₆ alkyl, (C₂-C₆) alkenyl, halogen;
alternatively, R₆ and R_{6′} form a 3-6 membered ring together with the carbon atom connected to it, and the ring can also optionally contain 0, 1, 2 heteroatoms selected from N, O, and S;
wherein X₁ represents N or CH;
wherein X₂ represents N or CR₇;
wherein X₃ represents N or CR₈;
wherein R₇ represents hydrogen, halogen, (C₁-C₆) alkyl, (C₂-C₆) alkenyl, (C₃-C₈) cycloalkyl, (C₁-C₆) alkoxy;
wherein R represents hydrogen, halogen, (C₁-C₆) alkyl, (C₂-C₆) alkenyl, —(C₀-C₆ alkylene) (C₃-C₈) cycloalkyl, —(C₀-C₆ alkylene)(4-10 membered) heterocycloalkyl, —(C₀-C₆ alkylene) (C₆-C₁₀) aryl, —(C₀-C₆ alkylene) (5-10 membered) heteroaryl, alternatively, R₈ can form (5-10 membered) cycloalkyl or (5-10 membered) heterocycloalkyl together with adjacent R₃;
wherein R^{L} and R^{L′} independently represent hydrogen, (C₁-C₆) alkyl, (C₃-C₆) alkenyl, (C₃-C₆) cycloalkyl, (C₀-C₆ alkylene) (C₆-C₁₀) aryl, —(C₀-C₆ alkylene) (5-10) membered heteroaryl, —(C₀-C₆) alkylene-(CR^{M}R^{M′})—(C₀-C₆) alkyl, —(C₀-C₆) alkylene-(CR^{M}R^{M′})-halogen;
alternatively, R^{L} and R^{L′} form a 4-8 membered ring together with the nitrogen atom connected to it, which can additionally contain 0, 1, or 2 heteroatoms selected from nitrogen, oxygen, and sulfur;
wherein the ring can also be optionally fused to another 5-6 membered carbocycle, 5-6 membered cycloheteroalkane, 5-6 membered aromatic heterocycle or benzene ring to form a fused ring bicyclic system;
or the ring can also be connected to another (4-6 membered) ring carbocycle or (4-6 membered) heterocycle through a spiro carbon atom to form a spirobicyclic ring system; wherein the fused ring bicyclic system or spirocyclic bicyclic system can be optionally substituted by 0, 1, 2, 3 members selected from halogen, cyano, (C₁-C₆) alkyl, (C₂-C₆) alkenyl, —NRₐR_{a′}, —ORₐ, —SRₐ, —(C₁-C₆ alkylene) hydroxyl, —C(O)Rₐ, —N(Rₐ)C(O)Rₐ, —N(Rₐ)C(O)ORₐ, —N(Rₐ)SO₂Rₐ, —C(O)ORₐ, —C(O)NRₐRa′, —S(O)2NRaRa′, —S(O)Ra, —S(O)2Ra;
wherein R^{M} and R^{M′} independently represent hydrogen, C₁-C₆ alkyl;
alternatively, R^{M} and R^{M′} form a 3-8 membered ring together with the carbon atom connected to it, and the ring can also optionally contain 0, 1, or 2 heteroatoms selected from N, O, and S;
For the above defined alkyl, cycloalkyl, heterocycloalkyl, aryl, heteroaryl, it can be optionally substituted by 0, 1, 2, 3 substituents selected from the following: (C₁-C₆) alkyl, (C₂-C₆) alkenyl, halo (C₁-C₆) alkyl, halo (C₁-C₆) alkoxy, —(C₁-C₆ alkylene)-O—(C₁-C₆) alkyl, (C₃-C₈) cycloalkyl, halogenated (C₃-C₈) cycloalkyl, halogen, —CN, oxo, —NRₐR_{a′}, —ORₐ, —SRₐ, —(C₁-C₆ alkylene) hydroxyl, —C(O)Rₐ, —N(Rₐ)C(O)Rₐ, —NRₐC(O)ORₐ, —NRₐSO₂Rₐ, —C(O)ORₐ, —C(O)NRₐR_{a′}, —S(O)₂NRₐR_{a′}, —S(O)Rₐ, —S(O)₂Rₐ, —P(O)RₐR_{a′}; wherein Rₐ and R_{a′} each independently represent hydrogen, (C₁-C₆) alkyl, (C₂-C₆) alkenyl, (C₃-C₈) cycloalkyl; or when Rₐ and R_{a′} are connected together on the N atom, they can form a 4-7 membered cycloheteroalkane together with the N atom connected to it; wherein m and n represent 0, 1, 2, 3.

In addition, the present invention also provides a compound having the following formula (II) or a pharmaceutically acceptable salt, isotopic derivative, and stereoisomer:

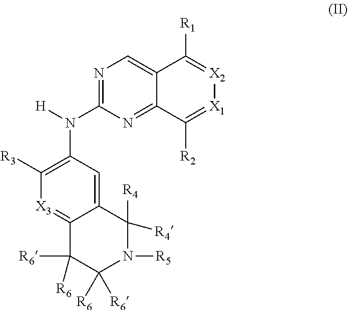

wherein, R₁, R₂, R₃, R₄, R_{4′}, R₅, R_{5′}, R₆, R₆, X₁, X₂, X₃ have the definition of formula (I).

In the preferred technical solution of the present invention, wherein, R₂ represents (C₁-C₆) alkyl, —(C₀-C₆ alkylene) (C₆-C₁₀) aryl, —(C₀-C₆ alkylene) (5-10) membered heteroaryl, —(C₀-C₆ alkylene) (4-10 membered) heterocycloalkyl, —(C₀-C₆ alkylene) (C₃-C₈) cycloalkyl; wherein, the alkyl, aryl, heteroaryl, heterocycloalkyl, and cycloalkyl can be optionally substituted by 0, 1, or 2 members selected from halogen, C₁-C₆ alkyl, —ORₐ, —SRₐ, —(C₁-C₆ alkyl)hydroxyl, halo(C₁-C₆)alkyl, halo(C₁-C₆)alkoxy, —(C₁-C₆ alkylene)-O—(C₁-C₆)alkyl, C₃-C₆ cycloalkyl, oxo, —NRₐR_{a′}, C(O)Rₐ, —N(Rₐ)C(O)Rₐ, —NRₐC(O)ORₐ, —NRₐSO₂Rₐ, —C(O)ORₐ, —C(O)NRₐR_{a′}, —S(O)2NRₐR_{a′}, —S(O)Rₐ, —S(O)₂Rₐ, —P(O)RₐR_{a′}.

In the preferred technical solution of the present invention, wherein R₂ represents NR^{L}R^{L′}, wherein R^{L} represents hydrogen or C₁-C₆ alkyl; R^{L′} represents C₁-C₆ alkyl, (C₃-C₆) cycloalkyl, (C₀-C₆ alkylene) (C₆-C₁₀) aryl, —(C₀-C₆ alkylene) (5-10) membered heteroaryl, wherein said R^{L} and R^{L′} can be independently optionally substituted by 0, 1, 2 substituents selected from halogen, hydroxyl, C₁-C₆ alkyl, halo (C₁-C₆) alkyl, ORₐ, cyano.

In the preferred technical solution of the present invention, wherein R₂ represents NR^{L}R^{L′}, wherein said R^{L}, R^{L′} form a 4-8 membered ring together with the nitrogen atom connected to it, and the ring may additionally contain 0, 1, 2 heteroatoms selected from nitrogen, oxygen, sulfur;
wherein the ring can also be optionally fused to another 5-6 membered carbocycle, 5-6 membered cycloheteroalkane, 5-6 membered aromatic heterocycle or benzene ring to form a fused ring bicyclic system;
or the ring can also be connected to another (4-6 membered) ring carbocycle or (4-6 membered) heterocycle through a spiro carbon atom to form a spirobicyclic ring system;
wherein the fused ring bicyclic system or spirocyclic bicyclic system can be optionally substitute by 0, 1, 2, 3 members selected from halogen, cyano, (C₁-C₆) alkyl, (C₂-C₆) alkenyl, oxo, —NRₐR_{a′}, —ORₐ, —SRₐ, —C(O)Rₐ, —N(Rₐ)C(O)Rₐ, —N(Rₐ)C(O)ORₐ, —N(Rₐ)SO₂Rₐ, C(O)ORₐ, —C(O)NRₐR_{a′}, —S(O)₂NRₐR_{a′}, —S(O)Rₐ, —S(O)₂Rₐ.

In the preferred technical solution of the present invention, wherein R₂ represents NR^{L}R^{L′}, wherein R^{L} represents hydrogen or C₁-C₆ alkyl; R^{L′} represents —(C₀-C₆ alkylene)-(CR^{M}R^{M′})—(C₀-C₆) alkyl, —(C₀-C₆ alkylene)-(CR^{M}R^{M′})—(C₀-C₆) alkyl, —(C₀-C₆ alkylene)-(CR^{M}R^{M′})-halogen, wherein R^{M} and R^{M′} independently represent hydrogen, C₁-C₆ alkyl;
alternatively, R^{M} and R^{M′} form a 3-8 membered ring together with the carbon atom connected to it, and the ring can also optionally contain 0, 1, 2 heteroatoms selected from N, O, S or oxo, —NRₐ group.

In the preferred technical solution of the present invention, wherein, R₁ represents hydrogen, C₁-C₆ alkyl, halogen, ORₐ, NRₐR_{a′}, cyano, —SO₂Rₐ, halogenated (C₁-C₆) alkyl, (C₃-C₆) cycloalkyl; preferably hydrogen, C₁-C₆ alkyl, halogen, halo(C₁-C₆)alkyl; more preferably hydrogen, C₁-C₆ alkyl.

In the preferred technical solution of the present invention, wherein X₂ represents CR₇, wherein R₇ represents hydrogen, halogen, hydroxyl, cyano, (C₁-C₆) alkyl, (C₃-C₆) cycloalkyl, halogenated (C₁-C₆)alkyl.

In addition, the present invention also provides a compound with the structure of the following formula (III) or a pharmaceutically acceptable salt, isotope derivative, stereoisomer

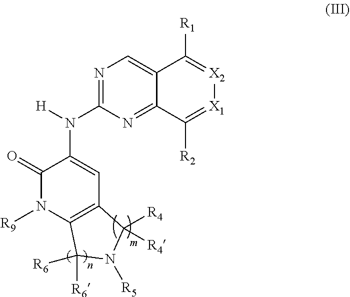

wherein R1 represents hydrogen, halogen, hydroxyl, (C₁-C₆) alkyl, (C₂-C₆) alkenyl, (C₁-C₆) alkoxy;
wherein R₂ represents hydrogen, halogen, hydroxyl, (C₁-C₆) alkyl, (C₂-C₆) alkenyl, —(C₀-C₆ alkylene) (C₁-C₆) alkoxy, —(C₀-C₆) alkylene (C6-C10) aryl, —(C0-C6 alkylene) (5-10) membered heteroaryl, —(C₀-C₆ alkylene) (4-10 membered) heterocycloalkyl, —(C₀-C₆ alkylene) (C₃-C₈) cycloalkyl, —NR^{L}R^{L′}, —OR^{L}, —SR^{L};
   wherein R₃ represents hydrogen, halogen, hydroxyl, (C₁-C₆) alkyl, (C₂-C₆) alkenyl, halogenated (C₁-C₆) alkyl, —(C₀-C₆ alkylene) (C₃-C₈) cycloalkyl, —(C₀-C₆ alkylene) (4-8 members) heterocycloalkyl, (C₁-C₆) alkoxy, —(C₀-C₆ alkylene) (C₃-C₈) cycloalkyloxy, —(C₀-C₆ alkylene) (4-8 membered) heterocycloalkyloxy;
wherein R₄ and R_{4′} independently represent hydrogen, (C₁-C₆) alkyl, (C₂-C₆) alkenyl, halogen;
   alternatively, R₄ and R_{4′} form a 3-6 membered ring together with the carbon atom connected to it, and the ring can also optionally contain 0, 1, 2 heteroatoms selected from N, O, and S;
   wherein R₅ represents hydrogen, C₁-C₆ alkyl, (C₃-C₆) alkenyl, (C₃-C₈) cycloalkyl, (4-8 membered) heterocycloalkyl;
   wherein R₆ and R_{6′} independently represent hydrogen, C₁-C₆ alkyl, (C₂-C₆) alkenyl, halogen;
   alternatively, R₆ and R_{6′} form a 3-6 membered ring together with the carbon atom connected to it, and the ring can also optionally contain 0, 1, 2 heteroatoms selected from N, O, and S;
   wherein X₁ represents N or CH;
   wherein X₂ represents N or CR₇;
   wherein R₇ represents hydrogen, halogen, (C₁-C₆) alkyl, (C₂-C₆) alkenyl, (C₃-C₈) cycloalkyl, (C₁-C₆) alkoxy;
   wherein R represents hydrogen, (C₁-C₆) alkyl, (C₃-C₆) alkenyl, —(C₀-C₆ alkylene) (C₃-C₈) cycloalkyl, —(C₀-C₆ alkylene) (4-10 membered) heterocycloalkyl, —(C₀-C₆ alkylene) (C₆-C₁₀) aryl, —(C₀-C₆ alkylene) (5-10) membered heteroaryl;
   wherein R^{L} and R^{L′} independently represent hydrogen, (C₁-C₆) alkyl, (C₃-C₆) alkenyl, (C₃-C₆) cycloalkyl, (C₀-C₆ alkylene) (C₆-C₁₀) aryl, —(C₀-C₆ alkylene) (5-10) membered heteroaryl, —(C₀-C₆) alkylene-(CR^{M}R^{M′})—(C₀-C₆) alkyl, —(C₀-C₆) alkylene-(CR^{M}R^{M′})-halogen;
   alternatively, R^{L} and R^{L′} form a 4-8 membered ring together with the nitrogen atom connected to it, and the ring may additionally contain 0, 1, 2 heteroatoms selected from nitrogen, oxygen, sulfur or oxo, —NRₐ groups and this ring may also optionally be fused to another 5-6 membered carbocycle, 5-6 cycloheteroalkane, 5-6 membered aromatic heterocycle or benzene ring to form a fused ring bicyclic system;
   or the ring can also be connected to another (4-6 membered) ring carbocycle or (4-6 membered) heterocycle through a spiro carbon atom to form a spirobicyclic ring system;
   wherein R^{M} and R^{M′} independently represent hydrogen, C₁-C₆ alkyl;
   alternatively, R^{M} and R^{M′} form a 3-8 membered ring together with the carbon atom connected to it, and the ring can also optionally contain 0, 1, 2 heteroatoms selected from N, O, S or oxo, —NRₐ group;
   For the above-mentioned defined alkyl, ring, cycloalkyl, heterocycloalkyl, aryl, heteroaryl, it can be optionally substituted by 0, 1, 2, 3 substituents selected from the following: (C₁-C₆) alkyl, (C₂-C₆) alkenyl, halo (C₁-C₆) alkyl, halo (C₁-C₆) alkoxy, (C₃-C₈) cycloalkyl, —(C₁-C₆ alkylene) —O—(C₁-C₆) alkyl, halogenated (C₃-C₈) cycloalkyl, halogen, —CN, oxo, —NRₐR_{a′}, —ORₐ, —SRₐ, —(C₁-C₆ alkyl) hydroxy, —C(O)Rₐ, —N(Rₐ)C(O)Rₐ, —NRₐC(O)ORₐ, —NRₐSO₂Rₐ, —C(O)ORₐ, —C(O)NRₐR_{a′}, —S(O)₂NRₐR_{a′}, —S(O)Rₐ, —S(O)₂Rₐ, —P(O)RₐR_{a′};
wherein Rₐ and R_{a′} each independently represent hydrogen, (C₁-C₆) alkyl, (C₂-C₆) alkenyl, (C₃-C₈) cycloalkyl, or Rₐ and R_{a′} can form 4-7 membered cycloheteroalkanes together with the N atom connected to it;
wherein m and n represent 0, 1, 2, 3.

In addition, the present invention also provides a compound with the following formula (IV) or a pharmaceutically acceptable salt, isotope derivative, stereoisomer

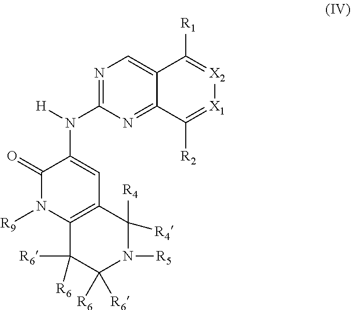

wherein R₁, R₂, R₃, R₄, R_{4′}, R₅, R_{5′}, R₆, R_{6′}, R₉, X₁, X₂ have the definition as formula (I).

In the above preferred technical solution, wherein R₂ represents (C₁-C₆) alkyl, —(C₀-C₆ alkylene) (C₆-C₁₀) aryl, —(C₀-C₆ alkylene) (5-10) membered heteroaryl, —(C₀-C₆ alkylene) (4-10 membered) heterocycloalkyl, —(C₀-C₆ alkylene) (C₃-C₈) cycloalkyl; wherein the alkyl, aryl, heteroaryl, cycloalkyl, heterocycloalkyl can be optionally substituted by 0, 1, 2 members selected from halogen, C₁-C₆ alkyl, —ORₐ, —SRₐ, halogenated (C₁-C₆) alkyl, halogenated (C₁-C₆) alkoxy, —(C₆-C₆ alkylene)-O—(C₁-C₆) alkyl, C₃-C₆ cycloalkyl, —NRₐR_{a′}, C(O)Rₐ, —N(Rₐ)C(O)Rₐ, —NRₐC(O)ORₐ, —NRₐSO₂Rₐ, —C(O)ORₐ, —C(O)NRₐR_{a′}, —S(O)₂NRₐR_{a′}, —S(O)Rₐ, —S(O)₂Rₐ, —P(O)RₐR_{a′}.

In the above preferred technical solution, wherein R₂ represents NR^{L}R^{L′}, wherein R^{L} represents hydrogen or C₁-C₆ alkyl; R^{L′} represents C₁-C₆ alkyl, (C₃-C₆) cycloalkyl, (C₀-C₆ alkylene) (C₆-C₁₀) aryl, —(C₀-C₆ alkylene) (5-10) membered heteroaryl, wherein, the R^{L} and R^{L′} can be independently and optionally substituted by 0, 1, 2 substituents selected from halogen, hydroxyl, C₁-C₆ alkyl, halo(C₁-C₆) alkyl, ORₐ, cyano, wherein Rₐ represents hydrogen, (C₁-C₆) alkyl.

In the above preferred technical solution, wherein R₂ represents NR^{L}R^{L′}, wherein said R^{L}, R^{L′} form a 4-8 membered ring together with the nitrogen atom connected to it, and the ring may additionally contain 0, 1, 2 heteroatom selected from nitrogen, oxygen, sulfur or oxo, —NRₐ group;
wherein the ring can also be optionally fused to another 5-6 membered carbocycle, 5-6 membered cycloheteroalkane, 5-6 membered aromatic heterocycle or benzene ring to form a fused ring bicyclic system;
or the ring can also be connected to another (4-6 membered) ring carbocycle or (4-6 membered) heterocycle through a spiro carbon atom to form a spirobicyclic ring system;
wherein said ring can be optionally substituted by 0, 1, 2, 3 members selected from halogen, cyano, (C₁-C₆) alkyl, oxo, —NRₐR_{a′}, —ORₐ, —SRₐ, —C(O)Rₐ, —N(Rₐ)C(O)Rₐ, —N(Rₐ)C(O)ORₐ, —N(Rₐ)SO₂Rₐ, —C(O)ORₐ, —C(O)NRₐR_{a′}, S(O)₂NRₐR_{a′}, —S(O)Rₐ, and —S(O)₂Rₐ, wherein Rₐ and R_{a′} each independently represent hydrogen or (C₁-C₆) alkyl.

In the above preferred technical solution, wherein R₂ represents —(C₀-C₆ alkylene) (C₆-C₁₀) aryl, —(C₀-C₆ alkylene) (5-10) membered heteroaryl, —(C₀-C₆ alkylene) (4-10 membered) heterocycloalkyl, —(C₀-C₆ alkylene) (C₃-C₈) cycloalkyl, wherein said R₂ can be optionally substituted by 0, 1, 2 members selected from halogen, C₁-C₆ alkyl, —ORₐ, —SRₐ, —(C₁-C₆ alkylene) hydroxyl, halogenated (C₁-C₆) alkyl, halogenated (C₁-C₆) alkoxy, —(C₁-C₆alkylene) —O—(C₁-C₆) alkyl, C₃-C₆ cycloalkyl, —NRₐR_{a′}, —C(O)Rₐ, —N(Rₐ)C(O)Rₐ, —NRₐC(O)ORₐ, —NRₐSO₂Rₐ, —C(O)ORₐ, —C(O)NRₐR_{a′}, —S(O)₂NRₐR_{a′}, —S(O)Rₐ, —S(O)₂Rₐ, —P(O)RₐR_{a′}, wherein Rₐ and R_{a′} each independently represent hydrogen, (C1-C6) alkyl.

In the above preferred technical solution, wherein R₂ represents halogen, C₁-C₆ alkyl, —ORₐ, —C(O)ORₐ, —C(O)NRₐR_{a′}, —(C₁-C₆ alkylene) hydroxyl, halogenated (C₁-C₆) alkoxy substituted (C₆-C₁₀) aryl, (5-10) membered heteroaryl, wherein, Rₐ, R_{a′} each independently represents hydrogen, (C₁-C₆) alkyl.

In the above-mentioned preferred technical solution, wherein, R₂ represents phenyl, pyridyl, pyrazolyl,

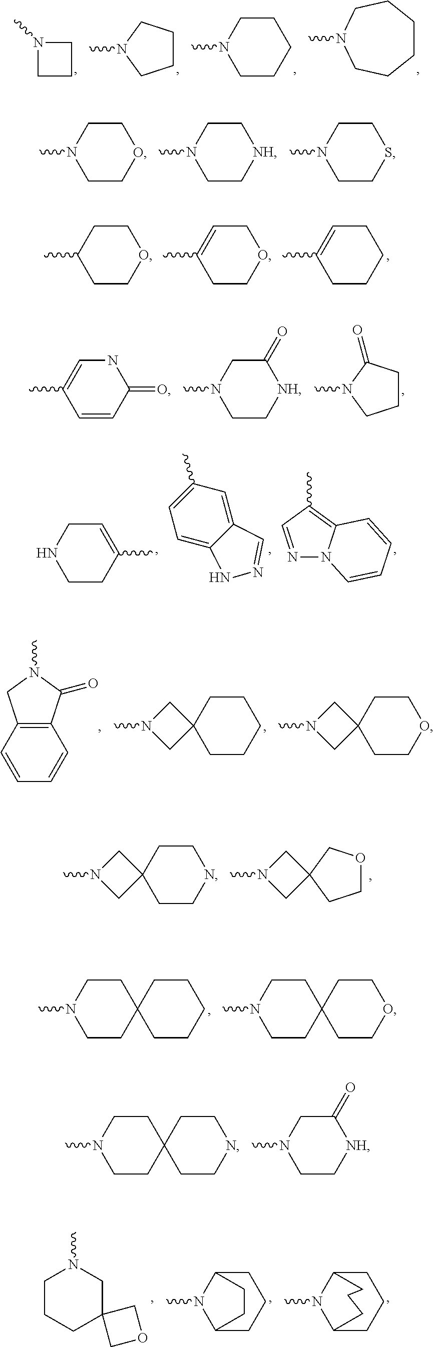

where the dotted line indicates the Junction site,
wherein the R₂ can be optionally substituted by members selected from halogen, C₁-C₆ alkyl, ORₐ, SRₐ, C₁-C₆ alkylene hydroxyl, —(C₁-C₆ alkylene)-O—(C₁-C₆) alkyl, —C(O)Rₐ, —C(O)ORₐ, —C(O)NRₐR_{a′}, —S(O)2NRₐR_{a′}, —S(O)Rₐ, halogenated (C1-C6) alkyl, halogenated (C1-C6)alkoxy.

In the above preferred technical solution, wherein R₂ represents NR^{L}R^{L′}, wherein R^{L} represents hydrogen or C₁-C₆ alkyl; R^{L′} represents —(C₀-C₆ alkylene)-(CR^{M}R^{M′})—(C₀-C₆) alkyl, —(C₀-C₆ alkylene)-(CR^{M}R^{M′})—(C₀-C₆) alkyl, —(C₀-C₆ alkylene) —(CR^{M}R^{M′})-halogen, wherein R^{M} and R^{M′} each independently represent hydrogen, C₁-C₆ alkyl;
alternatively, R^{M} and R^{M′} form a 3-8 membered ring together with the carbon atom connected to it, and the ring can also optionally contain 0, 1, 2 heteroatoms selected from N, O, S or oxo, —NRₐ group.

In the above preferred technical solution, wherein, R¹ represents hydrogen, C₁-C₆ alkyl, halogen, ORₐ, NRₐR_{a′}, cyano, —SO₂Rₐ, halogenated (C₁-C₆) alkyl, (C₃-C₆) cycloalkyl, wherein, Rₐ, R_{a′} each independently represent hydrogen, (C₁-C₆) alkyl; preferably hydrogen, C₁-C₆ alkyl, halogen, halogenated (C₁-C₆) alkyl; more preferably hydrogen, C₁-C₆ alkyl.

In the above preferred technical solution, wherein, X₂ represents CR₇, wherein R₇ represents hydrogen, halogen, hydroxyl, cyano, (C₁-C₆) alkyl, (C₃-C₆) cycloalkyl, halogenated (C₁-C₆) alkyl.

Specifically, the present invention provides compounds with the following structures:

| serial | |
|---|---|
| number | Compound structure |
| 1 | 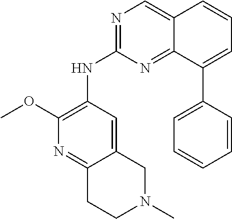 [SMILES: COc1nc2c(cc1Nc1ncc3cccc(-c4ccccc4)c3n1)CN(C)CC2] |
| 2 | 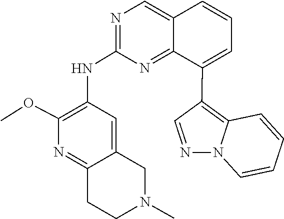 [SMILES: COc1nc2c(cc1Nc1ncc3cccc(-c4cnn5ccccc45)c3n1)CN(C)CC2] |
| 3 | 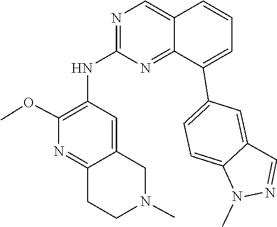 [SMILES: COc1nc2c(cc1Nc1ncc3cccc(-c4ccc5c(cnn5C)c4)c3n1)CN(C)CC2] |
| 4 | 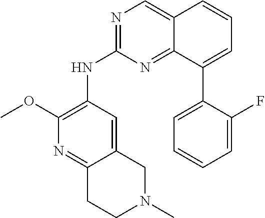 [SMILES: COc1nc2c(cc1Nc1ncc3cccc(-c4ccccc4F)c3n1)CN(C)CC2] |
| 5 | 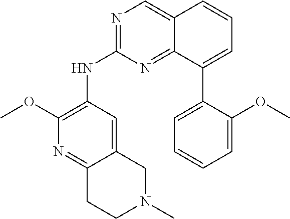 |
| 6 | 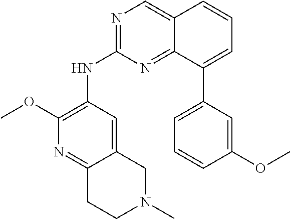 |
| 7 | 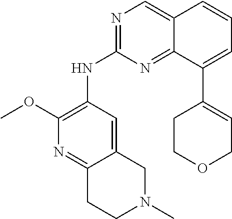 [SMILES: COc1nc2c(cc1Nc1ncc3cccc(C4=CCOCC4)c3n1)CN(C)CC2] |
| 8 | 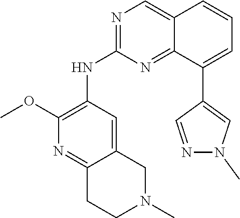 |
| 9 | 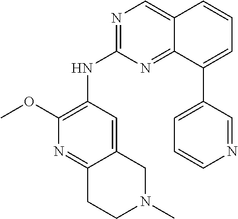 [SMILES: COc1nc2c(cc1Nc1ncc3cccc(-c4cccnc4)c3n1)CN(C)CC2] |
| 10 | 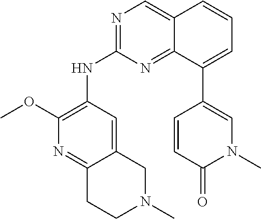 |
| 11 | 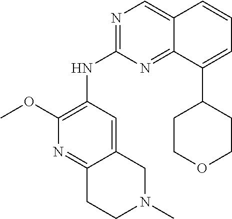 [SMILES: COc1nc2c(cc1Nc1ncc3cccc(C4CCOCC4)c3n1)CN(C)CC2] |
| 12 | 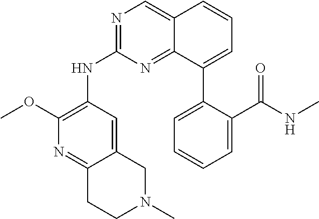 |
| 13 | 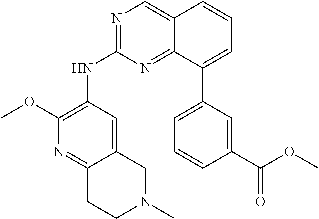 |
| 14 | 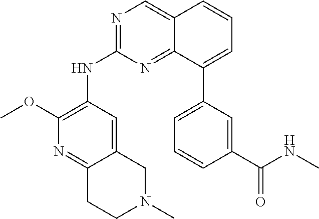 |
| 15 | 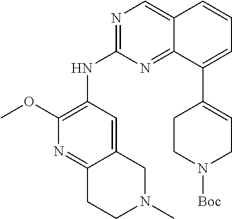 [SMILES: COc1nc2c(cc1Nc1ncc3cccc(C4=CCN(C(=O)OC(C)(C)C)CC4)c3n1)CN(C)CC2] |
| 16 | 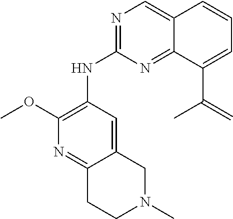 [SMILES: C=C(C)c1cccc2cnc(Nc3cc4c(nc3OC)CCN(C)C4)nc12] |
| 17 | 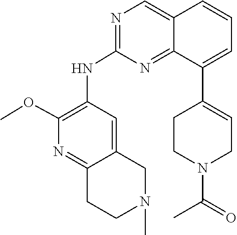 [SMILES: COc1nc2c(cc1Nc1ncc3cccc(C4=CCN(C(C)=O)CC4)c3n1)CN(C)CC2] |
| 18 | 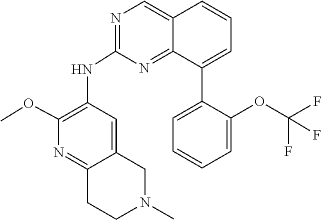 [SMILES: COc1nc2c(cc1Nc1ncc3cccc(-c4ccccc4OC(F)(F)F)c3n1)CN(C)CC2] |
| 19 | 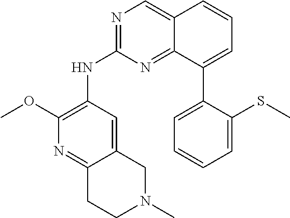 |
| 20 | 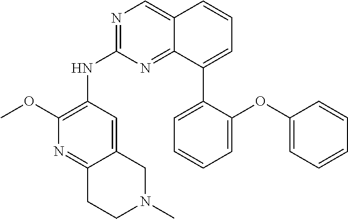 [SMILES: COc1nc2c(cc1Nc1ncc3cccc(-c4ccccc4Oc4ccccc4)c3n1)CN(C)CC2] |
| 21 | 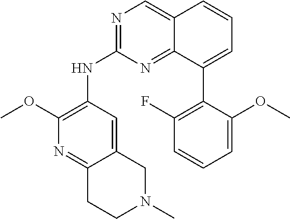 |
| 22 | 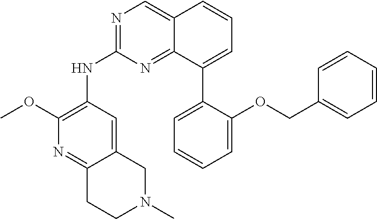 [SMILES: COc1nc2c(cc1Nc1ncc3cccc(-c4ccccc4OCc4ccccc4)c3n1)CN(C)CC2] |
| 23 | 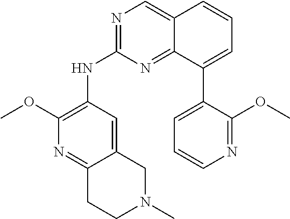 |
| 24 | 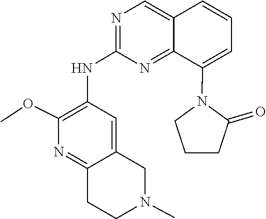 [SMILES: COc1nc2c(cc1Nc1ncc3cccc(N4CCCC4=O)c3n1)CN(C)CC2] |
| 25 | 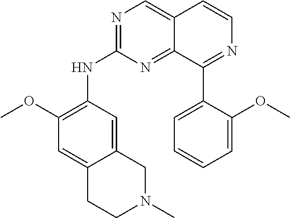 |
| 26 | 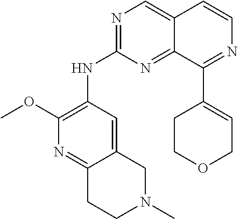 [SMILES: COc1nc2c(cc1Nc1ncc3ccnc(C4=CCOCC4)c3n1)CN(C)CC2] |
| 27 | 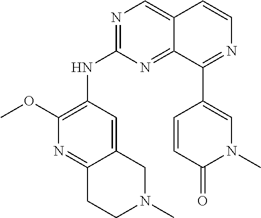 |
| 28 | 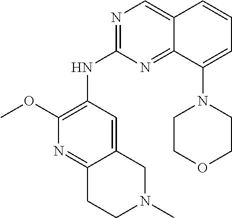 [SMILES: COc1nc2c(cc1Nc1ncc3cccc(N4CCOCC4)c3n1)CN(C)CC2] |
| 29 | 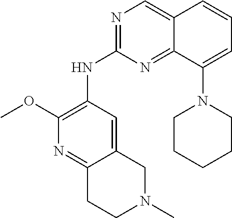 [SMILES: COc1nc2c(cc1Nc1ncc3cccc(N4CCCCC4)c3n1)CN(C)CC2] |
| 30 | 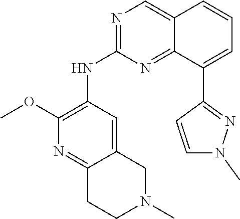 |
| 31 | 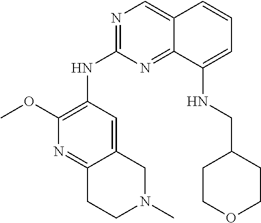 [SMILES: COc1nc2c(cc1Nc1ncc3cccc(NCC4CCOCC4)c3n1)CN(C)CC2] |
| 32 | 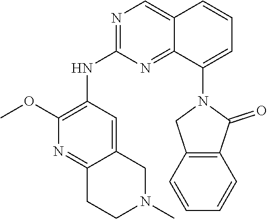 [SMILES: COc1nc2c(cc1Nc1ncc3cccc(N4Cc5ccccc5C4=O)c3n1)CN(C)CC2] |
| 33 | 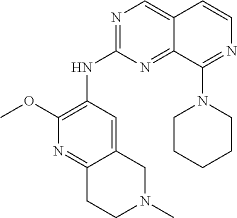 [SMILES: COc1nc2c(cc1Nc1ncc3ccnc(N4CCCCC4)c3n1)CN(C)CC2] |
| 34 | 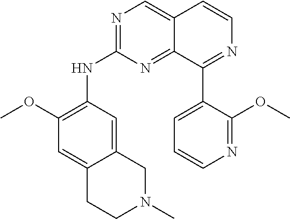 |
| 35 | 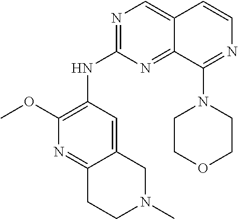 [SMILES: COc1nc2c(cc1Nc1ncc3ccnc(N4CCOCC4)c3n1)CN(C)CC2] |
| 36 | 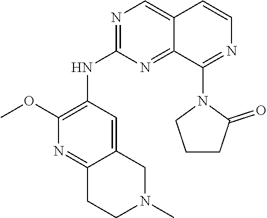 [SMILES: COc1nc2c(cc1Nc1ncc3ccnc(N4CCCC4=O)c3n1)CN(C)CC2] |
| 37 | 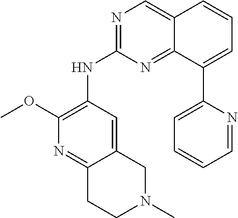 [SMILES: COc1nc2c(cc1Nc1ncc3cccc(-c4ccccn4)c3n1)CN(C)CC2] |
| 38 | 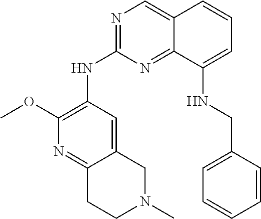 [SMILES: COc1nc2c(cc1Nc1ncc3cccc(NCc4ccccc4)c3n1)CN(C)CC2] |
| 39 | 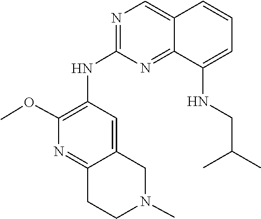 [SMILES: COc1nc2c(cc1Nc1ncc3cccc(NCC(C)C)c3n1)CN(C)CC2] |
| 40 | 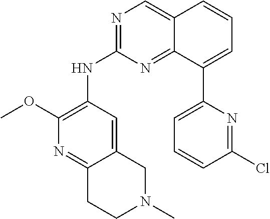 [SMILES: COc1nc2c(cc1Nc1ncc3cccc(-c4cccc(Cl)n4)c3n1)CN(C)CC2] |
| 41 | 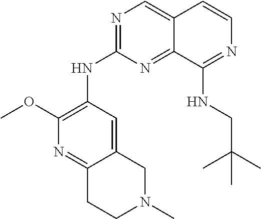 [SMILES: COc1nc2c(cc1Nc1ncc3ccnc(NCC(C)(C)C)c3n1)CN(C)CC2] |
| 42 | 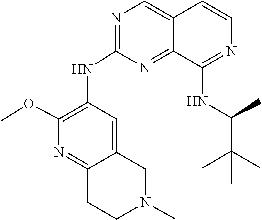 [SMILES: COc1nc2c(cc1Nc1ncc3ccnc(N[C@@H](C)C(C)(C)C)c3n1)CN(C)CC2] |
| 43 | 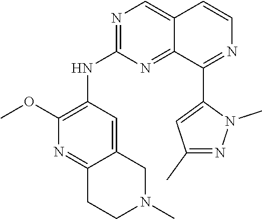 |
| 44 | 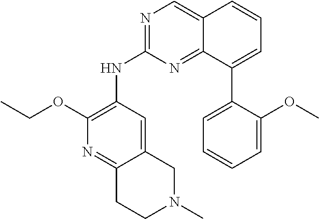 [SMILES: CCOc1nc2c(cc1Nc1ncc3cccc(-c4ccccc4OC)c3n1)CN(C)CC2] |
| 45 | 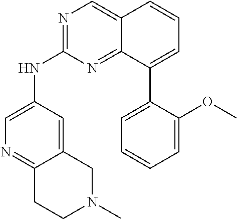 [SMILES: COc1ccccc1-c1cccc2cnc(Nc3cnc4c(c3)CN(C)CC4)nc12] |
| 46 | 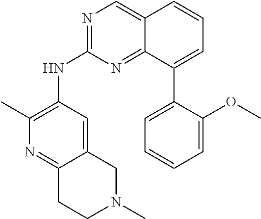 |
| 47 | 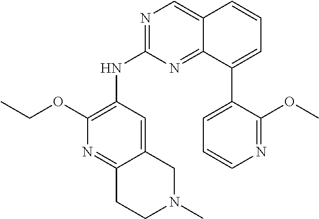 [SMILES: CCOc1nc2c(cc1Nc1ncc3cccc(-c4cccnc4OC)c3n1)CN(C)CC2] |
| 48 | 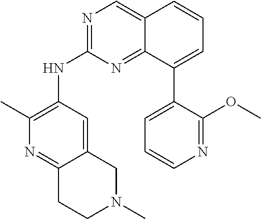 |
| 49 | 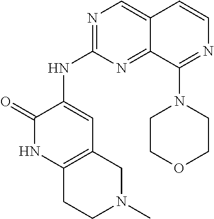 [SMILES: CN1CCc2[nH]c(=O)c(Nc3ncc4ccnc(N5CCOCC5)c4n3)cc2C1] |
| 50 | 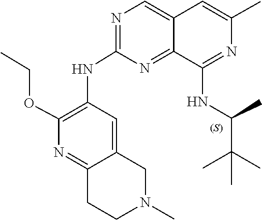 |
| 51 | 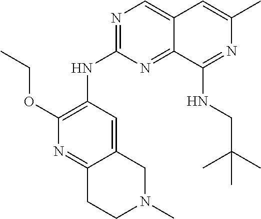 [SMILES: CCOc1nc2c(cc1Nc1ncc3cc(C)nc(NCC(C)(C)C)c3n1)CN(C)CC2] |
| 52 | 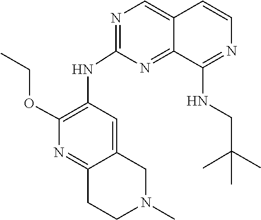 [SMILES: CCOc1nc2c(cc1Nc1ncc3ccnc(NCC(C)(C)C)c3n1)CN(C)CC2] |
| 53 | 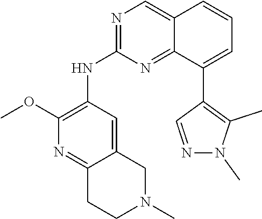 |
| 54 | 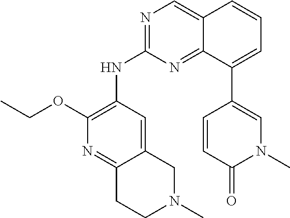 [SMILES: CCOc1nc2c(cc1Nc1ncc3cccc(-c4ccc(=O)n(C)c4)c3n1)CN(C)CC2] |
| 55 | 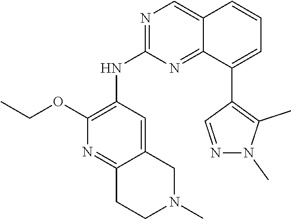 [SMILES: CCOc1nc2c(cc1Nc1ncc3cccc(-c4cnn(C)c4C)c3n1)CN(C)CC2] |
| 56 | 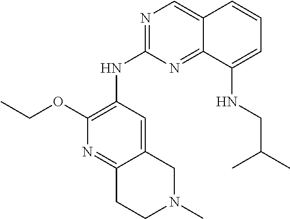 [SMILES: CCOc1nc2c(cc1Nc1ncc3cccc(NCC(C)C)c3n1)CN(C)CC2] |
| 57 | 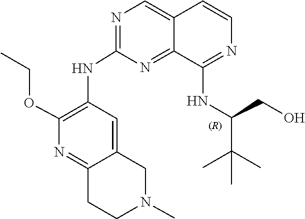 |
| 58 | 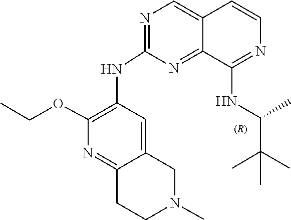 |
| 59 | 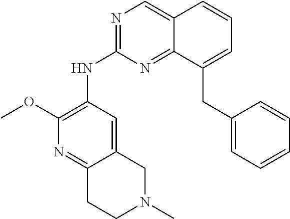 [SMILES: COc1nc2c(cc1Nc1ncc3cccc(Cc4ccccc4)c3n1)CN(C)CC2] |
| 60 | 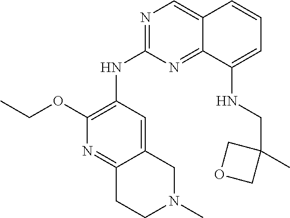 [SMILES: CCOc1nc2c(cc1Nc1ncc3cccc(NCC4(C)COC4)c3n1)CN(C)CC2] |
| 61 | 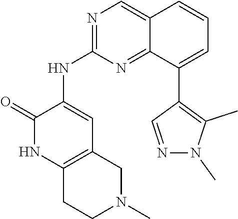 [SMILES: Cc1c(-c2cccc3cnc(Nc4cc5c([nH]c4=O)CCN(C)C5)nc23)cnn1C] |
| 62 | 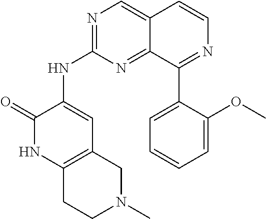 [SMILES: COc1ccccc1-c1nccc2cnc(Nc3cc4c([nH]c3=O)CCN(C)C4)nc12] |
| 63 | 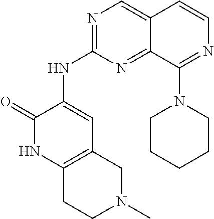 [SMILES: CN1CCc2[nH]c(=O)c(Nc3ncc4ccnc(N5CCCCC5)c4n3)cc2C1] |
| 64 | 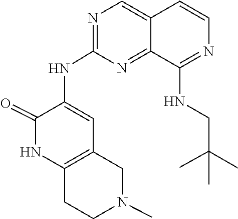 [SMILES: CN1CCc2[nH]c(=O)c(Nc3ncc4ccnc(NCC(C)(C)C)c4n3)cc2C1] |
| 65 | 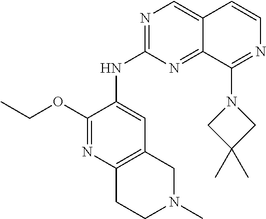 [SMILES: CCOc1nc2c(cc1Nc1ncc3ccnc(N4CC(C)(C)C4)c3n1)CN(C)CC2] |
| 66 | 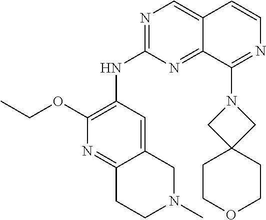 [SMILES: CCOc1nc2c(cc1Nc1ncc3ccnc(N4CC5(CCOCC5)C4)c3n1)CN(C)CC2] |
| 67 | 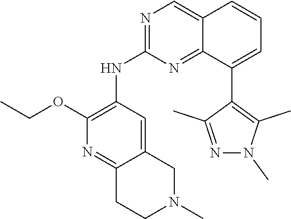 [SMILES: CCOc1nc2c(cc1Nc1ncc3cccc(-c4c(C)nn(C)c4C)c3n1)CN(C)CC2] |
| 68 | 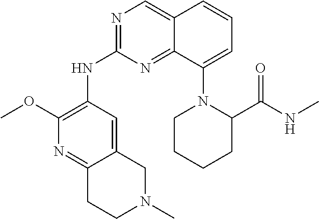 |
| 69 | 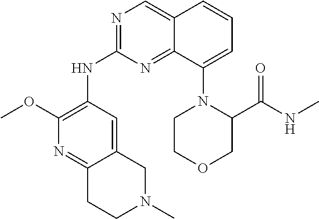 |
| 70 | 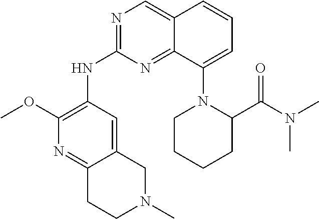 |
| 71 | 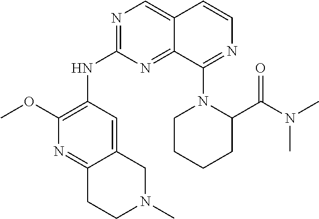 |
| 72 | 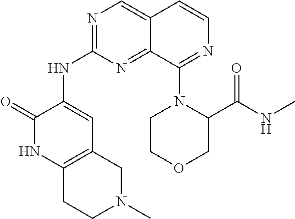 [SMILES: CNC(=O)C1COCCN1c1nccc2cnc(Nc3cc4c([nH]c3=O)CCN(C)C4)nc12] |
| 73 | 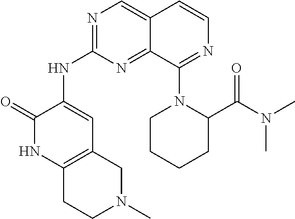 [SMILES: CN1CCc2[nH]c(=O)c(Nc3ncc4ccnc(N5CCCCC5C(=O)N(C)C)c4n3)cc2C1] |
| 74 | 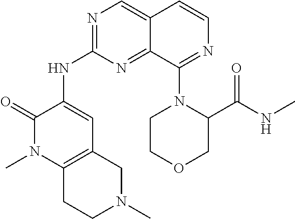 [SMILES: CNC(=O)C1COCCN1c1nccc2cnc(Nc3cc4c(n(C)c3=O)CCN(C)C4)nc12] |
| 75 | 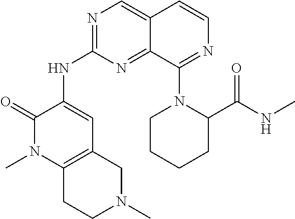 [SMILES: CNC(=O)C1CCCCN1c1nccc2cnc(Nc3cc4c(n(C)c3=O)CCN(C)C4)nc12] |
| 76 | 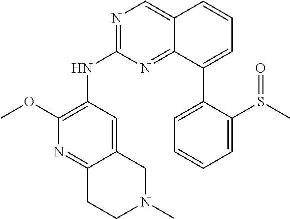 |
| 77 | 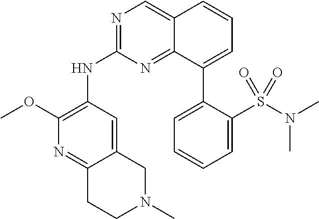 |
| 78 | 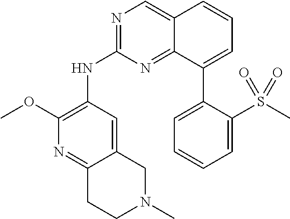 |
| 79 | 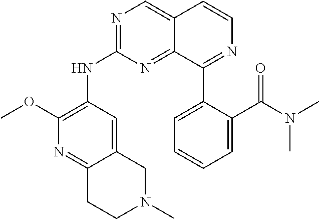 |
| 80 | 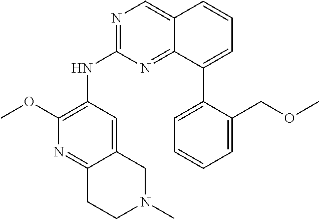 |
| 81 | 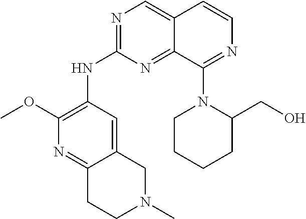 [SMILES: COc1nc2c(cc1Nc1ncc3ccnc(N4CCCCC4CO)c3n1)CN(C)CC2] |
| 82 | 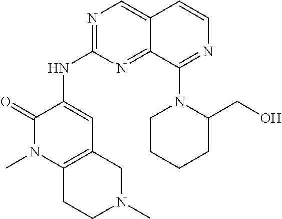 [SMILES: CN1CCc2c(cc(Nc3ncc4ccnc(N5CCCCC5CO)c4n3)c(=O)n2C)C1] |
| 83 | 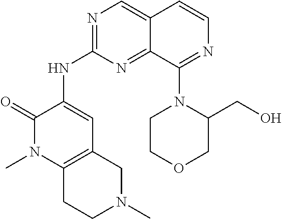 [SMILES: CN1CCc2c(cc(Nc3ncc4ccnc(N5CCOCC5CO)c4n3)c(=O)n2C)C1] |
| 84 | 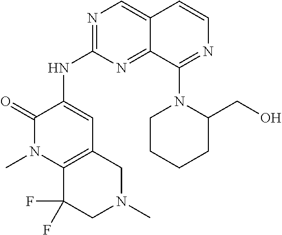 [SMILES: CN1Cc2cc(Nc3ncc4ccnc(N5CCCCC5CO)c4n3)c(=O)n(C)c2C(F)(F)C1] |
| 85 | 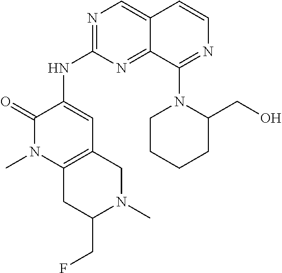 [SMILES: CN1Cc2cc(Nc3ncc4ccnc(N5CCCCC5CO)c4n3)c(=O)n(C)c2CC1CF] |
| 86 | 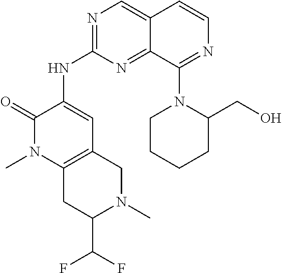 [SMILES: CN1Cc2cc(Nc3ncc4ccnc(N5CCCCC5CO)c4n3)c(=O)n(C)c2CC1C(F)F] |
| 87 | 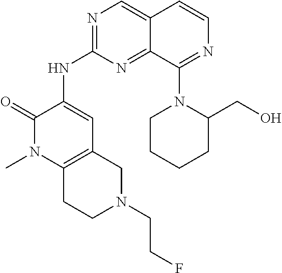 [SMILES: Cn1c2c(cc(Nc3ncc4ccnc(N5CCCCC5CO)c4n3)c1=O)CN(CCF)CC2] |
| 88 | 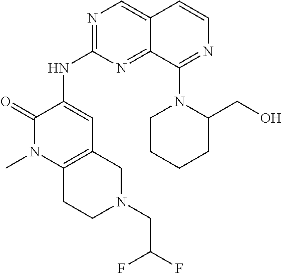 [SMILES: Cn1c2c(cc(Nc3ncc4ccnc(N5CCCCC5CO)c4n3)c1=O)CN(CC(F)F)CC2] |
| 89 | 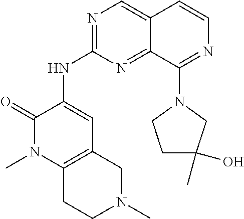 [SMILES: CN1CCc2c(cc(Nc3ncc4ccnc(N5CCC(C)(O)C5)c4n3)c(=O)n2C)C1] |
| 90 | 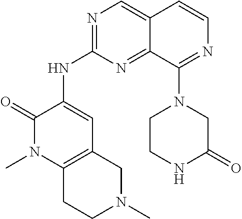 [SMILES: CN1CCc2c(cc(Nc3ncc4ccnc(N5CCNC(=O)C5)c4n3)c(=O)n2C)C1] |
| 91 | 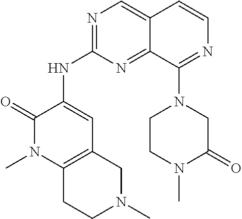 [SMILES: CN1CCc2c(cc(Nc3ncc4ccnc(N5CCN(C)C(=O)C5)c4n3)c(=O)n2C)C1] |
| 92 | 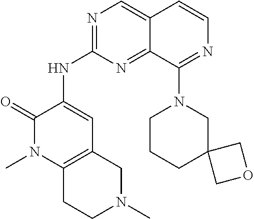 [SMILES: CN1CCc2c(cc(Nc3ncc4ccnc(N5CCCC6(COC6)C5)c4n3)c(=O)n2C)C1] |
| 93 | 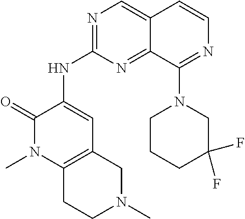 [SMILES: CN1CCc2c(cc(Nc3ncc4ccnc(N5CCCC(F)(F)C5)c4n3)c(=O)n2C)C1] |
| 94 | 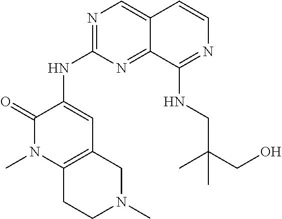 [SMILES: CN1CCc2c(cc(Nc3ncc4ccnc(NCC(C)(C)CO)c4n3)c(=O)n2C)C1] |
| 95 | 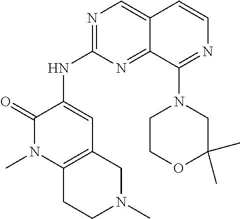 [SMILES: CN1CCc2c(cc(Nc3ncc4ccnc(N5CCOC(C)(C)C5)c4n3)c(=O)n2C)C1] |
| 96 | 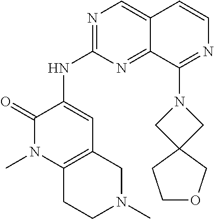 [SMILES: CN1CCc2c(cc(Nc3ncc4ccnc(N5CC6(CCOC6)C5)c4n3)c(=O)n2C)C1] |
| 97 | 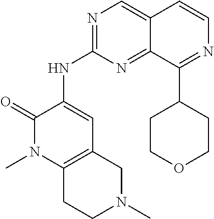 [SMILES: CN1CCc2c(cc(Nc3ncc4ccnc(C5CCOCC5)c4n3)c(=O)n2C)C1] |
| 98 | 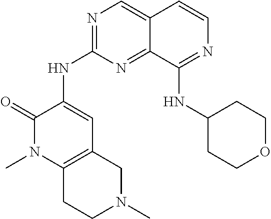 [SMILES: CN1CCc2c(cc(Nc3ncc4ccnc(NC5CCOCC5)c4n3)c(=O)n2C)C1] |
| 99 | 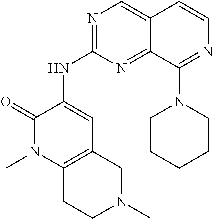 [SMILES: CN1CCc2c(cc(Nc3ncc4ccnc(N5CCCCC5)c4n3)c(=O)n2C)C1] |
| 100 | 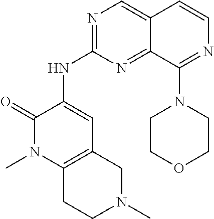 [SMILES: CN1CCc2c(cc(Nc3ncc4ccnc(N5CCOCC5)c4n3)c(=O)n2C)C1] |
| 101 | 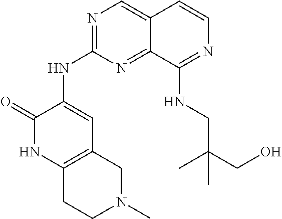 [SMILES: CN1CCc2[nH]c(=O)c(Nc3ncc4ccnc(NCC(C)(C)CO)c4n3)cc2C1] |
| 102 | 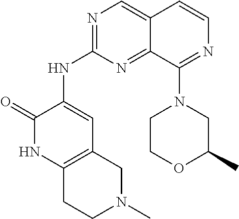 [SMILES: C[C@@H]1CN(c2nccc3cnc(Nc4cc5c([nH]c4=O)CCN(C)C5)nc23)CCO1] |
| 103 | 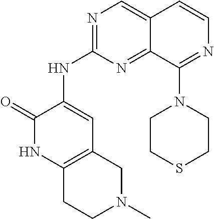 [SMILES: CN1CCc2[nH]c(=O)c(Nc3ncc4ccnc(N5CCSCC5)c4n3)cc2C1] |
| 104 | 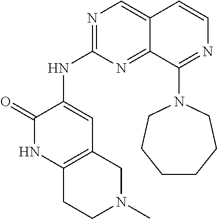 [SMILES: CN1CCc2[nH]c(=O)c(Nc3ncc4ccnc(N5CCCCCC5)c4n3)cc2C1] |
| 105 | 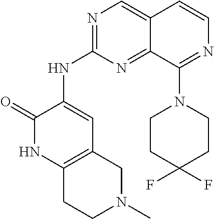 [SMILES: CN1CCc2[nH]c(=O)c(Nc3ncc4ccnc(N5CCC(F)(F)CC5)c4n3)cc2C1] |
| 106 | 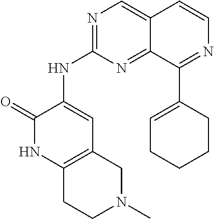 [SMILES: CN1CCc2[nH]c(=O)c(Nc3ncc4ccnc(C5=CCCCC5)c4n3)cc2C1] |
| 107 | 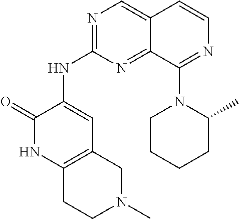 [SMILES: C[C@@H]1CCCCN1c1nccc2cnc(Nc3cc4c([nH]c3=O)CCN(C)C4)nc12] |
| 108 | 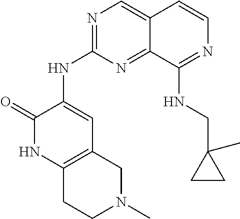 [SMILES: CN1CCc2[nH]c(=O)c(Nc3ncc4ccnc(NCC5(C)CC5)c4n3)cc2C1] |
| 109 | 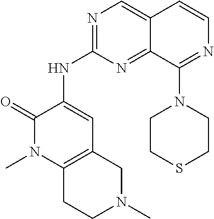 [SMILES: CN1CCc2c(cc(Nc3ncc4ccnc(N5CCSCC5)c4n3)c(=O)n2C)C1] |
| 110 | 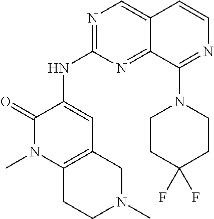 [SMILES: CN1CCc2c(cc(Nc3ncc4ccnc(N5CCC(F)(F)CC5)c4n3)c(=O)n2C)C1] |
| 111 | 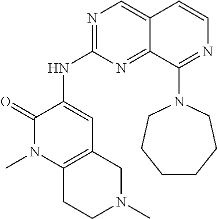 [SMILES: CN1CCc2c(cc(Nc3ncc4ccnc(N5CCCCCC5)c4n3)c(=O)n2C)C1] |
| 112 | 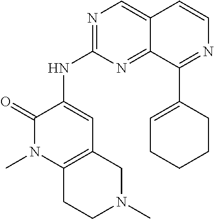 [SMILES: CN1CCc2c(cc(Nc3ncc4ccnc(C5=CCCCC5)c4n3)c(=O)n2C)C1] |
| 113 | 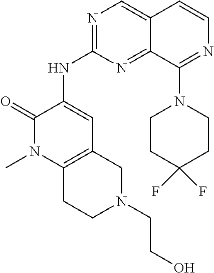 [SMILES: Cn1c2c(cc(Nc3ncc4ccnc(N5CCC(F)(F)CC5)c4n3)c1=O)CN(CCO)CC2] |
| 114 | 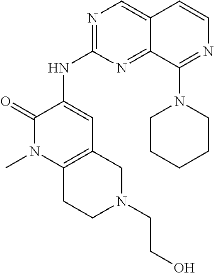 [SMILES: Cn1c2c(cc(Nc3ncc4ccnc(N5CCCCC5)c4n3)c1=O)CN(CCO)CC2] |
| 115 | 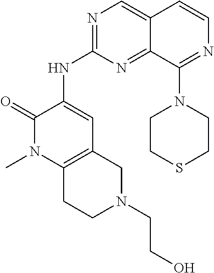 [SMILES: Cn1c2c(cc(Nc3ncc4ccnc(N5CCSCC5)c4n3)c1=O)CN(CCO)CC2] |
| 116 | 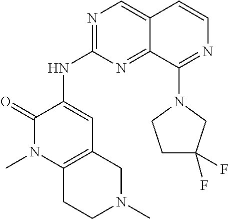 [SMILES: CN1CCc2c(cc(Nc3ncc4ccnc(N5CCC(F)(F)C5)c4n3)c(=O)n2C)C1] |
| 117 | 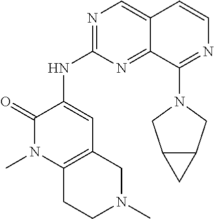 [SMILES: CN1CCc2c(cc(Nc3ncc4ccnc(N5CC6CC6C5)c4n3)c(=O)n2C)C1] |
| 118 | 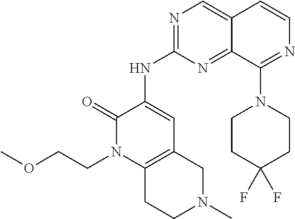 [SMILES: COCCn1c2c(cc(Nc3ncc4ccnc(N5CCC(F)(F)CC5)c4n3)c1=O)CN(C)CC2] |
| 119 | 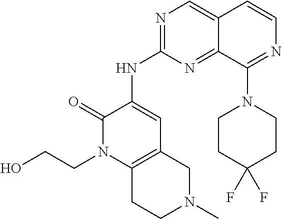 [SMILES: CN1CCc2c(cc(Nc3ncc4ccnc(N5CCC(F)(F)CC5)c4n3)c(=O)n2CCO)C1] |
| 120 | 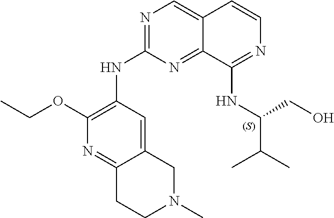 |
| 121 | 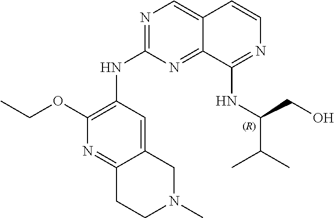 |
| 122 | 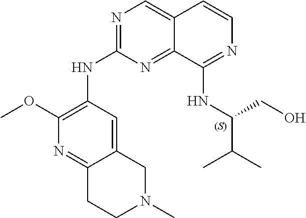 |
| 123 | 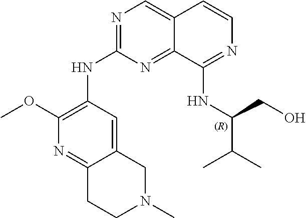 |
| 124 | 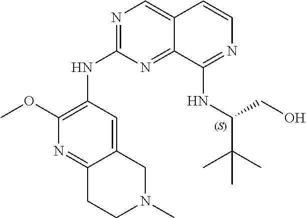 |
| 125 | 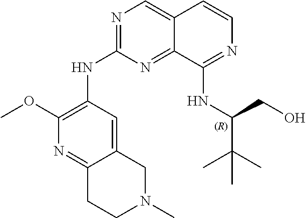 |
| 126 | 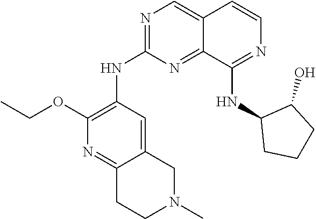 [SMILES: CCOc1nc2c(cc1Nc1ncc3ccnc(N[C@@H]4CCC[C@H]4O)c3n1)CN(C)CC2] |
| 127 | 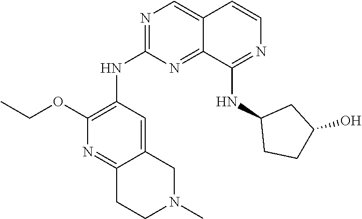 [SMILES: CCOc1nc2c(cc1Nc1ncc3ccnc(N[C@@H]4CC[C@@H](O)C4)c3n1)CN(C)CC2] |
| 128 | 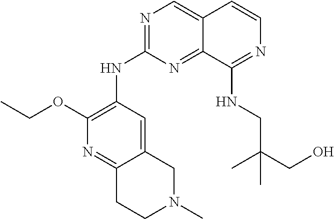 [SMILES: CCOc1nc2c(cc1Nc1ncc3ccnc(NCC(C)(C)CO)c3n1)CN(C)CC2] |
| 129 | 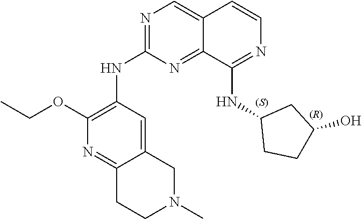 |
| 130 | 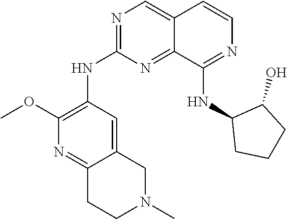 [SMILES: COc1nc2c(cc1Nc1ncc3ccnc(N[C@@H]4CCC[C@H]4O)c3n1)CN(C)CC2] |
| 131 | 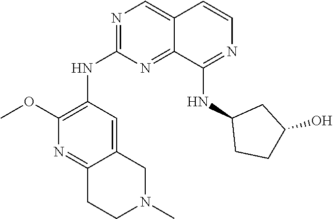 [SMILES: COc1nc2c(cc1Nc1ncc3ccnc(N[C@@H]4CC[C@@H](O)C4)c3n1)CN(C)CC2] |
| 132 | 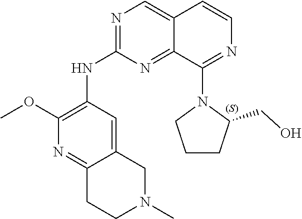 |
| 133 | 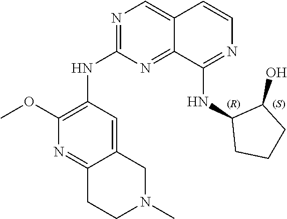 |
| 134 | 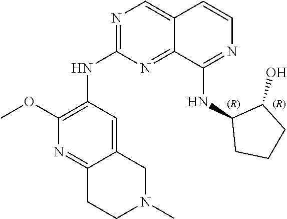 |
| 135 | 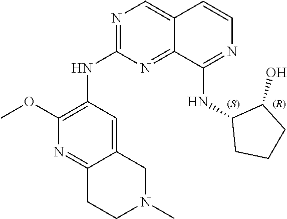 |
| 136 | 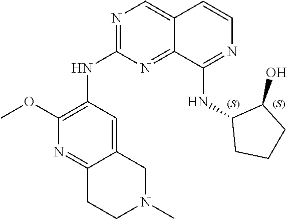 |
| 137 | 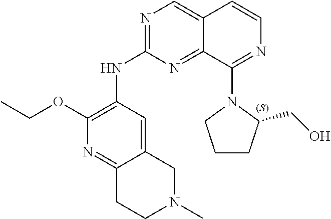 |
| 138 | 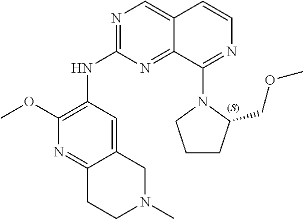 |
| 139 | 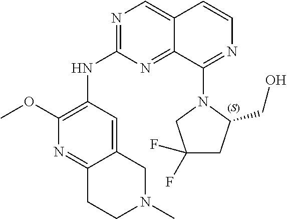 |
| 140 | 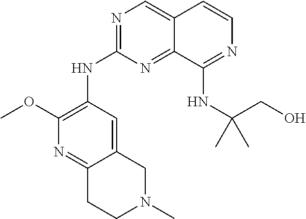 [SMILES: COc1nc2c(cc1Nc1ncc3ccnc(NC(C)(C)CO)c3n1)CN(C)CC2] |
| 141 | 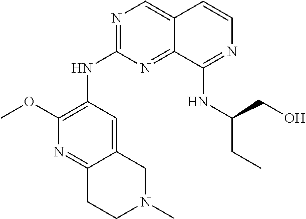 [SMILES: CC[C@H](CO)Nc1nccc2cnc(Nc3cc4c(nc3OC)CCN(C)C4)nc12] |
| 141 | 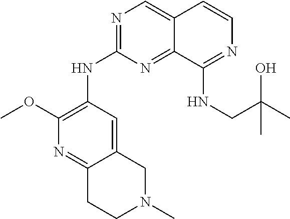 [SMILES: COc1nc2c(cc1Nc1ncc3ccnc(NCC(C)(C)O)c3n1)CN(C)CC2] |
| 143 | 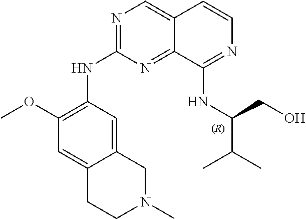 |

It is particularly noted that, herein, when referring to a “compound” having a specific structural formula, generally also encompassing its stereoisomers, diastereomers, enantiomers, racemic mixtures and isotopes derivative.

It is well known to those skilled in the art that a compound's salt, solvate, and hydrate are alternative forms of the compound, and they can all be converted into the compound under certain conditions. When referring to a compound, it generally includes its pharmaceutically acceptable salt, and further includes its solvate and hydrate.

Similarly, when referring to a compound herein, its prodrugs, metabolites and nitroxides are also generally included.

The pharmaceutically acceptable salts of the present invention may be formed using, for example, the following inorganic or organic acids: “Pharmaceutically acceptable salt” means a salt which, within the scope of reasonable medical judgment, is suitable for use in contact with tissues of humans and lower animals, without undue toxicity, irritation, allergic reaction, etc., can be called a reasonable benefit/risk ratio. The salts can be prepared in situ during the final isolation and purification of the compounds of the invention, or alone by reacting the free base or acid with a suitable reagent, as outlined below. For example, a free base function can be reacted with a suitable acid. In addition, when the compound of the present invention bears an acidic moiety, suitable pharmaceutically acceptable salts thereof may include metal salts, such as alkali metal salts (such as sodium or potassium salts); and alkaline earth metal salts (such as calcium or magnesium salts). Examples of pharmaceutically acceptable non-toxic acid addition salts are salts formed by amino acids with inorganic acids (e.g., hydrochloric, hydrobromic, phosphoric, sulfuric, and perchloric) or organic acids (e.g., acetic, oxalic, maleic, tartaric, citric acid, succinic acid or malonic acid), or salts formed by using other methods known in the art such as ion exchange. Other pharmaceutically acceptable salts include adipate, sodium alginate, ascorbate, aspartate, benzenesulfonate, benzoate, bisulfate, borate, butyrate, camphorate, camphorsulfonate, citrate, cyclopentanepropionate, digluconate, lauryl sulfate, ethylate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate Salt, maleate, malonate, methanesulfonate, 2-naphthalenesulfonate, nicotinate, nitrate, oleate, oxalate, palmitate, pamoate, pectate Salt, persulfate, 3-phenylpropionate, phosphate, bitter salt, pivalate, propionate, stearate, succinate, sulfate, tartrate, thiocyanate, p-toluene sulfonate, undecanoate, valerate, etc. Representative alkali or alkaline earth metal salts include those of sodium, lithium, potassium, calcium, magnesium, and the like. Other pharmaceutically acceptable salts include, where appropriate, nontoxic ammonium salts, quaternary ammonium salts, and amine cations formed with counterions, for example, halides, hydroxides, carboxylates, sulfates, phosphates, nitrates, lower alkylsulfonates and arylsulfonates.

The pharmaceutically acceptable salts of the present invention can be prepared by conventional methods, for example, by dissolving the compound of the present invention in a water-miscible organic solvent (such as acetone, methanol, ethanol and acetonitrile), adding an excess of aqueous solution of organic acid or inorganic acid aqueous solution, so that the salt is precipitated from the resulting mixture, the solvent and remaining free acid are removed therefrom, and the precipitated salt is isolated.

The precursors or metabolites described in the present invention may be precursors or metabolites known in the art, as long as the precursors or metabolites are transformed into compounds through in vivo metabolism. For example, “prodrugs” refer to those prodrugs of the compounds of the present invention which, within the scope of sound medical judgment, are suitable for use in contact with tissues of humans and lower animals without undue toxicity, irritation, allergic response, etc., and qualified as having a reasonable benefit/risk ratio and valid for its intended use. The term “prodrug” refers to a compound that is rapidly transformed in vivo to yield the parent compound of the above formula, for example by in vivo metabolism, or N-demethylation of a compound of the invention.

“Solvate” as used herein means a physical association of a compound of the present invention with one or more solvent molecules, whether organic or inorganic. This physical association includes hydrogen bonding. In some cases, for example, when one or more solvent molecules are incorporated into the crystal lattice of a crystalline solid, solvates will be able to be isolated. Solvent molecules in solvates may exist in regular and/or disordered arrangements. Solvates may contain stoichiometric or non-stoichiometric amounts of solvent molecules. “Solvate” encompasses both solution-phase and isolatable solvates. Exemplary solvates include, but are not limited to, hydrates, ethanolates, methanolates, and isopropanolates. Solvation methods are well known in the art.

The “stereoisomerism” described in the present invention is divided into conformational isomerism and configurational isomerism, and configurational isomerism can also be divided into cis-trans isomerism and optical isomerism (that is, optical isomerism). Due to the rotation or twisting of carbon and carbon single bonds in organic molecules of a certain configuration, a stereoisomerism phenomenon in which each atom or atomic group of the molecule has a different arrangement in space, the common structures are alkanes and cycloalkanes. Such as the chair conformation and boat conformation that appear in the structure of cyclohexane. “Stereoisomer” means when a compound of the present invention contains one or more asymmetric centers and is thus available as racemates and racemic mixtures, single enantiomers, diastereomeric mixtures and single diastereomers. The compound of the present invention has an asymmetric center, and each asymmetric center can produce two optical isomers, and the scope of the present invention includes all possible optical isomers and diastereoisomer mixtures and pure or partially pure compounds. The compounds described herein may exist in tautomeric forms having different points of attachment of hydrogens by displacement of one or more double bonds. For example, a ketone and its enol form are keto-enol tautomers. Each tautomer and mixtures thereof are included in the compounds of the present invention. Enantiomers, diastereoisomers, racemates, mesoisomers, cis-trans isomers, tautomers, geometric isomers, epimers of all compounds of formula (I) Conformants and their mixtures, etc., are included in the scope of the present invention.

The “isotopic derivatives” of the present invention refer to molecules that are labeled with isotopes of the compounds in this patent. Isotopes commonly used for isotopic labeling are: hydrogen isotopes, ²H and ³H; carbon isotopes: ¹¹C, ¹³C and ¹⁴C; chlorine isotopes: ³⁵Cl and ³⁷Cl; fluorine isotopes: ¹⁸F; iodine isotopes: ¹²³I and ¹²⁵I; nitrogen isotopes: ¹³N and ¹⁵N; Oxygen isotopes: ¹⁵O, ¹⁷O and ¹⁸O and sulfur isotope ³⁵S. These isotope-labeled compounds can be used to study the distribution of pharmaceutical molecules in tissues. Especially deuterium ³H and carbon ¹³C are more widely used due to their easy labeling and convenient detection. Substitution of certain heavy isotopes, such as deuterium (²H), can enhance metabolic stability, prolong half-life and thus provide therapeutic advantages for dose reduction. Isotopically labeled compounds are generally synthesized starting from labeled starting materials and carried out in the same way as non-isotopically labeled compounds using known synthetic techniques.

The present invention also provides the use of the compound of the present invention in the preparation of medicaments for preventing and/or treating cancer, tumor, inflammatory disease, autoimmune disease or immune-mediated disease.

In addition, the present invention provides a pharmaceutical composition for preventing and/or treating cancer, tumor, inflammatory disease, autoimmune disease, neurodegenerative disease, attention-related disease or immune-mediated disease, which comprises the compounds of present invention as active ingredients.

In addition, the present invention provides a method for preventing and/or treating cancer, tumor, inflammatory disease, autoimmune disease, neurodegenerative disease, attention-related disease or immune-mediated disease, which comprises administering a compound of the present invention tot a mammal in need thereof.

Representative examples of inflammatory, autoimmune, and immune-mediated diseases may include, but are not limited to, arthritis, rheumatoid arthritis, spondyloarthritis, gouty arthritis, osteoarthritis, juvenile arthritis, Other Arthritis Conditions, Lupus, Systemic Lupus Erythematosus (SLE), Skin Related Disorders, Psoriasis, Eczema, Dermatitis, Atopic Dermatitis, Pain, Pulmonary Disease, Lung Inflammation, Adult Respiratory Distress Syndrome (ARDS), pulmonary sarcoidosis, chronic pulmonary inflammatory disease, chronic obstructive pulmonary disease (COPD), cardiovascular disease, atherosclerosis, myocardial infarction, congestive heart failure, myocardial ischemia-reperfusion injury, inflammatory bowel disease, Crohn's disease, ulcerative colitis, irritable bowel syndrome, asthma, Sjogren's syndrome, autoimmune thyroid disease, urticaria (rubella), multiple sclerosis, scleroderma, organ transplant rejection, xenograft, idiopathic thrombocytopenic purpura (ITP), Parkinson's disease, Alzheimer's disease, diabetes-related diseases, inflammation, pelvic inflammatory disease, allergic rhinitis, allergic bronchitis, allergic sinusitis, leukemia, lymphoma, B Cell Lymphoma, T Cell Lymphoma, Myeloma, Acute Lymphocytic Leukemia (ALL), Chronic Lymphocytic Leukemia (CLL), Acute Myeloid Leukemia (AML), Chronic Myelogenous Leukemia (CML), Hairy Cell Leukemia, He Jie King's disease, non-Hodgkin's lymphoma, multiple myeloma, myelodysplastic syndrome (MDS), myeloproliferative neoplasm (MPN), diffuse large B-cell lymphoma, and follicular lymphoma.

Representative examples of cancer or tumor may include, but are not limited to, skin cancer, bladder cancer, ovarian cancer, breast cancer, stomach cancer, pancreatic cancer, prostate cancer, colon cancer, lung cancer, bone cancer, brain cancer, neuroblastoma, rectal cancer, colon cancer, familial adenomatous polyposis carcinoma, hereditary nonpolyposis colorectal cancer, esophagus cancer, lip cancer, larynx cancer, hypopharyngeal cancer, tongue cancer, salivary gland cancer, stomach cancer, adenocarcinoma, medullary thyroid cancer, Papillary thyroid cancer, renal cancer, renal parenchymal cancer, ovarian cancer, cervical cancer, uterine body cancer, endometrial cancer, choriocarcinoma, pancreatic cancer, prostate cancer, testicular cancer, urinary cancer, melanoma, brain tumors such as Glioblastoma, astrocytoma, meningioma, medulloblastoma and peripheral neuroectodermal tumor, Hodgkin lymphoma, non-Hodgkin lymphoma, Burkitt lymphoma, acute lymphoblastic leukemia (ALL), chronic lymphocytic leukemia (CLL), acute myeloid leukemia (AML), chronic myelogenous leukemia (CML), adult T-cell leukemia lymphoma, diffuse large B-cell lymphoma (DLBCL), hepatocellular carcinoma, gallbladder Carcinoma, bronchial carcinoma, small cell lung cancer, non-small cell lung cancer, multiple myeloma, basal cell tumor, teratoma, retinoblastoma, choroidal melanoma, seminoma, rhabdomyosarcoma, craniopharyngioma, osteosarcoma, chondrosarcoma, sarcoma, liposarcoma, fibrosarcoma, Ewing sarcoma, or plasmacytoma.

When the compound of the present invention or a pharmaceutically acceptable salt thereof is administered in combination with another anticancer agent or immune checkpoint inhibitor for the treatment of cancer or tumors, the compound of the present invention or a pharmaceutically acceptable salt thereof can provide enhanced anticancer effects.

Representative examples of anticancer agents useful in the treatment of cancer or tumors may include, but are not limited to, cell signal transduction inhibitors, chlorambucil, melphalan, cyclophosphamide, ifosfamide, busulfan, carbamate, Mustin, lomustine, streptozotocin, cisplatin, carboplatin, oxaliplatin, dacarbazine, temozolomide, procarbazine, methotrexate, fluorouracil, cytarabine, gemcitabine, Mercaptopurine, fludarabine, vinblastine, vincristine, vinorelbine, paclitaxel, docetaxel, topotecan, irinotecan, etoposide, trabectedin, dactinomycin, doxorubicin, epirubicin, daunorubicin, mitoxantrone, bleomycin, mitomycin C, ixabepilone, tamoxifen, flutamide, gonadorelin analogs, methadone Progesterone, prednisone, dexamethasone, methylprednisolone, thalidomide, interferon alpha, leucovorin, sirolimus, sirolimus ester, everolimus, afatinib, alisertib, amuvatinib, apatinib, axitinib, bortezomib, bosutinib, britinib, cabozantinib, cediranib, crenolanib, kezhuotinib, dabrafenib, dabrafenib, Cotinib, danucitinib, dasatinib, dovitinib, erlotinib, foretinib, ganetespib, gefitinib, ibrutinib, icotinib, imatinib, iniparib, la Patinib, lenvatinib, linifanib, linsitinib, masitinib, momelotinib, motisanib, neratinib, nilotinib, niraparib, oprozomib, olaparib, pazopanib, pictilisib, ponatinib, quizartinib, regorafenib, rigosertib, rucaparib, ruxolitinib, saracatinib, saridegib, sorafenib, sunitinib, tiratinib, tivantinib, tivozanib, tofacitinib, Trametinib, vandetanib, veliparib, vemurafenib, vimodegib, volasertib, alemtuzumab, bevacizumab, berentuzumab vedotin, catumaxumab Antibodies, cetuximab, denosumab, gemtuzumab, ipilimumab, nimotuzumab, ofatumumab, panitumumab, rituximab, tositumumab Monoclonal antibody, trastuzumab, PI3K inhibitor, CSF1R inhibitor, A2A and/or A2B receptor antagonist, IDO inhibitor, anti-PD-1 antibody, anti-PD-L1 antibody, LAG3 antibody, TIM-3 antibody and an anti-CTLA-4 antibody or any combination thereof.

Compounds of the present invention, or pharmaceutically acceptable salts thereof, provide enhanced therapeutic effect.

Representative examples of therapeutic agents useful in the treatment of inflammatory, autoimmune, and immune-mediated diseases can include, but are not limited to, steroidal agents (e.g., prednisone, prednisone, prednisone, methylphenidate, cortisone, cortisone, hydroxycortisone, betamethasone, dexamethasone, etc.), methotrexate, leflunomide, anti-TNFα agents (e.g., etanercept, infliximab, adalib monoclonal antibody, etc.), calcineurin inhibitors (eg, tacrolimus, pimecrolimus, etc.), and antihistamines (eg, diphenhydramine, hydroxyzine, loratadine, ebazan Tin, ketotifen, cetirizine, levocetirizine, fexofenadine, etc.), and at least one therapeutic agent selected from them can be included in the pharmaceutical composition of the present invention.

The compound of the present invention or a pharmaceutically acceptable salt thereof can be administered orally or parenterally as an active ingredient, and its effective amount ranges from 0.1 to 2,000 mg/kg body weight/day in mammals including humans (about 70 kg in body weight), Preferably 1 to 1,000 mg/kg body weight/day, and administered in single or 4 divided doses per day, or with or without a scheduled time. The dose of the active ingredient can be adjusted according to a number of relevant factors such as the condition of the subject to be treated, the type and severity of the disease, the rate of administration and the doctor's opinion. In some cases, amounts less than the above dosages may be appropriate. Amounts greater than the above doses may be used if no deleterious side effects are caused and such amounts may be administered in divided doses daily.

In addition, the present invention also provides a method for preventing and/or treating tumors, cancers, viral infections, organ transplant rejection, neurodegenerative diseases, attention-related diseases or autoimmune diseases, which comprises administering a compound of the present invention or a pharmaceutical composition of the present invention to a mammal in need thereof.

The pharmaceutical composition of the present invention can be formulated into dosage forms for oral administration or parenteral administration (including intramuscular, intravenous and subcutaneous routes, intratumoral injection) according to any of the conventional methods, such as tablets, granules, powders, capsules, syrups, emulsions, microemulsions, solutions or suspensions.

Pharmaceutical compositions of the present invention for oral administration can be prepared by mixing the active ingredient with carriers such as cellulose, calcium silicate, corn starch, lactose, sucrose, dextrose, calcium phosphate, stearic acid, magnesium stearate, calcium stearate, gelatin, talc, surfactant, suspending agent, emulsifier and diluent. Examples of carriers employed in the injectable compositions of the present invention are water, saline solution, glucose solution, glucose-like solution, alcohol, glycol, ether (e.g., polyethylene glycol 400), oil, fatty acids, fatty acid esters, glycerides, surfactants, suspending and emulsifying agents.

Other features of the invention will become apparent in the course of the description of exemplary embodiments of the invention which are given to illustrate the invention and are not intended to be limiting thereof, the following examples were prepared, separated and characterized using the methods disclosed in the invention.

The compounds of the present invention can be prepared in a variety of ways known to those skilled in the art of organic synthesis, using the methods described below as well as synthetic methods known in the art of synthetic organic chemistry or by variations thereof known to those skilled in the art to synthesize compounds of the invention. Preferred methods include, but are not limited to, those described below. Reactions are performed in solvents or solvent mixtures appropriate to the kit materials used and to the transformations effected. Those skilled in the art of organic synthesis will appreciate that the functionality present on the molecule is consistent with the proposed transitions. This sometimes requires judgment to alter the order of synthetic steps or starting materials to obtain the desired compound of the invention.

### DETAILED DESCRIPTION

### Terms

Unless otherwise stated, the terms used in the present application, including the specification and claims, are defined as follows. It must be noted that in the specification and appended claims, the singular form “a” and “an” includes plural references unless the context clearly dictates otherwise. If not stated otherwise, conventional methods of mass spectrometry, NMR, HPLC, protein chemistry, biochemistry, recombinant DNA techniques and pharmacology are used. In this application, the use of “or” or “and” means “and/or” if not stated otherwise.

In the description and claims, a given chemical formula or name shall cover all stereo and optical isomers and racemates in which such isomers exist. Unless otherwise indicated, all chiral (enantiomers and diastereoisomers) and racemic forms are within the scope of the invention. Many geometric isomers of C═C double bonds, C═N double bonds, ring systems, etc. may also exist in the compounds, and all such stable isomers are encompassed within the present invention. The present invention describes cis- and trans-(or E- and Z-) geometric isomers of the compounds of the invention and which may be isolated as a mixture of isomers or as separated isomeric forms. The compounds of the invention may be isolated in optically active or racemic forms. All methods used to prepare the compounds of the invention and intermediates prepared therein are considered part of the invention. When preparing enantiomeric or diastereomeric products, they may be separated by customary methods, for example by chromatography or fractional crystallization. Depending on the process conditions, the end products of the invention are obtained in free (neutral) or salt form. The free forms and salts of these end products are within the scope of the present invention. A compound can be converted from one form to another, if desired. A free base or acid can be converted into a salt; a salt can be converted into the free compound or another salt; a mixture of isomeric compounds of the invention can be separated into the individual isomers. The compounds of the invention, their free forms and salts, may exist in various tautomeric forms in which the hydrogen atoms are transposed to other parts of the molecule and thus the chemical bonds between the atoms of the molecule are rearranged. It is to be understood that all tautomeric forms which may exist are included within the present invention.

Unless otherwise defined, the definitions of the substituents in the present invention are independent and not interrelated, for example, for R^{a} (or R^{a′}) in the substituents, they are independent in the definitions of different substituents. Specifically, when one definition is selected for R^{a} (or R^{a′}) in one substituent, it does not mean that R^{a} (or R^{a′}) has the same definition in other substituents. More specifically, for example (but not exhaustively) for NR^{a}R^{a′}, when R^{a} (or R^{a′}) is defined from hydrogen, it does not mean that in —C(O)—NR^{a}R^{a′}, R^{a} (or R^{a′}) must be hydrogen.

Unless otherwise defined, when a substituent is noted as “optionally substituted”, the substituent is selected from, for example, the following substituents, such as alkyl, cycloalkyl, aryl, heterocyclyl, halogen, hydroxy, alkoxy, oxo, alkanoyl, aryloxy, alkanoyloxy, amino, alkylamino, arylamino, arylalkylamino, disubstituted amine groups (wherein 2 amino substituents are selected from alkyl, aryl or arylalkyl), alkanoylamino, aroylamino, aralkanoylamino, substituted alkanoylamino, substituted arylamino, substituted aralkanoylamino, thio, alkylthio, arylthio, arylalkylthio, arylthiocarbonyl, arylalkylthiocarbonyl, alkylsulfonyl, arylsulfonyl, arylalkylsulfonyl, sulfonylamino such as —SO₂NH₂, substituted sulfonylamino, nitro, cyano, carboxyl, carbamoyl such as —CONH₂, substituted carbamoyl such as —CONH alkyl, —CONH aryl, —CONH arylalkyl or with two In the case of one substituent selected from alkyl, aryl or arylalkyl, alkoxycarbonyl, aryl, substituted aryl, guanidino, heterocyclyl such as indolyl, imidazolyl, furyl, Thienyl, thiazolyl, pyrrolidinyl, pyridyl, pyrimidinyl, pyrrolidinyl, piperidinyl, morpholinyl, piperazinyl, homopiperazinyl, etc. and substituted heterocyclic groups.

The term “alkyl” or “alkylene” as used herein is intended to include both branched and straight chain saturated aliphatic hydrocarbon groups having the indicated number of carbon atoms. For example, “C₁-C₆ alkyl” means an alkyl group having 1 to 6 carbon atoms. Examples of alkyl groups include, but are not limited to, methyl (Me), ethyl (Et), propyl (such as n-propyl and isopropyl), butyl (such as n-butyl, isobutyl, t-butyl), and pentyl (eg n-pentyl, isopentyl, neopentyl).

The term “alkenyl” denotes a straight or branched chain hydrocarbon group containing one or more double bonds and generally having a length of 2 to 20 carbon atoms. For example, “C₂-C₆ alkenyl” contains two to six carbon atoms. Alkenyl groups include, but are not limited to, e.g. vinyl, propenyl, butenyl, 1-methyl-2-buten-1-yl, and the like.

The term “alkynyl” denotes a straight or branched chain hydrocarbon group containing one or more triple bonds and generally having a length of 2 to 20 carbon atoms. For example, “C₂-C₆ alkynyl” contains two to six carbon atoms. Representative alkynyl groups include, but are not limited to, for example, ethynyl, 1-propynyl, 1-butynyl, and the like.

The term “alkoxy” or “alkyloxy” refers to —O-alkyl. “C₁-C₆ alkoxy” (or alkyloxy) is intended to include C₁, C₂, C₃, C₄, C₅, C₆ alkoxy. Examples of alkoxy include, but are not limited to, methoxy, ethoxy, propoxy (e.g., n-propoxy and isopropoxy), and tert-butoxy. Similarly, “alkylthio” or “thioalkoxy” denotes an alkyl group as defined above having the indicated number of carbon atoms attached through a sulfur bridge; e.g. methyl-S— and ethyl-S—.

The term “carbonyl” refers to an organic functional group (C═O) consisting of two atoms, carbon and oxygen, linked by a double bond.

The term “aryl”, alone or as part of a larger moiety such as “aralkyl”, “aralkoxy” or “aryloxyalkyl”, refers to a single ring having a total of 5 to 12 ring members, a bicyclic or tricyclic ring system, wherein at least one ring in the system is aromatic and wherein each ring in the system contains 3 to 7 ring members. In certain embodiments of the invention, “aryl” refers to an aromatic ring system which includes, but is not limited to, phenyl, biphenyl, indanyl, 1-naphthyl, 2-naphthyl, and tetrahydronaphthyl. The term “aralkyl” or “arylalkyl” refers to an alkyl residue attached to an aryl ring. Non-limiting examples include benzyl, phenethyl, and the like. A fused aryl group can be attached to another group at a suitable position on the cycloalkyl ring or aromatic ring. For example dashed lines drawn from ring systems indicate that bonds may be attached to any suitable ring atom.

The term “cycloalkyl” refers to a monocyclic or bicyclic cyclic alkyl group. Monocyclic cyclic alkyl refers to C₃-C₈ cyclic alkyl, including but not limited to cyclopropyl, cyclobutyl, cyclopentyl, cyclohexyl and norbornyl. Branched cycloalkyl groups such as 1-methylcyclopropyl and 2-methylcyclopropyl are included within the definition of “cycloalkyl”. Bicyclic cyclic alkyl groups include bridged, spiro, or fused ring cycloalkyls.

The term “cycloalkenyl” refers to a monocyclic or bicyclic cyclic alkenyl group. Monocyclic cyclic alkenyl refers to C₃-C₈ cyclic alkenyl, including but not limited to cyclopropenyl, cyclobutenyl, cyclopentenyl, cyclohexenyl and norbornenyl. Branched cycloalkenyl groups such as 1-methylcyclopropenyl and 2-methylcyclopropenyl are included within the definition of “cycloalkenyl”. Bicyclic cyclic alkenyl groups include bridged, spiro, or fused ring cyclic alkenyl groups.

“Halo” or “halogen” includes fluoro, chloro, bromo and iodo. “Haloalkyl” is intended to include branched and straight chain saturated aliphatic hydrocarbon groups having the indicated number of carbon atoms substituted with one or more halogens. Examples of haloalkyl include, but are not limited to, fluoromethyl, difluoromethyl, trifluoromethyl, trichloromethyl, pentafluoroethyl, pentachloroethyl, 2,2,2-trifluoroethyl, heptafluoropropyl and heptachloropropyl. Examples of haloalkyl also include “fluoroalkyl” intended to include branched and straight-chain saturated aliphatic hydrocarbon groups having the specified number of carbon atoms and substituted with 1 or more fluorine atoms.

“Haloalkoxy” or “haloalkyloxy” means a haloalkyl group as defined above having the indicated number of carbon atoms attached through an oxygen bridge. For example, “haloC₁-C₆ alkoxy” is intended to include C₁, C₂, C₃, C₄, C₅, C₆ haloalkoxy. Examples of haloalkoxy include, but are not limited to, trifluoromethoxy, 2,2,2-trifluoroethoxy, and pentafluoroethoxy. Similarly, “haloalkylthio” or “thiohaloalkoxy” denotes a haloalkyl group as defined above having the indicated number of carbon atoms attached through a sulfur bridge; for example trifluoromethyl-S— and pentafluoroethyl —S—.

In the present disclosure, the expression Cₓ₁-Cₓ₂ is used when referring to some substituent groups, which means that the number of carbon atoms in the substituent groups may be x1 to x2. For example, C₀-C₈ means that the group contains 0, 1, 2, 3, 4, 5, 6, 7 or 8 carbon atoms, C₁-C₈ means that the group contains 1, 2, 3, 4, 5, 6, 7 or 8 carbon atoms, C₂-C₈ means that the group contains 2, 3, 4, 5, 6, 7 or 8 carbon atoms, C₃-C₈ means that the group contains 3, 4, 5, 6, 7 or 8 carbon atoms, C₄-C₈ means that the group contains 4, 5, 6, 7 or 8 carbon atoms, C₀-C₆ means that the group contains 0, 1, 2, 3, 4, 5 or 6 carbon atoms, C₁-C₆ means that the group contains 1, 2, 3, 4, 5 or 6 carbon atoms, C₂-C₆ means that the group contains 2, 3, 4, 5 or 6 carbon atoms, C₃-C₆ means that the group contains 3, 4, 5 or 6 carbon atoms.

In this disclosure, the expression “x1-x2 membered ring” is used when referring to cyclic groups such as aryl, heteroaryl, cycloalkyl, and heterocycloalkyl, which means that the number of the ring atoms of the group can be x1 to x2. For example, the 3-12 membered cyclic group may be a 3, 4, 5, 6, 7, 8, 9, 10, 11 or 12 membered ring, and the number of ring atoms may be 3, 4, 5, 6, 7, 8, 9, 10, 11 or 12; 3-6-membered ring means that the cyclic group can be 3, 4, 5 or 6-membered ring, and the number of ring atoms can be 3, 4, 5 or 6; 3-8 membered ring means that the cyclic group can be 3, 4, 5, 6, 7 or 8 membered ring, and the number of ring atoms can be 3, 4, 5, 6, 7 or 8; 3-9 A membered ring means that the cyclic group can be a 3, 4, 5, 6, 7, 8 or 9-membered ring, and the number of ring atoms can be 3, 4, 5, 6, 7, 8 or 9; 4-7 membered ring means that the cyclic group can be a 4, 5, 6 or 7-membered ring, and the number of ring atoms can be 4, 5, 6 or 7; 5-8-membered ring means that the cyclic group can be 5, 6, 7 or 8-membered ring, the number of ring atoms can be 5, 6, 7 or 8; 5-12 membered ring means that the ring group can be 5, 6, 7, 8, 9, 10, 11 or 12-membered ring, the number of ring atoms can be 5, 6, 7, 8, 9, 10, 11 or 12; 6-12 membered ring means that the ring group can be 6, 7, 8, 9, 10, 11- or 12-membered rings may have 6, 7, 8, 9, 10, 11 or 12 ring atoms. The ring atoms may be carbon atoms or heteroatoms, for example selected from N, O and S. When the ring is heterocyclic, the heterocyclic ring may contain 1, 2, 3, 4, 5, 6, 7, 8, 9, 10 or more ring heteroatoms, for example selected from N, O and S of heteroatoms.

In the present disclosure, the one or more halogens may each be independently selected from fluorine, chlorine, bromine and iodine.

The term “heteroaryl” means a stable 3-membered, 4-membered, 5-membered, 6-membered, or 7-membered aromatic monocyclic or aromatic bicyclic ring or 7-, 8-, 9-, 10-, 11-, 12-membered aromatic polycyclic heterocycles that are fully unsaturated, partially unsaturated, and contain carbon atoms and 1, 2, 3, or 4 heteroatoms independently selected from N, O, and S; and include any of the following polycyclic groups in which any heterocyclic ring as defined above is fused to a benzene ring. Nitrogen and sulfur heteroatoms can be optionally oxidized. The nitrogen atom is substituted or unsubstituted (i.e. N or NR, where R is H or another substituent if defined). A heterocycle can be attached to its pendant group at any heteroatom or carbon atom that results in a stable structure. The heterocyclyl groups described herein may be substituted on carbon or nitrogen atoms if the resulting compound is stable. The nitrogen in the heterocycle can optionally be quaternized. Preferably, when the total number of S and O atoms in the heterocycle exceeds 1, then these heteroatoms are not adjacent to each other. Preferably, the total number of S and O atoms in the heterocycle is not greater than one. When the term “heterocycle” is used, it is intended to include heteroaryl. Examples of heteroaryl groups include, but are not limited to, acridinyl, azetidinyl, aziocinyl, benzimidazolyl, benzofuryl, benzothiofuranyl, benzothienyl, benzoxazolyl, benzoxazolinyl, benzothiazolyl, benzotriazolyl, benzotetrazolyl, benzisoxazolyl, benzisothiazolyl, benzimidazolyl, carbazolyl, 4aH-carbazolyl, carbolinyl, chromanyl, chromenyl, cinnolinyl, decahydroquinolyl, 2H,6H-1,5,2-dithiazinyl, dihydrofuro[2, 3-b]Tetrahydrofuryl, furyl, furanyl, imidazolidinyl, imidazolinyl, imidazolyl, 1H-indazolyl, imidazopyridyl, indolenyl, indolinyl, Indolazinyl, indolyl, 3H-indolyl, isatinoyl, isobenzofuryl, isochromanyl, isoindazolyl, isoindolinyl, isoindolyl, isoindolyl Quinolinyl, isothiazolyl, isothiazolopyridyl, isoxazolyl, isoxazolopyridyl, methylenedioxyphenyl, morpholinyl, diazanaphthyl, octahydroisoquinoline oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolopyridyl, oxazolidinyl, diazaphenyl, oxindolyl, pyrimidinyl, phenanthridinyl, phenanthrolinyl, phenazinyl, phenothiazinyl, phenoxathiyl, phenoxazinyl, phthalazinyl, piperazinyl, piperidinyl, piperidinyl, 4-piperidinyl, piperonyl, pteridinyl, purinyl, Pyranyl, pyrazinyl, pyrazolidinyl, pyrazolinyl, pyrazolopyridyl, pyrazolyl, pyridazinyl, pyridoxazolyl, pyridimidazolyl, pyridothiazolyl, pyridyl, pyrimidinyl, pyrrolidinyl, pyrrolinyl, 2-pyrrolidonyl, 2H-pyrrolyl, pyrrolyl, quinazolinyl, quinolinyl, 4H-quinazinyl, quinoxalinyl, quinuclidinyl, Tetrazolyl, tetrahydrofuryl, tetrahydroisoquinolyl, tetrahydroquinolyl, 6H-1,2,5-thiadiazinyl, 1,2,3-thiadiazolyl, 1,2,4-Thiadiazolyl, 1,2,5-thiadiazolyl, 1,3,4-thiadiazolyl, thianthryl, thiazolyl, thienyl, thiazolopyridyl, thienothiazolyl, thienooxa Azolyl, thienoimidazolyl, thienyl, triazinyl, 1,2,3-triazolyl, 1,2,4-triazolyl, 1,2,5-triazolyl, 1,3,4-Triazolyl and xanthenyl, quinolinyl, isoquinolyl, phthalazinyl, quinazolinyl, indolyl, isoindolyl, indolinyl, 1H-indazolyl, benzo Imidazolyl, 1,2,3,4-tetrahydroquinolinyl, 1,2,3,4-tetrahydroisoquinolinyl, 5,6,7,8-tetrahydro-quinolinyl, 2,3-dihydro-benzofuryl, chromanyl, 1,2,3,4-tetrahydro-quinoxalinyl and 1,2,3,4-tetrahydro-quinazolinyl. The term “heteroaryl” may also include biaryl structures formed by the above-defined “aryl” and a monocyclic “heteroaryl”, such as but not limited to “-phenylbipyridyl-”, “-“phenyl bipyrimidyl”, “-pyridyl biphenyl”, “-pyridyl bipyrimidyl-”, “-pyrimidyl biphenyl-”; wherein the present invention also includes condensed rings containing, for example, the above-mentioned heterocycles and spiro compounds.

As used herein, the term “heterocycloalkyl” refers to a monocyclic heterocycloalkyl system, or a bicyclic heterocycloalkyl system, and also includes spiroheterocycle or bridged heterocycloalkyl. Monocyclic heterocycloalkyl refers to a 3-8 membered, saturated or unsaturated but non-aromatic cyclic alkyl system containing at least one selected from O, N, S, and P. A bicyclic heterocycloalkyl system refers to a heterocycloalkyl fused to a phenyl, or a cycloalkyl, or a cycloalkenyl, or a heterocycloalkyl, or a heteroaryl.

The term “bridged cycloalkyl” as used herein refers to polycyclic compounds sharing two or more carbon atoms. Such “bridged cycloalkyl” can be divided into bicyclic bridged ring hydrocarbons and polycyclic bridged ring hydrocarbons. The former is composed of two alicyclic rings sharing more than two carbon atoms; the latter is a bridged ring hydrocarbon composed of more than three rings.

The term “spirocycloalkyl” as used herein refers to polycyclic hydrocarbons in which the monocyclic rings share one carbon atom (called the spiro atom).

The term “bridged ring heterogroup” used herein refers to a polycyclic compound sharing two or more carbon atoms, and the ring contains at least one atom selected from O, N, and S. It can be divided into bicyclic bridged heterocycles and polycyclic bridged heterocycles.

The term “heterospirocyclyl” used herein refers to a polycyclic hydrocarbon that shares one carbon atom (called a spiro atom) between monocyclic rings, and the ring contains at least one atom selected from O, N, and S.

The term “substituted” as used herein means that at least one hydrogen atom is replaced by a non-hydrogen group, provided that normal valences are maintained and that the substitution results in a stable compound. A ring double bond, as used herein, is a double bond formed between two adjacent ring atoms (e.g., C═C, C═N or N═N).

Where nitrogen atoms (e.g. amines) are present on compounds of the invention, these nitrogen atoms can be converted to N-oxides by treatment with oxidizing agents (e.g. mCPBA and/or hydrogen peroxide) to obtain other compounds of the invention. Accordingly, both shown and claimed nitrogen atoms are considered to cover both the shown nitrogen and its N-oxides to obtain the derivatives of the present invention.

When any variable occurs more than one time in any composition or formula of a compound, its definition on each occurrence is independent of its definition at every other occurrence. Thus, for example, if a group is shown to be substituted with 0-3 R, then said group may be optionally substituted with up to three R groups, and R at each occurrence is independently selected from the definition of R. Also, combinations of substituents and/or variables are permissible only if such combinations result in stable compounds.

The term “patient” as used herein refers to an organism being treated by the methods of the present invention. Such organisms preferably include, but are not limited to, mammals (e.g., murine, simian, equine, bovine, porcine, canine, feline, etc.) and most preferably refer to humans.

As used herein, the term “effective amount” means the amount of a drug or agent (i.e., a compound of the invention) that will elicit the biological or medical response of a tissue, system, animal or human being sought, e.g., by a researcher or clinician. Furthermore, the term “therapeutically effective amount” means an amount that results in improved treatment, cure, prevention or alleviation of a disease, disorder or side effect, or a reduction in or the rate of disease progression. An effective amount may be given in one or more administrations, applications or doses and is not intended to be limited by a particular formulation or route of administration. The term also includes within its scope amounts effective to enhance normal physiological function.

As used herein, the term “treating” includes any effect that results in amelioration of a condition, disease, disorder, etc., such as alleviation, reduction, regulation, amelioration or elimination, or amelioration of the symptoms thereof.

The term “pharmaceutically acceptable” is used herein to refer to those compounds, substances, compositions and/or dosage forms: within the scope of sound medical judgment, they are suitable for use in contact with human and animal tissues without excessive toxicity, irritation, allergic reactions, and/or other problems or complications, commensurate with a reasonable benefit/risk ratio.

The phrase “pharmaceutically acceptable carrier” as used herein means a pharmaceutical substance, composition or vehicle, such as a liquid or solid filler, diluent, excipient, manufacturing aid (e.g. lubricant, talc, magnesium stearate, calcium stearate or zinc stearate or stearic acid) or solvent-encapsulated substances involved in the carrying or transport of a subject compound from one organ or body part to another. Each carrier must be “acceptable” in the sense of being compatible with the other ingredients of the formulation and not injurious to the patient.

The term “pharmaceutical composition” means a composition comprising a compound of the present invention together with at least one other pharmaceutically acceptable carrier. “Pharmaceutically acceptable carrier” means a medium generally accepted in the art for the delivery of biologically active agents to animals, particularly mammals, including (i.e.) adjuvants, excipients or vehicles, such as diluents, preservatives, fillers, flow regulators, disintegrants, wetting agents, emulsifiers, suspending agents, sweeteners, flavoring agents, fragrances, antibacterial agents, antifungal agents, lubricants and dispersants, depending on mode of administration and nature of dosage form.

### Certain Pharmaceutical and Medical Terms

The term “acceptable”, as used herein, means that a formulation ingredient or active ingredient does not have an undue adverse effect on health for the general purpose of treatment.

The term “cancer”, as used herein, refers to an abnormal growth of cells that cannot be controlled and, under certain conditions, is capable of metastasizing (spreading). Cancers of this type include, but are not limited to, solid tumors (e.g., bladder, bowel, brain, chest, uterus, heart, kidney, lung, lymphoid tissue (lymphoma), ovary, pancreas or other endocrine organs (eg, thyroid), prostate, skin (melanoma), or blood cancer (such as non-leukemic leukemia).

The term “administration in combination” or similar terms, as used herein, refers to the administration of several selected therapeutic agents to a patient, in the same or different modes of administration at the same or different times.

The term “enhancing” or “capable of enhancing”, as used herein, means that the desired result can be increased or prolonged, either in potency or duration. Thus, in relation to enhancing the therapeutic effect of a drug, the term “capable of potentiating” refers to the ability of the drug to increase or prolong its potency or duration in the system. As used herein, “potency value” refers to the ability to maximize the enhancement of another therapeutic drug in an ideal system.

The term “immune disease” refers to a disease or condition of an adverse or deleterious reaction to an endogenous or exogenous antigen. The result is usually dysfunction of the cells, or destruction thereof and dysfunction, or destruction of organs or tissues that may produce immune symptoms.

The terms “kit” and “product packaging” are synonymous.

The term “subject” or “patient” includes mammals and non-mammals. Mammals include, but are not limited to, mammals: humans, non-human primates such as orangutans, apes, and monkeys; agricultural animals such as cattle, horses, goats, sheep, and pigs; domestic animals such as rabbits and dogs; experimental animals include rodents, such as rats, mice and guinea pigs. Non-mammalian animals include, but are not limited to, birds, fish, and the like. In a preferred embodiment, the selected mammal is a human.

The term “treatment”, “course of treatment” or “therapy” as used herein includes alleviating, suppressing or improving the symptoms or conditions of a disease; inhibiting the development of complications; improving or preventing the underlying metabolic syndrome; inhibiting the development of diseases or symptoms, Such as controlling the development of a disease or condition; alleviating a disease or a symptom; causing a disease or a symptom to regress; alleviating a complication caused by a disease or a symptom, or preventing and/or treating a sign caused by a disease or a symptom.

As used herein, a certain compound or pharmaceutical composition, after administration, can improve a certain disease, symptom or situation, especially improve its severity, delay the onset, slow down the progression of the disease, or reduce the duration of the disease. Circumstances that may be attributable to or related to the administration, whether fixed or episodic, continuous or intermittent.

### Route of Administration

Suitable routes of administration include, but are not limited to, oral, intravenous, rectal, aerosol, parenteral, ocular, pulmonary, transdermal, vaginal, ear canal, nasal administration and topical administration. In addition, by way of example only, parenteral administration includes intramuscular injection, subcutaneous injection, intravenous injection, intramedullary injection, intraventricular injection, intraperitoneal injection, intralymphatic injection, and intranasal injection.

In one aspect, the compounds described herein are administered locally rather than systemically. In certain embodiments, the depot formulation is administered by implantation (e.g., subcutaneously or intramuscularly) or by intramuscular injection. Additionally, in another embodiment, the drug is administered via a targeted drug delivery system. For example, liposomes coated with organ-specific antibodies. In such embodiments, the liposomes are selectively directed to and taken up by specific organs.

### Pharmaceutical Composition and Dosage

The present invention also provides pharmaceutical compositions comprising a therapeutically effective amount of one or more compounds of the present invention formulated together with one or more pharmaceutically acceptable carriers (additives) and/or diluents, and optionally one or more of the other therapeutic agents described above. The compounds of the present invention may be administered for any of the above uses by any suitable means, for example orally, such as tablets, pills, powders, granules, elixirs, tinctures, suspensions (including nanosuspensions, microsuspensions, spray-dried dispersions), syrups and emulsions; sublingually; buccally; parenterally, such as by subcutaneous, intravenous, intramuscular or intrasternal injection or infusion techniques (e.g., as sterile injectable aqueous or nonaqueous solutions or suspensions liquid form); nasally, including to the nasal membranes, such as by inhalation spray; topically, such as in the form of a cream or ointment; or rectally, such as in the form of a suppository; or by intratumoral injection. They can be administered alone, but generally will be administered using a pharmaceutical carrier selected on the basis of the chosen route of administration and standard pharmaceutical practice.

Pharmaceutical carriers are formulated according to a number of factors within the purview of those skilled in the art. These factors include, but are not limited to: the type and nature of the active agent being formulated; the subject to whom the composition containing the active agent is to be administered; the intended route of administration of the composition; and the therapeutic indication being targeted. Pharmaceutical carriers include aqueous and non-aqueous liquid media and various solid and semisolid dosage forms.

Such carriers may include many different ingredients and additives other than the active agent, which are included in the formulation for various reasons known to those skilled in the art, such as to stabilize the active agent, binders, and the like. A description of suitable pharmaceutical carriers and the factors involved in the selection of the carrier can be found in several readily available sources, such as Allen L. V. Jr. et al. Remington: The Science and Practice of Pharmacy (2 Volumes), 22nd Edition (2012), Pharmaceutical Press.

Dosage regimens for the compounds of the present invention will of course vary depending on known factors such as the pharmacodynamic properties of the particular agent and its mode and route of administration; the species, age, sex, health, medical condition and weight of the recipient; nature and extent of symptoms; type of concomitant therapy; frequency of therapy; route of administration, patient's renal and hepatic function, and desired effects. As a general guide, the daily oral dosage of each active ingredient should be from about 0.001 mg/day to about 10-5000 mg/day, preferably from about 0.01 mg/day to about 1000 mg/day, and most preferably From about 0.1 mg/day to about 250 mg/day. The most preferred dose intravenously will be about 0.01 mg/kg/minute to about 10 mg/kg/minute during a constant rate infusion. The compounds of the present invention may be administered in a single daily dose, or the total daily dose may be administered in divided doses of two, three or four times daily.

The compounds are usually formulated with a suitable pharmaceutical diluent, excipient or carrier (herein collectively referred to as drug carriers) in the form of mixtures for administration.

Dosage forms (pharmaceutical compositions) suitable for administration may contain from about 1 milligram to about 2000 milligrams of active ingredient per dosage unit. In these pharmaceutical compositions, the active ingredient will generally be present in an amount of about 0.1-95% by weight, based on the total weight of the composition.

A typical capsule for oral administration contains at least one compound of the invention (250 mg), lactose (75 mg) and magnesium stearate (15 mg). The mixture was passed through a 60 mesh screen and packed into size 1 gelatin capsules.

A typical injectable formulation can be prepared by aseptically placing at least one compound of the present invention (250 mg) in a vial, lyophilizing in a sterile manner and sealing. For use, the vial contents are mixed with 2 mL of normal saline to produce an injectable formulation.

Included within the scope of the present invention are pharmaceutical compositions comprising (alone or in combination with a pharmaceutical carrier) a therapeutically effective amount of at least one compound of the present invention as an active ingredient. Optionally, compounds of the invention may be used alone, in combination with other compounds of the invention, or in combination with one or more other therapeutic agents (eg, anticancer agents or other pharmaceutically active substances).

Irrespective of the chosen route of administration, the compounds of the invention (which may be used in suitably hydrated form) and/or the pharmaceutical compositions of the invention are formulated into pharmaceutical dosage forms by conventional methods known to those skilled in the art.

Actual dosage levels of the active ingredients in the pharmaceutical compositions of the invention can be varied to obtain an amount of active ingredient that is effective to achieve the desired therapeutic response, composition, and mode of administration for a particular patient without being toxic to the patient.

The selected dosage level will depend on a variety of factors, including the activity of the particular compound of the invention employed, or its ester, salt or amide; the route of administration; the time of administration; the rate of excretion of the particular compound employed; and the rate and extent of absorption; duration of treatment; other drugs, compounds and/or substances used in combination with the particular compound used; factors well known in the medical art such as age, sex, weight, condition, general health and prior medical history of the patient being treated.

A physician or veterinarian having ordinary skill in the art can readily determine and prescribe the effective amount of the pharmaceutical composition required. For example, the physician or veterinarian can start the doses of the compounds of the invention employed in the pharmaceutical composition at levels lower than required to achieve the desired therapeutic effect and gradually increase the dosage until the desired effect is achieved. In general, a suitable daily dose of a compound of the invention will be that amount of the compound at the lowest dose effective to produce a therapeutic effect. Such effective dosage will generally depend on the factors mentioned above. Typically, oral, intravenous, intracerebroventricular and subcutaneous doses of the compounds of this invention to patients range from about 0.01 to about 50 mg/kg body weight/day. If desired, an effective daily dose of the active compound may be administered in two, three, four, five, six or more sub-doses at appropriate intervals throughout the day, optionally in unit dosage form. In certain aspects of the invention, the administration is once daily.

Although the compounds of the present invention may be administered alone, it is preferred to administer the compounds in the form of pharmaceutical formulations (compositions).

### Kit/Product Packaging

Kits/product packaging are also described herein for use in the treatment of the above indications. These kits may consist of transporters, packs, or container boxes, which may be divided into compartments to accommodate one or more types of containers, such as vials, test tubes, and the like, each container containing a separate component in the method described above. Suitable containers include bottles, vials, syringes, test tubes and the like. Containers are made of acceptable materials such as glass or plastic.

For example, a container may contain one or more compounds described herein, either as a pharmaceutical composition or in admixture with other ingredients described herein. The container can have a sterile outlet (eg, the container can be an IV bag or bottle, the stopper of which can be pierced by a hypodermic needle). Such a kit may contain a compound, and instructions for use, labels or instructions for use described herein.

A typical kit may include one or more containers, each containing one or more materials (such as reagents, concentrated stock solutions, and/or or equipment). These materials include, but are not limited to, buffers, diluents, filters, needles, syringes, transporters, bags, containers, bottles and/or test tubes, accompanied by a list of contents and/or instructions for use, and inner packaging also accompanied by instructions. Instructions for the entire set are to be included.

Labels can be displayed on or closely associated with the container. The appearance of the label on the container means that the label letters, numbers or other features are pasted, molded, or engraved on the container; the label can also appear in the container box or shipping box containing various containers, such as in the product insert. A label may be used to indicate a specific therapeutic use of the contents. The label may also bear instructions for use of the contents, such as described in the methods above. All features described in this specification (including any stated claims, abstract and drawings), and/or all steps involved in any method or process, may exist in any combination, unless certain features or steps are mutually exclusive in the same combination.

The above-mentioned features mentioned in the present invention, or the features mentioned in the embodiments can be combined arbitrarily. All the features disclosed in the specification of this case can be used in combination with any combination, and each feature disclosed in the specification can be replaced by any alternative feature that can provide the same, equivalent or similar purpose. Therefore, unless otherwise specified, the disclosed features are only general examples of equivalent or similar features.

Below in conjunction with specific embodiment, further illustrate the present invention. It should be understood that these examples are only used to illustrate the present invention and are not intended to limit the scope of the present invention. For the experimental methods without specific conditions indicated in the following examples, usually follow the conventional conditions or the conditions suggested by the manufacturer. All percentages, ratios, ratios, or parts are by weight unless otherwise indicated.

The unit of weight volume percentage in the present invention is well known to those skilled in the art, for example, it refers to the weight of solute in 100 ml of solution. Unless otherwise defined, all professional and scientific terms used herein have the same meanings as commonly understood by those skilled in the art. In addition, any methods and materials similar or equivalent to those described can be applied to the method of the present invention. The preferred implementation methods and materials described herein are for demonstration purposes only.

### EXAMPLE

General Process

When the preparation route is not included, the raw materials and reagents used in the present invention are known products, which can be synthesized according to methods known in the art, or can be obtained by purchasing commercially available products. All commercially available reagents were used without further purification.

Room temperature means 20-30° C.

If here is no special description in the reaction examples, the reactions are all carried out under a nitrogen atmosphere. The nitrogen atmosphere refers to a nitrogen balloon of about 1 L connected to the reaction flask.

The hydrogenation reaction is usually vacuumized and filled with hydrogen, and the operation is repeated 3 times. The hydrogen atmosphere means that the reaction bottle is connected with a hydrogen balloon of about 1 L.

Microwave reactions use the Biotage® Initiator⁺ Microwave Reactor.

The structures of the compounds of the present invention were determined by nuclear magnetic resonance (NMR) and mass spectroscopy (MS). NMR shifts (δ) are given in units of 10⁻⁶ (ppm). The determination of NMR is to use (Bruker Ascend™ 500 type) nuclear magnetic analyzer, the measurement solvent is deuterated dimethyl sulfoxide (DMSO-d6), deuterated chloroform (CDCl3), deuterated methanol (CD3OD), and the internal standard is tetramethylsilane (TMS). The following abbreviations are used for the multiplicity of NMR signals: s=singlet, brs=broad, d=doublet, t=triplet, m=multiplet. Coupling constants are listed as J values, measured in Hz.

For LC-MS determination, a Thermo liquid mass spectrometer (UltiMate 3000+MSQ PLUS) was used. For HPLC measurement, a Thermo high pressure liquid chromatograph (UltiMate 3000) was used. For Reverse-Phase Preparative Chromatography a Thermo (UltiMate 3000) reverse-phase preparative chromatograph was used. The flash column chromatography uses Agela (FS-9200T) automatic column passing machine, and the silica gel prepacked column uses Santai SEPAFLASH® prepacked column. Yantai Huanghai HSGF254 or Qingdao GF254 silica gel plates are used for thin-layer chromatography silica gel plates, and the specifications of thin-layer chromatography separation and purification products are 0.4 mm to 0.5 mm.

The synthetic method of some intermediates in the present invention is as follows: Intermediate 1

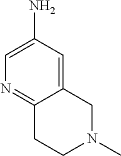

Intermediate 1 was prepared by the following steps:

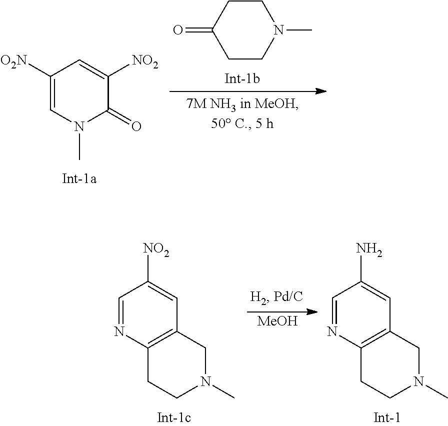

Step 1: Dissolve 1-methyl-3,5-dinitropyridin-2-one Int-1a (1.0 g, 5.02 mmol) in methanol (50 mL), add ammonia methanol solution (7 mol/L, 8.61 mL, 60.27 mmol) and 1-methylpiperidin-4-one Int-1b (625 mg, 5.52 mmol). The reaction mixture was heated to 50° C. and stirred for 5 hours. After cooling to room temperature, let stand for 48 hours, concentrate the reaction solution under reduced pressure, add ethyl acetate (50 mL) to the residue, and filter. The filtrate was concentrated under reduced pressure to obtain a red solid Int-1c (1.0 g), which was directly used in the next reaction. ESI-MS (m/z): 194.4 [M+H]⁺; ¹HNMR (500 MHz, DMSO-d6) δ 9.14 (d, J=2.5 Hz, 1H), 8.36 (d, J=2.5 Hz, 1H), 3.64 (s, 2H), 3.02 (t, J=6.0 Hz, 2H), 2.74 (t, J=6.0 Hz, 2H), 2.39 (s, 3H).

Step 2: Dissolve the compound Int-1c (1.0 g) obtained in the previous step in methanol (30 mL), add 10% Pd—C (400 mg), and react at room temperature under hydrogen atmosphere for 6 hours. Palladium carbon was removed by filtration, and the filtrate was concentrated to obtain a yellow solid Int-1 (800 mg, yield 94.70%). ESI-MS (m/z): 164.2 [M+H]⁺.

### Intermediate 2

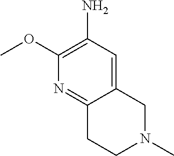

Intermediate 2 was prepared by the following steps:

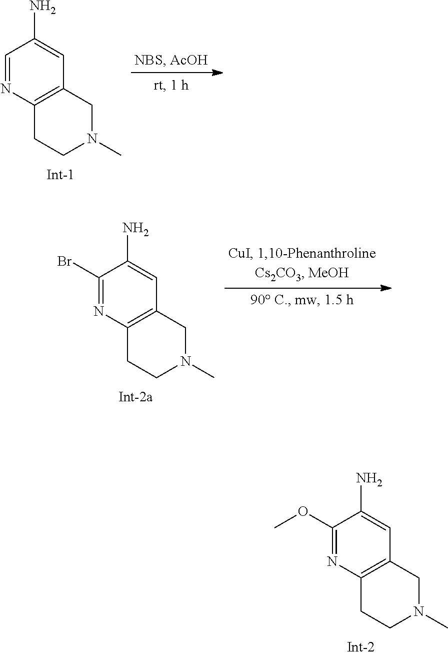

Step 1: Dissolve compound Int-1 (100 mg, 0.61 mmol) in acetic acid (3 mL), add N-bromosuccinimide (109 mg, 0.61 mmol), and stir the reaction mixture at room temperature for 1 Hour. The reaction was quenched by adding saturated aqueous sodium bicarbonate until no bubbles were produced, the aqueous phase was extracted with methanol/dichloromethane (1/20, 50 mL×2), the organic phases were combined, dried over anhydrous sodium sulfate, filtered and concentrated to obtain the compound Int-2a (38 mg, 25% yield). ESI-MS (m/z): 242.3 [M+H]⁺; 1HNMR (500 MHz, DMSO-d6) δ 6.77 (s, 1H), 5.25 (s, 2H), 3.37 (s, 2H), 2.69 (t, J=6.0 Hz, 2H), 2.60 (t, J=6.0 Hz, 2H), 2.32 (s, 3H).

Step 2: Dissolve compound Int-2a (37 mg, 0.15 mmol) in methanol (1 mL), add cuprous iodide (3 mg, 0.015 mmol), 1,10-phenanthroline (3 mg, 0.03 mmol) and cesium carbonate (99 mg, 0.30 mmol). The reaction mixture was replaced with nitrogen and heated to 100° C. by microwave and stirred for 2 hours. The reaction was cooled to room temperature, the reaction solution was concentrated, and the residue was purified by preparative thin-layer chromatography (methanol/dichloromethane/triethylamine=1/10/0.1) to obtain a yellow solid Int-2 (20 mg, yield 67%). ESI-MS (m/z): 194.5 [M+H]⁺; ¹HNMR (500 MHz, DMSO-d6) δ 6.54 (s, 1H), 4.68 (s, 2H), 3.80 (s, 3H), 3.30 (s, 2H), 2.64 (t, J=5.6 Hz, 2H), 2.59 (t, J=5.7 Hz, 2H), 2.31 (s, 3H).

### Intermediate 3

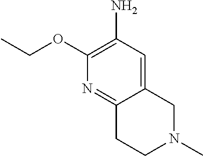

Intermediate 3 was prepared by the following steps:

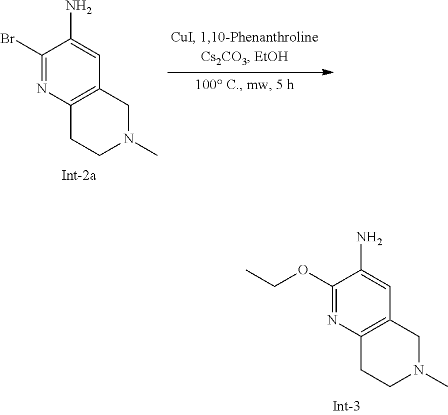

Step 1: Dissolve compound Int-2a (230 mg, 0.94 mmol) in ethanol (2 mL), add cuprous iodide (18 mg, 0.095 mmol), 1,10-phenanthroline (34 mg, 0.18 mmol) and cesium carbonate (619 mg, 1.90 mmol). The reaction mixture was replaced with nitrogen and heated to 100° C. by microwave and stirred for 5 hours. The reaction was cooled to room temperature, filtered, the filtrate was concentrated, and the residue was purified by silica gel column chromatography (methanol/dichloromethane/triethylamine=1/50/0.1) to obtain Int-3 as a yellow solid (113 mg, yield 57%). ESI-MS (m/z): 208.5 [M+H]⁺.

### Intermediate 4

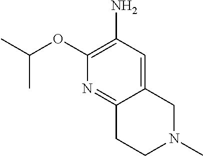 [SMILES: CC(C)Oc1nc2c(cc1N)CN(C)CC2]

Intermediate 4 was prepared by the following steps:

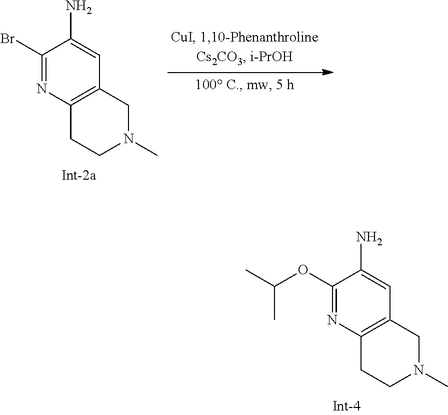 [SMILES: CN1CCc2nc(Br)c(N)cc2C1 ; CC(C)Oc1nc2c(cc1N)CN(C)CC2]

Step 1: Dissolve compound Int-2a (200 mg, 0.82 mmol) in isopropanol (2 mL), add cuprous iodide (15 mg, 0.082 mmol), 1,10-phenanthroline (29 mg, 0.16 mmol) and cesium carbonate (538 mg, 1.65 mmol). The reaction mixture was replaced with nitrogen and heated to 110° C. by microwave and stirred for 5 hours. After the reaction was cooled to room temperature, the reaction solution was concentrated, and the residue was separated by preparative thin-layer chromatography (methanol/dichloromethane/triethylamine=1/10/0.1) to obtain a yellow oil Int-4 (21 mg, yield 11%). ESI-MS (m/z): 222.5 [M+H]⁺.

### Intermediate 5

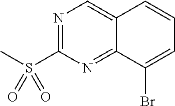

Intermediate 5 was prepared by the following steps:

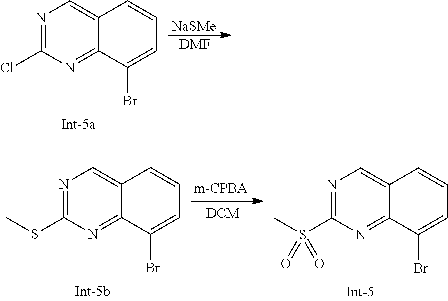

Step 1: Add 20% sodium methylthiolate aqueous solution (1.58 g, 4.52 mmol) dropwise to a solution of 2-chloro-8-bromoquinazoline Int-5a (1.0 g, 4.11 mmol) in DMF (10 mL) at 0° C. ice bath. The reaction mixture was stirred for an additional 30 minutes under the ice bath, then water (100 mL) was added. The reaction solution was filtered, the filter cake was washed with cold water, and dried to obtain a yellow solid Int-5b (1.02 g, yield 97%). ESI-MS (m/z): 255.2 [M+H]⁺.

Step 2: At 0° C. ice bath, add m-chloroperoxybenzoic acid (1.95 g, content 85%, 9.59 mmol,) to a suspension of compound Int-5b (1.02 g, 4.00 mmol) in dichloromethane (20 mL). The reaction mixture was raised to room temperature and stirred for 2 hours. The reaction solution was diluted with water, and the mixture was extracted with dichloromethane. The organic phase was washed with water, dried over anhydrous sodium sulfate, filtered and concentrated. The residue was purified by silica gel column chromatography (petroleum ether/ethyl acetate=1/1 to 100% ethyl acetate) to obtain Int-5 as a white solid (1.05 g, yield 91%). ESI-MS (m/z): 287.1 [M+H]⁺.

### Intermediate 6

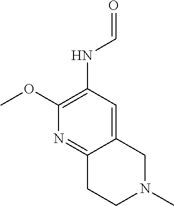

Intermediate 6 was prepared by the following steps:

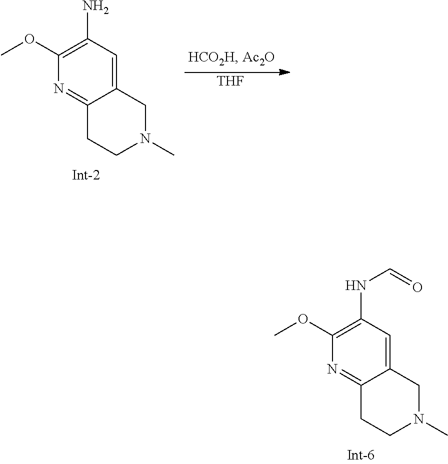

Step 1: Formic acid (2.14 g, 46.57 mmol, 1.76 mL) was added dropwise to acetic anhydride (3.17 g, 31.05 mmol, 2.93 mL) in an ice bath at 0° C., then raised to room temperature and stirred for 1 hour. Then the mixture was re-cooled to 0° C., added dropwise to a solution of Int-2 (500 mg, 2.59 mmol) in tetrahydrofuran (10 mL) (0° C.), and then raised to room temperature and stirred for 30 minutes. The reaction solution was diluted with dichloromethane and washed three times with saturated sodium bicarbonate solution. The organic phase was dried over anhydrous sodium sulfate, filtered and concentrated. The residue was purified by silica gel column chromatography (dichloromethane/methanol=10/1) to obtain Int-6 as a white solid (550 mg, yield 96%). ESI-MS (m/z): 222.5 [M+H]⁺.

### Intermediate 7

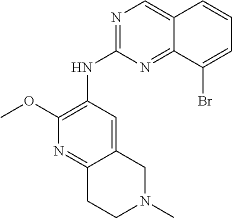 [SMILES: COc1nc2c(cc1Nc1ncc3cccc(Br)c3n1)CN(C)CC2]

Intermediate 7 was prepared by the following steps:

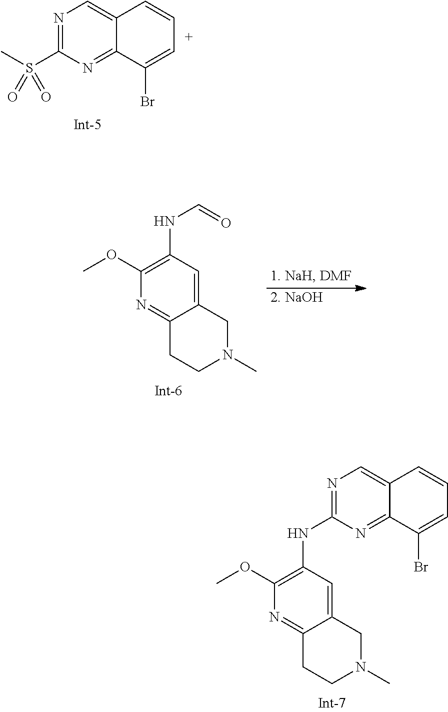

Step 1: At 0° C. ice bath, add sodium hydride (45 mg, content 60%, 1.13 mmol) to a solution of Int-6 (250 mg, 1.13 mmol) in anhydrous DMF (5 mL). After the mixture was stirred at room temperature for 20 minutes, it was cooled to 0° C., a solution of Int-5 (356 mg, 1.24 mmol) in anhydrous DMF (5 mL) was added, and stirred at room temperature for 2 hours. Then, 2N aqueous sodium hydroxide solution (3 mL) and methanol (3 mL) were added to the reaction solution, and stirred at room temperature for 1 hour. The reaction solution was diluted with 50 mL of water, filtered, the filter cake was washed with water, and dried to obtain a yellow solid Int-7 (440 mg, yield 97%). ESI-MS (m/z): 400.2 [M+H]⁺.

### Intermediate 8

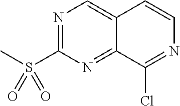

Intermediate 8 was prepared by the following steps:

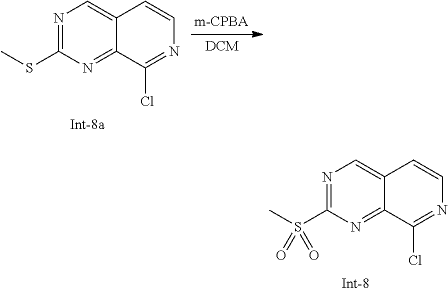

Step 1: Dissolve compound Int-8a (300 mg, 1.42 mmol) in dichloromethane (10 mL), add m-CPBA (604 mg, content 85%, 2.98 mmol) under ice bath. After the feeding is finished, the reaction was continued for 4 hours under ice bath, and the raw materials were detected by LCMS to be completely reacted. The reaction solution was concentrated, and the residue was purified by silica gel column chromatography to obtain a light yellow solid Int-8 (300 mg, yield 86%). ESI-MS (m/z): 244.3 [M+H]⁺.

### Intermediate 9

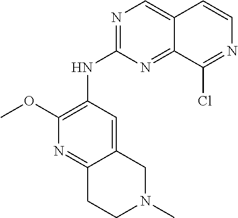 [SMILES: COc1nc2c(cc1Nc1ncc3ccnc(Cl)c3n1)CN(C)CC2]

Intermediate 9 was prepared by the following steps:

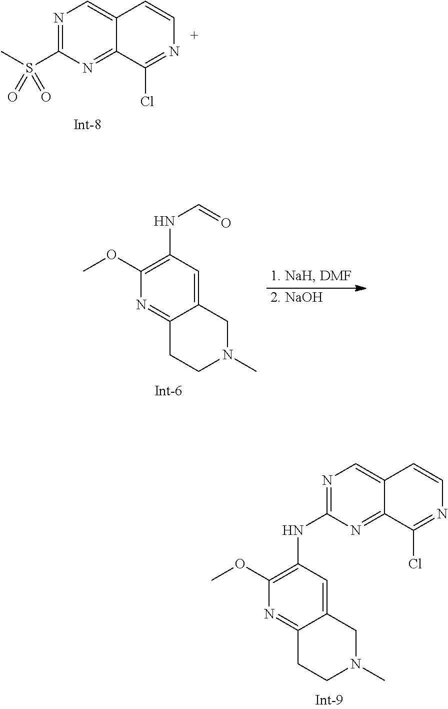

Step 1: Compound Int-6 (230 mg, 1.04 mmol) was dissolved in anhydrous DMF (10 mL), and NaH (42 mg, content 60%, 1.04 mmol) was added under ice bath. After the mixture was stirred at room temperature for 30 minutes, it was cooled to 0° C., and a solution of Int-8 (244 mg, 1.14 mmol) in DMF (3 mL) was added dropwise. After the dropwise addition, the mixture was reacted at room temperature for 2 hours, and the raw materials were detected by LCMS to be completely reacted. 0.1N NaOH solution (1 mL) was added to the reaction solution, and stirred at room temperature for 1 hour. The reaction solution was poured into water (40 mL), and a yellow solid precipitated out. The solid was collected by filtration and dried to obtain Int-9 (230 mg, yield 62%). ESI-MS (m/z): 357.2 [M+H]⁺.

### Intermediate 10

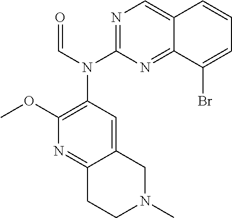 [SMILES: COc1nc2c(cc1N(C=O)c1ncc3cccc(Br)c3n1)CN(C)CC2]

Intermediate 10 was prepared by the following steps:

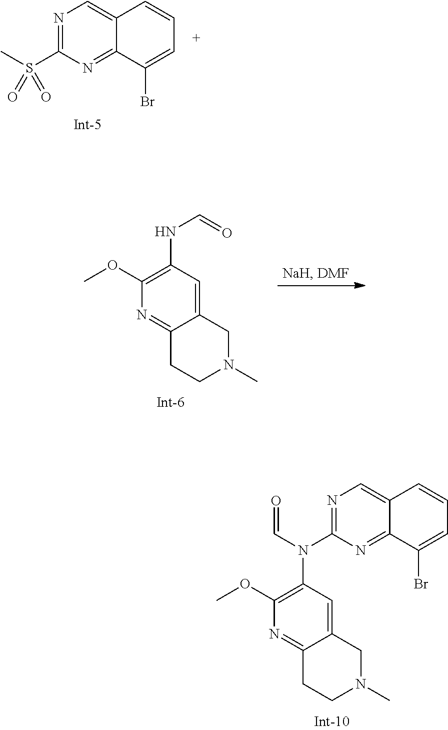

Step 1: Add sodium hydride (45 mg, content 60%, 1.13 mmol) to a solution of Int-6 (250 mg, 1.13 mmol) in anhydrous DMF (5 mL) at 0° C. ice bath. After the reaction mixture was stirred at room temperature for 20 minutes, it was cooled to 0° C., a solution of Int-5 (356 mg, 1.24 mmol) in anhydrous DMF (5 mL) was added, and the reaction mixture was warmed to room temperature and stirred for 2 hours. Water (50 mL) was added to quench the reaction, and the mixture was extracted with ethyl acetate. The organic phases were combined, washed with saturated brine, dried over anhydrous sodium sulfate, filtered and concentrated. The residue was purified by silica gel column chromatography (dichloromethane/methanol=20/1) to obtain Int-10 (380 mg, yield 78%) as a yellow solid. ESI-MS (m/z): 428.2 [M+H]⁺.

### Intermediate 11

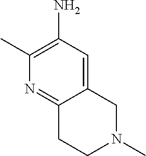

Intermediate 11 was prepared by the following steps:

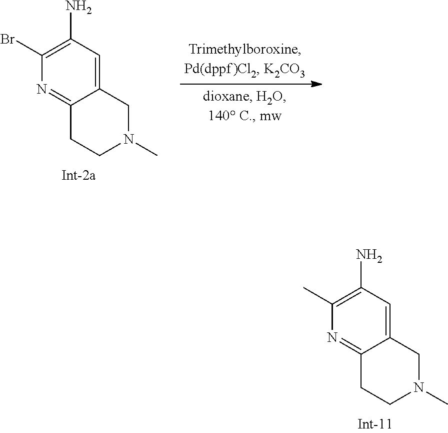

Step 1: Dissolve compound Int-2a (100 mg, 0.41 mmol) and trimethylboroxine (148 mg, 1.19 mmol) in dioxane (1.5 mL) and water (0.15 mL), and add potassium carbonate (171 mg, 1.24 mmol), Pd(dppf)Cl₂ (30 mg, 0.041 mmol). After the reaction system was replaced with nitrogen, it was heated to 140° C. with microwave and stirred for 1 hour. The reaction was cooled to room temperature, the reaction mixture was filtered through celite, and the filtrate was concentrated. The residue was purified by silica gel column chromatography (methanol/dichloromethane=1/20) to obtain Int-11 (50 mg, yield 68%) as a yellow solid. ESI-MS (m/z): 178.6 [M+H]⁺.

### Intermediate 12

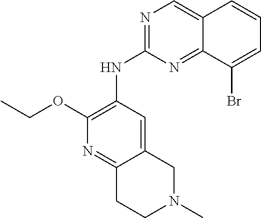 [SMILES: CCOc1nc2c(cc1Nc1ncc3cccc(Br)c3n1)CN(C)CC2]

Starting from Int-3, Int-12 can be obtained by adopting the reaction steps similar to intermediates 6 and 7. ESI-MS (m/z): 414.2 [M+H]⁺.

### Intermediate 13

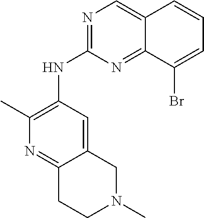 [SMILES: Cc1nc2c(cc1Nc1ncc3cccc(Br)c3n1)CN(C)CC2]

Starting from Int-11, Int-13 can be obtained by adopting the reaction steps similar to intermediates 6 and 7. ESI-MS (m/z): 384.2 [M+H]⁺.

### Intermediate 14

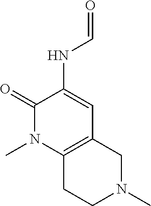 [SMILES: CN1CCc2c(cc(NC=O)c(=O)n2C)C1]

Intermediate 14 was prepared by the following steps:

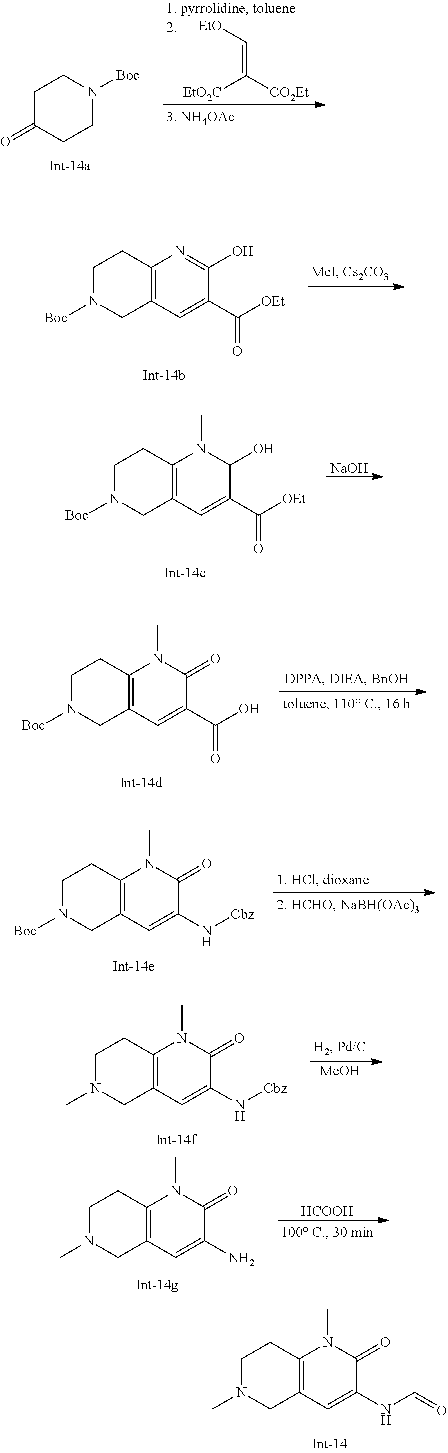

Step 1: Dissolve compound Int-14a (5 g, 25.09 mmol) and tetrahydropyrrole (2.68 g, 37.64 mmol, 3.13 mL) in toluene (50 mL), and heat to reflux for 18 hours with a water separator. The reaction solution was concentrated, the residue was dissolved in 1,4-dioxane (50 mL), and then diethyl ethoxymethylene malonate (5.97 g, 27.60 mmol, 5.53 mL) was added, and the reaction mixture was heated to reflux and stirred for 6 hours. After the reaction solution was cooled to room temperature, ammonium acetate (3.29 g, 42.66 mmol) was added, followed by heating to reflux for 1 hour. The reaction solution was concentrated, and the residue was separated and purified by silica gel column chromatography (100% ethyl acetate) to obtain a yellow solid compound Int-14b (2.3 g, yield 28%). ESI-MS (m/z): 323.4 [M+H]⁺; ¹HNMR (500 MHz, DMSO-d6) δ 11.99 (s, 1H), 7.90 (s, 1H), 4.25 (s, 2H), 4.19 (q, J=7.1 Hz, 2H), 3.54 (t, J=5.8 Hz, 2H), 2.60 (t, J=5.9 Hz, 2H), 1.42 (s, 9H), 1.25 (t, J=7.1 Hz, 3H).

Step 2: Compound Int-14b (1.1 g, 3.41 mmol) was dissolved in DMF (20 mL), and cesium carbonate (1.67 g, 5.12 mmol) and iodomethane (484 mg, 3.41 mol) were successively added at 0° C. ice bath. The mixture was warmed to room temperature and stirred for 1 hour. After the reaction was complete, water was added to quench the reaction, and the aqueous phase was extracted with ethyl acetate. The organic phases were combined, washed with saturated brine, dried over anhydrous sodium sulfate, filtered, and concentrated to obtain Int-14c (1.1 g, yield 95%) as a yellow oily liquid. ESI-MS (m/z): 337.3 [M+H]⁺; ₁H NMR (500 MHz, DMSO-d6) δ 7.84 (s, 1H), 4.28 (s, 2H), 4.19 (q, J=7.1 Hz, 2H), 3.57 (t, J=5.9 Hz, 2H), 3.40 (s, 3H), 2.80 (t, J=5.9 Hz, 2H), 1.41 (s, 9H), 1.24 (t, J=7.1 Hz, 3H).

Step 3: Compound Int-14c (1.1 g, 3.27 mmol) was dissolved in ethanol (10 mL), 1N aqueous sodium hydroxide solution (9.8 mL) was added, and the mixture was stirred at room temperature for 2 hours. Adjust the pH value to 6 with 6N hydrochloric acid aqueous solution, and then dilute with water (100 mL). The precipitate was filtered, the filter cake was washed with water, and dried to obtain compound Int-14d (830 mg, yield 82%) as a yellow solid. ESI-MS (m/z): 309.3 [M+H]⁺.

Step 4: Dissolve compound Int-14d (830 mg, 2.69 mmol) in toluene (10 mL), add diphenylphosphoryl azide (2.22 g, 8.08 mmol), benzyl alcohol (873 mg, 8.08 mmol) and N,N-Diisopropylethylamine (1.39 mg, 10.77 mmol), the reaction mixture was heated to 120° C. and stirred for 16 hours. The reaction solution was concentrated, and the residue was separated and purified by silica gel column chromatography (petroleum ether/ethyl acetate=1/1) to obtain a yellow solid compound Int-14e (930 mg, yield 83%). ESI-MS (m/z): 414.3 [M+H]⁺; ¹H NMR (500 MHz, methanol-d4) δ 7.84 (s, 1H), 7.48-7.31 (m, 6H), 5.21 (s, 2H), 4.36 (s, 2H), 3.71 (t, J=6.3 Hz, 2H), 3.56 (s, 3H), 2.80-2.77 (m, 2H), 1.50 (s, 9H).

Step 5: Dissolve compound Int-14e (930 mg, 2.25 mmol) in dichloromethane (10 mL), add hydrochloric acid/dioxane solution (4 N, 2.25 mL), and stir the reaction mixture at room temperature for 1 hour. The reaction solution was concentrated, the residue was dissolved in methanol (10 mL), aqueous formaldehyde (1.09 g, 11.25 mmol, content 35%) and sodium triacetoxyborohydride (1.43 g, 6.75 mmol) were added, and the reaction mixture was stirred at room temperature for 2 hours. Adjust the pH value to 8 with saturated aqueous sodium bicarbonate solution, dilute with water (50 mL), and extract the aqueous phase with a mixed solvent of dichloromethane and methanol (v/v=10/1). The organic phases were combined, dried over anhydrous sodium sulfate, filtered, and concentrated to obtain a yellow solid compound Int-14f (700 mg, yield 95%). ESI-MS (m/z): 328.3 [M+H]⁺; 1H NMR (500 MHz, DMSO-d6) δ 8.29 (s, 1H), 7.58 (s, 1H), 7.44-7.31 (m, 5H), 5.15 (s, 2H), 3.45 (s, 3H), 3.26-3.22 (m, 2H), 2.73 (t, J=5.7 Hz, 2H), 2.60 (t, J=5.8 Hz, 2H), 2.31 (s, 3H).

Step 6: Compound Int-14f (700 mg, 2.14 mmol) was dissolved in methanol (10 mL), 10% palladium on carbon (70 mg) was added, and the mixture was stirred at room temperature under hydrogen atmosphere for 1 hour. The palladium carbon was filtered off with celite, the filter cake was washed with methanol, and the filtrate was concentrated to obtain Int-14g (380 mg, yield 92%) as a yellow solid. ESI-MS (m/z): 194.4 [M+H]⁺.

Step 7: Compound Int-14g (100 mg, 0.52 mmol) was dissolved in formic acid (2 mL), and the reaction solution was stirred at 100° C. for 30 min. After the reaction was complete, the reaction solution was concentrated, and the residue was separated by column chromatography (DCM/MeOH=20:1) to obtain compound Int-14 (90 mg, 0.41 mmol), yield 78%, light yellow solid. ESI-MS (m/z): 222.5 [M+H]⁺.

### Intermediate 15

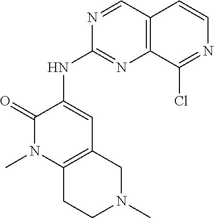 [SMILES: CN1CCc2c(cc(Nc3ncc4ccnc(Cl)c4n3)c(=O)n2C)C1]

Intermediate 15 was prepared by the following steps:

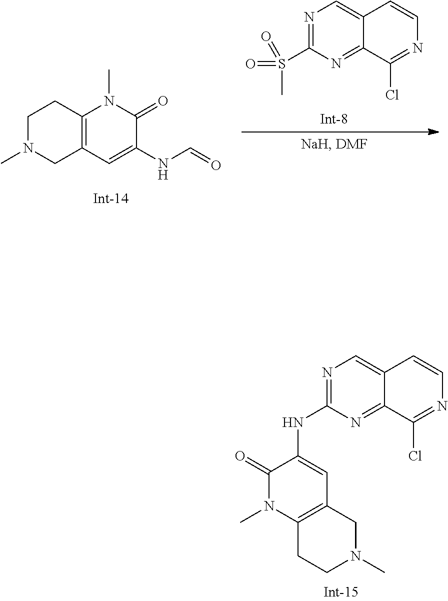 [SMILES: CN1CCc2c(cc(NC=O)c(=O)n2C)C1 ; CS(=O)(=O)c1ncc2ccnc(Cl)c2n1 ; CN1CCc2c(cc(Nc3ncc4ccnc(Cl)c4n3)c(=O)n2C)C1]

Step 1: Dissolve Int-14 (82 mg, 0.37 mmol) in DMF (3 mL), add NaH (30 mg, 0.74 mmol) under ice-cooling, and stir the reaction system at 0° C. for 1 h. Then, DMF (1 mL) solution of Int-8 (90 mg, 0.37 mmol) was added, and the reaction solution was stirred at room temperature for 1 h. After the reaction was complete, the reaction solution was poured into water (50 mL), filtered with suction and dried to obtain compound Int-15 (85 mg, 0.24 mmol), yield 64.5%, light yellow solid. ESI-MS (m/z): 357.2 [M+H]⁺.

### Intermediate 16

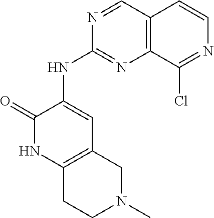 [SMILES: CN1CCc2[nH]c(=O)c(Nc3ncc4ccnc(Cl)c4n3)cc2C1]

Intermediate 16 was prepared by the following steps:

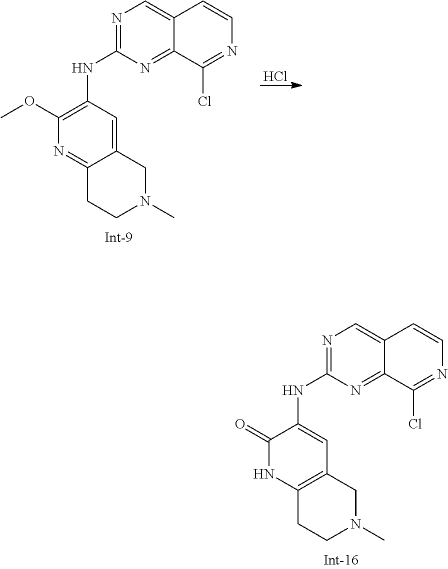 [SMILES: COc1nc2c(cc1Nc1ncc3ccnc(Cl)c3n1)CN(C)CC2 ; Cl ; CN1CCc2[nH]c(=O)c(Nc3ncc4ccnc(Cl)c4n3)cc2C1]

Compound Int-9 (500 mg, 1.4 mmol) was dissolved in dichloromethane (20 mL), and a solution of BBr₃ in dichloromethane (1M, 1.68 mL, 4.2 mmol) was slowly added dropwise at 0° C. After the addition was complete, it was slowly warmed up to room temperature overnight, and LCMS monitored the complete reaction of the starting material. The reaction solution was quenched with methanol, concentrated, and the residual solid was slurried with ethyl acetate, filtered, and dried to obtain 550 mg of compound Int-16 (HBr salt), brown solid, yield 92.64%, ESI-MS (m/z): 343.3 [M+H]⁺.

### Intermediate 17

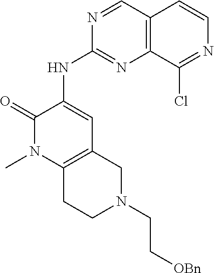 [SMILES: Cn1c2c(cc(Nc3ncc4ccnc(Cl)c4n3)c1=O)CN(CCOCc1ccccc1)CC2]

Intermediate 17 was prepared by the following steps:

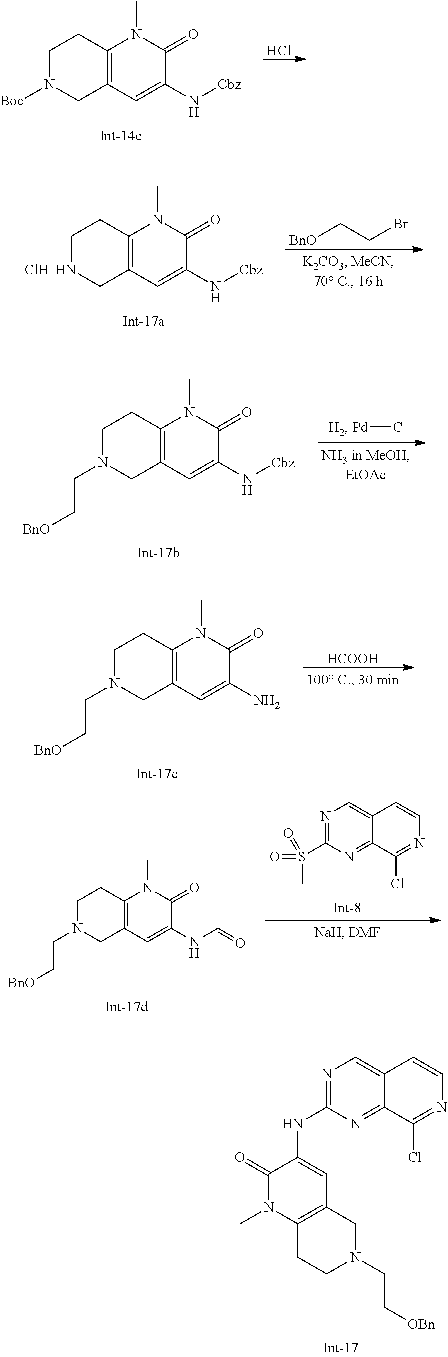

Step 1: Int-14e (200 mg, 0.48 mmol) was added to hydrochloric acid/dioxane (3 mL), and the reaction solution was stirred at room temperature for 2 h. After the reaction was complete, the reaction solution was concentrated to obtain Int-17a (160 mg, 0.46 mmol), with a yield of 94%, as a pale yellow solid. ESI-MS (m/z): 314.3 [M+H]⁺.

Step 2: Int-17a (160 mg, 0.46 mmol) was dissolved in acetonitrile (5 mL), and potassium carbonate (190 mg, 1.38 mmol) and benzyl 2-bromoethyl ether (198 mg, 0.92 mmol), the reaction system was stirred at 70° C. for 16 h. After the reaction was complete, water was added to quench the reaction, and the aqueous phase was extracted with ethyl acetate. The organic phases were combined, washed with saturated brine, dried over anhydrous sodium sulfate, filtered and concentrated. The residue was separated by column chromatography (DCM/MeOH=30/1) to obtain compound Int-17b (160 mg, 0.36 mmol), yield 78%, light yellow solid. ESI-MS (m/z): 448.2 [M+H]⁺.

Step 3: Dissolve compound Int-17b (160 mg, 0.36 mmol) in a mixed solution of ethyl acetate (3 mL) and ammonia/methanol (3 mL), add palladium on carbon (32 mg, 20% wt), and the mixture was stirred at room temperature under hydrogen atmosphere for 1 hour. After the reaction was complete, the palladium on carbon was filtered out with diatomaceous earth, the filter cake was washed with methanol, and the filtrate was concentrated to obtain compound Int-17c (100 mg, 0.32 mmol), with a yield of 89.2%, a light yellow solid. ESI-MS (m/z): 314.3 [M+H]⁺.

Step 4: Compound Int-17c (100 mg, 0.32 mmol) was dissolved in formic acid (2 mL), and the reaction solution was stirred at 100° C. for 30 min. After the reaction was complete, the reaction solution was concentrated, and the residue was separated by column chromatography (DCM:MeOH=20:1) to obtain compound Int-17d (75 mg, 0.22 mmol), yield 68.8%, light yellow solid. ESI-MS (m/z): 342.3 [M+H]⁺.

Step 5: Dissolve Int-17d (75 mg, 0.22 mmol) in DMF (3 mL), add NaH (44 mg, 1.10 mmol) under ice bath, and stir the reaction system at 0° C. for 1 h. Then, Int-8 (53.5 mg, 0.22 mmol) was dissolved in DMF (1 mL), and the reaction solution was stirred at room temperature for 1 h. After the reaction was complete, the reaction solution was poured into water (50 mL), filtered with suction and dried to obtain compound Int-17 (85 mg, 0.18 mmol), yield 81%, light yellow solid. ESI-MS (m/z): 477.1 [M+H]⁺.

### Intermediate 18

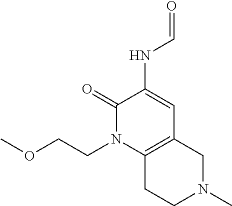

Intermediate 18 was prepared by the following steps:

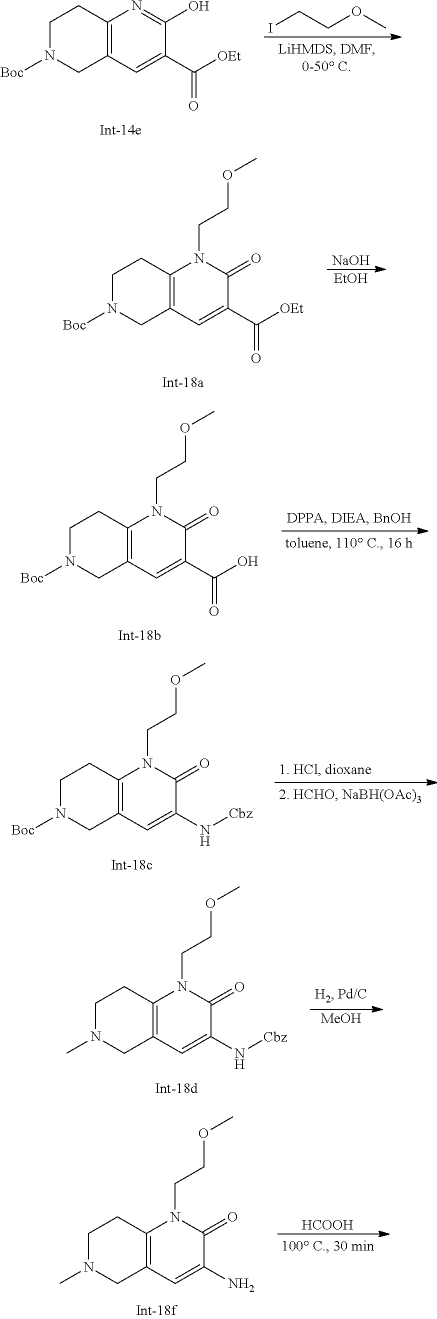

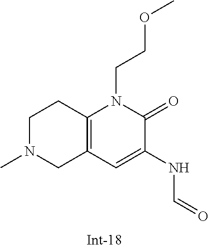 [SMILES: COCCn1c2c(cc(NC=O)c1=O)CN(C)CC2]

Step 1: Compound Int-14e (1.28 g, 3.97 mmol) was dissolved in DMF (15 mL), lithium bistrimethylsilylamide (1 mol/L in THF, 4.76 mL) was added dropwise under ice bath, and the mixture was stirred at 0° C. for 30 min. Then 1-iodo-2-methoxyethane (739 mg, 3.97 mmol) was added, raised to 50° C. and stirred for 16 hours. After the reaction was complete, water was added to quench the reaction, and the aqueous phase was extracted with ethyl acetate. The organic phases were combined, washed with saturated brine, dried over anhydrous sodium sulfate, filtered and concentrated. The residue was separated by column chromatography (PE/EA=1/2) to obtain compound Int-18a (600 mg, 1.58 mmol), yield 39.7%, light yellow solid. ESI-MS (m/z): 381.6 [M+H]⁺.

Step 2: Dissolve compound Int-18a (850 mg, 2.23 mmol) in ethanol (10 mL), add 1N aqueous sodium hydroxide solution (6.7 mL), and stir the mixture at room temperature for 2 hours. Adjust the pH to 6 with 6N aqueous hydrochloric acid, and dilute with water (60 mL). The precipitate was filtered, the filter cake was washed with water, and dried to obtain a yellow solid compound Int-18b (700 mg, 1.99 mmol), with a yield of 88.9%, a light yellow solid. ESI-MS (m/z): 353.3 [M+H]⁺.

Step 3: Dissolve compound Int-18b (700 mg, 1.99 mmol) in toluene (10 mL), add diphenylphosphoryl azide (1.64 g, 5.96 mmol), benzyl alcohol (644 mg, 5.96 mmol) and N,N-Diisopropylethylamine (1.03 g, 7.95 mmol), and the mixture was heated to 120° C. and stirred for 16 hours. The reaction solution was concentrated, and the residue was separated by column chromatography (PE/EA=2/3) to obtain a yellow solid compound Int-18c (650 mg, 1.42 mmol), yield 71.5%, light yellow solid. ESI-MS (m/z): 458.4 [M+H]⁺.

Step 4: Dissolve compound Int-18c (650 mg, 1.42 mmol) in dichloromethane (5 mL), add hydrochloric acid/dioxane solution (4 mol/L, 1.42 mL), and stir the mixture at room temperature 1 hour. The reaction solution was concentrated, the residue was dissolved in methanol (5 mL), aqueous formaldehyde (415 mg, 4.26 mmol, 35% purity) and sodium triacetoxyborohydride (903 mg, 4.26 mmol) were added, and the mixture was stirred at room temperature 2 hours. The pH value was adjusted to 8 with saturated aqueous sodium bicarbonate solution, diluted with water (30 mL), and the aqueous phase was extracted with a mixed solvent of dichloromethane and methanol (v/v=10/1). The organic phases were combined, dried over anhydrous sodium sulfate, filtered, and concentrated to obtain compound Int-18d (500 mg, 1.35 mmol) as a yellow solid, with a yield of 94.7%, as a light yellow solid. ESI-MS (m/z): 372.4 [M+H]⁺.

Step 5: Compound Int-18d (500 mg, 1.09 mmol) was dissolved in methanol (10 mL), 10% palladium on carbon (50 mg, 10% wt) was added, and the mixture was stirred at room temperature under hydrogen atmosphere for 2 hours. The palladium carbon was filtered out with celite, the filter cake was washed with methanol, and the filtrate was concentrated to obtain a yellow solid compound Int-18f (250 mg, 1.05 mmol), with a yield of 96.4%, a light yellow solid. ESI-MS (m/z): 238.6 [M+H]⁺.

Step 6: Compound Int-18f (120 mg, 0.51 mmol) was dissolved in formic acid (2 mL), and the reaction solution was stirred at 100° C. for 30 min. After the reaction was complete, the reaction solution was concentrated, and the residue was separated by silica gel column chromatography (DCM:MeOH=20:1) to obtain compound Int-18 (70 mg, 0.26 mmol), yield 52.2%, light yellow solid. ESI-MS (m/z): 266.4 [M+H]⁺.

The synthetic method of embodiment compound among the present invention is as follows:

### Example 1

### N-(2-methoxy-6-methyl-5,6,7,8-tetrahydro-1,6-naphthyridin-3-yl)-8-phenylquinolin-2-amine

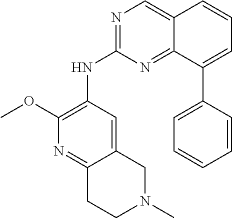 [SMILES: COc1nc2c(cc1Nc1ncc3cccc(-c4ccccc4)c3n1)CN(C)CC2]

Compound 1 was prepared by the following steps:

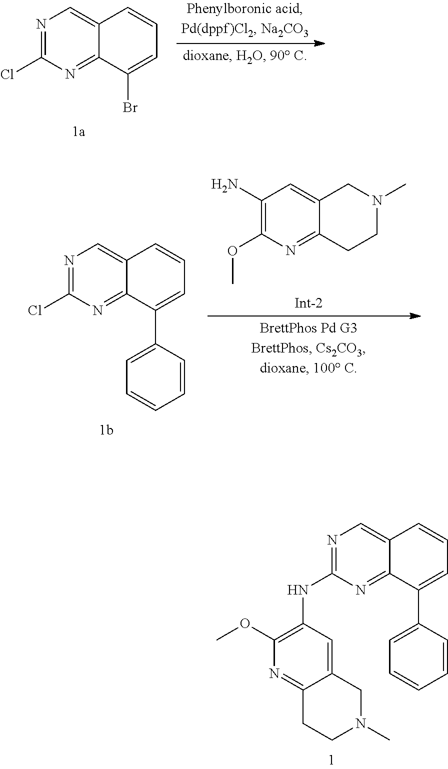 [SMILES: Clc1ncc2cccc(Br)c2n1 ; Clc1ncc2cccc(-c3ccccc3)c2n1 ; COc1nc2c(cc1N)CN(C)CC2 ; COc1nc2c(cc1Nc1ncc3cccc(-c4ccccc4)c3n1)CN(C)CC2]

Step 1: Dissolve 2-chloro-8-bromoquinazoline 1a (200 mg, 0.82 mmol) and phenylboronic acid (120 mg, 0.98 mmol) in a mixed solvent of 1,4-dioxane (5 mL) and water (0.5 mL), sodium carbonate (348 mg, 3.29 mmol) and Pd(dppf)Cl₂ (30 mg, 0.041 mmol) were added, and the reaction system was heated to 90° C. and stirred for 18 hours after replacing nitrogen. After the reaction solution was cooled to room temperature, the reaction solution was filtered through celite, and the filtrate was concentrated. The residue was purified by silica gel column chromatography (petroleum ether/ethyl acetate=20/1) to obtain yellow solid 1b (170 mg, yield 85%). ESI-MS (m/z): 241.3 [M+H]⁺.

Step 2: Compound 1b (18 mg, 0.077 mmol) and Int-2 (15 mg, 0.077 mmol) were dissolved in 1,4-dioxane (3 mL), and BrettPhos Pd G3 (7 mg, 7.7 umol), BrettPhos (8 mg, 15 umol) and cesium carbonate (50 mg, 0.15 mmol) were added. The reaction system was replaced with nitrogen and then heated to 100° C. and stirred for 18 hours. After the reaction solution was cooled to room temperature, the reaction solution was filtered through celite, and the filtrate was concentrated. The residue was purified by Prep-HPLC to obtain white solid 1 (8 mg, yield 25%). ESI-MS (m/z): 398.3 [M+H]⁺; ¹HNMR (500 MHz, DMSO-d6) δ 9.38 (s, 1H), 8.25 (s, 1H), 8.14 (s, 1H), 7.97 (dd, J=8.0, 1.4 Hz, 1H), 7.83 (dd, J=7.2, 1.5 Hz, 1H), 7.68-7.63 (m, 2H), 7.55-7.43 (m, 4H), 3.90 (s, 3H), 3.14 (s, 2H), 2.72 (t, J=5.9 Hz, 2H), 2.62 (t, J=5.9 Hz, 2H), 2.37 (s, 3H).

### Example 2

### N-(2-methoxy-6-methyl-5,6,7,8-tetrahydro-1,6-naphthyridin-3-yl)-8-(pyrazolo[1,5-a]pyridin-3-yl)quinazolin-2-amine

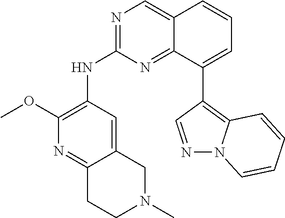 [SMILES: COc1nc2c(cc1Nc1ncc3cccc(-c4cnn5ccccc45)c3n1)CN(C)CC2]

Compound 2 can be obtained by using pyrazolo[1,5-a]pyridine-3-boronic acid pinacol ester instead of phenylboronic acid in the first step in Example 1, and using a similar method and reaction steps. ESI-MS (m/z): 437.4 [M+H]⁺; 1HNMR (500 MHz, DMSO-d6) δ 9.40 (s, 1H), 8.83 (d, J=7.0 Hz, 1H), 8.42 (s, 1H), 8.20 (s, 1H), 8.07 (s, 1H), 8.00-7.91 (m, 2H), 7.59 (d, J=8.9 Hz, 1H), 7.52 (t, J=7.6 Hz, 1H), 7.22-7.15 (m, 1H), 6.98 (t, J=6.7 Hz, 1H), 3.89 (s, 3H), 2.91 (s, 2H), 2.70 (t, J=5.9 Hz, 2H), 2.59 (t, J=5.8 Hz, 2H), 2.34 (s, 3H).

### Example 3

### N-(2-methoxy-6-methyl-5,6,7,8-tetrahydro-1,6-naphthyridin-3-yl)-8-(1-methyl-1H-indazole-5-yl)quinazolin-2-amine

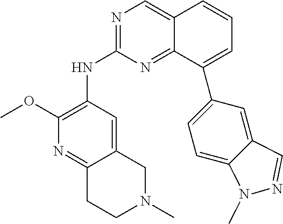 [SMILES: COc1nc2c(cc1Nc1ncc3cccc(-c4ccc5c(cnn5C)c4)c3n1)CN(C)CC2]

Compound 3 can be obtained by using 1-methylindazole-5-boronic acid instead of phenylboronic acid in the first step in Example 1, and using a similar method and reaction steps. ESI-MS (m/z): 452.2 [M+H]⁺; ¹HNMR (500 MHz, DMSO-d6) δ 9.38 (s, 1H), 8.13-8.09 (m, 3H), 7.97-7.93 (m, 2H), 7.86 (dd, J=7.2, 1.4 Hz, 1H), 7.78 (d, J=8.6 Hz, 1H), 7.65 (dd, J=8.5, 1.5 Hz, 1H), 7.51 (t, J=7.6 Hz, 1H), 4.14 (s, 3H), 3.89 (s, 3H), 2.66 (t, J=6.0 Hz, 2H), 2.54 (s, 2H), 2.53-2.52 (m, 2H), 2.18 (s, 3H).

### Example 4

### 8-(2-fluorophenyl)-N-(2-methoxy-6-methyl-5,6,7,8-tetrahydro-1,6-naphthyridin-3-yl)quinazoline-2-amine

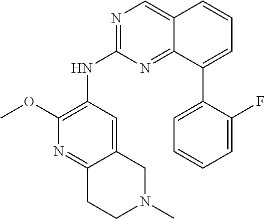 [SMILES: COc1nc2c(cc1Nc1ncc3cccc(-c4ccccc4F)c3n1)CN(C)CC2]

Compound 4 can be obtained by replacing the phenylboronic acid in the first step in Example 1 with 2-fluorophenylboronic acid, and using a similar method and reaction steps. ESI-MS (m/z): 416.3 [M+H]⁺; 1HNMR (500 MHz, DMSO-d6) δ 9.40 (s, 1H), 8.14 (s, 1H), 8.10 (s, 1H), 8.04 (d, J=8.0 Hz, 1H), 7.84 (d, J=7.0 Hz, 1H), 7.60-7.50 (m, 3H), 7.46-7.35 (m, 2H), 3.91 (s, 3H), 3.08 (s, 2H), 2.71 (d, J=5.9 Hz, 2H), 2.62 (t, J=5.9 Hz, 2H), 2.39 (s, 3H).

### Example 5

### N-(2-methoxy-6-methyl-5,6,7,8-tetrahydro-1,6-naphthyridin-3-yl)-8-(2-methoxyphen yl)quinazole Lin-2-amine

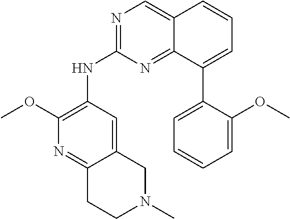

Compound 5 can be obtained by using 2-methoxyphenylboronic acid instead of phenylboronic acid in the first step in Example 1, and using a similar method and reaction steps. ESI-MS (428.2): m/z [M+H]⁺; 1HNMR (500 MHz, DMSO-d6) δ 9.35 (s, 1H), 8.11 (s, 1H), 8.01 (s, 1H), 7.99-7.94 (m, 1H), 7.72 (d, J=7.1 Hz, 1H), 7.50-7.45 (m, 2H), 7.29 (dd, J=7.4, 1.6 Hz, 1H), 7.21 (d, J=8.3 Hz, 1H), 7.11 (t, J=7.4 Hz, 1H), 3.91 (s, 3H), 3.61 (s, 3H), 3.06 (s, 2H), 2.70 (t, J=5.9 Hz, 2H), 2.60 (t, J=5.9 Hz, 2H), 2.39 (s, 3H).

### Example 6

### N-(2-methoxy-6-methyl-5,6,7,8-tetrahydro-1,6-naphthyridin-3-yl)-8-(3-methoxyphen yl)quinazole Lin-2-amine

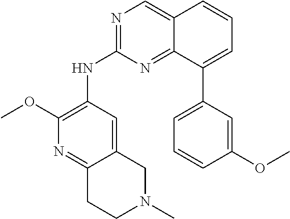

Compound 6 can be obtained by using 3-methoxyphenylboronic acid instead of phenylboronic acid in the first step in Example 1, and using a similar method and reaction steps. ESI-MS (m/z): 428.3 [M+H]⁺; 1HNMR (500 MHz, DMSO-d6) δ 9.38 (s, 1H), 8.32 (s, 1H), 8.14 (s, 1H), 7.98 (d, J=8.1 Hz, 1H), 7.84 (d, J=7.0 Hz, 1H), 7.55-7.43 (m, 2H), 7.20 (s, 2H), 7.05 (br s, 1H), 3.91 (s, 3H), 3.77 (s, 3H), 3.16 (s, 2H), 2.74 (br s, 2H), 2.66 (br s, 2H), 2.39 (s, 3H).

### Example 7

### 8-(3,6-dihydro-2H-pyran-4-yl)-N-(2-methoxy-6-methyl-5,6,7,8-tetrahydro-1,6-naphthalene pyridin-3-yl)quinazolin-2-amine

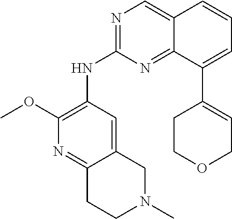 [SMILES: COc1nc2c(cc1Nc1ncc3cccc(C4=CCOCC4)c3n1)CN(C)CC2]

Compound 7 can be obtained by using 3,6-dihydro-2H-pyran-4-boronic acid pinacol ester instead of phenylboronic acid in the first step in Example 1, and using a similar method and reaction steps. ESI-MS (m/z): 404.1 [M+H]⁺; 1HNMR (500 MHz, DMSO-d6) δ 9.30 (s, 1H), 8.33 (s, 1H), 8.30 (s, 1H), 7.85 (dd, J=8.0, 1.4 Hz, 1H), 7.65 (dd, J=7.2, 1.4 Hz, 1H), 7.37 (t, J=7.6 Hz, 1H), 6.04 (s, 1H), 4.25 (q, J=2.7 Hz, 2H), 3.90 (s, 3H), 3.83 (t, J=5.4 Hz, 2H), 3.50 (s, 2H), 2.79 (t, J=6.0 Hz, 2H), 2.68 (t, J=5.9 Hz, 2H), 2.60 (s, 2H), 2.37 (s, 3H).

### Example 8

### N-(2-methoxy-6-methyl-5,6,7,8-tetrahydro-1,6-naphthyridin-3-yl)-8-(1-methyl-1H-pyrazole-4-yl)quinazolin-2-amine

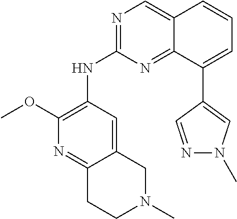

Using 1-methyl-4-(4,4,5,5-tetramethyl-1,3,2-dioxaborolan-2-yl)-1H-pyrazole to replace phenylboronic acid in the first step in Example 1, compound 8 can be obtained with similar methods and reaction steps. ESI-MS (m/z): 402.3 (M+H)+; 1HNMR (500 MHz, DMSO-d6) δ 9.28 (s, 1H), 8.65 (s, 1H), 8.28 (s, 1H), 8.07 (s, 1H), 8.06-8.00 (m, 2H), 7.76 (d, J=7.5 Hz, 1H), 7.36 (t, J=7.5 Hz, 1H), 3.87 (s, 3H), 3.85 (s, 3H), 3.46 (s, 2H), 2.83 (t, J=6.0 Hz, 2H), 2.70 (t, J=6.0 Hz, 2H), 2.38 (s, 3H).

### Example 9

### N-(2-methoxy-6-methyl-5,6,7,8-tetrahydro-1,6-naphthyridin-3-yl)-8-(pyridin-3-yl)quinazoline-2-amine

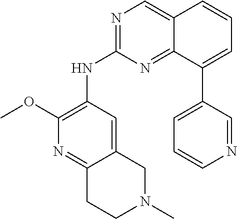 [SMILES: COc1nc2c(cc1Nc1ncc3cccc(-c4cccnc4)c3n1)CN(C)CC2]

Substituting pyridine-3-boronic acid for phenylboronic acid in the first step in Example 1, and using a similar method and reaction steps, compound 9 can be obtained. ESI-MS (m/z): 399.2 [M+H]⁺; 1HNMR (500 MHz, DMSO-d6) δ 9.41 (s, 1H), 8.88 (s, 1H), 8.66 (d, J=5.0 Hz, 1H), 8.25 (s, 1H), 8.16 (s, 1H), 8.08 (d, J=7.5 Hz, 1H), 8.03 (d, J=8.0 Hz, 1H), 7.92 (d, J=7.0 Hz, 1H), 7.58-7.52 (m, 2H), 3.91 (s, 3H), 3.23 (s, 2H), 2.74 (t, J=6.0 Hz, 2H), 2.65 (t, J=6.0 Hz, 2H), 2.40 (s, 3H).

### Example 10

### 5-(2-((2-methoxy-6-methyl-5,6,7,8-tetrahydro-1,6-naphthyridin-3-yl)amino)quinazolin-8-yl)-1-Methylpyridin-2(1H)-one

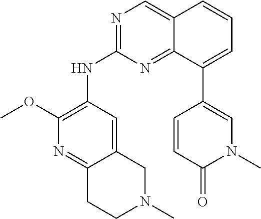

Replace the phenylboronic acid in the first step in Example 1 with 1-methyl-6-oxo-1,6-dihydropyridine-3-boronic acid pinacol ester, and use similar methods and reaction steps to obtain compound 10. ESI-MS (m/z): 429.3 [M+H]⁺; 1H NMR (500 MHz, DMSO-d6) δ 9.36 (s, 1H), 8.29 (s, 1H), 8.26 (s, 1H), 7.99 (d, J=2.5 Hz, 1H), 7.93 (dd, J=8.0, 1.5 Hz, 1H), 7.83 (dd, J=7.0, 1.5 Hz, 1H), 7.80 (dd, J=9.5, 2.5 Hz, 1H), 7.46 (dd, J=8.0, 7.5 Hz, 1H), 6.48 (d, J=9.5 Hz, 1H), 3.91 (s, 3H), 3.48 (s, 3H), 3.24 (s, 2H), 2.76 (t, J=6.0 Hz, 2H), 2.64 (t, J=6.0 Hz, 2H), 2.36 (s, 3H).

### Example 11

### N-(2-methoxy-6-methyl-5,6,7,8-tetrahydro-1,6-naphthyridin-3-yl)-8-(tetrahydro-2H-pyran-4-base) quinazolin-2-amine

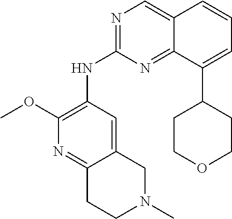 [SMILES: COc1nc2c(cc1Nc1ncc3cccc(C4CCOCC4)c3n1)CN(C)CC2]

Compound 11 was prepared by the following steps:

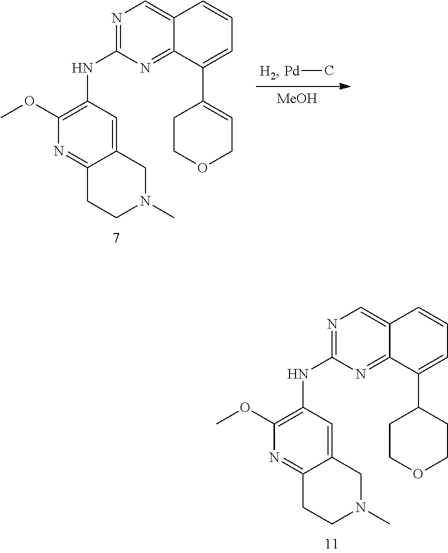

Step 1: Dissolve compound 7 (25 mg, 61 umol) in methanol 2 m (2 mL), add 10% palladium on carbon (15 mg), and stir the mixture at room temperature under hydrogen atmosphere for 16 hours. The reaction solution was filtered through celite, and the filtrate was concentrated. The residue was purified by Prep-HPLC to obtain compound 11 (1.83 mg, yield 7%). ESI-MS (m/z): 406.1 [M+H]⁺; 1HNMR (500 MHz, DMSO-d6) δ 9.30 (d, J=2.8 Hz, 1H), 8.52 (s, 1H), 8.24 (s, 1H), 7.80 (d, J=8.1 Hz, 1H), 7.73 (d, J=6.7 Hz, 1H), 7.39 (t, J=7.7 Hz, 1H), 4.05 (d, J=11.3 Hz, 2H), 3.93 (s, 3H), 3.79 (br s, 1H), 3.56 (br s, 4H), 2.79 (br s, 2H), 2.70 (br s, 2H), 2.39 (s, 3H), 1.80 (br s, 4H).

### Example 12

### 2-(2-((2-methoxy-6-methyl-5,6,7,8-tetrahydro-1,6-naphthyridin-3-yl)amino)quinazolin-8-yl)-N-methylbenzamide

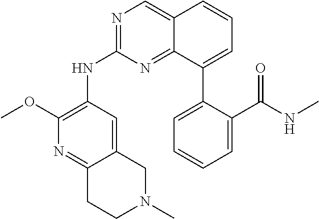

Compound 12 was prepared by the following steps:

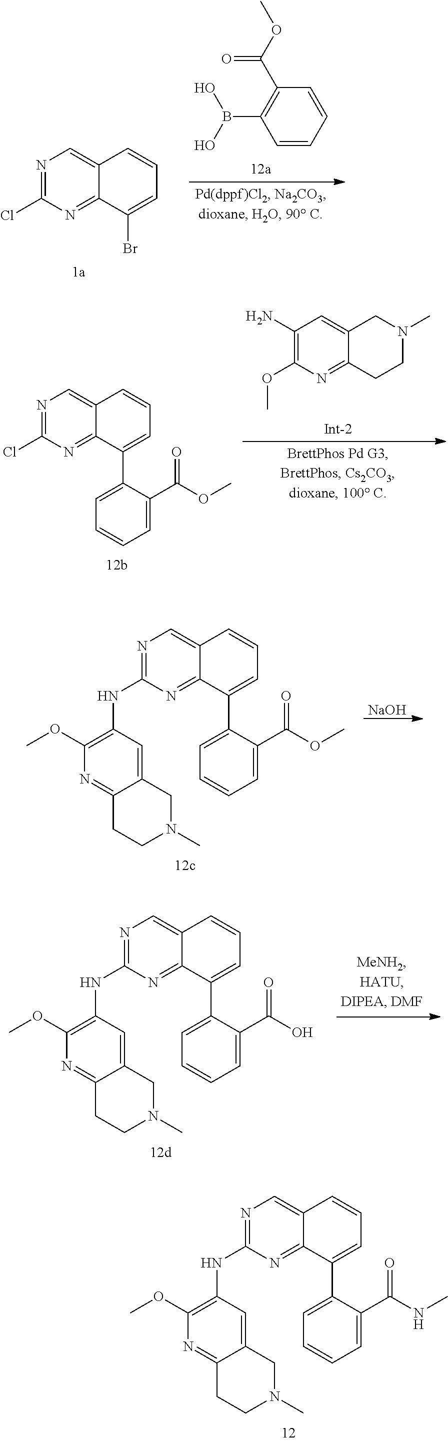

Step 1: Dissolve 2-chloro-8-bromoquinazoline 1a (100 mg, 0.41 mmol) and 2-(methoxycarbonyl)phenylboronic acid 12a (88 mg, 0.49 mmol) in a mixed solvent of 1,4-dioxane (5 mL) and water (0.5 mL), add sodium carbonate (87 mg, 0.82 mmol) and Pd(dppf)Cl₂ (15 mg, 0.020 mmol), heat the reaction system to 90° after replacing nitrogen, and the reaction system was stirred for 18 hours. After the reaction solution was cooled to room temperature, the reaction solution was filtered through celite, and the filtrate was concentrated. The residue was purified by silica gel column chromatography (petroleum ether/ethyl acetate=5/1) to obtain white solid 12b (55 mg, yield 44%). ESI-MS (m/z): 299.2 [M+H]⁺.

Step 2: Dissolve compound 12b (45 mg, 0.15 mmol) and Int-2 (29 mg, 0.15 mmol) in 1,4-dioxane (5 mL), add BrettPhos Pd G3 (13 mg, 15 umol), BrettPhos (8 mg, 15 umol) and cesium carbonate (98 mg, 0.30 mmol). The reaction system was replaced with nitrogen and then heated to 100° C. and stirred for 18 hours. After the reaction solution was cooled to room temperature, the reaction solution was filtered through celite, and the filtrate was concentrated. The residue was purified by Prep-TLC (dichloromethane/methanol=10/1) to obtain compound 12c (15 mg, yield 21%). ESI-MS (m/z): 456.2 [M+H]⁺.

Step 3: Dissolve compound 12c (15 mg, 32 umol) in a mixed solvent of methanol (0.5 mL) and tetrahydrofuran (0.5 mL), add 1N aqueous NaOH (0.2 mL), and stir the reaction mixture at room temperature overnight. The reaction solution was concentrated to obtain compound 12d (10 mg, crude product), which was directly used in the next reaction. ESI-MS (m/z): 442.2 [M+H]⁺.

Step 4: Dissolve compound 12d (10 mg) obtained in the previous step in DMF (2 mL), add HATU (10 mg, 27 umol) and DIPEA (29 mg, 226 umol), and stir the reaction mixture at room temperature for 5 minutes, and methylamine hydrochloride (7.6 mg, 113 umol) was added. The reaction mixture was stirred at room temperature for 30 minutes, diluted with water and extracted with ethyl acetate. The organic phase was washed with saturated brine, dried over anhydrous sodium sulfate, filtered and concentrated. The residue was purified by Prep-HPLC to obtain compound 12 (1 mg, yield 10%). ESI-MS (m/z): 455.2 [M+H]⁺; 1H NMR (500 MHz, DMSO-d6) δ 9.34 (s, 1H), 8.11 (s, 1H), 8.06 (s, 1H), 7.93 (d, J=8.0 Hz, 1H), 7.77-7.73 (m, 1H), 7.68 (d, J=7.5 Hz, 1H), 7.62 (d, J=7.5 Hz, 1H), 7.58 (t, J=7.5 Hz, 1H), 7.52 (t, J=7.5 Hz, 1H), 7.49-7.42 (m, 2H), 3.90 (s, 3H), 3.11 (s, 2H), 2.73-2.70 (m, 2H), 2.65-2.62 (m, 2H), 2.43 (d, J=4.5 Hz, 3H), 2.39 (s, 3H).

### Example 13

### 3-(2-((2-methoxy-6-methyl-5,6,7,8-tetrahydro-1,6-naphthyridin-3-yl)amino)quinazolin-8-yl) methyl benzoate

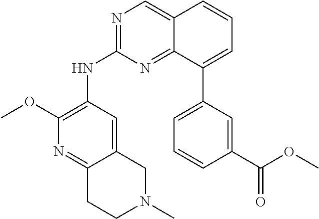

Compound 13 can be obtained by using 3-(methoxycarbonyl)phenylboronic acid instead of phenylboronic acid in the first step in Example 1, and using a similar method and reaction steps. ESI-MS (m/z): 456.2 [M+H]⁺; ¹H NMR (500 MHz, DMSO-d6) δ 9.41 (s, 1H), 8.24 (t, J=2.0 Hz, 1H), 8.20 (s, 1H), 8.10 (s, 1H), 8.08 (dt, J=7.5, 1.5 Hz, 1H), 8.02 (dd, J=8.0, 1.5 Hz, 1H), 7.92 (dt, J=7.5, 1.5 Hz, 1H), 7.88 (dd, J=7.0, 1.5 Hz, 1H), 7.71 (t, J=7.5 Hz, 1H), 7.53 (t, J=7.5 Hz, 1H), 3.90 (s, 3H), 3.85 (s, 3H), 2.98 (s, 2H), 2.72 (t, J=6.0 Hz, 2H), 2.66-2.60 (m, 2H), 2.35 (s, 3H).

### Example 14

### 3-(2-((2-methoxy-6-methyl-5,6,7,8-tetrahydro-1,6-naphthyridin-3-yl)amino)quinazolin-8-yl)-N-methylbenzamide

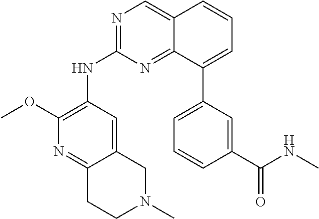

Compound 14 can be obtained by replacing the raw material 12c in the third step in Example 12 with compound 13 and using a similar method and reaction steps. ESI-MS (m/z): 455.2 [M+H]⁺; 1HNMR (500 MHz, DMSO-d6) δ 9.40 (s, 1H), 8.52 (q, J=4.5 Hz, 1H), 8.16 (s, 1H), 8.15 (s, 1H), 8.13 (t, J=1.5 Hz, 1H), 8.02-7.97 (m, 2H), 7.87 (dd, J=7.0, 1.5 Hz, 1H), 7.80 (dt, J=7.5, 1.5 Hz, 1H), 7.62 (t, J=7.5 Hz, 1H), 7.53 (t, J=7.5 Hz, 1H), 3.90 (s, 3H), 3.01 (s, 2H), 2.79 (d, J=4.5 Hz, 3H), 2.70 (t, J=6.0 Hz, 2H), 2.58 (t, J=6.0 Hz, 2H), 2.34 (s, 3H).

### Example 15

### 4-(2-((2-methoxy-6-methyl-5,6,7,8-tetrahydro-1,6-naphthyridin-3-yl)amino)quinazolin-8-yl)-3,6-Dihydropyridine-1(2H)-carboxylic acid tert-butyl ester

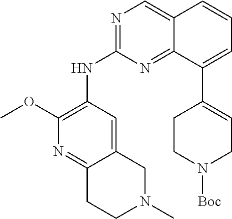 [SMILES: COc1nc2c(cc1Nc1ncc3cccc(C4=CCN(C(=O)OC(C)(C)C)CC4)c3n1)CN(C)CC2]

Compound 15 can be obtained by using N-Boc-1,2,5,6-tetrahydropyridine-4-boronic acid pinacol ester instead of phenylboronic acid in the first step in Example 1, and using a similar method and reaction steps. ESI-MS (m/z): 503.3 [M+H]⁺; 1HNMR (500 MHz, DMSO-d6) δ 9.29 (s, 1H), 8.44 (s, 1H), 8.12 (s, 1H), 7.85 (dd, J=8.0, 1.6 Hz, 1H), 7.64 (dd, J=7.4, 1.7 Hz, 1H), 7.35 (t, J=7.6 Hz, 1H), 5.96 (s, 1H), 4.00 (br s, 2H), 3.89 (s, 3H), 3.50 (t, J=5.6 Hz, 2H), 3.45 (s, 2H), 2.80 (t, J=6.1 Hz, 2H), 2.69 (t, J=6.0 Hz, 2H), 2.58 (br s, 2H), 2.37 (s, 2H), 1.45 (s, 9H).

### Example 16

### N-(2-methoxy-6-methyl-5,6,7,8-tetrahydro-1,6-naphthyridin-3-yl)-8-(prop-1-en-2-yl) quinazolin-2-amine

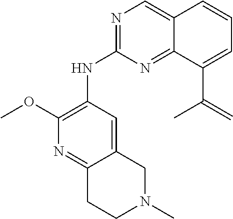 [SMILES: C=C(C)c1cccc2cnc(Nc3cc4c(nc3OC)CCN(C)C4)nc12]

Compound 16 can be obtained by replacing the phenylboronic acid in the first step in Example 1 with isopropenylboronic acid pinacol ester, and using a similar method and reaction steps. ESI-MS (m/z): 362.3 [M+H]⁺; 1HNMR (500 MHz, DMSO-d6) δ 9.31 (s, 1H), 8.54 (s, 1H), 8.26 (s, 1H), 7.87 (dd, J=8.0, 1.5 Hz, 1H), 7.68 (dd, J=7.2, 1.5 Hz, 1H), 7.38 (t, J=7.6 Hz, 1H), 5.35 (t, J=1.9 Hz, 1H), 5.20 (d, J=2.2 Hz, 1H), 3.92 (s, 3H), 3.46 (s, 2H), 2.79 (t, J=6.0 Hz, 2H), 2.68 (t, J=5.9 Hz, 2H), 2.40 (s, 3H), 2.26 (s, 3H).

### Example 17

### 1-(4-(2-((2-methoxy-6-methyl-5,6,7,8-tetrahydro-1,6-naphthyridin-3-yl)amino)quinazoline-8-yl)-3,6-dihydropyridin-1(2H)-yl)ethan-1-one

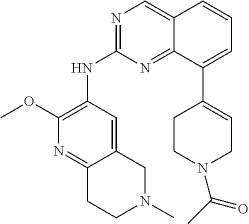 [SMILES: COc1nc2c(cc1Nc1ncc3cccc(C4=CCN(C(C)=O)CC4)c3n1)CN(C)CC2]

Compound 17 was prepared by the following steps:

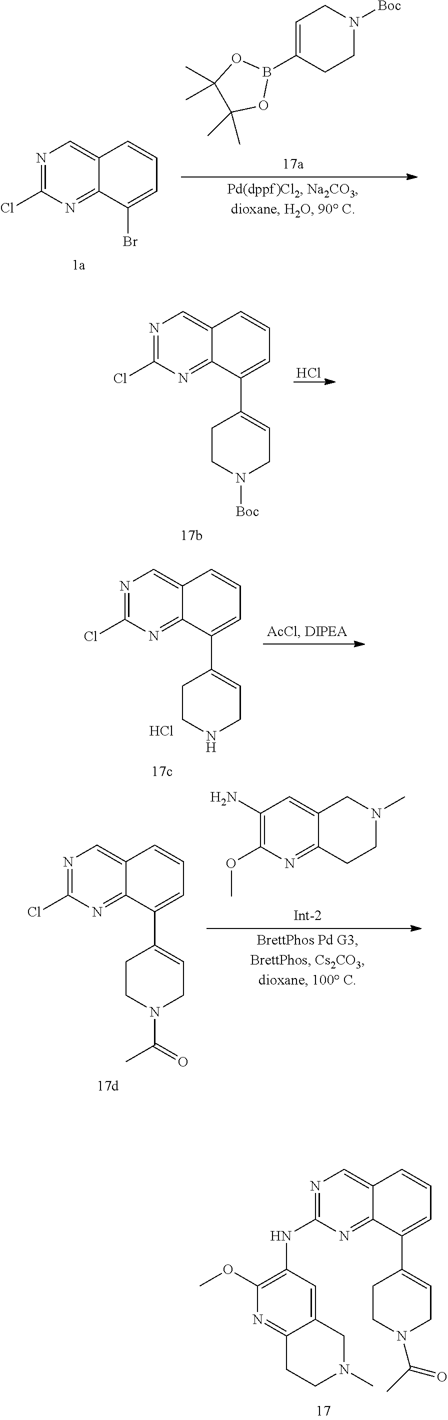

Step 1: Dissolve 2-chloro-8-bromoquinazoline 1a (220 mg, 0.90 mmol) and compound 17a (307 mg, 0.99 mmol) in a mixed solvent 1 of 4-dioxane (4 mL) and water (0.4 mL), add sodium carbonate (191 mg, 1.81 mmol) and Pd(dppf)Cl₂ (66 mg, 90 umol). The reaction system was replaced with nitrogen and heated to 90° C. and stirred for 16 hours. After the reaction solution was cooled to room temperature, the reaction solution was filtered through celite, and the filtrate was concentrated. The residue was separated by column chromatography (petroleum ether/ethyl acetate=5/1) to obtain compound 17b (260 mg, yield 83%). ESI-MS (m/z): 346.3 [M+H]⁺.

Step 2: Dissolve compound 17b (200 mg, 0.57 mmol) in dichloromethane (2 mL), add dioxane hydrochloride solution (4N, 0.72 mL), and stir the reaction mixture at room temperature for 16 hours. The reaction solution was concentrated to obtain compound 17c (160 mg, crude product), which was directly used in the next reaction.

Step 3: the compound 17c (160 mg) obtained in the previous step was dissolved in tetrahydrofuran (5 mL), and N,N-diisopropylethylamine (219 mg, 1.70 mmol, 0.29 mL) and acetyl chloride (67 mg, 0.85 mmol) were added successively at 0° C., and the reaction mixture was stirred at 0° C. for 1 hour. The reaction solution was diluted with ethyl acetate, washed with water, and the organic phase was dried over anhydrous sodium sulfate, filtered and concentrated. The residue was purified by silica gel column chromatography (dichloromethane/methanol=10/1) to obtain compound 17d (120 mg, two-step conversion yield 72%). ESI-MS (m/z): 288.3 [M+H]⁺.

Step 4: Dissolve compound 17d (44 mg, 0.15 mmol) and Int-2 (20 mg, 0.10 mmol) in 1,4-dioxane (2 mL), add BrettPhos Pd G3 (9 mg, 10 umol), BrettPhos (11 mg, 20 umol) and cesium carbonate (67 mg, 0.20 mmol). The reaction system was replaced with nitrogen and then heated to 100° C. and stirred for 16 hours. After the reaction solution was cooled to room temperature, the reaction solution was filtered through celite, and the filtrate was concentrated. The residue was purified by Prep-TLC, and the crude product was purified by Prep-HPLC to obtain compound 17 (1.45 mg, yield 3%). ESI-MS (m/z): 445.4 [M+H]⁺; 1HNMR (500 MHz, DMSO-d6) δ 9.29 (d, J=1.7 Hz, 1H), 8.40 (s, 0.5H), 8.35 (s, 0.5H), 8.18 (s, 0.5H), 8.14 (s, 0.5H), 7.91-7.81 (m, 1H), 7.65 (t, J=6.1 Hz, 1H), 7.37 (dd, J=7.7, 3.4 Hz, 1H), 5.96 (d, J=3.4 Hz, 1H), 4.17 (br s, 1H), 4.09 (br s, 1H), 3.89 (s, 3H), 3.62 (t, J=5.7 Hz, 1H), 3.58 (t, J=5.6 Hz, 1H), 3.40 (s, 2H), 2.84-2.74 (m, 2H), 2.70-2.55 (m, 4H), 2.36 (s, 3H), 2.08 (s, 3H).

### Example 18

### N-(2-methoxy-6-methyl-5,6,7,8-tetrahydro-1,6-naphthyridin-3-yl)-8-(2-trifluoromethoxy)phenyl) quinazolin-2-amine

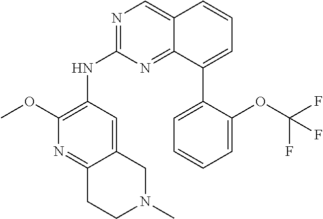 [SMILES: COc1nc2c(cc1Nc1ncc3cccc(-c4ccccc4OC(F)(F)F)c3n1)CN(C)CC2]

Compound 18 can be obtained by using 2-(trifluoromethoxy)phenylboronic acid instead of the phenylboronic acid in the first step in Example 1, and using a similar method and reaction steps. ESI-MS (m/z): 482.3 [M+H]⁺; 1HNMR (500 MHz, DMSO-d6) δ 9.40 (s, 1H), 8.11 (s, 1H), 8.04 (dd, J=8.0, 1.5 Hz, 1H), 7.99 (s, 1H), 7.80-7.77 (m, 1H), 7.68-7.63 (m, 1H), 7.60-7.51 (m, 4H), 3.90 (s, 3H), 3.00 (s, 2H), 2.70 (t, J=6.0 Hz, 2H), 2.64-2.60 (m, 2H), 2.39 (s, 3H).

### Example 19

### N-(2-methoxy-6-methyl-5,6,7,8-tetrahydro-1,6-naphthyridin-3-yl)-8-(2-(methylthio) phenyl) Quinazolin-2-amine

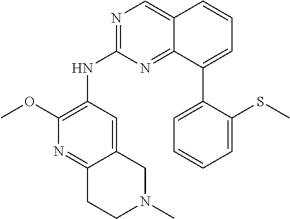

Compound 19 can be obtained by replacing the phenylboronic acid in the first step in Example 1 with 2-methylthiophenylboronic acid, and using a similar method and reaction steps. ESI-MS (m/z): 444.2 [M+H]⁺; 1HNMR (500 MHz, DMSO-d6) δ 9.37 (s, 1H), 8.08 (s, 1H), 8.04 (s, 1H), 8.00 (dd, J=8.0, 1.5 Hz, 1H), 7.72 (dd, J=7.5, 1.5 Hz, 1H), 7.53-7.47 (m, 3H), 7.35-7.31 (m, 1H), 7.26 (d, J=7.5 Hz, 1H), 3.90 (s, 3H), 3.08-2.99 (m, 2H), 2.69 (t, J=6.0 Hz, 2H), 2.63-2.58 (m, 2H), 2.40 (s, 3H), 2.29 (s, 3H).

### Example 20

### N-(2-methoxy-6-methyl-5,6,7,8-tetrahydro-1,6-naphthyridin-3-yl)-8-(2-phenoxyphenyl)quinazolin-2-amine

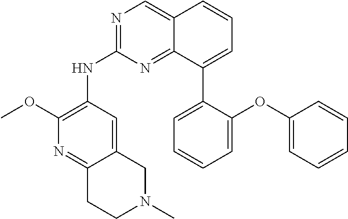 [SMILES: COc1nc2c(cc1Nc1ncc3cccc(-c4ccccc4Oc4ccccc4)c3n1)CN(C)CC2]

Compound 20 can be obtained by replacing the phenylboronic acid in the first step in Example 1 with 2-phenoxyphenylboronic acid and using a similar method and reaction steps. ESI-MS (m/z): 490.4 [M+H]⁺; 1HNMR (500 MHz, DMSO-d6) δ 9.32 (s, 1H), 8.22 (s, 1H), 8.10 (s, 1H), 7.92 (d, J=8.0 Hz, 1H), 7.77 (d, J=7.0 Hz, 1H), 7.53-7.48 (m, 2H), 7.42 (t, J=7.5 Hz, 1H), 7.35 (t, J=7.5 Hz, 1H), 7.11-7.05 (m, 3H), 6.91 (t, J=7.5 Hz, 1H), 6.73 (d, J=8.0 Hz, 2H), 3.93 (s, 3H), 3.09 (s, 2H), 2.77-2.72 (m, 2H), 2.70-2.61 (m, 2H), 2.39 (s, 3H).

### Example 21

### 8-(2-fluoro-6-methoxyphenyl)-N-(2-methoxy-6-methyl-5,6,7,8-tetrahydro-1,6-naphthyridine-3-base) quinazolin-2-amine

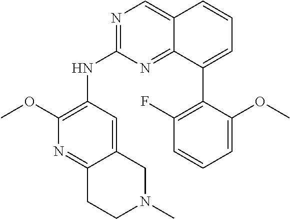

Compound 21 can be obtained by replacing the phenylboronic acid in the first step in Example 1 with 2-fluoro-6-methoxyphenylboronic acid, and using a similar method and reaction steps. ESI-MS (m/z): 446.2 [M+H]⁺; 1HNMR (500 MHz, DMSO-d6) δ 9.37 (s, 1H), 8.06 (s, 1H), 8.03 (s, 1H), 8.01 (d, J=8.0 Hz, 1H), 7.75 (d, J=7.0 Hz, 1H), 7.54-7.48 (m, 2H), 7.08 (d, J=8.0 Hz, 1H), 7.01 (t, J=8.5 Hz, 1H), 3.91 (s, 3H), 3.65 (s, 3H), 3.06 (s, 2H), 2.70 (t, J=6.0 Hz, 2H), 2.60 (t, J=6.0 Hz, 2H), 2.40 (s, 3H).

### Example 22

### 8-(2-(benzyloxy)phenyl)-N-(2-methoxy-6-methyl-5,6,7,8-tetrahydro-1,6-naphthyridin-3-yl) quinazolin-2-amine

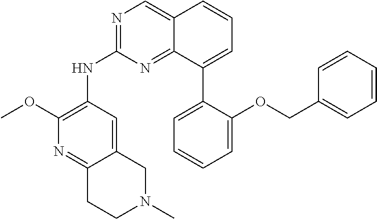 [SMILES: COc1nc2c(cc1Nc1ncc3cccc(-c4ccccc4OCc4ccccc4)c3n1)CN(C)CC2]

Compound 22 can be obtained by replacing the phenylboronic acid in the first step in Example 1 with 2-benzyloxyphenylboronic acid, and using a similar method and reaction steps. ESI-MS (m/z): 504.4 [M+H]⁺; 1H NMR (500 MHz, DMSO-d6) δ 9.36 (s, 1H), 8.13 (s, 1H), 8.02 (s, 1H), 7.96 (dd, J=8.0, 1.5 Hz, 1H), 7.78-7.75 (m, 1H), 7.50-7.43 (m, 2H), 7.34 (dd, J=7.5, 1.5 Hz, 1H), 7.25 (d, J=8.0 Hz, 1H), 7.16-7.09 (m, 2H), 7.08-7.04 (m, 2H), 6.98 (d, J=7.5 Hz, 2H), 4.97 (s, 2H), 3.90 (s, 3H), 2.97 (s, 2H), 2.69 (t, J=6.0 Hz, 2H), 2.58 (t, J=6.0 Hz, 2H), 2.36 (s, 3H).

### Example 23

### N-(2-methoxy-6-methyl-5,6,7,8-tetrahydro-1,6-naphthyridin-3-yl)-8-(2-methoxypyridin-3-yl) quinazolin-2-amine

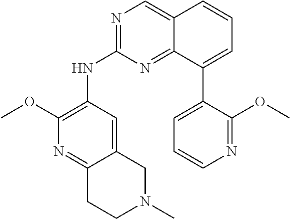

Compound 23 was prepared by the following steps:

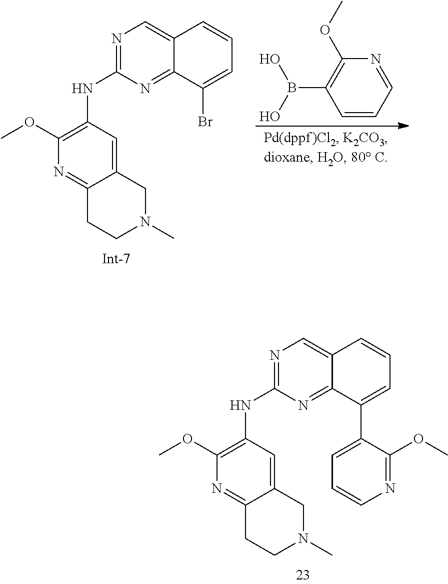

Step 1: Dissolve Int-7 (20 mg, 49 umol) and 2-methoxypyridyl-3-boronic acid (9 mg, 59 umol) in a mixed solvent of 1,4-dioxane (5 mL) and water (0.5 mL), and add potassium carbonate (13 mg, 99 umol) and Pd(dppf)Cl₂ (3 mg, 5 umol). The reaction system was replaced with nitrogen and heated to 80° C. and stirred overnight. After the reaction solution was cooled to room temperature, the reaction solution was filtered through celite, and the filtrate was concentrated. The residue was purified by Prep-HPLC to obtain yellow solid 23 (10 mg, yield 49.88%). ESI-MS (m/z): 429.3 [M+H]⁺; 1HNMR (500 MHz, DMSO-d6) δ 9.37 (s, 1H), 8.34-8.31 (m, 1H), 8.08 (s, 1H), 8.03 (s, 1H), 8.00 (d, J=8.0 Hz, 1H), 7.80 (d, J=7.0 Hz, 1H), 7.74 (dd, J=7.0, 2.0 Hz, 1H), 7.50 (t, J=7.5 Hz, 1H), 7.21-7.16 (m, 1H), 3.91 (s, 3H), 3.72 (s, 3H), 3.14 (s, 2H), 2.71 (t, J=6.0 Hz, 2H), 2.62 (t, J=6.0 Hz, 2H), 2.40 (s, 3H).

### Example 24

### 1-(2-((2-methoxy-6-methyl-5,6,7,8-tetrahydro-1,6-naphthyridin-3-yl)amino)quinazolin-8-yl) pyrrolidin-2-one

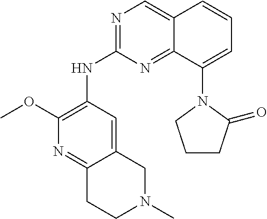 [SMILES: COc1nc2c(cc1Nc1ncc3cccc(N4CCCC4=O)c3n1)CN(C)CC2]

Compound 24 was prepared by the following steps:

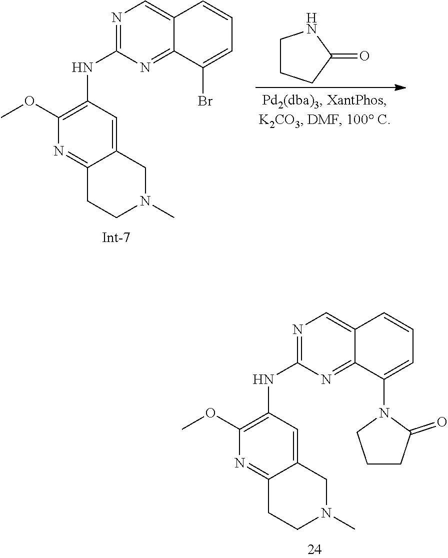 [SMILES: COc1nc2c(cc1Nc1ncc3cccc(Br)c3n1)CN(C)CC2 ; O=C1CCCN1 ; COc1nc2c(cc1Nc1ncc3cccc(N4CCCC4=O)c3n1)CN(C)CC2]

Step 1: Dissolve Int-7 (50 mg, 0.12 mmol) and 2-pyrrolidone (12 mg, 0.15 mmol) in DMF (5 mL), add XantPhos (14 mg, 25 umol), Pd₂(dba)₃ (11 mg, 12 umol) and potassium carbonate (34 mg, 0.25 mmol), the reaction system was replaced with nitrogen and then heated to 100° C. to react overnight, and the product formation was monitored by LCMS. The reaction solution was concentrated, and the residue was purified by Prep-TLC to obtain a crude product, and then purified by Prep-HPLC to obtain compound 24 (4 mg, yield 9%). ESI-MS (m/z): 405.1 [M+H]⁺; 1HNMR (500 MHz, DMSO-d6) δ 9.35 (d, J=2.3 Hz, 1H), 8.47 (s, 1H), 8.20 (s, 1H), 7.91 (d, J=8.0 Hz, 1H), 7.76 (d, J=7.5 Hz, 1H), 7.42 (t, J=8.1 Hz, 1H), 3.94-3.83 (m, 5H), 2.80 (br s, 2H), 2.70 (br s, 2H), 2.44-2.33 (m, 5H), 2.16 (br s, 2H).

### Example 25

### N-(2-methoxy-6-methyl-5,6,7,8-tetrahydro-1,6-naphthyridin-3-yl)-8-(2-methoxyphenyl)pyrido [3,4-d]pyrimidin-2-amine

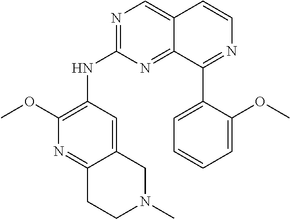

Compound 25 was prepared by the following steps:

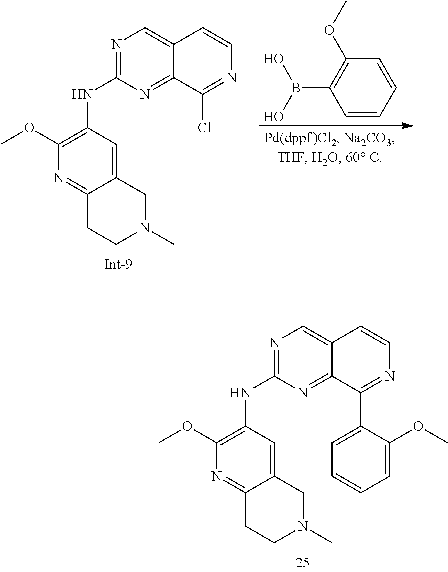

Step 1: Dissolve Int-9 (50 mg, 0.14 mmol) and 2-methoxyphenylboronic acid (32 mg, 0.21 mmol) in a mixed solution of THF (10 mL) and water (2 mL), and add [1,1′-bis(diphenylphosphino)ferrocene]dichloropalladium dichloromethane complex (11 mg, 14 umol), sodium carbonate (29 mg, 0.28 mmol). The reaction system was replaced with nitrogen, heated to 60° C. and stirred overnight, and the product formation was detected by LCMS. The reaction solution was concentrated, and the residue was purified by Prep-HPLC to obtain yellow solid 25 (24 mg, yield 41%). ESI-MS (m/z): 429.1 [M+H]⁺; 1HNMR (500 MHz, DMSO-d6) δ 9.49 (s, 1H), 8.55 (d, J=5.3 Hz, 1H), 8.33 (s, 1H), 8.05 (s, 1H), 7.85 (d, J=5.4 Hz, 1H), 7.58-7.50 (m, 1H), 7.34 (dd, J=7.4, 1.6 Hz, 1H), 7.23 (d, J=8.3 Hz, 1H), 7.14 (t, J=7.4 Hz, 1H), 3.90 (s, 3H), 3.59 (s, 3H), 3.08 (s, 2H), 2.71 (t, J=5.9 Hz, 2H), 2.61 (t, J=5.9 Hz, 2H), 2.40 (s, 3H).

### Example 26

### 8-(3,6-dihydro-2H-pyran-4-yl)-N-(2-methoxy-6-methyl-5,6,7,8-tetrahydro-1,6-naphthalene pyridin-3-yl)pyrido[3,4-d]pyrimido-2-amine

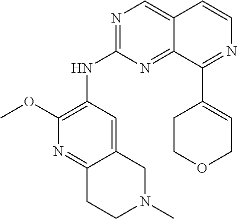 [SMILES: COc1nc2c(cc1Nc1ncc3ccnc(C4=CCOCC4)c3n1)CN(C)CC2]

Compound 26 can be obtained by using 3,6-dihydro-2H-pyran-4-boronic acid pinacol ester instead of 2-methoxyphenylboronic acid in the first step in Example 25, and using a similar method and reaction steps. ESI-MS (m/z): 404.1 [M+H]⁺; 1HNMR (500 MHz, DMSO-d6) δ 9.40 (s, 1H), 8.90 (s, 1H), 8.41 (d, J=5.1 Hz, 1H), 7.98 (s, 1H), 7.67 (d, J=5.1 Hz, 1H), 7.21 (s, 1H), 4.24 (q, J=2.9 Hz, 2H), 3.87 (s, 3H), 3.83 (t, J=5.4 Hz, 2H), 3.47 (s, 2H), 2.82 (t, J=6.0 Hz, 2H), 2.70 (t, J=5.9 Hz, 2H), 2.66 (dt, J=8.2, 4.2 Hz, 2H), 2.38 (s, 3H).

### Example 27

### 5-(2-((2-methoxy-6-methyl-5,6,7,8-tetrahydro-1,6-naphthyridin-3-yl)amino)pyrido[3,4-d]pyrimidino-8-yl)-1-methylpyridin-2(1H)-one

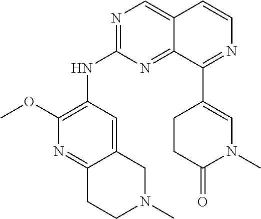

Replace the 2-methoxyphenylboronic acid in the first step in Example 25 with 1-methyl-6-oxo-1,6-dihydropyridine-3-boronic acid pinacol ester, and use a similar method and reaction steps, compound 27 can be obtained. ESI-MS (m/z): 430.1 [M+H]⁺; 1H NMR (500 MHz, DMSO-d6) δ 9.44 (s, 1H), 9.08 (s, 1H), 8.81 (br s, 1H), 8.45 (d, J=5.0 Hz, 1H), 8.32 (dd, J=9.9, 2.5 Hz, 1H), 7.89 (s, 1H), 7.71 (d, J=5.0 Hz, 1H), 6.44 (d, J=9.5 Hz, 1H), 3.85 (s, 3H), 3.38 (s, 3H), 3.36 (s, 2H), 2.82 (t, J=6.0 Hz, 2H), 2.68 (t, J=6.0 Hz, 2H), 2.36 (s, 3H).

### Example 28

### N-(2-methoxy-6-methyl-5,6,7,8-tetrahydro-1,6-naphthyridin-3-yl)-8-morpholinoquinazolin-2-amine

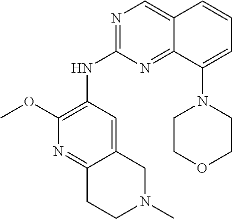 [SMILES: COc1nc2c(cc1Nc1ncc3cccc(N4CCOCC4)c3n1)CN(C)CC2]

Compound 28 was prepared by the following steps:

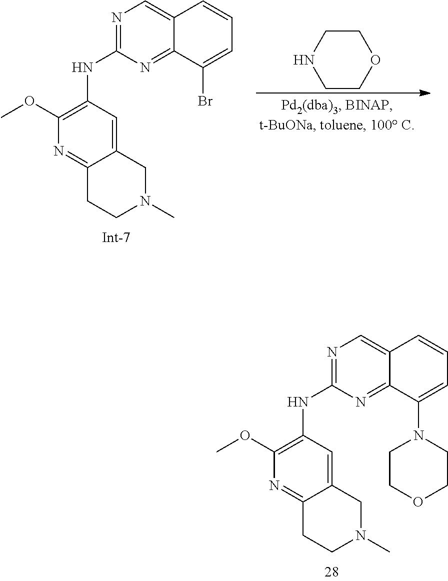 [SMILES: COc1nc2c(cc1Nc1ncc3cccc(Br)c3n1)CN(C)CC2 ; C1COCCN1 ; COc1nc2c(cc1Nc1ncc3cccc(N4CCOCC4)c3n1)CN(C)CC2]

Step 1: Dissolve Int-7 (40 mg, 99 umol) in toluene (5 mL), add Pd₂(dba)3 (4 mg, 4.9 umol), BINAP (9 mg, 14.9 umol), sodium tert-butoxide (19 mg, 199 umol) and morpholine (13 mg, 149 umol). The reaction system was replaced with nitrogen and heated to 100° C. and stirred overnight. The reaction solution was concentrated, and the residue was purified by Prep-TLC (dichloromethane/methanol=10/1) to obtain a crude product, which was further purified by Prep-HPLC to obtain a yellow solid 28 (23 mg, yield 57%). ESI-MS (m/z): 407.0 [M+H]⁺; 1HNMR (500 MHz, DMSO-d6) δ 9.25 (s, 1H), 8.30 (s, 1H), 8.24 (s, 1H), 7.52 (dd, J=8.0, 1.5 Hz, 1H), 7.31 (t, J=7.5 Hz, 1H), 7.24 (dd, J=7.5, 1.5 Hz, 1H), 3.90 (s, 3H), 3.79 (t, J=4.5 Hz, 4H), 3.52 (s, 2H), 3.23 (t, J=4.5 Hz, 4H), 2.81 (t, J=6.0 Hz, 2H), 2.70 (t, J=6.0 Hz, 2H), 2.39 (s, 3H).

### Example 29

### N-(2-methoxy-6-methyl-5,6,7,8-tetrahydro-1,6-naphthyridin-3-yl)-8-(piperidin-1-yl) quinazoline-2-amine

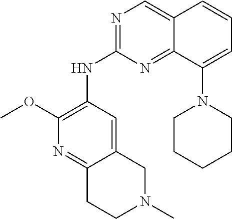 [SMILES: COc1nc2c(cc1Nc1ncc3cccc(N4CCCCC4)c3n1)CN(C)CC2]

Compound 29 can be obtained by replacing the morpholine in the first step in Example 28 with piperidine, and using a similar method and reaction steps. MS (ESI): m/z 405.2 [M+H]⁺; 1HNMR (500 MHz, DMSO-d6) δ 9.23 (s, 1H), 8.49 (s, 1H), 8.17 (s, 1H), 7.49 (dd, J=7.5, 1.5 Hz, 1H), 7.30 (t, J=7.5 Hz, 1H), 7.25 (dd, J=7.5, 1.5 Hz, 1H), 3.92 (s, 3H), 3.51 (s, 2H), 3.15 (t, J=5.0 Hz, 4H), 2.80 (t, J=6.0 Hz, 2H), 2.69 (t, J=6.0 Hz, 2H), 2.38 (s, 3H), 1.76-1.70 (m, 4H), 1.62-1.57 (m, 2H).

### Example 30

### N-(2-methoxy-6-methyl-5,6,7,8-tetrahydro-1,6-naphthyridin-3-yl)-8-(1-methyl-1H-pyrazole-3-yl)quinazolin-2-amine

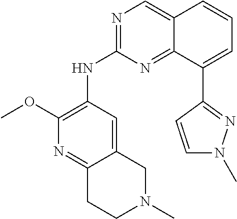

Compound 30 can be obtained by using 1-methylpyrazole-3-boronic acid pinacol ester instead of 2-methoxy-3-pyridineboronic acid in the first step in Example 23, and using a similar method and reaction steps. ESI-MS (m/z): 402.2 [M+H]⁺; 1HNMR (500 MHz, DMSO-d6) δ 9.32 (s, 1H), 8.38 (s, 1H), 8.37 (s, 1H), 8.27 (dd, J=7.5, 1.5 Hz, 1H), 7.89 (dd, J=7.5, 1.5 Hz, 1H), 7.78 (d, J=2.0 Hz, 1H), 7.43 (t, J=7.5 Hz, 1H), 7.11 (d, J=2.0 Hz, 1H), 3.95 (s, 3H), 3.90 (s, 3H), 3.43 (s, 2H), 2.80 (t, J=6.0 Hz, 2H), 2.70 (t, J=6.0 Hz, 2H), 2.42 (s, 3H).

### Example 31

### N2-(2-methoxy-6-methyl-5,6,7,8-tetrahydro-1,6-naphthyridin-3-yl)-N8-((tetrahydro-2H-pyran-4-yl)methyl)quinazoline-2,8-diamine

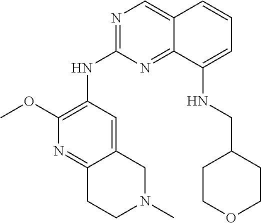 [SMILES: COc1nc2c(cc1Nc1ncc3cccc(NCC4CCOCC4)c3n1)CN(C)CC2]

Compound 31 can be obtained by replacing the morpholine in the first step in Example 28 with 4-aminomethyltetrahydropyran and using a similar method and reaction steps. ESI-MS (m/z): 435.0 [M+H]⁺; 1H NMR (500 MHz, DMSO-d6) δ 9.18 (s, 1H), 8.34 (s, 1H), 8.21 (s, 1H), 7.21 (t, J=8.0 Hz, 1H), 7.10 (dd, J=8.0, 1.0 Hz, 1H), 6.82 (d, J=7.5 Hz, 1H), 5.67 (t, J=6.0 Hz, 1H), 3.93 (s, 3H), 3.92-3.88 (m, 2H), 3.48 (s, 2H), 3.37-3.34 (m, 2H), 3.14 (t, J=6.0 Hz, 2H), 2.79 (t, J=6.0 Hz, 2H), 2.68 (t, J=6.0 Hz, 2H), 2.39 (s, 3H), 2.01-1.92 (m, 1H), 1.76-1.70 (m, 2H), 1.39-1.29 (m, 2H).

### Example 32

### 2-(2-((2-methoxy-6-methyl-5,6,7,8-tetrahydro-1,6-naphthyridin-3-yl)amino)quinazolin-8-yl) isoindolin-1-one

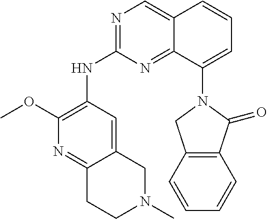 [SMILES: COc1nc2c(cc1Nc1ncc3cccc(N4Cc5ccccc5C4=O)c3n1)CN(C)CC2]

Compound 32 can be obtained by using isoindolin-1-one instead of 2-pyrrolidone in the first step in Example 24, and using a similar method and reaction steps. ESI-MS (m/z): 453.1 [M+H]⁺; 1HNMR (500 MHz, DMSO-d6) δ 9.43 (s, 1H), 8.34 (s, 1H), 8.11 (s, 1H), 8.02 (dd, J=8.1, 1.4 Hz, 1H), 7.97 (dd, J=7.4, 1.5 Hz, 1H), 7.88 (d, J=7.5 Hz, 1H), 7.78-7.70 (m, 2H), 7.66-7.61 (m, 1H), 7.53 (t, J=7.7 Hz, 1H), 5.08 (s, 2H), 3.89 (s, 3H), 3.42-3.35 (m, 2H), 2.65 (t, J=6.0 Hz, 2H), 2.49-2.45 (m, 2H), 2.12 (s, 3H).

### Example 33

### N-(2-methoxy-6-methyl-5,6,7,8-tetrahydro-1,6-naphthyridin-3-yl)-8-(piperidin-1-yl) pyrido[3,4-d]pyrimidin-2-amine

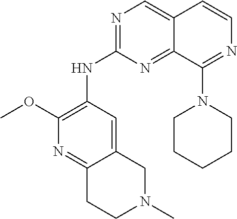 [SMILES: COc1nc2c(cc1Nc1ncc3ccnc(N4CCCCC4)c3n1)CN(C)CC2]

Compound 33 was prepared by the following steps:

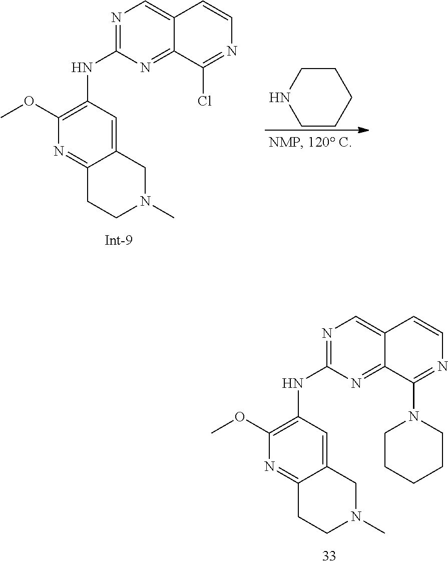 [SMILES: COc1nc2c(cc1Nc1ncc3ccnc(Cl)c3n1)CN(C)CC2 ; C1CCNCC1 ; COc1nc2c(cc1Nc1ncc3ccnc(N4CCCCC4)c3n1)CN(C)CC2]

Step 1: Dissolve Int-9 (50 mg, 140 umol) in N-methylpyrrolidone (5 mL), and add piperidine (178 mg, 2.1 mmol). The reaction solution was heated to 120° C. and stirred for 2 hours, and LCMS detected that the reaction was complete. The reaction solution was concentrated, and the residue was purified by Prep-HPLC to obtain yellow solid 33 (31 mg, yield 55%). ESI-MS (m/z): 406.5 [M+H]⁺; 1HNMR (500 MHz, DMSO-d6) δ 9.23 (s, 1H), 8.59 (s, 1H), 8.01-7.90 (m, 2H), 7.12 (d, J=5.4 Hz, 1H), 3.87 (s, 3H), 3.69 (br s, 4H), 3.48 (s, 2H), 2.80 (t, J=6.0 Hz, 2H), 2.68 (t, J=6.0 Hz, 2H), 2.38 (s, 3H), 1.59 (br s, 6H).

### Example 34

### N-(2-methoxy-6-methyl-5,6,7,8-tetrahydro-1,6-naphthyridin-3-yl)-8-(2-methoxypyridin-3-yl) pyrido[3,4-d]pyrimidin-2-amine

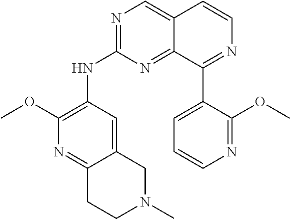

Compound 34 can be obtained by using 2-methoxypyridyl-3-boronic acid instead of piperidine in the first step in Example 25, and using a similar method and reaction steps. ESI-MS (m/z): 430.3 [M+H]⁺; ¹HNMR (500 MHz, DMSO-d6) δ 9.51 (d, J=1.9 Hz, 1H), 8.57 (dd, J=5.3, 1.9 Hz, 1H), 8.42 (d, J=2.0 Hz, 1H), 8.38 (dt, J=4.1, 2.0 Hz, 1H), 7.99 (s, 1H), 7.89 (dd, J=5.4, 2.0 Hz, 1H), 7.82 (dt, J=7.0, 2.0 Hz, 1H), 7.21 (ddd, J=7.1, 5.0, 1.9 Hz, 1H), 3.90 (d, J=1.9 Hz, 3H), 3.71 (d, J=1.9 Hz, 3H), 3.16 (s, 2H), 2.71 (d, J=6.1 Hz, 2H), 2.66-2.60 (m, 2H), 2.41 (d, J=2.0 Hz, 3H).

### Example 35

### N-(2-methoxy-6-methyl-5,6,7,8-tetrahydro-1,6-naphthyridin-3-yl)-8-morpholinopyrido[3,4-d]pyrimidin-2-amine

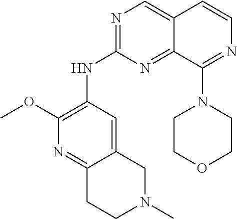 [SMILES: COc1nc2c(cc1Nc1ncc3ccnc(N4CCOCC4)c3n1)CN(C)CC2]

Using morpholine instead of piperidine in the first step in Example 33, and using a similar method and reaction steps, compound 35 can be obtained. ESI-MS (m/z): 408.3 [M+H]⁺; ¹HNMR (500 MHz, DMSO-d6) δ 9.27 (s, 1H), 8.73 (s, 1H), 7.97 (d, J=5.4 Hz, 1H), 7.85 (s, 1H), 7.20 (d, J=5.4 Hz, 1H), 3.86 (s, 3H), 3.70 (dt, J=8.8, 4.6 Hz, 8H), 3.48 (s, 2H), 2.81 (t, J=5.9 Hz, 2H), 2.69 (t, J=5.9 Hz, 2H), 2.38 (s, 3H).

### Example 36

### 1-(2-((2-methoxy-6-methyl-5,6,7,8-tetrahydro-1,6-naphthyridin-3-yl)amino)pyrido[3,4-d]pyrimidin-8-yl)pyrrolidin-2-one

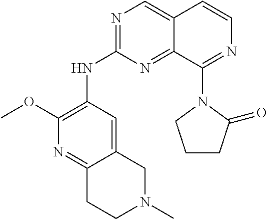 [SMILES: COc1nc2c(cc1Nc1ncc3ccnc(N4CCCC4=O)c3n1)CN(C)CC2]

Compound 36 can be obtained by replacing Int-7 in the first step in Example 24 with Int-9 and using a similar method and reaction steps. ESI-MS (m/z): 405.3 [M+H]⁺; ¹HNMR (500 MHz, DMSO-d6) δ 9.49 (s, 1H), 8.69 (s, 1H), 8.34 (d, J=5.3 Hz, 1H), 8.32 (s, 1H), 7.80 (d, J=5.3 Hz, 1H), 3.96 (t, J=6.9 Hz, 2H), 3.91 (s, 3H), 3.49 (s, 2H), 2.80 (t, J=6.0 Hz, 2H), 2.69 (t, J=5.9 Hz, 2H), 2.54 (t, J=8.0 Hz, 2H), 2.39 (s, 3H), 2.21 (t, J=7.4 Hz, 2H).

### Example 37

### N-(2-methoxy-6-methyl-5,6,7,8-tetrahydro-1,6-naphthyridin-3-yl)-8-(pyridin-2-yl)quinazoline-2-amine

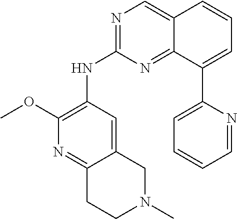 [SMILES: COc1nc2c(cc1Nc1ncc3cccc(-c4ccccn4)c3n1)CN(C)CC2]

Compound 37 was prepared by the following steps:

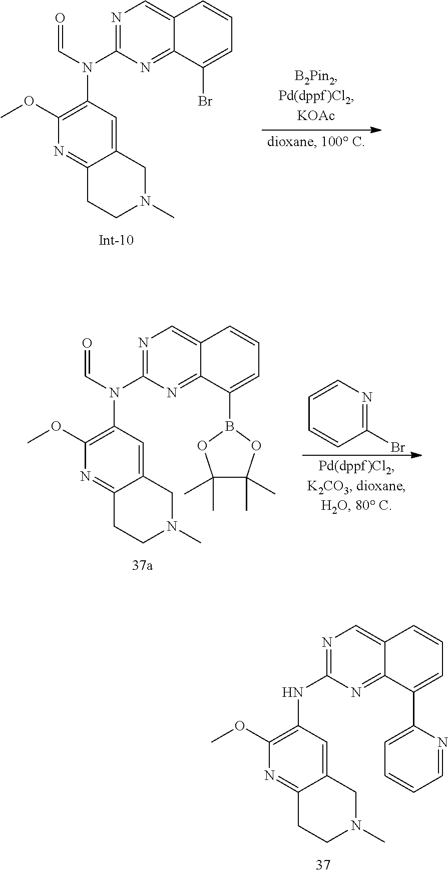

Step 1: Dissolve Int-10 (40 mg, 93 umol) and pinacol diborate (28 mg, 0.11 mmol) in 1,4-dioxane (4 mL), and add potassium acetate (27 mg, 0.28 mmol) and Pd(dppf)Cl₂ (6 mg, 9 umol). The reaction system was replaced with nitrogen and then heated to 100° C. and stirred for 3 hours. After the reaction solution was cooled to room temperature, the reaction solution was filtered through celite, and the filter cake was washed with 1,4-dioxane (1 mL). The filtrate containing compound 37a was used directly in the next reaction without further purification.

Step 2: Add 2-bromopyridine (14 mg, 93 umol) and water (0.5 mL) to the filtrate of compound 37a obtained in the previous step, then add potassium carbonate (25 mg, 186 umol) and Pd(dppf)Cl₂ (6 mg, 9 umol). The reaction system was replaced with nitrogen and heated to 100° C. and stirred overnight. After the reaction solution was cooled to room temperature, the reaction solution was filtered through celite, and the filtrate was concentrated. The residue was purified by Prep-HPLC to obtain compound 37 (6 mg, yield 17%). ESI-MS (m/z): 399.3 [M+H]⁺; ¹HNMR (500 MHz, DMSO-d6) δ 9.39 (s, 1H), 8.77-8.74 (m, 1H), 8.32 (s, 1H), 8.19 (s, 1H), 8.17 (dd, J=7.5, 1.5 Hz, 1H), 8.10 (d, J=8.0 Hz, 1H), 8.03 (dd, J=8.0, 1.5 Hz, 1H), 7.89 (td, J=7.5, 2.0 Hz, 1H), 7.53 (t, J=7.5 Hz, 1H), 7.47-7.43 (m, 1H), 3.90 (s, 3H), 3.23 (s, 2H), 2.76 (t, J=6.0 Hz, 2H), 2.65 (t, J=6.0 Hz, 2H), 2.38 (s, 3H).

### Example 38

### N8-Benzyl-N2-(2-methoxy-6-methyl-5,6,7,8-tetrahydro-1,6-naphthyridin-3-yl)quinazoline-2,8-diamine

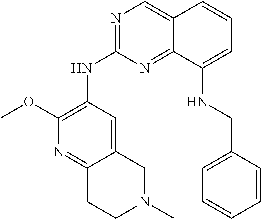 [SMILES: COc1nc2c(cc1Nc1ncc3cccc(NCc4ccccc4)c3n1)CN(C)CC2]

Compound 38 can be obtained by replacing the morpholine in the first step in Example 28 with benzylamine, and using a similar method and reaction steps. ESI-MS (m/z): 427.3 [M+H]⁺; ¹HNMR (500 MHz, DMSO-d6) δ 9.20 (s, 1H), 8.25 (s, 1H), 8.20 (s, 1H), 7.46 (d, J=7.5 Hz, 2H), 7.41-7.36 (m, 2H), 7.32-7.28 (m, 1H), 7.19 (t, J=8.0 Hz, 1H), 7.13 (dd, J=8.0, 2.0 Hz, 1H), 6.83 (dd, J=7.5, 2.0 Hz, 1H), 6.08 (t, J=5.5 Hz, 1H), 4.48 (d, J=5.5 Hz, 2H), 3.92 (s, 3H), 3.26 (s, 2H), 2.76 (t, J=6.0 Hz, 2H), 2.64 (t, J=6.0 Hz, 2H), 2.34 (s, 3H).

### Example 39

### N8-isobutyl-N2-(2-methoxy-6-methyl-5,6,7,8-tetrahydro-1,6-naphthyridin-3-yl)quinazoline-2,8-diamine

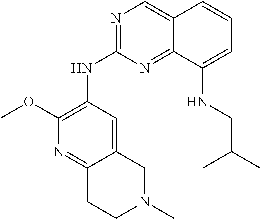 [SMILES: COc1nc2c(cc1Nc1ncc3cccc(NCC(C)C)c3n1)CN(C)CC2]

Compound 39 can be obtained by replacing the morpholine in the first step in Example 28 with isobutylamine and using a similar method and reaction steps. ESI-MS (m/z): 393.3 [M+H]⁺; 1H NMR (500 MHz, DMSO-d6) δ 9.18 (s, 1H), 8.33 (s, 1H), 8.25 (s, 1H), 7.20 (t, J=8.0 Hz, 1H), 7.09 (dd, J=8.0, 1.0 Hz, 1H), 6.79 (d, J=7.5 Hz, 1H), 5.68 (t, J=6.0 Hz, 1H), 3.92 (s, 3H), 3.49 (s, 2H), 3.07 (t, J=6.0 Hz, 2H), 2.79 (t, J=6.0 Hz, 2H), 2.68 (t, J=6.0 Hz, 2H), 2.37 (s, 3H), 2.04-1.97 (m, 1H), 1.03 (d, J=6.5 Hz, 6H).

### Example 40

### 8-(6-chloropyridin-2-yl)-N-(2-methoxy-6-methyl-5,6,7,8-tetrahydro-1,6-naphthyridin-3-yl)quinazolin-2-amine

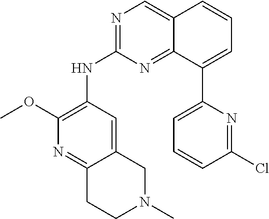 [SMILES: COc1nc2c(cc1Nc1ncc3cccc(-c4cccc(Cl)n4)c3n1)CN(C)CC2]

Compound 40 can be obtained by using 2-bromo-6-chloropyridine instead of 2-bromopyridine in the second step in Example 37, and using a similar method and reaction steps. ESI-MS (m/z): 433.2 [M+H]⁺; ¹HNMR (500 MHz, DMSO-d6) δ 9.40 (s, 1H), 8.45 (s, 1H), 8.19-8.15 (m, 2H), 8.09 (s, 1H), 8.06 (dd, J=8.0, 2.0 Hz, 1H), 7.94 (t, J=8.0 Hz, 1H), 7.58 (d, J=8.0 Hz, 1H), 7.53 (t, J=7.5 Hz, 1H), 3.89 (s, 3H), 3.27 (s, 2H), 2.77 (t, J=6.0 Hz, 2H), 2.67 (t, J=6.0 Hz, 2H), 2.39 (s, 3H).

### Example 41

### N2-(2-methoxy-6-methyl-5,6,7,8-tetrahydro-1,6-naphthyridin-3-yl)-N8-neopentylpyrido[3,4-d]pyrimidine-2,8-diamine

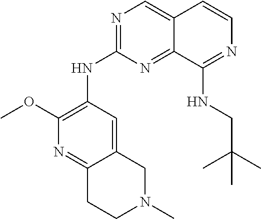 [SMILES: COc1nc2c(cc1Nc1ncc3ccnc(NCC(C)(C)C)c3n1)CN(C)CC2]

Compound 41 can be obtained by replacing piperidine in the first step in Example 33 with neopentylamine, and using a similar method and reaction steps. ESI-MS (m/z): 408.3 [M+H]⁺; ¹HNMR (500 MHz, DMSO-d6) δ 9.18 (s, 1H), 8.56 (s, 1H), 8.22 (s, 1H), 7.78 (d, J=5.7 Hz, 1H), 6.86 (d, J=5.7 Hz, 1H), 6.64-6.60 (m, 1H), 3.91 (s, 3H), 3.47 (s, 2H), 3.38 (d, J=6.3 Hz, 2H), 2.79 (t, J=5.9 Hz, 2H), 2.68 (d, J=5.8 Hz, 2H), 2.35 (s, 3H), 0.98 (s, 9H).

### Example 42

### (S)—N8-(3,3-dimethylbut-2-yl)-N2-(2-methoxy-6-methyl-5,6,7,8-tetrahydro-1,6-naphthyridin-3-yl)pyrido[3,4-d]pyrimidine-2,8-diamine

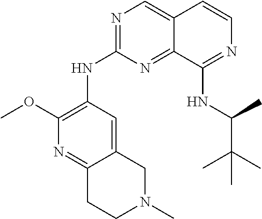 [SMILES: COc1nc2c(cc1Nc1ncc3ccnc(N[C@@H](C)C(C)(C)C)c3n1)CN(C)CC2]

Compound 42 can be obtained by using (S)-3,3-dimethyl-2-butylamine instead of 2-pyrrolidone in the first step in Example 24, and using a similar method and reaction steps. ESI-MS (m/z): 422.4 [M+H]⁺; ¹HNMR (500 MHz, DMSO-d6) δ 9.18 (s, 1H), 8.65 (s, 1H), 8.10 (s, 1H), 7.78 (d, J=5.7 Hz, 1H), 6.85 (d, J=5.6 Hz, 1H), 6.33 (d, J=9.6 Hz, 1H), 4.18-4.10 (m, 1H), 3.90 (s, 3H), 3.50-3.42 (m, 2H), 2.80 (t, J=5.9 Hz, 2H), 2.68 (t, J=5.9 Hz, 2H), 2.36 (s, 3H), 1.13 (d, J=6.6 Hz, 3H), 0.97 (s, 9H).

### Example 43

### 8-(1,3-Dimethyl-1H-pyrazol-5-yl)-N-(2-methoxy-6-methyl-5,6,7,8-tetrahydro-1,6-naphthyridin-3-yl)pyrido[3,4-d]pyrimidin-2-amine

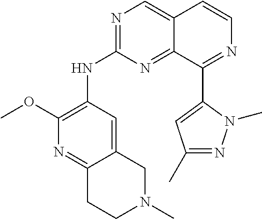

Using 1,3-dimethyl-1H-pyrazole-5-boronic acid pinacol ester to replace 2-methoxyphenylboronic acid in the first step in Example 25, with similar methods and reaction steps, compound 43 can be obtained. ESI-MS (m/z): 417.2 [M+H]⁺; ¹HNMR (500 MHz, DMSO-d6) δ 9.49 (s, 1H), 8.77 (s, 1H), 8.57 (d, J=5.2 Hz, 1H), 8.20 (s, 1H), 7.85 (d, J=5.3 Hz, 1H), 6.71 (s, 1H), 3.90 (s, 3H), 3.85 (s, 3H), 3.39 (s, 2H), 2.79 (t, J=5.9 Hz, 2H), 2.67 (t, J=5.9 Hz, 2H), 2.39 (s, 3H), 2.26 (s, 3H).

### Example 44

### N-(2-ethoxy-6-methyl-5,6,7,8-tetrahydro-1,6-naphthyridin-3-yl)-8-(2-methoxyphenyl)quinazolin-2-amine

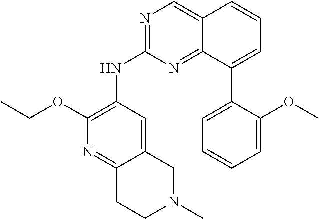 [SMILES: CCOc1nc2c(cc1Nc1ncc3cccc(-c4ccccc4OC)c3n1)CN(C)CC2]

Substitute 2-methoxyphenylboronic acid for phenylboronic acid in the first step in Example 1, then replace Int-2 for the second step with Int-3, and use similar methods and reaction steps to obtain compound 44. ESI-MS (m/z): 442.2 [M+H]⁺; ¹HNMR (500 MHz, DMSO-d6) δ 9.36 (s, 1H), 8.12 (s, 1H), 7.99-7.92 (m, 2H), 7.73 (dd, J=7.1, 1.6 Hz, 1H), 7.52-7.46 (m, 2H), 7.30 (dd, J=7.4, 1.7 Hz, 1H), 7.22 (d, J=8.1 Hz, 1H), 7.12 (td, J=7.4, 1.0 Hz, 1H), 4.35 (q, J=7.0 Hz, 2H), 3.61 (s, 3H), 3.06 (s, 2H), 2.69 (t, J=5.8 Hz, 2H), 2.60 (t, J=5.8 Hz, 2H), 2.40 (s, 3H), 1.37 (t, J=7.0 Hz, 3H).

### Example 45

### 8-(2-methoxyphenyl)-N-(6-methyl-5,6,7,8-tetrahydro-1,6-naphthyridin-3-yl)quinazolin-2-amine

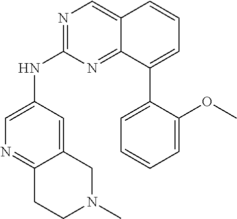 [SMILES: COc1ccccc1-c1cccc2cnc(Nc3cnc4c(c3)CN(C)CC4)nc12]

Substitute 2-methoxyphenylboronic acid for phenylboronic acid in the first step in Example 1, then replace Int-2 for the second step with Int-1, and use similar methods and reaction steps to obtain compound 45. ESI-MS (m/z): 398.2 [M+H]⁺; ¹HNMR (500 MHz, Chloroform-d) δ 9.12 (s, 1H), 8.23-8.19 (m, 1H), 8.16-8.12 (m, 1H), 7.79-7.74 (m, 2H), 7.50-7.42 (m, 2H), 7.37 (dd, J=7.4, 1.7 Hz, 1H), 7.30-7.28 (m, 1H), 7.15-7.08 (m, 2H), 3.67 (s, 3H), 3.40-3.26 (m, 2H), 3.03-2.95 (m, 2H), 2.87-2.75 (m, 2H), 2.55 (s, 3H).

### Example 46

### N-(2,6-dimethyl-5,6,7,8-tetrahydro-1,6-naphthyridin-3-yl)-8-(2-methoxyphenyl)quinazoline-2-amine

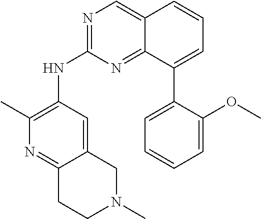

Using 2-methoxyphenylboronic acid to replace the phenylboronic acid in the first step in Example 1, and then replacing Int-2 in the second step with Int-11, compound 46 can be obtained by using similar methods and reaction steps. ESI-MS (m/z): 412.2 [M+H]⁺; ¹H NMR (500 MHz, Chloroform-d) δ 9.12 (s, 1H), 8.34 (s, 1H), 7.78-7.74 (m, 2H), 7.48-7.41 (m, 2H), 7.36 (d, J=7.4 Hz, 1H), 7.14-7.06 (m, 2H), 3.65 (s, 3H), 3.22 (s, 2H), 2.94 (t, J=5.9 Hz, 2H), 2.74 (t, J=5.6 Hz, 2H), 2.55 (s, 3H), 2.50 (s, 3H).

### Example 47

### N-(2-ethoxy-6-methyl-5,6,7,8-tetrahydro-1,6-naphthyridin-3-yl)-8-(2-methoxypyridin-3-yl) quinazolin-2-amine

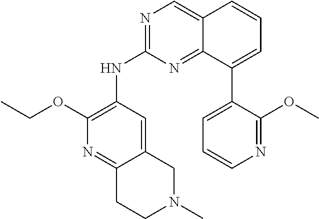 [SMILES: CCOc1nc2c(cc1Nc1ncc3cccc(-c4cccnc4OC)c3n1)CN(C)CC2]

Compound 47 was prepared by the following steps:

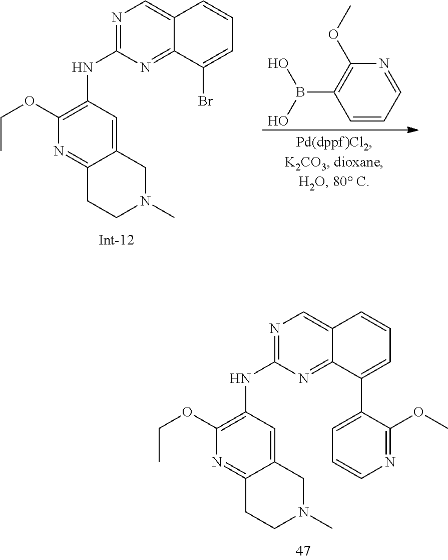 [SMILES: CCOc1nc2c(cc1Nc1ncc3cccc(Br)c3n1)CN(C)CC2 ; COc1ncccc1B(O)O ; CCOc1nc2c(cc1Nc1ncc3cccc(-c4cccnc4OC)c3n1)CN(C)CC2]

Dissolve Int-12 (30 mg, 74 umol) and 2-methoxypyridyl-3-boronic acid (14 mg, 94 umol) in the mixed solvent of 1,4-dioxane (5 mL) and water (0.5 mL), add sodium carbonate (23 mg, 217 umol) and Pd(dppf)Cl₂ (5 mg, 7 umol), and the reaction system was replaced with nitrogen and heated to 90° C. and stirred overnight. After the reaction solution was cooled to room temperature, the reaction solution was filtered through celite, and the filtrate was concentrated. The residue was purified by Prep-HPLC to obtain yellow solid 47 (9 mg, yield 28%). ESI-MS (m/z): 443.2 [M+H]⁺; ¹HNMR (500 MHz, DMSO-d6) δ 9.36 (s, 1H), 8.32 (d, J=5.0 Hz, 1H), 8.03 (s, 1H), 8.01-7.98 (m, 2H), 7.79 (d, J=7.1 Hz, 1H), 7.73 (d, J=7.2 Hz, 1H), 7.49 (t, J=7.6 Hz, 1H), 7.18 (dd, J=7.1, 5.1 Hz, 1H), 4.34 (q, J=7.0 Hz, 2H), 3.71 (s, 3H), 3.12 (s, 2H), 2.69 (t, J=5.7 Hz, 2H), 2.60 (t, J=5.8 Hz, 2H), 2.40 (s, 3H), 1.36 (t, J=7.0 Hz, 3H).

### Example 48

### N-(2,6-dimethyl-5,6,7,8-tetrahydro-1,6-naphthyridin-3-yl)-8-(2-methoxypyridin-3-yl)quinazolin-2-amine

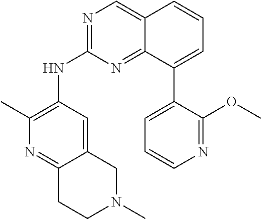

Compound 47 can be obtained by replacing Int-7 in the first step in Example 23 with Int-13 and using a similar method and reaction steps. ESI-MS (m/z): 413.3 [M+H]⁺; ¹HNMR (500 MHz, DMSO-d6) δ 9.34 (s, 1H), 8.78 (s, 1H), 8.24 (d, J=5.0 Hz, 1H), 7.94 (d, J=8.0 Hz, 1H), 7.83 (s, 1H), 7.73 (d, J=7.1 Hz, 1H), 7.69 (d, J=7.2 Hz, 1H), 7.43 (t, J=5.0 Hz, 1H), 7.09 (dd, J=7.2, 5.0 Hz, 1H), 3.72 (s, 3H), 3.25 (s, 2H), 2.76 (t, J=5.8 Hz, 2H), 2.63 (t, J=5.9 Hz, 2H), 2.41 (s, 3H), 2.39 (s, 3H).

### Example 49

### 6-methyl-3-((8-morpholinopyrido[3,4-d]pyrimidin-2-yl)amino)-5,6,7,8-tetrahydro-1, 6-naphthyridine-2(1H)-one

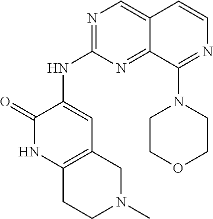 [SMILES: CN1CCc2[nH]c(=O)c(Nc3ncc4ccnc(N5CCOCC5)c4n3)cc2C1]

Compound 49 was prepared by the following steps:

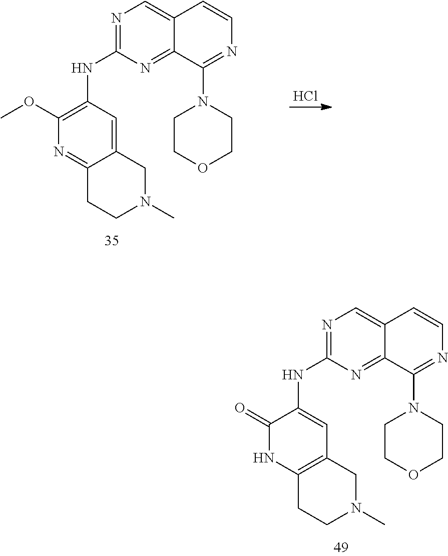 [SMILES: COc1nc2c(cc1Nc1ncc3ccnc(N4CCOCC4)c3n1)CN(C)CC2 ; Cl ; CN1CCc2[nH]c(=O)c(Nc3ncc4ccnc(N5CCOCC5)c4n3)cc2C1]

Step 1: Dissolve compound 35 (20 mg, 0.049 mmol) in 1,4-dioxane (5 mL), add concentrated hydrochloric acid (0.2 mL), and react at 100° C. for 2 hours. LCMS shows that the reaction of the starting material is complete. The reaction solution was directly concentrated, and the residue was purified by Prep-HPLC to obtain compound 49 (8 mg, yield 42%). ESI-MS (m/z): 394.2 [M+H]⁺; ¹HNMR (500 Hz, DMSO-d6) S 11.95 (br s, 1H), 9.33 (s, 1H), 8.47 (s, 1H), 8.06 (d, J=5.4 Hz, 1H), 7.98 (s, 1H), 7.28 (d, J=5.4 Hz, 1H), 3.88-3.83 (m, 4H), 3.79-3.74 (m, 4H), 3.35-3.32 (m, 2H), 2.63-2.58 (m, 4H), 2.37 (s, 3H).

### Example 50

### (S)—N8-(3,3-dimethylbut-2-yl)-N2-(2-ethoxy-6-methyl-5,6,7,8-tetrahydro-1,6-Naphthyridin-3-yl)-6-methylpyrido[3,4-d]pyrimidine-2,8-diamine

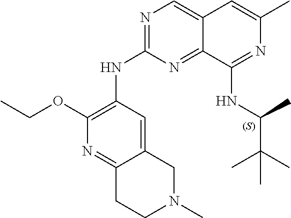

Compound 50 was prepared by the following steps:

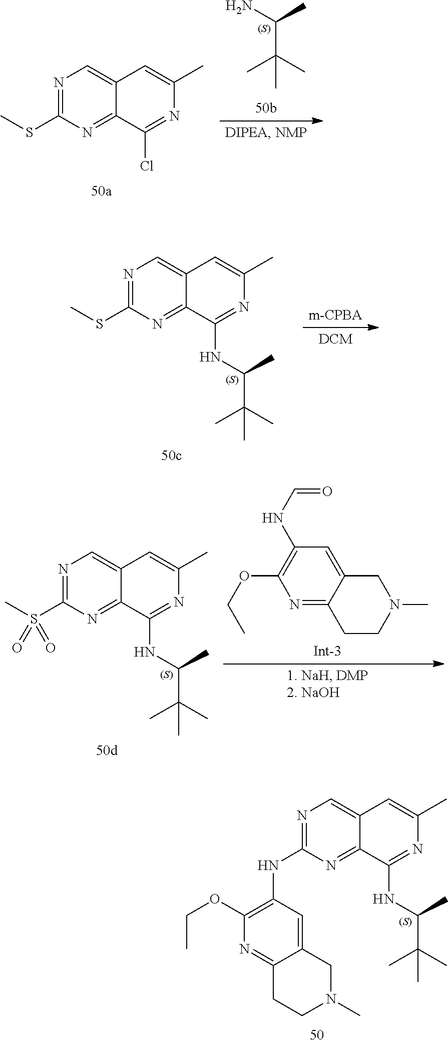

Step 1: Compound 8-chloro-6-methyl-2-(methylthio)pyrido[3,4-d]pyrimidine 50a (50 mg, 0.22 mmol) and (S)-3,3-bis Methyl-2-butylamine 50b (112 mg, 1.11 mmol) were dissolved in N-methylpyrrolidone (4 mL), diisopropylethylamine (143 mg, 1.11 mmol) was added, and the reaction mixture was heated to 130° C. by microwave to react for 5 hours, and the formation of the product was detected by LCMS. The reaction solution was poured into water (15 mL), extracted with ethyl acetate (10 mL*2), the organic phases were combined, washed 3 times with saturated brine, dried over sodium sulfate, filtered and concentrated to obtain compound 50c (60 mg, yield 93%). ESI-MS (m/z): 291.4 [M+H]⁺.

Step 2: Dissolve compound 50c (60 mg, 0.20 mmol) in dichloromethane (10 mL), add m-CPBA (105 mg, content 85%, 0.51 mmol) under ice bath, then rise to room temperature and react overnight, LCMS detected that the reaction of raw materials was complete. The reaction solution was concentrated, and the residue was purified by Prep-TLC to obtain compound 50d (16 mg, yield 22%). ESI-MS (m/z): 323.4 [M+H]⁺.

Step 3: Compound 50d (16 mg, 0.07 mmol) was dissolved in anhydrous DMF (3 mL), NaH (3 mg, content 60%, 0.07 mmol) was added under ice bath, stirring was continued for 30 minutes under ice bath, and Int-3 (15 mg, 0.047 mmol) in DMF (2 mL) was added dropwise and reacted at room temperature for 2 hours. LCMS detected that the raw material was completely reacted. The reaction solution was directly prepared by Prep-HPLC to obtain compound 50 (2 mg, yield 11%). ESI-MS (m/z): 450.3 [M+H]⁺; ¹HNMR (500 MHz, DMSO-d6) δ 9.10 (s, 1H), 8.39 (s, 1H), 8.16 (s, 1H), 6.69 (d, J=1.0 Hz, 1H), 6.29 (d, J=9.4 Hz, 1H), 4.36 (qd, J=7.0, 1.0 Hz, 2H), 4.15 (dd, J=9.5, 6.7 Hz, 1H), 3.57-3.40 (m, 2H), 2.78 (t, J=5.8 Hz, 2H), 2.69 (d, J=6.0 Hz, 2H), 2.38-2.35 (m, 6H), 1.33 (t, J=7.1 Hz, 3H), 1.14 (d, J=6.7 Hz, 3H), 0.98 (s, 9H).

### Example 51

### N2-(2-ethoxy-6-methyl-5,6,7,8-tetrahydro-1,6-naphthyridin-3-yl)-6-methyl-N8-neopentylpyrido[3,4-d]pyrimidine-2,8-diamine

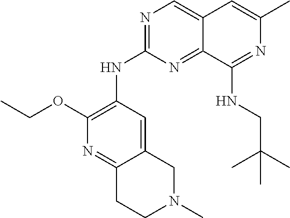 [SMILES: CCOc1nc2c(cc1Nc1ncc3cc(C)nc(NCC(C)(C)C)c3n1)CN(C)CC2]

Compound 51 can be obtained by replacing (S)-3,3-dimethyl-2-butylamine in the first step in Example 50 with neopentylamine and using a similar method and reaction steps. ESI-MS (m/z): 436.2 [M+H]⁺; ¹HNMR (500 MHz, DMSO-d6) δ 9.10 (s, 1H), 8.31 (s, 1H), 8.26 (s, 1H), 6.69 (d, J=1.0 Hz, 1H), 6.55 (t, J=6.1 Hz, 1H), 4.36 (q, J=7.0 Hz, 2H), 3.46 (s, 2H), 3.37 (d, J=6.2 Hz, 2H), 2.77 (t, J=5.9 Hz, 2H), 2.66 (t, J=5.8 Hz, 2H), 2.35 (s, 6H), 1.34 (t, J=6.6 Hz, 3H), 0.99 (s, 9H).

### Example 52

### N2-(2-ethoxy-6-methyl-5,6,7,8-tetrahydro-1,6-naphthyridin-3-yl)-N8-neopentylpyrido[3,4-d]pyrimidine-2,8-diamine

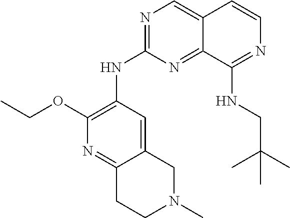 [SMILES: CCOc1nc2c(cc1Nc1ncc3ccnc(NCC(C)(C)C)c3n1)CN(C)CC2]

The two reactants in the first step in Example 50 (S)-3,3-dimethyl-2-butylamine and 8-chloro-6-methyl-2-(methylthio)pyrido[3, 4-d]pyrimidine was replaced by neopentylamine and 8-chloro-2-(methylsulfanyl)pyrido[3,4-d]pyrimidine, respectively, and compound 52 could be obtained by using similar methods and reaction steps. ESI-MS (m/z): 422.3 [M+H]⁺; ¹HNMR (500 MHz, DMSO-d6) δ 9.19 (s, 1H), 8.46 (s, 1H), 8.25 (s, 1H), 7.79 (d, J=5.6 Hz, 1H), 6.87 (d, J=5.7 Hz, 1H), 6.63 (t, J=6.3 Hz, 1H), 4.37 (q, J=7.0 Hz, 2H), 3.47 (s, 2H), 3.39 (d, J=6.3 Hz, 2H), 2.77 (t, J=5.9 Hz, 2H), 2.67 (t, J=5.9 Hz, 2H), 2.35 (s, 3H), 1.33 (t, J=7.0 Hz, 3H), 0.98 (s, 9H).

### Example 53

### 8-(1,5-dimethyl-1H-pyrazol-4-yl)-N-(2-methoxy-6-methyl-5,6,7,8-tetrahydro-1,6-Naphthyridin-3-yl)quinazolin-2-amine

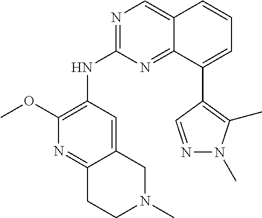

With 1,5-dimethyl-1H-pyrazole-4-boronic acid pinacol ester to replace the 2-methoxy-3-pyridineboronic acid in the first step in Example 23, with similar methods and reaction steps, compound 53 was obtained. ESI-MS (m/z): 416.3 [M+H]⁺; ¹HNMR (500 MHz, DMSO-d6) δ 9.34 (s, 1H), 8.41 (s, 1H), 8.13 (s, 1H), 7.89 (dd, J=8.0, 1.5 Hz, 1H), 7.71 (dd, J=7.0, 1.5 Hz, 1H), 7.65 (s, 1H), 7.46 (dd, J=8.0, 7.0 Hz, 1H), 3.91 (s, 3H), 3.86 (s, 3H), 3.34 (s, 2H), 2.76 (t, J=6.0 Hz, 2H), 2.66 (t, J=6.0 Hz, 2H), 2.40 (s, 3H), 2.19 (s, 3H).

### Example 54

### 5-(2-((2-ethoxy-6-methyl-5,6,7,8-tetrahydro-1,6-naphthyridin-3-yl)amino)quinazolin-8-yl)-1-methylpyridin-2(1H)-one

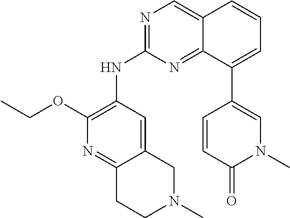 [SMILES: CCOc1nc2c(cc1Nc1ncc3cccc(-c4ccc(=O)n(C)c4)c3n1)CN(C)CC2]

Use 1-methyl-6-oxo-1,6-dihydropyridine-3-boronic acid pinacol ester to replace 2-methoxy-3-pyridineboronic acid in the first step in Example 47, and use a similar method and reaction steps, compound 54 can be obtained. ESI-MS (m/z): 443.3 [M+H]⁺; ¹HNMR (500 MHz, Chloroform-d) δ 9.12 (s, 1H), 8.48 (s, 1H), 7.86 (s, 1H), 7.78 (d, J=9.3 Hz, 1H), 7.74 (d, J=8.0 Hz, 1H), 7.69 (d, J=7.2 Hz, 1H), 7.60 (d, J=2.3 Hz, 1H), 7.38 (t, J=7.6 Hz, 1H), 6.71 (d, J=9.4 Hz, 1H), 4.45 (q, J=7.1 Hz, 2H), 3.63 (s, 3H), 3.36 (s, 2H), 2.89 (t, J=5.9 Hz, 2H), 2.76 (t, J=5.8 Hz, 2H), 2.55 (s, 3H), 1.43 (t, J=7.1 Hz, 3H).

### Example 55

### 8-(1,5-dimethyl-1H-pyrazol-4-yl)-N-(2-ethoxy-6-methyl-5,6,7,8-tetrahydro-1,6-naphthyridin-3-yl)quinazolin-2-amine

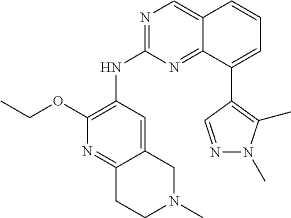 [SMILES: CCOc1nc2c(cc1Nc1ncc3cccc(-c4cnn(C)c4C)c3n1)CN(C)CC2]

With 1,5-dimethyl-1H-pyrazole-4-boronic acid pinacol ester to replace the 2-methoxy-3-pyridineboronic acid in the first step in Example 47, with similar methods and reaction steps, compound 54 was obtained. ESI-MS (m/z): 430.2 [M+H]⁺; ¹HNMR (500 MHz, Chloroform-d) δ 9.11 (s, 1H), 8.58 (s, 1H), 7.84 (s, 1H), 7.76 (s, 1H), 7.70 (d, J=8.0 Hz, 1H), 7.66 (d, J=7.1 Hz, 1H), 7.38 (t, J=7.6 Hz, 1H), 4.44 (q, J=7.1 Hz, 2H), 3.94 (s, 3H), 3.50 (s, 2H), 2.90 (t, J=5.8 Hz, 2H), 2.80 (t, J=5.9 Hz, 2H), 2.55 (s, 3H), 2.24 (s, 3H), 1.43 (t, J=7.1 Hz, 3H).

### Example 56

### N2-(2-ethoxy-6-methyl-5,6,7,8-tetrahydro-1,6-naphthyridin-3-yl)-N8-isobutylquinazoline-2,8-diamine

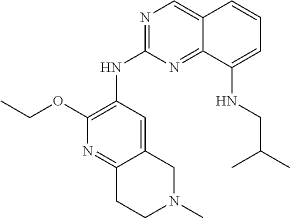 [SMILES: CCOc1nc2c(cc1Nc1ncc3cccc(NCC(C)C)c3n1)CN(C)CC2]

Compound 56 was prepared by the following steps:

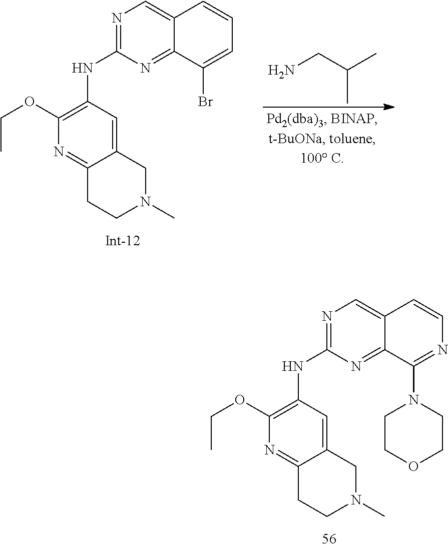

Step 1: Dissolve Int-12 (40 mg, 96 umol) in toluene (5 mL), add Pd2(dba)3 (4 mg, 4.9 umol), BINAP (9 mg, 14.9 umol), sodium tert-butoxide (18 mg, 193 umol) and isobutylamine (11 mg, 144 umol). The reaction system was replaced with nitrogen and heated to 100° C. and stirred overnight. The reaction solution was concentrated, and the residue was purified by Prep-TLC (dichloromethane/methanol=10/1) to obtain a crude product, which was further purified by Prep-HPLC to obtain a yellow solid 56 (23 mg, yield 59%). ESI-MS (m/z): 407.2 [M+H]⁺; ¹H NMR (500 MHz, DMSO-d6) δ 9.19 (s, 1H), 8.37 (s, 1H), 8.15 (s, 1H), 7.21 (t, J=8.0 Hz, 1H), 7.10 (d, J=8.0 Hz, 1H), 6.80 (d, J=7.5 Hz, 1H), 5.67 (t, J=6.0 Hz, 1H), 4.38 (q, J=7.0 Hz, 2H), 3.48 (s, 2H), 3.07 (t, J=6.0 Hz, 2H), 2.77 (t, J=6.0 Hz, 2H), 2.67 (t, J=6.0 Hz, 2H), 2.37 (s, 3H), 2.04-1.96 (m, 1H), 1.36 (t, J=7.0 Hz, 3H), 1.03 (d, J=6.5 Hz, 6H).

### Example 57

### (R)-2-((2-((2-ethoxy-6-methyl-5,6,7,8-tetrahydro-1,6-naphthyridin-3-yl)amino)pyrido[3,4-d]pyrimidin-8-yl)amino)-3,3-dimethylbutan-1-ol

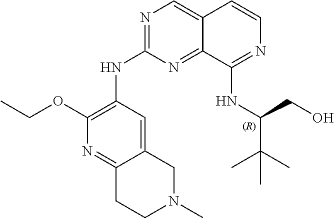

The two reactants in the first step in Example 50 (S)-3,3-dimethyl-2-butylamine and 8-chloro-6-methyl-2-(methylthio)pyrido[3, 4-d]pyrimidine is replaced by D-tert-leucinol and 8-chloro-2-(methylsulfanyl)pyrido[3,4-d]pyrimidine respectively, with similar methods and reaction steps, compound 57 can be obtained. ESI-MS (m/z): 452.3 [M+H]⁺; ¹HNMR (500 MHz, DMSO-d6) δ 9.20 (s, 1H), 8.44 (s, 1H), 8.30 (s, 1H), 7.78 (d, J=5.4 Hz, 1H), 6.86 (d, J=5.4 Hz, 1H), 6.72 (d, J=9.8 Hz, 1H), 4.73 (t, J=5.0 Hz, 1H), 4.37 (q, J=7.0 Hz, 2H), 4.15 (dd, J=9.7, 4.7 Hz, 1H), 3.73-3.60 (m, 2H), 3.53-3.38 (m, 2H), 2.81-2.73 (m, 2H), 2.70-2.62 (m, 2H), 2.36-2.33 (m, 3H), 1.35 (t, J=7.0 Hz, 3H), 1.01 (s, 9H).

### Example 58

### (R)—N8-(3,3-dimethylbut-2-yl)-N2-(2-ethoxy-6-methyl-5,6,7,8-tetrahydro-1,6-Naphthyridin-3-yl)pyrido[3,4-d]pyrimidine-2,8-diamine

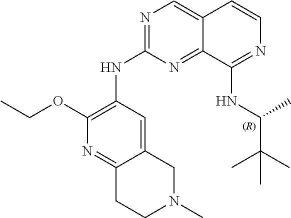

The two reactants in the first step in Example 50 (S)-3,3-dimethyl-2-butylamine and 8-chloro-6-methyl-2-(methylthio)pyrido[3,4-d]pyrimidine were replaced by (R)-3,3-dimethyl-2-butylamine and 8-chloro-2-(methylsulfanyl)pyrido[3,4-d]pyrimidine, respectively. Compound 58 can be obtained by similar methods and reaction steps. ESI-MS (m/z): 436.1 [M+H]⁺; ¹HNMR (500 MHz, DMSO-d6) δ 9.18 (s, 1H), 8.53 (s, 1H), 8.13 (s, 1H), 7.79 (d, J=5.7 Hz, 1H), 6.86 (d, J=5.7 Hz, 1H), 6.32 (d, J=9.5 Hz, 1H), 4.36 (q, J=6.9 Hz, 2H), 4.16 (q, J=9.4, 6.5 Hz, 1H), 3.53-3.39 (m, 2H), 2.78 (t, J=5.8 Hz, 2H), 2.67 (t, J=5.6 Hz, 2H), 2.36 (s, 3H), 1.32 (t, J=7.0 Hz, 3H), 1.14 (d, J=6.6 Hz, 3H), 0.98 (s, 9H).

### Example 59

### 8-benzyl-N-(2-methoxy-6-methyl-5,6,7,8-tetrahydro-1,6-naphthyridin-3-yl)quinazolin-2-amine

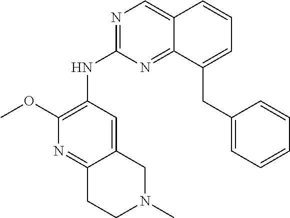 [SMILES: COc1nc2c(cc1Nc1ncc3cccc(Cc4ccccc4)c3n1)CN(C)CC2]

Compound 59 was prepared by the following steps:

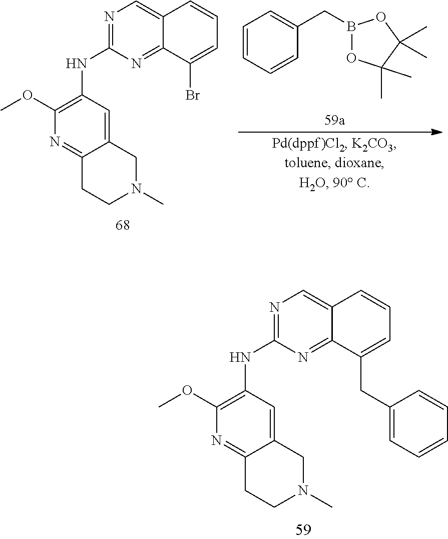

Step 1: Dissolve Int-7 (30 mg, 74 umol) in a mixed solvent of toluene/1,4-dioxane/water (10/1/1, 6 mL), add potassium carbonate (31 mg, 0.22 mmol), Pd(dppf)Cl₂ (5 mg, 7 umol) and 2-benzyl-4,4,5,5-tetramethyl-1,3,2-dioxaborolane 59a (32 mg, 0.14 mmol). The reaction system was replaced with nitrogen and heated to 90° C. and stirred overnight. After the reaction solution was cooled to room temperature, the reaction solution was filtered through celite, and the filtrate was concentrated. The residue was purified by Prep-TLC (dichloromethane/methanol=20/1) to obtain a crude product, which was then separated by Prep-HPLC to obtain a white solid 59 (6 mg, yield 21%). ESI-MS (m/z): 412.3 [M+H]⁺; ¹HNMR (500 MHz, DMSO-d6) δ 9.31 (s, 1H), 8.40 (s, 1H), 8.25 (s, 1H), 7.84 (dd, J=8.0, 1.5 Hz, 1H), 7.72 (dd, J=7.0, 1.5 Hz, 1H), 7.37 (t, J=7.5 Hz, 1H), 7.25-7.19 (m, 4H), 7.17-7.13 (m, 1H), 4.41 (s, 2H), 3.92 (s, 3H), 3.28 (s, 2H), 2.76 (t, J=6.0 Hz, 2H), 2.64 (t, J=6.0 Hz, 2H), 2.34 (s, 3H).

### Example 60

### N2-(2-ethoxy-6-methyl-5,6,7,8-tetrahydro-1,6-naphthyridin-3-yl)-N8-((3-methyloxetane-3-yl)methyl)quinazoline-2,8-diamine

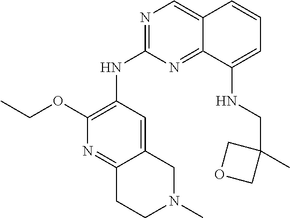 [SMILES: CCOc1nc2c(cc1Nc1ncc3cccc(NCC4(C)COC4)c3n1)CN(C)CC2]

The morpholine in the first step in Example 56 was replaced by 3-methyl-3-aminomethyl-1-oxetane, and compound 60 can be obtained by a similar methods and reaction steps. ESI-MS (m/z): 435.1 [M+H]⁺; ¹HNMR (500 MHz, DMSO-d6) δ 9.20 (s, 1H), 8.34 (s, 1H), 8.14 (s, 1H), 7.24 (t, J=8.0 Hz, 1H), 7.14 (dd, J=8.0, 1.5 Hz, 1H), 6.94 (dd, J=7.5, 1.5 Hz, 1H), 5.85 (t, J=6.0 Hz, 1H), 4.51 (d, J=5.5 Hz, 2H), 4.40-4.35 (m, 4H), 3.47 (s, 2H), 3.45 (d, J=6.0 Hz, 2H), 2.77 (t, J=6.0 Hz, 2H), 2.67 (t, J=6.0 Hz, 2H), 2.37 (s, 3H), 1.39 (s, 3H), 1.36 (t, J=7.0 Hz, 3H).

### Example 61

### 3-((8-(1,5-dimethyl-1H-pyrazol-4-yl)quinazolin-2-yl)amino)-6-methyl-5,6,7,8-tetrahydro-1,6-naphthyridin-2(1H)-one

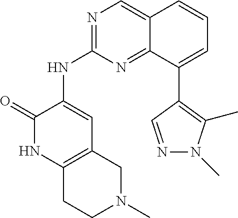 [SMILES: Cc1c(-c2cccc3cnc(Nc4cc5c([nH]c4=O)CCN(C)C5)nc23)cnn1C]

Compound 61 was prepared by the following steps:

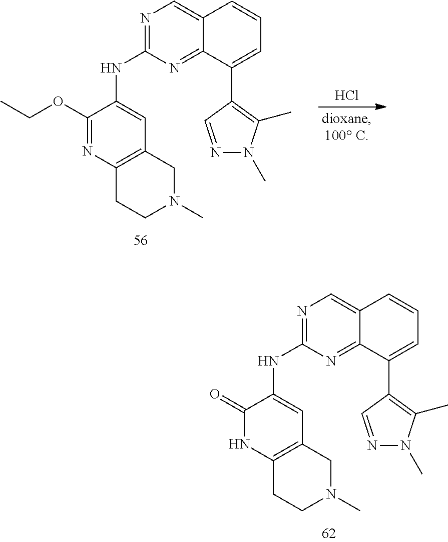 [SMILES: CCOc1nc2c(cc1Nc1ncc3cccc(-c4cnn(C)c4C)c3n1)CN(C)CC2 ; Cl ; Cc1c(-c2cccc3cnc(Nc4cc5c([nH]c4=O)CCN(C)C5)nc23)cnn1C]

Step 1: Compound 56 (30 mg, 69 umol) was dissolved in 1,4-dioxane (5 mL), concentrated hydrochloric acid (0.2 mL) was added, and the reaction mixture was heated to 100° C. and stirred for 3 hours. The reaction solution was concentrated, the residue solid was washed with ethyl acetate, and then purified by Prep-HPLC to obtain compound 61 (3 mg, yield 12%). ESI-MS (m/z): 402.3 [M+H]⁺; ¹HNMR (500 MHz, DMSO-d6) δ 11.83 (br s, 1H), 9.35 (s, 1H), 8.23 (d, J=9.3 Hz, 2H), 7.90 (d, J=8.0 Hz, 1H), 7.72 (d, J=7.2 Hz, 1H), 7.67 (s, 1H), 7.50-7.43 (m, 1H), 3.87 (s, 3H), 3.14 (s, 2H), 2.60-2.55 (m, 4H), 2.37 (s, 3H), 2.23 (s, 3H).

### Example 62

### 3-((8-(2-methoxyphenyl)pyrido[3,4-d]pyrimidin-2-yl)amino)-6-methyl-5,6,7,8-tetrahydro-1,6-naphthyridin-2(1H)-one

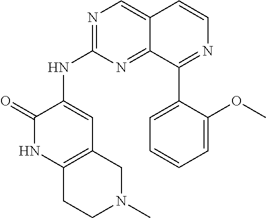 [SMILES: COc1ccccc1-c1nccc2cnc(Nc3cc4c([nH]c3=O)CCN(C)C4)nc12]

Compound 62 can be obtained by replacing the raw material 56 in the first step in Example 61 with compound 25 and using a similar methods and reaction steps. ESI-MS (m/z): 415.0 [M+H]⁺.

### Example 63

### 6-methyl-3-((8-(piperidin-1-yl)pyrido[3,4-d]pyrimidin-2-yl)amino)-5,6,7,8-tetrahydro-1, 6-naphthyridin-2(1H)-one

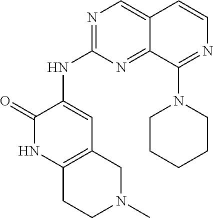 [SMILES: CN1CCc2[nH]c(=O)c(Nc3ncc4ccnc(N5CCCCC5)c4n3)cc2C1]

Compound 63 can be obtained by replacing the raw material 35 in the first step in Example 49 with compound 33 and using a similar methods and reaction steps. ESI-MS (m/z): 392.0 [M+H]⁺; ¹HNMR (500 MHz, DMSO-d6) δ 11.95 (br s, 1H), 9.29 (s, 1H), 8.41 (s, 1H), 8.18 (s, 1H), 8.10 (s, 1H), 8.03 (d, J=5.3 Hz, 1H), 7.20 (d, J=5.3 Hz, 1H), 3.80-3.70 (m, 4H), 3.32 (s, 2H), 2.65-2.56 (m, 4H), 2.36 (s, 3H), 1.80-1.60 (m, 6H).

### Example 64

### 6-methyl-3-((8-(neopentylamino)pyrido[3,4-d]pyrimidin-2-yl)amino)-5,6,7,8-tetrahydro-1,6-naphthyridin-2(1H)-one

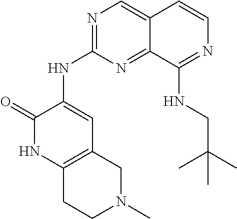 [SMILES: CN1CCc2[nH]c(=O)c(Nc3ncc4ccnc(NCC(C)(C)C)c4n3)cc2C1]

Compound 64 can be obtained by replacing the raw material 35 in the first step in Example 49 with compound 41 and using a similar methods and reaction steps. ESI-MS (m/z): 394.1 [M+H]⁺; ¹HNMR (500 MHz, DMSO-d6) δ 11.94 (s, 1H), 9.22 (s, 1H), 8.43 (s, 1H), 8.21 (s, 1H), 7.83 (d, J=5.6 Hz, 1H), 6.89 (d, J=5.6 Hz, 1H), 6.77-6.74 (m, 1H), 3.43 (d, J=6.2 Hz, 2H), 3.28 (s, 2H), 2.59 (br s, 4H), 2.32 (s, 3H), 1.01 (s, 9H).

### Example 65

### 8-(3,3-dimethylazetidin-1-yl)-N-(2-ethoxy-6-methyl-5,6,7,8-tetrahydro-1,6-Naphthyridin-3-yl)pyrido[3,4-d]pyrimidin-2-amine

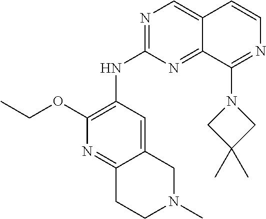 [SMILES: CCOc1nc2c(cc1Nc1ncc3ccnc(N4CC(C)(C)C4)c3n1)CN(C)CC2]

The two reactants in the first step in Example 50 (S)-3,3-dimethyl-2-butylamine and 8-chloro-6-methyl-2-(methylthio)pyrido[3,4-d]pyrimidine were replaced by 3,3-dimethylazetidine and 8-chloro-2-(methylsulfanyl)pyrido[3,4-d]pyrimidine, respectively. In a similar manner and reaction steps, compound 65 can be obtained. ESI-MS (m/z): 420.3 [M+H]⁺; ¹H NMR (500 MHz, DMSO-d6) δ 8.41 (s, 1H), 8.02 (s, 1H), 7.83 (d, J=5.5 Hz, 1H), 6.93 (d, J=5.5 Hz, 1H), 4.34 (q, J=7.0 Hz, 2H), 3.99 (br s, 4H), 3.44 (s, 2H), 2.78 (t, J=6.0 Hz, 2H), 2.67 (t, J=5.9 Hz, 2H), 2.37 (s, 4H), 1.32 (t, J=7.0 Hz, 3H), 1.29 (s, 6H).

### Example 66

### N-(2-ethoxy-6-methyl-5,6,7,8-tetrahydro-1,6-naphthyridin-3-yl)-8-(7-oxa-2-azaspiro [3.5]non-2-yl)pyrido[3,4-d]pyrimidin-2-amine

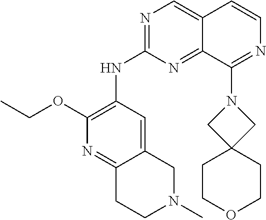 [SMILES: CCOc1nc2c(cc1Nc1ncc3ccnc(N4CC5(CCOCC5)C4)c3n1)CN(C)CC2]

The two reactants in the first step in Example 50 (S)-3,3-dimethyl-2-butylamine and 8-chloro-6-methyl-2-(methylthio)pyrido[3,4-d]pyrimidine was replaced by 7-oxa-2-azaspiro[3.5]nonane and 8-chloro-2-(methylsulfanyl)pyrido[3,4-d]pyrimidine, respectively, with Compound 66 can be obtained by similar method and reaction steps. ESI-MS (m/z): 462.2 [M+H]⁺; ¹H NMR (500 MHz, DMSO-d6) δ 9.17 (d, J=1.9 Hz, 1H), 8.48 (s, 1H), 7.95 (s, 1H), 7.82 (d, J=5.5 Hz, 1H), 6.92 (d, J=5.5 Hz, 1H), 4.33 (q, J=7.0 Hz, 2H), 4.02 (br s, 4H), 3.60-3.52 (m, 4H), 3.47 (s, 2H), 2.78 (t, J=5.9 Hz, 2H), 2.68 (t, J=5.9 Hz, 2H), 2.37 (s, 3H), 1.76-1.72 (m, 4H), 1.30 (t, J=7.0 Hz, 3H).

### Example 67

### N-(2-ethoxy-6-methyl-5,6,7,8-tetrahydro-1,6-naphthyridin-3-yl)-8-(1,3,5-trimethyl-1H-pyrazol-4-yl)quinazolin-2-amine

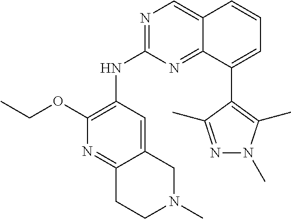 [SMILES: CCOc1nc2c(cc1Nc1ncc3cccc(-c4c(C)nn(C)c4C)c3n1)CN(C)CC2]

Replace the 2-methoxy-3-pyridineboronic acid in the first step in Example 47 with 1,3,5-trimethyl-1H-pyrazole-4-boronic acid pinacol ester, and use a similar method and reaction steps, compound 67 can be obtained. ESI-MS (m/z): 444.3 [M+H]⁺; ¹HNMR (500 MHz, DMSO-d6) δ 9.36 (s, 1H), 8.34 (s, 1H), 8.01 (s, 1H), 7.94 (d, J=8.1 Hz, 1H), 7.68 (d, J=7.1 Hz, 1H), 7.48 (t, J=6.1 Hz, 1H), 4.36 (q, J=7.0 Hz, 2H), 3.80 (s, 3H), 3.42-3.37 (m, 1H), 3.22-3.15 (m, 1H), 2.77-2.70 (m, 2H), 2.68-2.58 (m, 2H), 2.40 (s, 3H), 2.07 (s, 3H), 1.94 (s, 3H), 1.37 (t, J=7.0 Hz, 3H).

### Example 68

### 1,6-dimethyl-3-((8-(piperidin-1-yl)pyrido[3,4-d]pyrimidin-2-yl)amino)-5,6,7,8-tetrahydro-1,6-naphthyridin-2(1H)-one

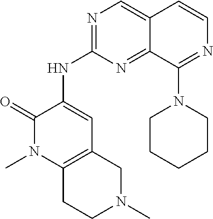 [SMILES: CN1CCc2c(cc(Nc3ncc4ccnc(N5CCCCC5)c4n3)c(=O)n2C)C1]

Compound 68 was prepared by the following steps:

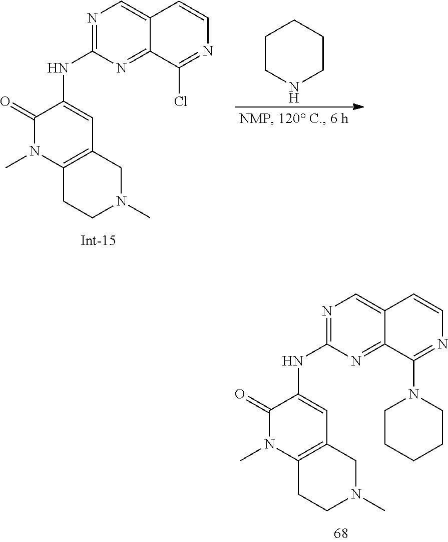 [SMILES: CN1CCc2c(cc(Nc3ncc4ccnc(Cl)c4n3)c(=O)n2C)C1 ; C1CCNCC1 ; CN1CCc2c(cc(Nc3ncc4ccnc(N5CCCCC5)c4n3)c(=O)n2C)C1]

Step 1: Dissolve Int-15 (25 mg, 70 umol) in N-methylpyrrolidone (0.5 mL), and add piperidine (30 mg, 0.35 mmol). The reaction solution was heated to 160° C. and stirred for 6 hours, and the LCMS detected that the reaction was complete. The reaction solution was concentrated, and the residue was purified by Prep-HPLC to obtain yellow solid 68 (8 mg, yield 28%). ESI-MS (m/z): 405.4 [M+H]⁺; ¹H NMR (500 MHz, DMSO-d6) δ 9.30 (s, 1H), 8.51 (s, 1H), 8.11 (s, 1H), 8.03 (d, J=5.3 Hz, 1H), 7.21 (d, J=5.3 Hz, 1H), 3.73 (t, J=5.3 Hz, 4H), 3.53 (s, 3H), 3.35 (s, 2H), 2.78 (t, J=5.8 Hz, 2H), 2.65 (t, J=5.8 Hz, 2H), 2.36 (s, 3H), 1.76-1.71 (m, 4H), 1.70-1.65 (m, 2H).

### Example 69

### 1,6-dimethyl-3-((8-morpholinopyrido[3,4-d]pyrimidin-2-yl)amino)-5,6,7,8-tetrahydro-1,6-naphthyridin-2(1H)-one

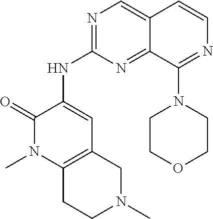 [SMILES: CN1CCc2c(cc(Nc3ncc4ccnc(N5CCOCC5)c4n3)c(=O)n2C)C1]

Substituting morpholine for piperidine in the first step in Example 68, and using a similar method and reaction steps, compound 69 can be obtained. ESI-MS (m/z): 408.3 [M+H]⁺; H NMR (500 MHz, DMSO-d6) δ 9.34 (s, 1H), 8.56 (s, 1H), 8.07 (d, J=5.3 Hz, 1H), 7.99 (s, 1H), 7.29 (d, J=5.4 Hz, 1H), 3.85 (t, 4H), 3.77 (t, J=4.8 Hz, 4H), 3.53 (s, 3H), 3.36 (s, 2H), 2.79 (t, J=6.0 Hz, 2H), 2.65 (t, J=5.9 Hz, 2H), 2.38 (s, 3H).

### Example 70

### 3-((8-((3-hydroxy-2,2-dimethylpropyl)aminopyrido[3,4-d]pyrimidin-2-yl)amino)-6-methyl-5,6, 7,8-tetrahydro-1,6-naphthyridin-2(1H)-one

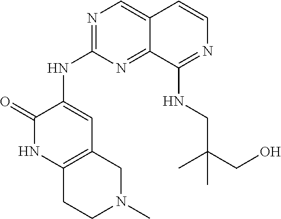 [SMILES: CN1CCc2[nH]c(=O)c(Nc3ncc4ccnc(NCC(C)(C)CO)c4n3)cc2C1]

Compound 70 was prepared by the following steps:

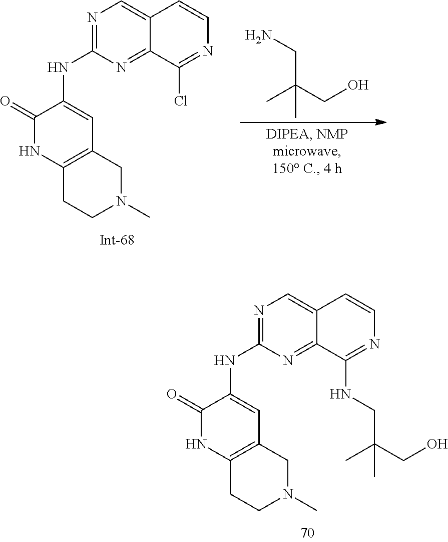

Step 1: Dissolve Int-16 (50 mg, 118 umol, HBr salt) in N-methylpyrrolidone (4 mL), add 3-amino-2, 2-dimethyl-1-propanol (61 mg, 0.59 mmol) and N, N-diisopropylethylamine (76 mg, 0.59 mmol). The reaction solution was heated to 150° C. and stirred for 4 hours in a microwave reactor, and the reaction was detected by LCMS to be complete. The reaction solution was concentrated, and the residue was purified by Prep-HPLC to obtain yellow solid 70 (16 mg, yield 33%). ESI-MS (m/z): 410.3 [M+H]⁺; ¹H NMR (500 MHz, DMSO-d6) δ 11.91 (s, 1H), 9.22 (s, 1H), 8.42 (s, 1H), 8.22 (s, 1H), 7.81 (d, J=5.5 Hz, 1H), 7.23 (s, 1H), 6.88 (d, J=5.5 Hz, 1H), 5.06 (t, J=5.6 Hz, 1H), 3.44 (d, J=5.8 Hz, 2H), 3.34 (s, 2H), 3.28 (d, J=5.0 Hz, 2H), 2.59 (s, 4H), 2.33 (s, 3H), 0.95 (s, 6H).

### Example 71

### (R)-6-methyl-3-((8-(2-methylmorpholinyl)pyrido[3,4-d]pyrimidin-2-yl)amino)-5,6,7,8-tetrahydro-1,6-naphthyridin-2(1H)-one

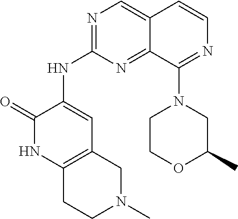 [SMILES: C[C@@H]1CN(c2nccc3cnc(Nc4cc5c([nH]c4=O)CCN(C)C5)nc23)CCO1]

Compound 71 can be obtained by using (R)-2-methylmorpholine instead of 3-amino-2, 2-dimethyl-1-propanol in the first step in Example 70, and using a similar method and reaction steps. ESI-MS (m/z): 408.2 [M+H]⁺; ¹H NMR (500 MHz, DMSO-d6) δ 11.96 (s, 1H), 9.32 (s, 1H), 8.46 (s, 1H), 8.10-7.91 (m, 2H), 7.27 (d, J=5.4 Hz, 1H), 4.56-4.40 (m, 2H), 3.97 (d, J=10.4 Hz, 1H), 3.86-3.72 (m, 2H), 3.35-3.30 (m, 2H), 3.02 (t, J=12.0 Hz, 1H), 2.76-2.66 (m, 1H), 2.63-2.52 (m, 4H), 2.37 (s, 3H), 1.15 (d, J=6.2 Hz, 3H).

### Example 72

### 6-methyl-3-((8-thiomorpholinopyrido[3,4-d]pyrimidin-2-yl)amino)-5,6,7,8-tetrahydro-1,6-naphthalene pyridin-2(1H)-one

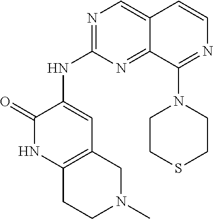 [SMILES: CN1CCc2[nH]c(=O)c(Nc3ncc4ccnc(N5CCSCC5)c4n3)cc2C1]

Compound 72 can be obtained by replacing 3-amino-2,2-dimethyl-1-propanol in the first step in Example 70 with thiomorpholine, and using a similar method and reaction steps. ESI-MS (m/z): 410.2 [M+H]⁺; ¹H NMR (500 MHz, DMSO-d6) δ 11.96 (s, 1H), 9.33 (s, 1H), 8.45 (s, 1H), 8.10-8.02 (m, 2H), 7.26 (d, J=5.4 Hz, 1H), 4.09 (d, J=5.4 Hz, 4H), 2.80 (d, J=5.0 Hz, 4H), 2.60 (s, 4H), 2.36 (s, 4H).

### Example 73

### 3-((8-(azepan-1-yl)pyrido[3,4-d]pyrimidin-2-yl)amino)-6-methyl-5,6,7,8-tetrahydro-1,6-naphthyridin-2(1H)-one

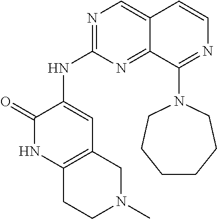 [SMILES: CN1CCc2[nH]c(=O)c(Nc3ncc4ccnc(N5CCCCCC5)c4n3)cc2C1]

Compound 73 can be obtained by replacing the 3-amino-2,2-dimethyl-1-propanol in the first step in Example 70 with cyclohexyl imine, and using a similar methods and reaction steps. ESI-MS (m/z): 405.4 [M+H]⁺; ¹H NMR (500 MHz, DMSO-d6) δ 9.11 (s, 1H), 8.12 (s, 1H), 7.90 (d, J=5.5 Hz, 1H), 6.92 (d, J=5.5 Hz, 1H), 4.20-4.13 (m, 4H), 3.46 (s, 2H), 2.82-2.72 (m, 4H), 2.51 (s, 3H), 1.91-1.84 (m, 4H), 1.65-1.58 (m, 4H).

### Example 74

### 3-((8-(4,4-difluoropiperidin-1-yl)pyrido[3,4-d]pyrimidin-2-yl)amino)-6-methyl-5,6,7,8-tetrahydro-1,6-naphthyridin-2(1H)-one

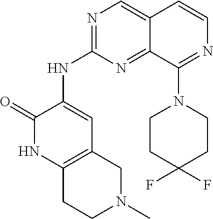 [SMILES: CN1CCc2[nH]c(=O)c(Nc3ncc4ccnc(N5CCC(F)(F)CC5)c4n3)cc2C1]

Compound 74 can be obtained by using 4,4-difluoropiperidine instead of 3-amino-2,2-dimethyl-1-propanol in the first step in Example 70, and using a similar method and reaction steps. ESI-MS (m/z): 428.2 [M+H]⁺; ¹H NMR (500 MHz, DMSO-d6) δ 12.03 (s, 1H), 9.35 (s, 1H), 8.49 (s, 1H), 8.09-8.06 (m, 2H), 7.31 (d, J=5.5 Hz, 1H), 3.95-3.89 (m, 4H), 2.65-2.57 (m, 4H), 2.36 (s, 3H), 2.22-2.12 (m, 4H).

### Example 75

### 3-((8-(cyclohex-1-en-1-yl)pyrido[3,4-d]pyrimidin-2-yl)amino)-6-methyl-5,6,7,8-tetra hydro-1,6-naphthyridin-2(1H)-one

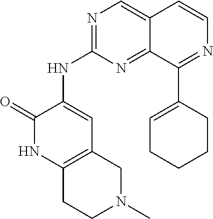 [SMILES: CN1CCc2[nH]c(=O)c(Nc3ncc4ccnc(C5=CCCCC5)c4n3)cc2C1]

Compound 75 was prepared by the following steps:

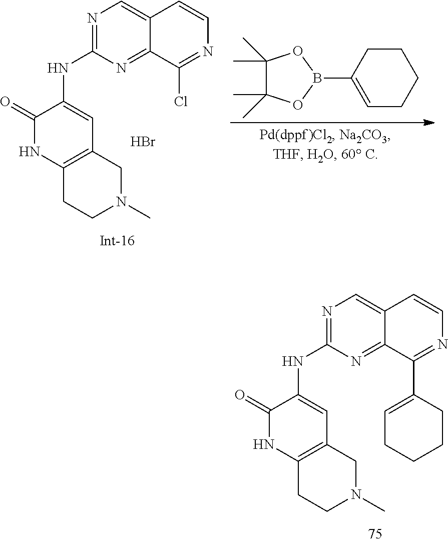

Step 1: Dissolve Int-16 (50 mg, 118 umol, HBr salt) and cyclohexene-1-boronic acid pinacol ester (27 mg, 129 umol) in a mixed solvent of THF (10 mL) and water (2 mL). Sodium carbonate (25 mg, 236 umol) and Pd(dppf)Cl₂ (10 mg, 12 umol) were added, and the reaction system was replaced with nitrogen and heated to 60° C. and stirred overnight. After the reaction solution was cooled to room temperature, the reaction solution was filtered through celite, and the filtrate was concentrated. The residue was purified by Prep-HPLC to obtain yellow solid 75 (1.1 mg, yield 2%). ESI-MS (m/z): 389.3 [M+H]⁺; ¹HNMR (500 MHz, DMSO-d₆) δ 12.01 (br s, 1H), 9.43 (s, 1H), 8.48-8.45 (m, 2H), 8.32 (s, 1H), 7.70 (d, J=5.2 Hz, 1H), 6.68 (s, 1H), 3.30 (s, 2H), 2.65-2.58 (m, 6H), 2.40-2.30 (m, 5H), 1.86-1.70 (m, 4H).

### Example 76

### (R)-6-methyl-3-((8-(2-methylpiperidin-1-yl)pyrido[3,4-d]piperidin-2-yl)amino)-5,6,7,8-tetrahydro-1,6-naphthyridin-2(1H)-one

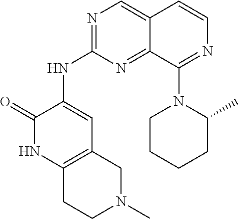 [SMILES: C[C@@H]1CCCCN1c1nccc2cnc(Nc3cc4c([nH]c3=O)CCN(C)C4)nc12]

Compound 76 can be obtained by replacing the 3-amino-2,2-dimethyl-1-propanol in the first step in Example 70 with R-2-methylpiperidine, and using a similar methods and reaction steps. ESI-MS (m/z): 405.4 [M+H]⁺; ¹H NMR (500 MHz, DMSO-d6) δ 11.97 (s, 1H), 9.30 (s, 1H), 8.41 (s, 1H), 8.08 (s, 1H), 8.03 (d, J=5.3 Hz, 1H), 7.19 (d, J=5.3 Hz, 1H), 5.13 (br s, 1H), 4.15-4.06 (m, 1H), 3.28-3.22 (m, 2H), 2.65-2.55 (m, 4H), 2.35 (s, 3H), 2.02-1.94 (m, 1H), 1.80-1.75 (m, 2H), 1.70-1.57 (m, 3H), 1.10 (d, J=6.8 Hz, 3H).

### Example 77

### 6-methyl-3-((8-(((1-methylcyclopropyl)methyl)amino)pyrido[3,4-d]pyrimidin-2-yl)amino)-5,6,7,8-tetrahydro-1,6-naphthridine-2(1H)-one

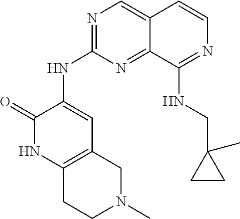 [SMILES: CN1CCc2[nH]c(=O)c(Nc3ncc4ccnc(NCC5(C)CC5)c4n3)cc2C1]

Compound 77 can be obtained by using 1-methylcyclopropylethylamine instead of 3-amino-2,2-dimethyl-1-propanol in the first step in Example 70, and using a similar methods and reaction steps. ESI-MS (m/z): 392.2 [M+H]⁺; ¹H NMR (500 MHz, DMSO-d6) δ 11.95 (s, 1H), 9.23 (s, 1H), 8.42 (s, 1H), 8.23 (s, 1H), 7.83 (d, J=5.6 Hz, 1H), 6.90 (d, J=5.7 Hz, 1H), 6.74 (br s, 1H), 3.45 (d, J=5.7 Hz, 2H), 3.34 (s, 2H), 2.64-2.56 (m, 4H), 2.34 (s, 3H), 1.17 (s, 3H), 0.63-0.57 (m, 2H), 0.36-0.32 (m, 2H).

### Example 78

### 1,6-dimethyl-3-((8-thiomorpholinopyrido[3,4-d]pyrimidin-2-yl)amino)-5,6,7,8-tetrahydro-1,6-naphthyridine-2 (1H)-one

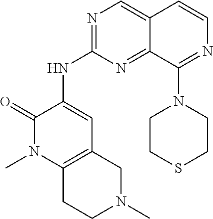 [SMILES: CN1CCc2c(cc(Nc3ncc4ccnc(N5CCSCC5)c4n3)c(=O)n2C)C1]

Compound 78 can be obtained by replacing the piperidine in the first step in Example 68 with thiomorpholine and using a similar methods and reaction steps. ESI-MS (m/z): 424.3 [M+H]⁺; ¹H NMR (500 MHz, DMSO-d6) δ 9.32 (s, 1H), 8.53 (s, 1H), 8.08 (s, 1H), 8.05 (d, J=5.5 Hz, 1H), 7.26 (d, J=5.5 Hz, 1H), 4.13-4.06 (m, 4H), 3.53 (s, 3H), 3.37 (s, 2H), 2.83-2.74 (m, 6H), 2.68-2.62 (m, 2H), 2.37 (s, 3H).

### Example 79

### 3-((8-(4,4-difluoropiperidin-1-yl)piperido[3,4-d]pyrimidin-2-yl)amino)-1,6-dimethyl-5,6,7,8-tetrahydro-1,6-naphthyridin-2(1H)-one

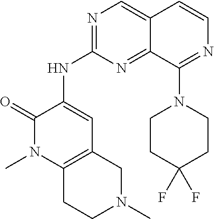 [SMILES: CN1CCc2c(cc(Nc3ncc4ccnc(N5CCC(F)(F)CC5)c4n3)c(=O)n2C)C1]

Compound 79 can be obtained by replacing the piperidine in the first step in Example 68 with 4, 4-difluoropiperidine, and using a similar methods and reaction steps. ESI-MS (m/z): 442.3 [M+H]⁺; ¹H NMR (500 MHz, DMSO-d6) δ 9.35 (s, 1H), 8.56 (s, 1H), 8.09 (s, 1H), 8.07 (d, J=5.5 Hz, 1H), 7.31 (d, J=5.5 Hz, 1H), 3.96-3.89 (m, 4H), 3.53 (s, 3H), 3.35 (s, 2H), 2.83-2.75 (in, 2H), 2.65 (t, J=6.0 Hz, 2H), 2.35 (s, 3H), 2.24-2.12 (m, 4H).

### Example 80

### 3-((8-(azepan-1-yl)pyrido[3,4-d]pyrimidin-2-yl)amino)-1,6-dimethyl-5,6,7,8-tetrahydro-1,6-naphthyridin-2(1H)-one

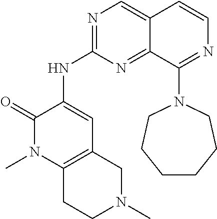 [SMILES: CN1CCc2c(cc(Nc3ncc4ccnc(N5CCCCCC5)c4n3)c(=O)n2C)C1]

Compound 79 can be obtained by replacing piperidine in the first step in Example 68 with cyclohexylimine, and using a similar method and reaction steps. ESI-MS (m/z): 420.3 [M+H]⁺; ¹H NMR (500 MHz, DMSO-d6) δ 9.11 (s, 1H), 8.12 (s, 1H), 7.90 (d, J=5.5 Hz, 1H), 6.92 (d, J=5.5 Hz, 1H), 4.20-4.13 (m, 4H), 3.46 (s, 2H), 2.82-2.72 (m, 4H), 2.51 (s, 3H), 1.91-1.84 (m, 4H), 1.65-1.58 (m, 4H).

### Example 81

### 3-((8-(cyclohex-1-en-1-yl)pyrido[3,4-d]pyrimidin-2-yl)amino)-1,6-dimethyl-5,6,7,8-tetrahydro-1,6-naphthyridin-2(1H)-one

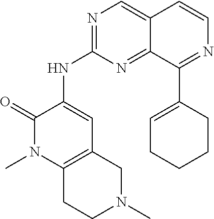 [SMILES: CN1CCc2c(cc(Nc3ncc4ccnc(C5=CCCCC5)c4n3)c(=O)n2C)C1]

Compound 81 can be obtained by replacing Int-16 in the first step in Example 75 with Int-15 and using a similar method and reaction steps. ESI-MS (m/z): 403.2 [M+H]⁺; ¹H NMR (500 MHz, DMSO-d₆) δ 9.44 (s, 1H), 8.56 (s, 1H), 8.46 (d, J=5.5 Hz, 1H), 8.34 (s, 1H), 7.70 (d, J=5.5 Hz, 1H), 6.70-6.65 (m, 1H), 3.54 (s, 3H), 3.36-3.34 (m, 2H), 2.83-2.76 (m, 2H), 2.68-2.64 (m, 2H), 2.64-2.58 (m, 2H), 2.36 (s, 3H), 2.35-2.30 (m, 2H), 1.86-1.80 (m, 2H), 1.80-1.73 (m, 2H).

### Example 82

### 3-((8-(4,4-difluoropiperidin-1-yl)pyrido[3,4-d]pyrimidin-2-yl)amino)-6-(2-hydroxyethyl)-1-methyl-5,6,7,8-tetrahydro-1,6-naphthyridin-2(1H)-one

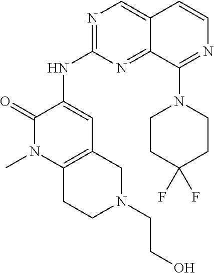 [SMILES: Cn1c2c(cc(Nc3ncc4ccnc(N5CCC(F)(F)CC5)c4n3)c1=O)CN(CCO)CC2]

Compound 82 was prepared by the following steps:

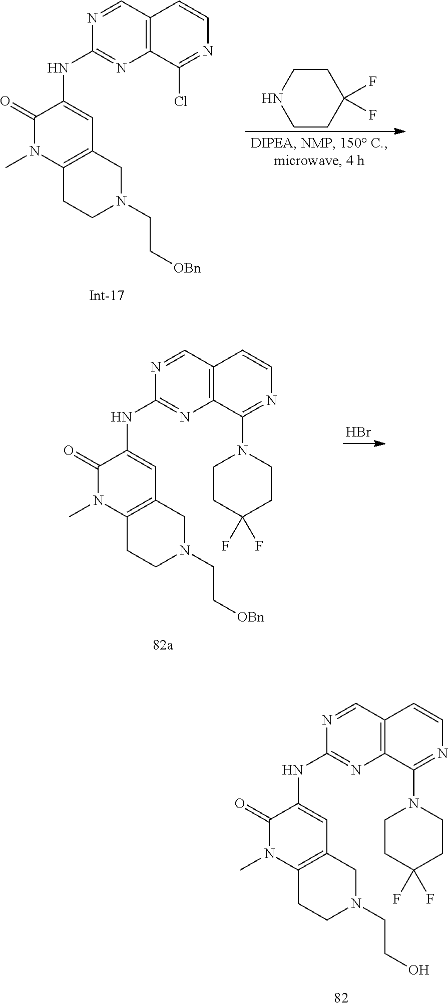 [SMILES: Cn1c2c(cc(Nc3ncc4ccnc(Cl)c4n3)c1=O)CN(CCOCc1ccccc1)CC2 ; FC1(F)CCNCC1 ; Cn1c2c(cc(Nc3ncc4ccnc(N5CCC(F)(F)CC5)c4n3)c1=O)CN(CCOCc1ccccc1)CC2 ; Br ; Cn1c2c(cc(Nc3ncc4ccnc(N5CCC(F)(F)CC5)c4n3)c1=O)CN(CCO)CC2]

Step 1: Dissolve Int-17 (40 mg, 83 umol) in N-methylpyrrolidone (2 mL), add 4, 4-difluoropiperidine (61 mg, 0.59 mmol) and N, N-diisopropylethylamine (54 mg, 0.42 mmol). The reaction solution was heated to 150° C. and stirred for 4 hours in a microwave reactor, and the reaction was detected by LCMS to be complete. The reaction solution was cooled to room temperature, water (50 mL) was added, and the resulting yellow solid was filtered and dried to obtain compound 82a (35 mg, yield 74%). ESI-MS (m/z): 562.2 [M+H]⁺.

Step 2: Intermediate 82a (10 mg, 17 umol) was dissolved in 48% hydrobromic acid aqueous solution (1 mL), and the reaction mixture was stirred at 50° C. for 30 minutes, and LCMS monitored that the reaction was complete. After the reaction solution was cooled to room temperature, it was adjusted to pH=7 with 1N NaOH aqueous solution, extracted with ethyl acetate, and the organic phase was dried over anhydrous sodium sulfate, filtered and concentrated. The residue was purified by reverse-phase preparative HPLC to obtain a yellow solid 82 (4.9 mg, 58% yield). ESI-MS (m/z): 472.2 [M+H]⁺; ¹H NMR (500 MHz, DMSO-d6) δ 9.36 (s, 1H), 8.57 (s, 1H), 8.11 (s, 1H), 8.08 (d, J=5.5 Hz, 1H), 7.32 (d, J=5.5 Hz, 1H), 4.53-4.49 (m, 1H), 3.95-3.89 (m, 4H), 3.62-3.57 (m, 2H), 3.53 (s, 3H), 3.46 (s, 2H), 2.80-2.73 (m, 4H), 2.58 (t, J=6.0 Hz, 2H), 2.25-2.14 (m, 4H).

### Example 83

### 6-(2-hydroxyethyl)-1-methyl-3-((8-(piperidin-1-yl)pyrido[3,4-d]pyrimidin-2-yl)amino)-5,6,7,8-tetrahydro-1,6-naphthyridin-2(1H)-one

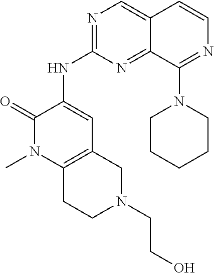 [SMILES: Cn1c2c(cc(Nc3ncc4ccnc(N5CCCCC5)c4n3)c1=O)CN(CCO)CC2]

Compound 81 can be obtained by replacing 4,4-difluoropiperidine in the first step in Example 82 with piperidine, and using a similar method and reaction steps. ESI-MS (m/z): 435.9 [M+H]⁺; ¹H NMR (500 MHz, DMSO-d6) δ 9.29 (s, 1H), 8.50 (s, 1H), 8.12 (s, 1H), 8.03 (d, J=5.5 Hz, 1H), 7.21 (d, J=5.5 Hz, 1H), 4.59-4.49 (m, 1H), 3.73-3.67 (m, 4H), 3.59 (t, J=6.0 Hz, 2H), 3.52 (s, 3H), 3.46 (s, 2H), 2.79-2.73 (m, 4H), 2.58 (t, J=6.0 Hz, 2H), 1.79-1.71 (m, 4H), 1.70-1.62 (m, 2H).

### Example 84

### 6-(2-hydroxyethyl)-1-methyl-3-((8-thiomorpholinopyrido[3,4-d]pyrimidin-2-yl)amino)-5,6,7, 8-tetrahydro-1,6-naphthyridin-2(1H)-one

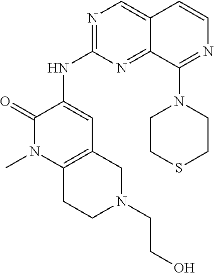 [SMILES: Cn1c2c(cc(Nc3ncc4ccnc(N5CCSCC5)c4n3)c1=O)CN(CCO)CC2]

Compound 81 can be obtained by replacing 4,4-difluoropiperidine in the first step in Example 82 with thiomorpholine and using a similar method and reaction steps. ESI-MS (m/z): 454.2 [M+H]⁺; ¹H NMR (500 MHz, DMSO-d6) δ 9.31 (s, 1H), 8.52 (s, 1H), 8.08 (s, 1H), 8.05 (d, J=5.5 Hz, 1H), 7.25 (d, J=5.5 Hz, 1H), 4.55-4.50 (m, 1H), 4.11-4.00 (m, 4H), 3.64-3.57 (m, 2H), 3.52 (s, 3H), 3.47 (s, 2H), 2.84-2.78 (m, 4H), 2.78-2.72 (m, 4H), 2.62-2.57 (m, 2H).

### Example 85

### 3-((8-(3,3-difluoropyrrolidin-1-yl)pyrido[3,4-d]pyrimidin-2-yl)amino)-1,6-dimethyl-5,6, 7,8-tetrahydro-1,6-naphthyridin-2(1H)-one

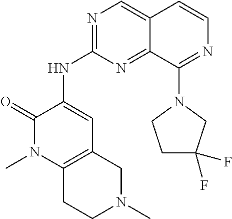 [SMILES: CN1CCc2c(cc(Nc3ncc4ccnc(N5CCC(F)(F)C5)c4n3)c(=O)n2C)C1]

Substituting 3,3-difluoropyrrolidine for piperidine in the first step in Example 68, and using a similar method and reaction steps, Compound 85 can be obtained. ESI-MS (m/z): 404.5 [M+H]⁺; ¹H NMR (500 MHz, DMSO-d6) δ 9.27 (s, 1H), 8.43 (s, 1H), 7.97 (d, J=5.5 Hz, 1H), 7.90 (s, 1H), 7.09 (d, J=5.5 Hz, 1H), 4.47-4.40 (m, 2H), 4.04 (t, J=7.0 Hz, 2H), 3.51 (s, 2H), 2.80-2.75 (m, 2H), 2.66-2.61 (m, 2H), 2.33 (s, 3H), 2.04-1.95 (m, 1H).

### Example 86

### 3-((8-(3-azabicyclo[3.1.0]hex-3-yl)pyrido[3,4-d]pyrimidin-2-yl)amino)-1,6-dimethyl-5,6,7,8-tetrahydro-1,6-naphthyridin-2(1H)-one

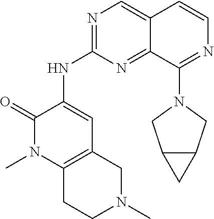 [SMILES: CN1CCc2c(cc(Nc3ncc4ccnc(N5CC6CC6C5)c4n3)c(=O)n2C)C1]

Using 3-aza-bicyclo[3.1.0]hexane to replace the piperidine in the first step in Example 68, compound 86 can be obtained by a similar method and reaction steps. ESI-MS (m/z): 404.5 [M+H]⁺; ¹H NMR (500 MHz, DMSO-d6) δ 9.21 (s, 1H), 8.32 (s, 1H), 7.98 (s, 1H), 7.92 (d, J=5.5 Hz, 1H), 6.99 (d, J=5.5 Hz, 1H), 4.45 (d, J=11.5 Hz, 2H), 3.69-3.66 (m, 2H), 3.52 (s, 3H), 3.36 (s, 2H), 2.80-2.75 (m, 2H), 2.67-2.62 (m, 2H), 2.36 (s, 3H), 1.71-1.67 (m, 2H), 0.75-0.70 (m, 1H), 0.36-0.32 (m, 1H).

### Example 87

### 3-((8-(4,4-difluoropiperidin-1-yl)pyrido[3,4-d]pyrimidin-2-yl)amino)-1-(2-methoxyethyl)-6-methyl-5,6,7,8-tetrahydro-1,6-naphthyridin-2(1H)-one

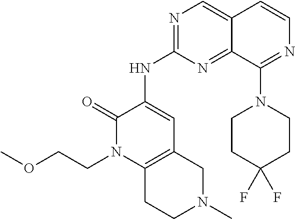 [SMILES: COCCn1c2c(cc(Nc3ncc4ccnc(N5CCC(F)(F)CC5)c4n3)c1=O)CN(C)CC2]

### Example 88

### 3-((8-(4,4-difluoropiperidin-1-yl)pyrido[3,4-d]pyrimidin-2-yl)amino)-1-(2-hydroxyethyl)-6-methyl-5,6,7,8-tetrahydro-1,6-naphthyridin-2(1H)-one

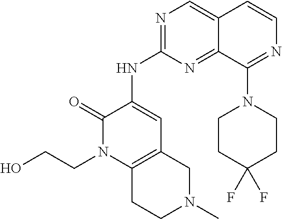 [SMILES: CN1CCc2c(cc(Nc3ncc4ccnc(N5CCC(F)(F)CC5)c4n3)c(=O)n2CCO)C1]

Compounds 87 and 88 were prepared by the following steps:

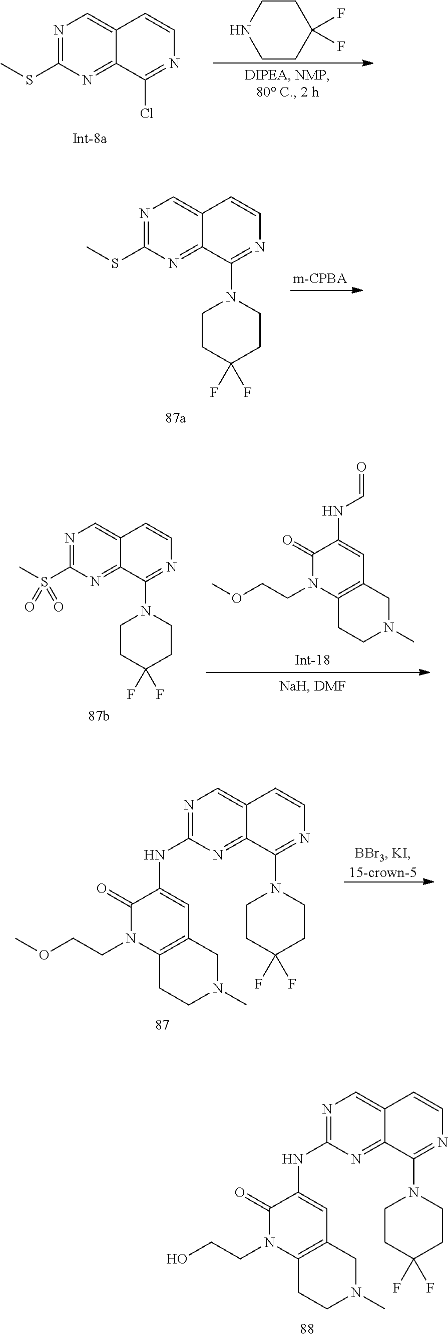

Step 1: Dissolve Int-8a (300 mg, 1.42 mmol) in NMP (5 mL), add 4, 4-difluoropiperidine (343 mg, 2.84 mmol) and N, N-diisopropyl Ethylamine (548 mg, 4.25 mmol), and the reaction solution was stirred at 80° C. for 2 hours. After the reaction was complete, add water (50 ml) to dilute, then extract with ethyl acetate (50 mL*2), combine the organic phases and wash with saturated brine (50 ml). Dry over anhydrous sodium sulfate, filter and concentrate. The residue was separated by silica gel column chromatography (petroleum ether/ethyl acetate=10/1) to obtain light yellow solid compound 87a (350 mg, yield 83%). MS (ESI): m/z 297.2 [M+H]⁺.

Step 2: Dissolve compound 87a (350 mg, 1.18 mmol) in dichloromethane (5 mL), add m-chloroperoxybenzoic acid (600 mg, content 84%, 2.95 mmol) under ice cooling. The react solution was stirred at room temperature for 16 hours. After the reaction is complete, the reaction solution was diluted with water (30 mL), then extracted with dichloromethane (30 mL*2), and the organic phases were combined and washed with saturated sodium thiosulfate solution (30 mL), saturated sodium carbonate solution (30 mL), and saturated brine (30 mL), respectively. Dry over anhydrous sodium sulfate, filter and concentrate. The residue was separated by silica gel column chromatography (petroleum ether/ethyl acetate=2/1) to obtain light yellow solid compound 87b (300 mg, yield 77%). MS (ESI): m/z 329.2 [M+H]⁺.

Step 3: The compound Int-18 (70 mg, 0.26 mmol) was dissolved in DMF (5 mL), and NaH (53 mg, content 60%, 1.32 mmol) was added thereto under ice cooling, and the reaction system was maintained at 0° C. and stirred for 1 hour. Then compound 87b (87 mg, 0.26 mmol) dissolved in DMF (1 mL) was added, and the reaction solution was stirred at room temperature for 1 hour. After the reaction was complete, the reaction solution was poured into water (50 mL), and the resulting solid was suction filtered and dried to obtain the crude product. The crude product was purified by reverse-phase preparative HPLC to obtain compound 87 (35 mg, yield 27%) as a light yellow solid. ESI-MS (m/z): 486.3 [M+H]⁺; ¹H NMR (500 MHz, DMSO-d6) δ 9.36 (s, 1H), 8.55 (s, 1H), 8.10 (s, 1H), 8.08 (d, J=5.5 Hz, 1H), 7.32 (d, J=5.5 Hz, 1H), 4.22 (t, J=5.5 Hz, 2H), 3.98-3.90 (m, 4H), 3.62 (t, J=5.5 Hz, 2H), 3.36 (s, 2H), 3.25 (s, 3H), 2.92-2.84 (m, 2H), 2.69-2.61 (m, 2H), 2.35 (s, 3H), 2.24-2.12 (m, 4H).

Step 4: Compound 87 (30 mg, 0.062 mmol) was dissolved in dichloromethane (5 mL), and NaI (9 mg, 0.062 mmol), 15-crown-5 (14 mg, 0.062 mmol), and boron tribromide (31 mg, 0.124 mmol) was added under ice bath. The reaction system was stirred at room temperature for 2 hours. After the reaction was complete, it was added to water (50 mL), and adjusted to pH=7 with saturated sodium bicarbonate solution. Extract with ethyl acetate (30 mL*3). Combine the organic phases and wash with saturated brine (50 mL).

Dry over anhydrous sodium sulfate, filter and concentrate. The residue was purified by reverse-phase preparative HPLC to obtain compound 88 (2 mg, yield 6.90%) as a white solid. ESI-MS (m/z): 472.4 [M+H]; ¹H NMR (500 MHz, DMSO-d6) δ 9.35 (s, 1H), 8.55 (s, 1H), 8.10 (s, 1H), 8.07 (d, J=5.5 Hz, 1H), 7.31 (d, J=5.5 Hz, 1H), 4.96 (s, 1H), 4.11 (t, J=5.5 Hz, 2H), 3.96-3.90 (2, 4H), 3.67 (t, J=6.0 Hz, 2H), 3.36 (s, 2H), 2.93-2.89 (m, 2H), 2.66-2.62 (m, 2H), 2.35 (s, 3H), 2.23-2.13 (m, 4H).

According to the synthetic route and the synthetic method of the intermediate described in the above examples, the following examples were obtained.

| Example | structure | analyze data |
|---|---|---|
| 89 | 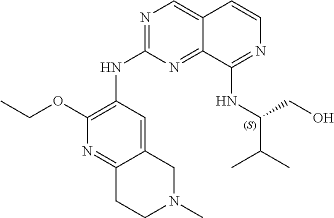 | ESI-MS (m/z): 437.4 [M + H]⁺; ¹H NMR (500 MHz, DMSO-d₆) δ 9.19 (s, 1H), 8.50 (s, 1H), 8.21 (s, 1H), 7.78 (d, J = 5.7 Hz, 1H), 6.87 (d, J = 5.6 Hz, 1H), 6.60 (d, J = 9.2 Hz, 1H), 4.84 (t, J = 5.2 Hz, 1H), 4.36 (q, J = 7.0 Hz, 2H), 4.12-4.04 (m, 1H), 3.69-3.63 (m, 1H), 3.56-3.41 (m, 3H), 2.77 (t, J = 6.0 Hz, 2H), 2.67 (t, J = 6.0 Hz, 2H), 2.36 (s, 3H), 2.16-2.07 (m, 1H), 1.34 (t, J = 7.0 Hz, 3H), 1.03-0.94 (m, 6H). |
| 90 | 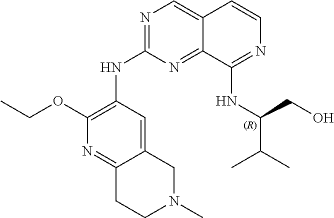 | ESI-MS (m/z): 437.4 [M + H]⁺; ¹H NMR (500 MHz, DMSO-d₆) δ 9.19 (s, 1H), 8.50 (s, 1H), 8.21 (s, 1H), 7.78 (d, J = 5.6 Hz, 1H), 6.87 (d, J = 5.6 Hz, 1H), 6.60 (d, J = 9.1 Hz, 1H), 4.83 (br s, 1H), 4.36 (q, J = 7.0 Hz, 2H), 4.11-4.04 (m, 1H), 3.68-3.63 (m, 1H), 3.56-3.42 (m, 3H), 2.77 (t, J = 5.9 Hz, 2H), 2.67 (t, J = 6.0 Hz, 2H), 2.36 (s, 3H), 2.15-2.06 (m, 1H), 1.34 (t, J = 7.0 Hz, 3H), 1.00-0.92 (m, 6H). |
| 91 | 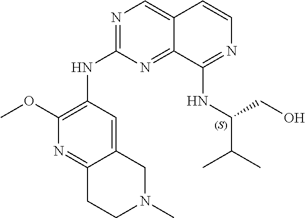 | ESI-MS (m/z): 424.3 [M + H]⁺; ¹H NMR (500 MHz, DMSO-d₆) δ 9.19 (s, 1H), 8.62 (s, 1H), 8.17 (s, 1H), 7.77 (d, J = 5.6 Hz, 1H), 6.86 (d, J = 5.7 Hz, 1H), 6.60 (d, J = 9.1 Hz, 1H), 4.84 (br s, 1H), 4.12-4.05 (m, 1H), 3.91 (s, 3H), 3.67-3.62 (m, 1H), 3.56-3.44 (m, 3H), 2.79 (t, J = 5.8 Hz, 2H), 2.67 (t, J = 5.9 Hz, 2H), 2.36 (s, 3H), 2.15-2.07 (m, 1H), 0.96 (t, J = 6.4 Hz, 6H). |
| 92 | 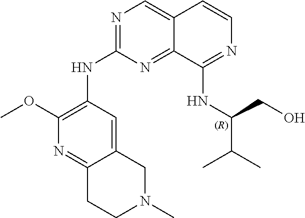 | ESI-MS (m/z): 424.3 [M + H]⁺; ¹H NMR (500 MHz, DMSO-d₆) δ 9.19 (s, 1H), 8.62 (s, 1H), 8.17 (s, 1H), 7.77 (d, J = 5.7 Hz, 1H), 6.86 (d, J = 5.7 Hz, 1H), 6.60 (d, J = 9.1 Hz, 1H), 4.83 (t, J = 5.2 Hz, 1H), 4.13-4.04 (m, 1H), 3.91 (s, 3H), 3.66-3.62 (m, 1H), 3.54-3.44 (m, 3H), 2.79 (t, J = 5.8 Hz, 2H), 2.68 (t, J = 5.9 Hz, 2H), 2.36 (s, 3H), 2.15-2.06 (m, 1H), 0.96 (t, J = 6.4 Hz, 6H). |
| 93 | 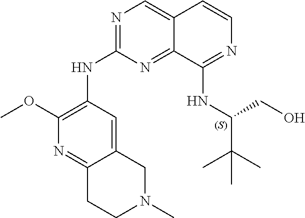 | ESI-MS (m/z): 437.4 [M + H]⁺; ¹H NMR (500 MHz, DMSO-d₆) δ 9.20 (s, 1H), 8.61 (s, 1H), 8.27 (s, 1H), 7.77 (d, J = 5.6 Hz, 1H), 6.86 (d, J = 5.6 Hz, 1H), 6.72 (d, J = 9.7 Hz, 1H), 4.75 (br s, 1H), 4.18-4.12 (m, 1H), 3.92 (s, 3H), 3.73-3.46 (m, 4H), 2.87-2.68 (m, 4H), 2.42 (s, 3H), 1.01 (s, 9H). |
| 94 | 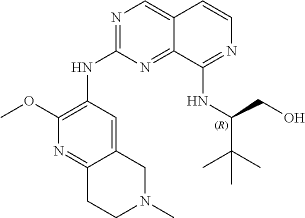 | ESI-MS (m/z): 437.4 [M + H]⁺; ¹H NMR (500 MHz, DMSO-d₆) δ 9.20 (s, 1H), 8.58 (s, 1H), 8.26 (s, 1H), 7.77 (d, J = 5.6 Hz, 1H), 6.86 (d, J = 5.6 Hz, 1H), 6.72 (d, J = 9.9 Hz, 1H), 4.75 (br s, 1H), 4.17-4.11 (m, 1H), 3.92 (s, 3H), 3.71-3.62 (m, 2H), 3.52-3.40 (m, 2H), 2.81-2.76 (m, 2H), 2.70-2.63 (m, 2H), 2.35 (s, 3H), 1.01 (s, 9H). |
| 95 | 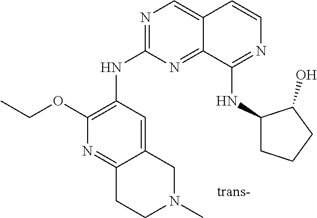 [SMILES: CCOc1nc2c(cc1Nc1ncc3ccnc(N[C@@H]4CCC[C@H]4O)c3n1)CN(C)CC2] | ESI-MS (m/z): 435.9 [M + H]⁺; ¹H NMR (500 MHz, DMSO-d₆) δ 9.19 (s, 1H), 8.49 (s, 1H), 8.19 (s, 1H), 7.80 (d, J = 5.6 Hz, 1H), 6.90 (d, J = 5.6 Hz, 1H), 6.53 (d, J = 6.5 Hz, 1H), 5.13 (d, J = 3.7 Hz, 1H), 4.35 (q, J = 7.0 Hz, 2H), 4.16-4.08 (m, 1H), 4.06-4.00 (m, 1H), 3.48 (s, 2H), 2.80-2.74 (m, 2H), 2.67 (t, J = 5.9 Hz, 2H), 2.37 (s, 3H), 2.26-2.18 (m, 1H), 1.96-1.87 (m, 1H), 1.84-1.66 (m, 2H), 1.62-1.50 (m, 2H), 1.33 (t, J = 7.0 Hz, 3H). |
| 96 | 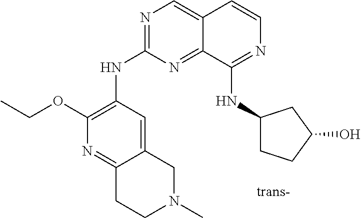 [SMILES: CCOc1nc2c(cc1Nc1ncc3ccnc(N[C@@H]4CC[C@@H](O)C4)c3n1)CN(C)CC2] | ESI-MS (m/z): 435.8 [M + H]⁺; ¹H NMR (500 MHz, DMSO-d₆) δ 9.18 (s, 1H), 8.45 (s, 1H), 8.22 (s, 1H), 7.81 (d, J = 5.7 Hz, 1H), 6.87 (d, J = 5.7 Hz, 1H), 6.58 (d, J = 7.5 Hz, 1H), 4.64-4.54 (m, 2H), 4.36 (q, J = 7.0 Hz, 2H), 4.28 (s, 1H), 3.48 (s, 2H), 2.77 (t, J = 5.8 Hz, 2H), 2.67 (t, J = 5.9 Hz, 2H), 2.37 (s, 3H), 2.28-2.17 (m, 1H), 2.09-1.95 (m, 2H), 1.77-1.69 (m, 1H), 1.60-1.46 (m, 2H), 1.33 (t, J = 7.0 Hz, 3H). |
| 97 | 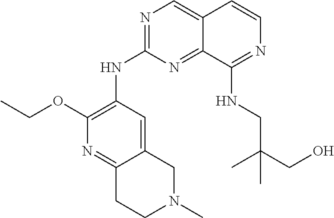 [SMILES: CCOc1nc2c(cc1Nc1ncc3ccnc(NCC(C)(C)CO)c3n1)CN(C)CC2] | ESI-MS (m/z): 437.5 [M + H]⁺; ¹H NMR (500 MHz, DMSO-d₆) δ 9.19 (s, 1H), 8.37 (s, 1H), 8.30 (s, 1H), 7.77 (d, J = 5.7 Hz, 1H), 7.06 (t, J = 6.0 Hz, 1H), 6.86 (d, J = 5.7 Hz, 1H), 5.03 (t, J = 5.9 Hz, 1H), 4.37 (q, J = 7.0 Hz, 2H), 3.51 (s, 2H), 3.42 (d, J = 6.0 Hz, 2H), 3.24 (d, J = 5.2 Hz, 2H), 2.77 (t, J = 6.0 Hz, 2H), 2.67 (t, J = 5.9 Hz, 2H), 2.36 (s, 3H), 1.35 (t, J = 7.0 Hz, 3H), 0.93 (s, 6H). |
| 98 | 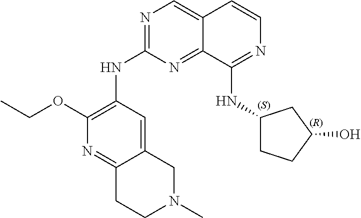 | ESI-MS (m/z): 435.5 [M + H]⁺; ¹H NMR (500 MHz, DMSO-d₆) δ 9.18 (s, 1H), 8.38 (s, 1H), 8.26 (s, 1H), 7.81 (d, J = 5.6 Hz, 1H), 6.98 (d, J = 8.4 Hz, 1H), 6.86 (d, J = 5.7 Hz, 1H), 4.82 (d, J = 3.0 Hz, 1H), 4.60-4.52 (m, 1H), 4.36 (q, J = 7.0 Hz, 2H), 4.27 (br s, 1H), 3.54 (s, 2H), 2.76 (d, J = 5.9 Hz, 2H), 2.67 (t, J = 5.9 Hz, 2H), 2.37 (s, 3H), 2.13-2.02 (m, 2H), 1.81-1.70 (m, 3H), 1.64-1.57 (m, 1H), 1.34 (t, J = 7.0 Hz, 3H). |
| 99 | 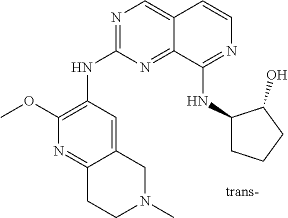 [SMILES: COc1nc2c(cc1Nc1ncc3ccnc(N[C@@H]4CCC[C@H]4O)c3n1)CN(C)CC2] | ESI-MS (m/z): 422.3 [M + H]⁺; ¹H NMR (500 MHz, DMSO-d₆) δ 9.19 (s, 1H), 8.60 (s, 1H), 8.18 (s, 1H), 7.80 (d, J = 5.8 Hz, 1H), 6.89 (d, J = 5.8 Hz, 1H), 6.55 (d, J = 6.4 Hz, 1H), 5.12 (br s, 1H), 4.15-4.08 (m, 1H), 4.06-4.00 (m, 1H), 3.90 (s, 3H), 3.49 (s, 2H), 2.79 (t, J = 7.0 Hz, 2H), 2.68 (t, J = 5.8 Hz, 2H), 2.37 (s, 3H), 2.25-2.16 (m, 1H), 1.96-1.86 (m, 1H), 1.82-1.65 (m, 2H), 1.60-1.48 (s, 2H). |
| 100 | 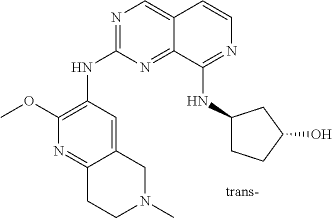 [SMILES: COc1nc2c(cc1Nc1ncc3ccnc(N[C@@H]4CC[C@@H](O)C4)c3n1)CN(C)CC2] | ESI-MS (m/z): 422.3 [M + H]⁺; ¹H NMR (500 MHz, DMSO-d₆) δ 9.18 (s, 1H), 8.54 (s, 1H), 8.22 (s, 1H), 7.80 (d, J = 5.7 Hz, 1H), 6.86 (d, J = 5.7 Hz, 1H), 6.61 (d, J = 7.4 Hz, 1H), 4.65-4.54 (m, 2H), 4.28 (br s, 1H), 3.90 (s, 3H), 3.49 (s, 2H), 2.79 (t, J = 5.9 Hz, 2H), 2.68 (t, J = 5.9 Hz, 2H), 2.37 (s, 3H), 2.26-2.17 (m, 1H), 2.05-1.92 (m, 2H), 1.76-1.67 (m, 1H), 1.56-1.46 (m, 2H). |
| 101 | 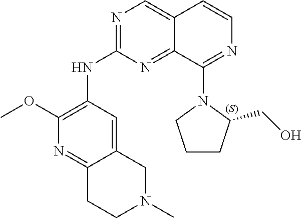 | ESI-MS (m/z): 422.4 [M + H]⁺; ¹H NMR (500 MHz, DMSO-d₆) δ 9.14 (s, 1H), 8.55 (s, 1H), 7.85-7.80 (m, 2H), 6.89 (d, J = 5.4 Hz, 1H), 4.74 (br s, 1H), 4.67 (t, J = 5.5 Hz, 1H), 4.07-4.00 (m, 1H), 3.86 (s, 3H), 3.83-3.75 (m, 1H), 3.53-3.48 (m, 1H), 3.43 (s, 2H), 3.33-3.25 (m, 2H), 2.79 (t, J = 6.0 Hz, 2H), 2.67 (t, J = 5.9 Hz, 2H), 2.37 (s, 3H), 2.02-1.88 (m, 3H), 1.84-1.75 (m, 1H). |
| 102 | 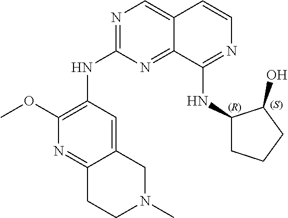 | ESI-MS (m/z): 422.3 [M + H]⁺; ¹H NMR (500 MHz, DMSO-d₆) δ 9.18 (s, 1H), 8.50 (s, 1H), 8.23 (s, 1H), 7.80 (d, J = 5.7 Hz, 1H), 6.98 (d, J = 7.5 Hz, 1H), 6.87 (d, J = 5.7 Hz, 1H), 5.10 (d, J = 4.5 Hz, 1H), 4.28-4.11 (m, 2H), 3.91 (s, 3H), 3.57-3.47 (m, 2H), 2.79 (t, J = 5.9 Hz, 2H), 2.68 (t, J = 5.9 Hz, 2H), 2.37 (s, 3H), 2.11-2.01 (m, 1H), 1.95-1.85 (m, 1H), 1.85-1.74 (m, 1H), 1.73-1.63 (m, 1H), 1.62-1.50 (m, 2H). |
| 103 | 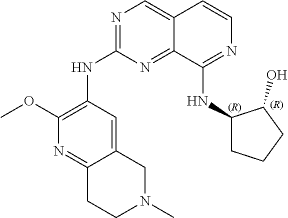 | ESI-MS (m/z): 422.3 [M + H]⁺; ¹H NMR (500 MHz, DMSO-d₆) δ 9.19 (s, 1H), 8.59 (s, 1H), 8.18 (s, 1H), 7.80 (d, J = 5.7 Hz, 1H), 6.89 (d, J = 5.7 Hz, 1H), 6.55 (d, J = 6.5 Hz, 1H), 5.11 (d, J = 3.7 Hz, 1H), 4.15-4.07 (m, 1H), 4.06-4.01 (m, 1H), 3.90 (s, 3H), 3.49 (s, 2H), 2.79 (t, J = 5.9 Hz, 2H), 2.68 (t, J = 5.9 Hz, 2H), 2.37 (s, 3H), 2.26-2.17 (m, 1H), 1.96-1.88 (m, 1H), 1.84-1.65 (m, 2H), 1.62-1.50 (m, 2H). |
| 104 | 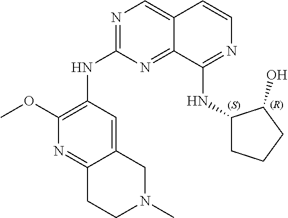 | ESI-MS (m/z): 422.5 [M + H]⁺; ¹H NMR (500 MHz, DMSO-d₆) δ 9.18 (s, 1H), 8.50 (s, 1H), 8.23 (s, 1H), 7.80 (d, J = 5.6 Hz, 1H), 6.98 (d, J = 7.5 Hz, 1H), 6.87 (d, J = 5.7 Hz, 1H), 5.13 (br s, 1H), 4.25-4.19 (m, 1H), 4.17-4.12 (m, 1H), 3.91 (s, 3H), 3.57-3.48 (m, 2H), 2.79 (t, J = 5.6 Hz, 2H), 2.68 (t, J = 5.9 Hz, 2H), 2.37 (s, 3H), 2.10-2.04 (m, 1H), 1.96-1.89 (m, 1H), 1.85-1.75 (m, 1H), 1.73-1.63 (m, 1H), 1.62-1.50 (m, 2H). |
| 105 | 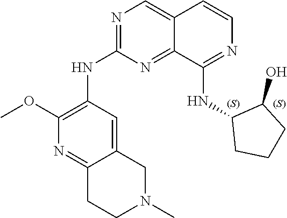 | ESI-MS (m/z): 422.4 [M + H]⁺; ¹H NMR (500 MHz, DMSO-d₆) δ 9.19 (s, 1H), 8.59 (s, 1H), 8.18 (s, 1H), 7.80 (d, J = 5.7 Hz, 1H), 6.89 (d, J = 5.7 Hz, 1H), 6.55 (d, J = 6.6 Hz, 1H), 5.11 (br s, 1H), 4.16-4.09 (m, 1H), 4.07-4.02 (m, 1H), 3.90 (s, 3H), 3.49 (s, 2H), 2.79 (t, J = 5.9 Hz, 2H), 2.68 (t, J = 5.9 Hz, 2H), 2.37 (s, 3H), 2.26-2.19 (m, 1H), 1.96-1.89 (m, 1H), 1.86-1.64 (m, 2H), 1.62-1.49 (m, 2H). |
| 106 | 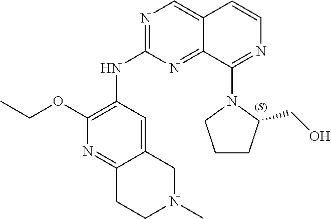 | ESI-MS (m/z): 436.2 [M + H]⁺; ¹H NMR (500 MHz, DMSO-d₆) δ 9.14 (s, 1H), 8.42 (s, 1H), 7.89-7.82 (m, 2H), 6.90 (d, J = 5.4 Hz, 1H), 4.74 (br s, 1H), 4.68 (t, J = 5.5 Hz, 1H), 4.36-4.29 (m, 2H), 4.11-4.04 (m, 1H), 3.84-3.78 (m, 1H), 3.56-3.50 (m, 1H), 3.42 (s, 2H), 3.35-3.28 (m, 1H), 2.77 (t, J = 5.9 Hz, 2H), 2.67 (t, J = 5.9 Hz, 2H), 2.36 (s, 3H), 2.02-1.90 (m, 3H), 1.86-1.79 (m, 1H), 1.30 (t, J = 7.0 Hz, 3H). |
| 107 | 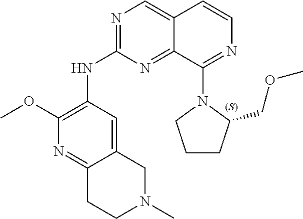 | ESI-MS (m/z): 435.5 [M + H]⁺; ¹H NMR (500 MHz, DMSO-d₆) δ 9.16 (s, 1H), 8.59 (s, 1H), 7.86 (d, J = 5.4 Hz, 1H), 7.80 (s, 1H), 6.91 (d, J = 5.4 Hz, 1H), 4.86 (br s, 1H), 4.07-3.96 (m, 1H), 3.86 (s, 3H), 3.80-3.72 (m, 1H), 3.43 (s, 2H), 3.41-3.35 (m, 1H), 3.25-3.17 (m, 1H), 3.15 (s, 3H), 2.79 (t, J = 5.9 Hz, 2H), 2.67 (t, J = 5.7 Hz, 2H), 2.37 (s, 3H), 2.00-1.89 (m, 3H), 1.86-1.78 (m, 1H). |
| 108 | 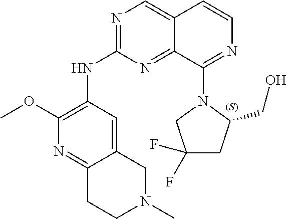 | ESI-MS (m/z): 458.3 [M + H]⁺; ¹H NMR (500 MHz, DMSO-d₆) δ 9.21 (s, 1H), 8.85 (s, 1H), 7.89 (d, J = 5.4 Hz, 1H), 7.73 (s, 1H), 7.05 (d, J = 5.4 Hz, 1H), 4.94-4.85 (m, 2H), 4.36-4.15 (m, 2H), 3.85 (s, 3H), 3.58-3.43 (m, 4H), 2.80 (t, J = 5.9 Hz, 2H), 2.67 (t, J = 5.9 Hz, 2H), 2.52-2.46 (m, 2H), 2.36 (s, 3H). |
| 109 | 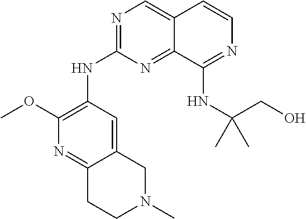 [SMILES: COc1nc2c(cc1Nc1ncc3ccnc(NC(C)(C)CO)c3n1)CN(C)CC2] | ESI-MS (m/z): 410.5 [M + H]⁺; ¹H NMR (500 MHz, DMSO-d₆) δ 9.17 (s, 1H), 8.55 (s, 1H), 8.18 (s, 1H), 7.78 (d, J = 5.7 Hz, 1H), 6.87 (d, J = 5.7 Hz, 1H), 6.73 (s, 1H), 5.33 (t, J = 5.8 Hz, 1H), 3.91 (s, 3H), 3.58 (d, J = 5.4 Hz, 2H), 3.52 (s, 2H), 2.79 (t, J = 5.8 Hz, 2H), 2.69 (t, J = 5.8 Hz, 2H), 2.37 (s, 3H), 1.44 (s, 6H). |
| 110 | 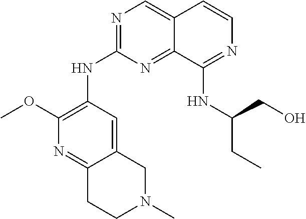 [SMILES: CC[C@H](CO)Nc1nccc2cnc(Nc3cc4c(nc3OC)CCN(C)C4)nc12] | ESI-MS (m/z): 410.5 [M + H]⁺; ¹H NMR (500 MHz, DMSO-d₆) δ 9.18 (s, 1H), 8.58 (s, 1H), 8.19 (s, 1H), 7.78 (d, J = 5.7 Hz, 1H), 6.86 (d, J = 5.7 Hz, 1H), 6.65 (d, J = 8.5 Hz, 1H), 4.92 (br s, 1H), 4.08-4.04 (m, 1H), 3.90 (s, 3H), 3.65-3.60 (m, 1H), 3.56-3.47 (m, 3H), 2.79 (t, J = 5.9 Hz, 2H), 2.68 (d, J = 5.8 Hz, 2H), 2.37 (s, 3H), 1.80-1.58 (m, 2H), 0.93 (d, J = 7.5 Hz, 3H). |
| 111 | 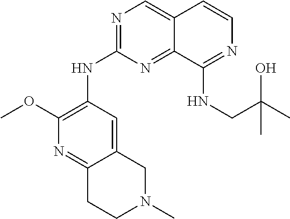 [SMILES: COc1nc2c(cc1Nc1ncc3ccnc(NCC(C)(C)O)c3n1)CN(C)CC2] | ESI-MS (m/z): 410.4 [M + H]⁺; ¹H NMR (500 MHz, DMSO-d₆) δ 9.19 (s, 1H), 8.52 (s, 1H), 8.23 (s, 1H), 7.78 (d, J = 5.6 Hz, 1H), 6.87 (d, J = 5.7 Hz, 1H), 6.79 (t, J = 5.5 Hz, 1H), 4.78 (s, 1H), 3.91 (s, 3H), 3.51 (s, 2H), 3.46 (d, J = 5.6 Hz, 2H), 2.79 (t, J = 5.9 Hz, 2H), 2.68 (t, J = 5.9 Hz, 2H), 2.37 (s, 3H), 1.20 (s, 6H). |

### Example 112

### (R)-2-((2-((6-methoxy-2-methyl-1,2,3,4 tetrahydroisoquinolin-7-yl)amino)amino)pyrido[3,4-d]pyrimidin-8-yl)amino)-3-methylbutan-1-ol

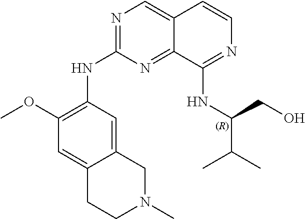

Compound 112 was prepared by the following steps:

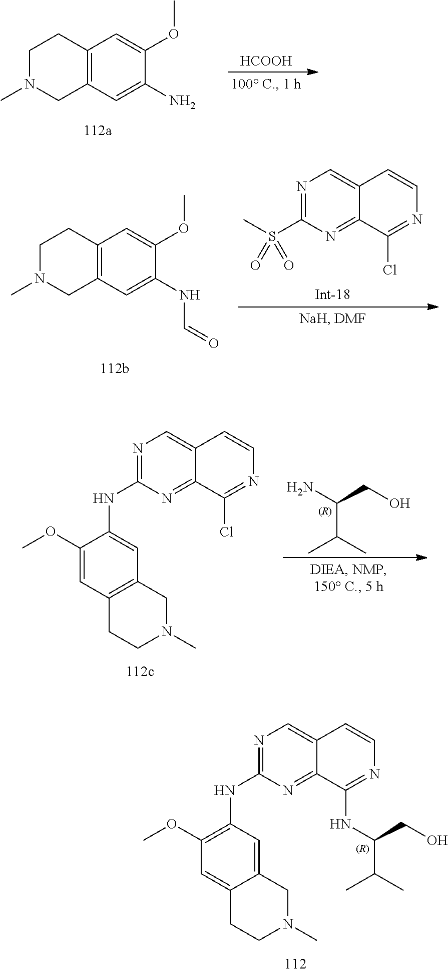

Step 1: Compound 112a (1.0 mg, 5.2 mmol) was dissolved in formic acid (10 mL), and the reaction solution was stirred at 100° C. for 1 hour. The completion of the reaction was monitored by LCMS, the reaction solution was concentrated, the residue was dissolved in dichloromethane, ammonia methanol solution (7M) was added, insoluble matter was removed by filtration, and the filtrate was concentrated to obtain a light yellow solid compound 112b (1.1 g, yield 96%). ESI-MS (m/z): 221.4 [M+H]⁺.

Step 2: Dissolve compound 112b (950 mg, 4.31 mmol) in dry DMF (5 mL), add sodium hydride (314 mg, content 60%, 8.21 mmol) under ice bath, and warm the reaction mixture to room temperature and stir for 1 hour, then the reaction mixture was cooled under ice bath, and Int-18 (1.0 g, 4.10 mmol, dissolved in 3 mL of dry DMF) was added. The reaction mixture was stirred for an additional 1 hour, and LCMS monitored the completion of the reaction. The reaction solution was diluted with water (100 mL), and the resulting yellow solid was collected by filtration and dried to obtain compound 112c (800 mg, yield 54%). ESI-MS (m/z): 356.3 [M+H]⁺.

Step 3: Dissolve compound 112c (50 mg, 140 umol) in N-methylpyrrolidone (3 mL), add D-valinol (73 mg, 702 umol) and N, N-diisopropylethylamine (91 mg, 702 umol). The reaction solution was heated to 150° C. and stirred for 5 hours in a microwave reactor, and the reaction was detected by LCMS to be complete. The reaction solution was cooled to room temperature, and directly purified by reverse-phase preparative HPLC to obtain compound 112 (17 mg, formate salt, yield 26%). ESI-MS (m/z): 423.5 [M+H]⁺; ¹H NMR (500 MHz, DMSO-d6) δ 9.15 (s, 1H), 8.39 (s, 1H), 8.17 (s, 1H), 7.94 (s, 1H), 7.76 (d, J=5.6 Hz, 1H), 6.85 (d, J=5.6 Hz, 1H), 6.82 (s, 1H), 6.59 (d, J=9.1 Hz, 1H), 4.12-4.04 (m, 1H), 3.84 (s, 3H), 3.67-3.63 (m, 1H), 3.55-3.50 (m, 2H), 2.81 (t, J=5.9 Hz, 2H), 2.62 (t, J=6.0 Hz, 2H), 2.36 (s, 3H), 2.16-2.06 (m, 1H), 1.00-0.94 (m, 6H).

HPK1 Inhibitor Biological Screening and Results

### Test Example 1: Detection of Compound's Ability to Inhibit HPK1 Kinase Activity (Method 1)

The required reagents are as follows

| | | | Storage |
|---|---|---|---|
| Reagent | brand | Item No. | Conditions |
| MOPS | Sigma | M1254-100G | RT |
| β-glycerol-phosphate | Sigma | 50020-100G | RT |
| EDTA | Ambion | AM9260G | RT |
| EGTA | Sigma | E3889 | RT |
| MgCl2 | Sigma | M1028-100ML | RT |
| DTT | Sigma | D0632-259 | −20° C. |
| ATP | Sigma | A7699-5G | −20° C. |
| HPK1 | Signalchem | M23-11G-10 | −80° C. |
| MBP | Signalchem | M42-51N | −80° C. |
| ADP-Glo Kinase Assay | Promega | V9102 | −20° C. |
| Kit-10,000 tests | | | |

Experimental Procedure

The specific operation is as follows: configure the enzymatic reaction system buffer (10 mM MOPS, pH 7.2, 5 mM β-glycerol-phosphate, 10 mM MgCl2, 0.8 mM EDTA, 2 mM EGTA, 0.1 mM DTT); the test compound (1 mM the compound stock solution in DMSO) was diluted with buffer to the highest concentration of 60 uM (containing 6% DMSO), and the compound with a concentration of 60 μM was initially diluted 5 times with a buffer containing 6% DMSO for a total of 8 points of gradient concentration; then dilute HPK1 kinase to 30 nM in buffer. Add 2 μl of HPK1 kinase diluent to each well of a Greiner 384-well microplate (Cat. No. 784075), and add 2 μl of buffer to the control well; add 1 μl of the diluted compound to the reaction well after brief centrifugation, and add 1 μl buffer solution containing 6% DMSO to the control well; after short centrifugation, place in a 25° C. constant temperature incubator (Shanghai Yiheng Scientific Instrument Co., Ltd., Cat. No.: LRH-150) and incubate for 20 minutes. Add 3 μl of reaction substrate (10 μM MBP and 20 μM ATP dissolved in distilled water) to each well, briefly centrifuge and incubate in a constant temperature incubator at 25° C. for 60 min, and use ADP-Glo Kinase Assay Kit to detect the enzymatic reaction activity, ADP-Glo Kinase Assay Kit detection is carried out according to the operating instructions of the kit. Data are described using the half maximal inhibitory concentration IC50 of the compound.

| Compound | | Compound | |
|---|---|---|---|
| number | IC50 (nM) | number | IC50 (nM) |
| 1 | <0.1 | 2 | <0.1 |
| 3 | <0.1 | 4 | <0.1 |
| 5 | <0.1 | 6 | <0.1 |
| 7 | 1.22 | 8 | 1.84 |
| 9 | 3.49 | 10 | 1.48 |
| 11 | 1.54 | 12 | 37.82 |
| 13 | <0.1 | 14 | 1.92 |
| 15 | 9.85 | 16 | 1.74 |
| 17 | <0.1 | 18 | <0.1 |
| 19 | <0.1 | 21 | <0.1 |
| 22 | 5.159 | 23 | <0.1 |
| 24 | <0.1 | 25 | 0.16 |
| 26 | 8.31 | 27 | 16.21 |
| 30 | 0.27 | 31 | 1.66 |
| 32 | 3.61 | 33 | 0.52 |
| 34 | 0.61 | 35 | 1.16 |
| 37 | 4.27 | 38 | 0.41 |
| 39 | <0.1 | 40 | <0.1 |
| 41 | <0.1 | 42 | <0.1 |
| 43 | 4.67 | 44 | <0.1 |
| 45 | <0.1 | 46 | 0.15 |
| 47 | 2.77 | 48 | 1.93 |
| 49 | 26.9 | 52 | <0.1 |
| 53 | <0.1 | 54 | 21.71 |
| 55 | 0.66 | 57 | 0.25 |
| 58 | <0.1 | 59 | <0.1 |
| 61 | <0.1 | 62 | 23.05 |
| 63 | 31.29 | 64 | <0.1 |
| 65 | 20.1 | 67 | 23.27 |
| 68 | 1.47 | 69 | 2.19 |
| 70 | 0.29 | 72 | 21.20 |
| 73 | 0.41 | 74 | 15.82 |
| 75 | <0.1 | 76 | <0.1 |
| 77 | <0.1 | 79 | <0.1 |
| 80 | 0.61 | 81 | 0.23 |
| 82 | <0.1 | 83 | 0.16 |
| 84 | 0.43 | 85 | 25.28 |
| 86 | 56.31 | 87 | 19.86 |
| 89 | <0.1 | 90 | <0.1 |
| 91 | 21.54 | 92 | 0.23 |
| 93 | <0.1 | 94 | 0.16 |
| 95 | 18.10 | 96 | 14.94 |
| 97 | 1.99 | 98 | 0.44 |
| 99 | <0.1 | 100 | <0.1 |
| 101 | <0.1 | 102 | <0.1 |
| 103 | <0.1 | 104 | <0.1 |
| 105 | <0.1 | 106 | 15.45 |
| 107 | <0.1 | 108 | <0.1 |
| 109 | <0.1 | 110 | <0.1 |
| 111 | <0.1 | 112 | <0.1 |

### Test Example 2: Detection of Compound's Activating Ability to Secrete Cytokine Interleukin-2 (IL-2) from Jurkat Cells (Method 2)

The required reagents and cells are as follows

Experimental Reagents:

| | | | Storage | |
|---|---|---|---|---|
| Reagent | brand | Item No. | Conditions | |
| RPMI1640 | BI | 01-100-1ACS | 4° | C. |
| FBS | BI | 04-002-1A | −80° | C. |
| BSA | Amresco | 0332-1KG | 4° | C. |
| Phosphate Buffered | BI | 02-024-1ACS | 4° | C. |
| Saline | | | | |
| Tween-20 | Solarbio | T8220 | RT | |
| Anti-human CD3 | invitrogen | 16-0037-85 | | |
| Antibody | | | | |
| Anti-human CD28 | invitrogen | 16-0289-85 | 4° | C. |
| Antibody | | | | |
| Human IL-2 DuoSet | R&D | DY202 | 4° | C. |
| ELISA | | | | |

Experimental Cells:

| cell | cell type | brand |
|---|---|---|
| Jurkat E6-1 | human T lymphocyte | Chinese Academy of |
| | leukemia cells | Sciences Cell Bank |

Experimental Procedure

The specific operation is as follows: the compound powder was dissolved in DMSO to 10 mM, 2 μl of the compound was added to 998 μl of RPMI 1640 medium (contained 10% FBS in this test), and the highest concentration point was obtained after vortexing. The compound solution was gradually diluted 3 times with 0.2% DMSO medium, with a total of 8 concentration points. That treated with RPMI 1640 medium solution containing 0.1% DMSO was used as a control. Add 1×10⁵ Jurkat E6-1 cells to each well of Corning 96-well cell culture plate (product number: 3599), then add an equal volume of compound dilution solution, add RPMI 1640 medium containing 0.2% DMSO to the control group, and place in a cell culture incubator (Thermo Fisher Scientific, model: 3111) to incubate for 1 h at 37° C. Then, 1 μg/ml Anti-human CD3 Antibody and 1 μg/ml Anti-human CD28 Antibody were added at a final concentration, and placed in a 37° C. cell culture incubator to incubate for 24 hours. The Human IL-2 DuoSet ELISA KIT was used to detect the IL-2 content in the cell supernatant, and the Human IL-2 DuoSet ELISA detection was performed according to the operating instructions of the kit. Data are described as the highest fold ratio of the stimulated signal of the compound to the signal of 0.1% DMSO.

Jurkat IL-2 ELISA Assay

| Compound | Concentration | IL-2 increase relative |
|---|---|---|
| number | (μM) | to DMSO control (%) |
| 1 | 3.3 | 674% |
| 2 | 1.1 | 552% |
| 3 | 1.1 | 520% |
| 4 | 3.3 | 1153% |
| 5 | 1.1 | 1025% |
| 6 | 3.3 | 555% |
| 7 | 10 | 2464% |
| 8 | 3.3 | 1018% |
| 9 | 3.3 | 998% |
| 10 | 10 | 1371% |
| 11 | 10 | 1183% |
| 12 | 10 | 623% |
| 13 | 10 | 270% |
| 14 | 10 | 562% |
| 15 | 10 | 228% |
| 16 | 3.3 | 1826% |
| 17 | 3.3 | 1182% |
| 18 | 3.3 | 393% |
| 19 | 1.1 | 1077% |
| 21 | 1.1 | 844% |
| 23 | 3.3 | 667% |
| 25 | 1.1 | 1265% |
| 26 | 10 | 7056% |
| 27 | 10 | 1371% |
| 28 | 10 | 212% |
| 29 | 10 | 325% |
| 30 | 3.3 | 1996% |
| 31 | 10 | 302% |
| 32 | 10 | 147% |
| 33 | 10 | 10672% |
| 34 | 10 | 4642% |
| 35 | 10 | 1098% |
| 36 | 10 | 169% |
| 37 | 10 | 906% |
| 38 | 10 | 217% |
| 39 | 10 | 876% |
| 40 | 3.3 | 492% |
| 41 | 10 | 1967% |
| 42 | 10 | 1502% |
| 43 | 10 | 1268% |
| 44 | 10 | 677% |
| 45 | 0.37 | 2885% |
| 46 | 1.1 | 2571% |
| 47 | 10 | 502% |
| 48 | 10 | 3073% |
| 49 | 10 | 215% |
| 50 | 10 | 327% |
| 51 | 10 | 175% |
| 52 | 10 | 1304% |
| 53 | 10 | 1270% |
| 54 | 10 | 1325% |
| 55 | 10 | 1821% |
| 56 | 10 | 171% |
| 57 | 10 | 2158% |
| 58 | 10 | 2098% |
| 60 | 10 | 971% |
| 61 | 10 | 474% |
| 62 | 10 | 1832% |
| 63 | 10 | 2603% |
| 64 | 3.3 | 5632% |
| 65 | 10 | 2107% |
| 66 | 10 | 519% |
| 67 | 10 | 4773% |

NA: indicates that no enhanced release of IL-2 was detected.

### Test Example 3: Detection of Compound's Activating Ability to Secrete Cytokine Interleukin-2 (IL-2) from Mouse Spleen Cells (Method 3)

The required reagents and cells are as follows

Experimental Reagents:

| | | | Storage | |
|---|---|---|---|---|
| Reagent | brand | Item No. | Conditions | |
| RPMI1640 | BI | 01-100-1ACS | 4° | C. |
| FBS | BI | 04-002-1A | −80° | C. |
| BSA | Amresco | 0332-1KG | 4° | C. |
| Phosphate Buffered | BI | 02-024-1ACS | 4° | C. |
| Saline | | | | |
| Tween-20 | Solarbio | T8220 | RT | |
| Concanavalin A | Sigma | C5275 | −20° | C. |
| Mouse IL-2 DuoSet | R&D | DY402 | 4° | C. |
| ELISA | | | | |

Experimental Animals:

| animal | animal age | brand |
|---|---|---|
| C57BL/6 | 6-8 weeks | Shanghai Slac Experimental |
| | | Animal Co., Ltd. |

Experimental Procedure

The specific operation is as follows: the compound powder was dissolved in DMSO to 10 mM, 2 μl of the compound was added to 998 μl of RPMI 1640 medium (contained 10% FBS in this test), and the highest concentration point was obtained after vortexing. The compound solution was gradually diluted 3 times with 0.2% DMSO medium, with a total of 8 concentration points. That treated with RPMI 1640 medium solution containing 0.1% DMSO was used as a control. Add 10⁵ mouse spleen cells to each well of Corning 96-well cell culture plate (product number: 3599), then add an equal volume of compound dilution, add RPMI 1640 medium containing 0.2% DMSO to the control group, and place the cells at 37° C. to incubate for 1 h in a cell culture incubator (Thermo Fisher Scientific, model: 3111). Then add Concanavalin A at a final concentration of 0.4 μg/ml and place in a cell culture incubator at 37° C. for 24 hours. Mouse IL-2 DuoSet ELISA KIT was used to detect the IL-2 content in the cell supernatant, and the Mouse IL-2 DuoSet ELISA detection was carried out according to the operating instructions of the kit. Data are described as the highest fold ratio of the stimulated signal of the compound to the signal of 0.1% DMSO.

Spleen IL-2 ELISA assay

| Compound | Concentration | % IL-2 increased (relative |
|---|---|---|
| number | (μM) | to DMSO control) |
| 68 | 10 | 170% |
| 69 | 3.3 | 220% |
| 70 | 3.3 | 190% |
| 71 | 3.3 | 160% |
| 72 | 3.3 | 210% |
| 73 | 10 | 160% |
| 75 | 3.3 | 150% |
| 76 | 3.3 | 190% |
| 77 | 1.11 | 340% |
| 78 | 3.3 | 330% |
| 79 | 3.3 | 130% |
| 80 | 3.3 | 130% |
| 81 | 3.3 | 150% |
| 82 | 0.0137 | 160% |
| 83 | 1.11 | 160% |
| 84 | 0.0046 | 110% |
| 85 | 10 | 260% |
| 86 | 10 | 250% |
| 89 | 3.3 | 150% |
| 90 | 10 | 310% |
| 91 | 1.1 | 160% |
| 92 | 0.37 | 160% |
| 93 | 3.3 | 150% |
| 96 | 10 | 200% |
| 97 | 3.3 | 170% |
| 98 | 3.3 | 190% |
| 100 | 3.3 | 190% |
| 101 | 3.3 | 210% |
| 102 | 3.3 | 180% |
| 103 | 10 | 180% |
| 104 | 0.0046 | 120% |
| 105 | 1.1 | 170% |
| 106 | 10 | 420% |
| 107 | 3.33 | 240% |
| 108 | 3.33 | 200% |
| 109 | 1.11 | 160% |
| 111 | 1.11 | 130% |
| 112 | 0.37 | 210% |

NA: indicates that no enhanced release of IL-2 was detected.

### Test Example 4: Detection of Compound's Activating Ability to Stimulate DC2.4 Cells to Secrete Cytokine Interleukin-6 (IL-6) (Method 3)

The required reagents and cells are as follows

Experimental Reagents:

| | | | Storage | |
|---|---|---|---|---|
| Reagent | brand | Item No. | Conditions | |
| RPMI1640 | BI | 01-100-1ACS | 4° | C. |
| FBS | BI | 04-002-1A | −80° | C. |
| BSA | Amresco | 0332-1KG | 4° | C. |
| Phosphate Buffered | BI | 02-024-1ACS | 4° | C. |
| Saline | | | | |
| Tween-20 | Solarbio | T8220 | RT | |
| LPS | Sigma | L3024 | −20° | C. |
| Mouse IL-6 DuoSet | R&D | DY406 | 4° | C. |
| ELISA | | | | |

Experimental Cells:

| cell | cell type | brand |
|---|---|---|
| DC2.4 | mouse bone marrow | Emdmillipore |
| | derived dendritic cells | |

Experimental Procedure

The specific operation is as follows: the compound powder was dissolved in DMSO to 10 mM, 2 μl of the compound was added to 998 μl of RPMI 1640 medium (contained 10% FBS in this test), and the highest concentration point was obtained after vortexing. The compound solution was gradually diluted 3 times with 0.2% DMSO medium, with a total of 8 concentration points. That treated with RPMI 1640 medium solution containing 0.1% DMSO was used as a control. Add 10⁵ DC2.4 cells to each well of Corning 96-well cell culture plate (product number: 3599), then add an equal volume of compound dilution, add RPMI 1640 medium containing 0.2% DMSO to the control group, and place the cells at 37° C. to incubate for 1 h in an incubator (Thermo Fisher Scientific, model: 3111). Subsequently, LPS was added at a final concentration of 3.2 ng/ml, and placed in a cell culture incubator at 37° C. for 24 hours. Mouse IL-6 DuoSet ELISA KIT was used to detect the IL-6 content in the cell supernatant, and the Mouse IL-2 DuoSet ELISA detection was carried out according to the operating instructions of the kit. Data are described as the highest fold ratio of the stimulated signal of the compound to the signal of 0.1% DMSO.

DC2.4 IL-6 ELISA assay

| Compound | Concentration | % IL-6 increased (relative |
|---|---|---|
| number | (μM) | to DMSO control) |
| 68 | 10 | 228.00% |
| 69 | 10 | 156.00% |
| 70 | 10 | 404.00% |
| 71 | 1.11 | 690.00% |
| 73 | 10 | 319.00% |
| 75 | 10 | 573.00% |
| 76 | 10 | 466.00% |
| 77 | 3.33 | 456.00% |
| 78 | 10 | 197.00% |
| 79 | 10 | 201.00% |
| 80 | 10 | 500.00% |
| 81 | 10 | 391.00% |
| 82 | 10 | 382.00% |
| 83 | 10 | 321.56% |
| 84 | 10 | 335.19% |
| 85 | 10 | 305.00% |
| 86 | 10 | 287.00% |
| 87 | 10 | 400.93% |
| 88 | 10 | 239.18% |
| 89 | 3.33 | 184.00% |
| 90 | 3.33 | 290.00% |
| 91 | 1.11 | 469.00% |
| 92 | 0.37 | 546.00% |
| 93 | 1.11 | 409.00% |
| 94 | 1.11 | 691.00% |
| 95 | 10 | 289.00% |
| 96 | 3.33 | 381.00% |
| 97 | 3.33 | 301.00% |
| 98 | 3.33 | 361.00% |
| 99 | 10 | 526.00% |
| 100 | 1.11 | 623.00% |
| 101 | 1.11 | 287.00% |
| 102 | 1.11 | 553.00% |
| 103 | 3.33 | 251.00% |
| 104 | 1.11 | 341.00% |
| 105 | 1.11 | 321.00% |
| 106 | 3.33 | 220.00% |
| 107 | 1.11 | 337.00% |
| 108 | 0.37 | 501.00% |
| 109 | 1.11 | 489.00% |
| 110 | 1.11 | 373.00% |
| 111 | 10 | 474.84% |

NA: indicates that no enhanced release of IL-6 was detected.

### Test Example 5: Detection of Compound's Activating Ability to Secrete Cytokine Interleukin-2 (IL-2) from PBMC (Method 5)

The required reagents and cells are as follows

Experimental Reagents:

| | | | Storage | |
|---|---|---|---|---|
| Reagent | brand | Item No. | Conditions | |
| RPMI1640 | BI | 01-100-1ACS | 4° | C. |
| FBS | BI | 04-002-1A | −80° | C. |
| BSA | Amresco | 0332-1KG | 4° | C. |
| Phosphate Buffered | BI | 02-024-1ACS | 4° | C. |
| Saline | | | | |
| Tween-20 | Solarbio | T8220 | RT | |
| Anti-human CD3 | invitrogen | 16-0037-85 | 4° | C. |
| Antibody | | | | |
| Anti-human CD28 | invitrogen | 16-0289-85 | 4° | C. |
| Antibody | | | | |
| Human IL-2 DuoSet | R&D | DY202 | 4° | C. |
| ELISA | | | | |

Experimental Cells:

| cell | cell type | brand |
|---|---|---|
| PBMC | NPB-MNC Y1232 | Allcells Biotechnology |
| | peripheral mononuclear cells | (Shanghai) Co., Ltd. |

Experimental Procedure

The specific operation is as follows: the compound powder was dissolved in DMSO to 10 mM, 2 μl of the compound was added to 998 μl of RPMI 1640 medium (contained 10% FBS in this test), and the highest concentration point was obtained after vortexing. The compound solution was gradually diluted 3 times with 0.2% DMSO medium, with a total of 8 concentration points. That treated with RPMI 1640 medium solution containing 0.1% DMSO was used as a control. 1×10˜ 5 PBMC cells were added to each well of Corning 96-well cell culture plate (item number: 3599), and then the same volume of compound diluent was added to the control group. RPMI 1640 medium containing 0.2% DMSO was added to the control group and incubated in a 37° C. cell incubator (Thermo Fisher Scientific, model: 3111) for 1 hour. Then add Anti-human CD3 Antibody and 1 μg/ml Anti-human CD28 Antibody at a final concentration of 0.1 μg/ml, and place in a 37° C. cell culture incubator to incubate for 24 hours. The Human IL-2 DuoSet ELISA KIT was used to detect the IL-2 content in the cell supernatant, and the Human IL-2 DuoSet ELISA detection was performed according to the operating instructions of the kit. Data are described as the highest fold ratio of the stimulated signal of the compound to the signal of 0.1% DMSO.

PBMC IL-2 ELISA assay

| Compound | Concentration | % IL-2 Increased (relative |
|---|---|---|
| number | (μM) | to DMSO control) |
| 68 | 10 | 345% |
| 69 | 10 | 255% |
| 70 | 10 | 392% |
| 72 | 3.3 | 367% |
| 73 | 10 | 307% |
| 74 | 1.1-10 | 246% |
| 75 | 3.3 | 246% |
| 76 | 10 | 241% |
| 77 | 3.3 | 404% |
| 78 | 10 | 241% |
| 79 | 10 | 327% |
| 80 | 10 | 165% |
| 81 | 3.3 | 208% |
| 85 | 10 | 339% |
| 86 | 10 | 359% |
| 87 | 10 | 277% |
| 88 | 3.3 | 210% |
| 90 | 1.1 | 159% |
| 91 | 0.37 | 220% |
| 92 | 0.37 | 182% |
| 93 | 1.1 | 213% |
| 94 | 1.1 | 278% |
| 95 | 3.3 | 259% |
| 96 | 3.3 | 217% |
| 97 | 3.3 | 238% |
| 98 | 1.1 | 184% |
| 99 | 1.1 | 236% |
| 100 | 0.37 | 239% |
| 101 | 0.37 | 135% |
| 103 | 3.3 | 167% |
| 104 | 0.37 | 139% |
| 105 | 1.1 | 176% |
| 106 | 3.3 | 172% |
| 107 | 1.1 | 184% |
| 111 | 0.37 | 163% |

NA: indicates that no enhanced release of IL-2 was detected.

1. A compound having the structure represented by formula I or a pharmaceutically acceptable salt, an isotopic derivative, a stereoisomer thereof: 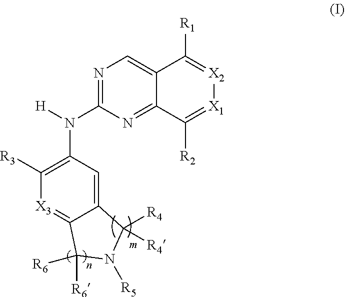
wherein R₁ represents hydrogen, halogen, (C₁-C₆) alkyl, (C₂-C₆) alkenyl, (C1-C6) alkoxy;
wherein R₂ represents hydrogen, halogen, hydroxyl, (C₁-C₆) alkyl, (C₂-C₆) alkenyl, —(C₀-C₆ alkylene) (C1-C6) alkoxy, —(C₀-C₆ alkylene) (C₆-C₁₀) aryl, —(C₀-C₆ alkylene) (5-10) membered heteroaryl, —(C₀-C₆ alkylene) (4-10 membered) heterocycloalkyl, —(C₀-C₆ alkylene) (C₃-C₈) cycloalkyl, —NR^{L}R^{L′}, —OR^{L′}, —SR^{L};
wherein R₃ represents hydrogen, halogen, hydroxyl, (C₁-C₆) alkyl, (C₂-C₆) alkenyl, —(C₀-C₆ alkylene) (C₃-C₈) cycloalkyl, —(C₀-C₆ alkylene) (4-8 membered) heterocycloalkyl, (C₁-C₆) alkoxy, —(C₀-C₆ alkylene) (4-8 membered) heterocycloalkyloxy;
wherein R₄ and R_{4′} independently represent hydrogen, C₁-C₆ alkyl, (C₂-C₆) alkenyl, halogen;
alternatively, R₄ and R_{4′} form a 3-6 membered ring together with the carbon atom connected to it, and the ring can also optionally contain 0, 1, 2 heteroatoms selected from N, O, and S;
wherein R₅ represents hydrogen, C₁-C₆ alkyl, (C₃-C₆) alkenyl, (C₃-C₈) cycloalkyl, (4-8 membered) heterocycloalkyl;
wherein R₆ and R_{6′} independently represent hydrogen, C₁-C₆ alkyl, (C₂-C₆) alkenyl, halogen;
alternatively, R₆ and R_{6′} form a 3-6 membered ring together with the carbon atom connected to it, and the ring can also optionally contain 0, 1, 2 heteroatoms selected from N, O, and S;
wherein X₁ represents N or CH;
wherein X₂ represents N or CR₇;
wherein X₃ represents N or CR₈;
wherein R₇ represents hydrogen, halogen, (C₁-C₆) alkyl, (C₂-C₆) alkenyl, (C₃-C₈) cycloalkyl;
wherein R represents hydrogen, halogen, (C₁-C₆) alkyl, (C₂-C₆) alkenyl, —(C₀-C₆ alkylene) (C₃-C₈) cycloalkyl, —(C₀-C₆ alkylene)(4-10 membered) heterocycloalkyl, —(C₀-C₆ alkylene) (C₆-C₁₀) aryl, —(C₀-C₆ alkylene) (5-10 membered) heteroaryl,
alternatively, R₈ can form (5-10 membered) cycloalkyl or (5-10 membered) heterocycloalkyl together with adjacent R₃;
wherein R^{L} and R^{L′} each independently represent hydrogen, (C₁-C₆) alkyl, (C₃-C₆) alkenyl, (C₃-C₆) cycloalkyl, (4-8 membered) heterocycloalkyl, (C₀-C₆ alkylene) (C₆-C₁₀) aryl, —(C₀-C₆ alkylene) (5-10) membered heteroaryl, —(C₀-C₆) alkylene-(CR^{M}R^{M′})—(C₀-C₆)alkyl, —(C₀-C₆)alkylene-(CR^{M}R^{M′})-halogen;
alternatively, R^{L} and R^{L′} form a 4-8 membered ring together with the nitrogen atom connected to it, which can additionally contain 0, 1, or 2 heteroatoms selected from nitrogen, oxygen, and sulfur;
wherein the ring can also be optionally fused to another 5-6 membered carbocycle, 5-6 cycloheteroalkane, 3-4 membered carbocycle, 3-4 cycloheteroalkane, 5-6 membered aromatic heterocycle or benzene ring to form a fused ring bicyclic system;
or the ring can also be connected to another (4-6 membered) ring carbocycle or (4-6 membered) heterocycle through a spiro carbon atom to form a spirobicyclic ring system;
wherein the fused ring bicyclic system or spirocyclic bicyclic system can be optionally substituted by 0, 1, 2, 3 members selected from halogen, cyano, (C₁-C₆) alkyl, (C₂-C₆) alkenyl, —NR^{a}R^{a′}, —OR^{a}, —SRₐ, —(C₁-C₆)alkylene-ORₐ, —(C₁-C₆)alkylene-SR^{a}, —(C₁-C₆ alkylene)hydroxyl, —C(O)Rₐ, —N(R^{a})C(O)R^{a}, —N(R^{a})C(O)OR^{a}, —N(R^{a})SO₂R^{a}, —C(O)OR^{a}, —C(O)NR^{a}R^{a′}, —S(O)₂NR^{a}R^{a′}, —S(O)R^{a}, —S(O)₂R^{a};
wherein R^{M} and R^{M′} independently represent hydrogen, C₁-C₆ alkyl;
alternatively, R^{M} and R^{M′} form a 3-8 membered ring together with the carbon atom connected to it, and the ring can also optionally contain 0, 1, or 2 heteroatoms selected from N, O, and S;
For the above defined alkyl, cycloalkyl, heterocycloalkyl, aryl, heteroaryl, it can be optionally substituted by 0, 1, 2, 3 substituents selected from the following: (C₁-C₆) alkyl, (C₂-C₆) alkenyl, halo (C₁-C₆) alkyl, halo (C₁-C₆) alkoxy, —(C1-C₆ alkylene)-O—(C₁-C₆) alkyl, (C₃-C₈) cycloalkyl, halogenated (C₃-C₈) cycloalkyl, halogen, —CN, oxo, —NRₐR_{a′}, —ORₐ, —SRₐ, —(C₁-C₆ alkylene) hydroxyl, —C(O)Rₐ, —N(Rₐ)C(O)Rₐ, —NRₐC(O)ORₐ, —NRₐSO₂Rₐ, —C(O)ORₐ, —C(O)NRₐR_{a′}, —S(O)₂NRₐR_{a′}, —S(O)Rₐ, —S(O)₂Rₐ, —P(O)RₐR_{a′};
wherein Rₐ and R_{a′} each independently represent hydrogen, (C₁-C₆) alkyl, (C₂-C₆) alkenyl, (C₃-C₈) cycloalkyl; or when Rₐ and R_{a′} are connected together on the N atom, they can form a 4-7 membered cycloheteroalkane together with the N atom connected to it;
wherein m and n represent 0, 1, 2, 3.

2. The compound or pharmaceutically acceptable salt, isotope derivative, stereoisomer thereof as claimed in claim 1, wherein R^{L} and R^{L′} independently represent hydrogen, (C₁-C₆) alkyl, (C₃-C₆) alkenyl, (C₃-C₆) cycloalkyl, (4-8 members) heterocycloalkyl, (C₀-C₆ alkylene) (C6-C10) aryl, —(C₀-C₆ alkylene) (5-10) membered heteroaryl, —(C₀-C₆) alkylene-(CR^{M}R^{M′})—(C₀-C₆) alkyl, —(C₀-C₆) alkylene-(CR^{M}R^{M′})-halogen;
alternatively, R^{L} and R^{L′} form a 4-8 membered ring together with the nitrogen atom connected to it, which can additionally contain 0, 1, or 2 heteroatoms selected from nitrogen, oxygen, and sulfur;
wherein the ring can also be optionally fused to another 5-6 membered carbocycle, 5-6 membered cycloheteroalkane, 5-6 membered aromatic heterocycle or benzene ring to form a fused ring bicyclic system;
or the ring can also be connected to another (4-6 membered) ring carbocycle or (4-6 membered) heterocycle through a spiro carbon atom to form a spirobicyclic ring system;
wherein the fused ring bicyclic system or spirocyclic bicyclic system can be optionally substituted by 0, 1, 2, 3 members selected from halogen, cyano, (C₁-C₆) alkyl, (C₂-C₆) alkenyl, —NRₐR_{a′}, —ORₐ, —SRₐ, —(C₁-C₆ alkylene) hydroxyl, —C(O)Rₐ, —N(Rₐ)C(O)Rₐ, —N(Rₐ)C(O)ORₐ, —N(Rₐ)SO₂Rₐ, —C(O)ORₐ, —C(O)NRₐR_{a′}, —S(O)2NRₐR_{a′}, —S(O)Rₐ, —S(O)2Rₐ; or
R^{L} and R^{L′} independently represent hydrogen, (C₁-C₆) alkyl, (C₃-C₆) alkenyl, (C₃-C₆) cycloalkyl, (C₀-C₆ alkylene) (C₆-C₁₀) aryl, —(C₀-C₆ alkylene) (5-10) member heteroaryl, —(C₀-C₆)alkylene-(CR^{M}R^{M′})—(C₀-C6)alkyl, —(C₀-C6)alkylene-(CR^{M′}R^{M′})-halogen;
alternatively, R^{L} and R^{L′} form a 4-8 membered ring together with the nitrogen atom connected to it, which can additionally contain 0, 1, or 2 heteroatoms selected from nitrogen, oxygen, and sulfur;
wherein the ring can also be optionally fused to another 5-6 membered carbocycle, 5-6 membered cycloheteroalkane, 5-6 membered aromatic heterocycle or benzene ring to form a fused ring bicyclic system;
or the ring can also be connected to another (4-6 membered) ring carbocycle or (4-6 membered) heterocycle through a spiro carbon atom to form a spirobicyclic ring system;
wherein the fused ring bicyclic system or spirocyclic bicyclic system can be optionally substituted by 0, 1, 2, 3 members selected from halogen, cyano, (C₁-C₆) alkyl, (C₂-C₆) alkenyl, —NRₐR_{a′}, —ORₐ, —SRₐ, —(C₁-C₆ alkylene) hydroxyl, —C(O)Rₐ, —N(Rₐ)C(O)Rₐ, —N(Rₐ)C(O)ORₐ, —N(Rₐ)SO₂Rₐ, —C(O)ORₐ, —C(O)NRₐRa′, —S(O)2NRaRa′, —S(O)Ra, —S(O)2Ra.

3. (canceled)

4. The compound or pharmaceutically acceptable salt, isotope derivative, stereoisomer thereof as claimed in claim 1 having the following formula (II) structure: 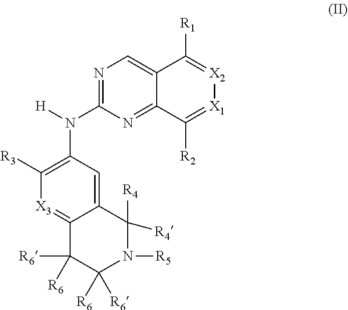
wherein R₁, R₂, R₃, R₄, R_{4′}, R₅, R_{5′}, R₆, R_{6′}, X₁, X₂, X₃ are as defined in claim 1.

5. The compound or pharmaceutically acceptable salt, isotope derivative, stereoisomer thereof as claimed in claim 1, wherein R₂ represents (C₁-C₆) alkyl, —(C₀-C₆ alkylene) (C₆-C₁₀) aryl, —(C₀-C₆ alkylene) (5-10) heteroaryl, —(C₀-C₆ alkylene) (4-10 membered) heterocycloalkyl, —(C₀-C₆ alkylene) (C₃-C₈) cycloalkyl; wherein, said alkyl, aryl, heteroaryl, heterocycloalkyl, cycloalkyl can be optionally substituted by 0, 1, 2 members selected from halogen, C₁-C₆ alkyl, —ORₐ, —SRₐ, —(C₁-C₆ alkylene) hydroxyl, halogenated (C₁-C₆) alkyl, halogenated (C₁-C₆) alkoxy, —(C₁-C₆ alkylene)-O—(C₁-C₆)alkyl, C₃-C₆ cycloalkyl, oxo, —NRₐR_{a′}, C(O)Rₐ, —N(Rₐ)C(O)Rₐ, —NRₐC(O) ORₐ, —NRₐSO2Rₐ, —C(O)ORₐ, —C(O)NRₐR_{a′}, —S(O)2NRₐR_{a′}, —S(O)Rₐ, —S(O)₂Rₐ, —P(O)RₐR_{a′}; or
R₂ represents NR^{L}R^{L′}, wherein R^{L} represents hydrogen or C₁-C₆ alkyl: R^{L′} represents C₁-C₆ alkyl, (C₃-C₆) cycloalkyl, (4-8 membered) heterocycloalkyl, (C₀-C₆ alkylene) (C₆-C₁₀) aryl, —(C₀-C₆ alkylene) (5-10) membered heteroaryl, wherein, said R^{L} and R^{L′} can be independently optionally substituted by 0, 1, 2 members selected from halogen, C₁-C₆ alkyl, halogenated (C₁-C₆) alkyl, ORₐ, cyano; or
R₂ represents NR^{L}R^{L′}, wherein said R^{L}, R^{L} form a 4-8 membered ring together with the nitrogen connected to it, which may additionally contain 0, 1, or 2 heteroatoms selected from nitrogen, oxygen, and sulfur;
wherein the ring can also be optionally fused to another 5-6 membered carbocycle, 5-6 cycloheteroalkane, 3-4 membered carbocycle, 3-4 cycloheteroalkane, 5-6 membered aromatic heterocycle or benzene ring to form a fused ring bicyclic system;
or the ring can also be connected to another (4-6 membered) ring carbocycle or (4-6 membered) heterocycle through a spiro carbon atom to form a spirobicyclic ring system;
wherein the fused ring bicyclic system or spirocyclic bicyclic system can be optionally substituted by 0, 1, 2, 3 members selected from halogen, cyano, (C₁-C₆) alkyl, (C₂-C₆) alkenyl, oxo, —NRₐR_{a′}, —ORₐ, —SRₐ, —(C₁-C₆)alkylene-ORₐ, —(C₁-C₆)alkylene-SRₐ, —C(O)Rₐ, —N(Rₐ)C(O)Rₐ, —N(Rₐ)C(O)ORₐ, —N(Rₐ)SO₂Rₐ, —C(O)ORₐ, —C(O)NRₐR_{a′}, —S(O)₂NRₐR_{a′}, —S(O)Rₐ, —S(O)₂Rₐ; or
R₂ represents NR^{L}R^{L′}, wherein R^{L} represents hydrogen or C₁-C₆ alkyl; R^{L′} represents —(C₀-C₆ alkylene)-(CR^{M}R^{M′})—(C₀-C₆)alkyl, —(C₀-C₆ alkylene)-(CR^{M}R^{M′})—(C₀-C₆)alkyl, —(C₀-C₆ alkylene)- (CR^{M}R^{M′})-halogen, wherein R^{M} and R^{M′} each independently represent hydrogen, C₁-C₆ alkyl;
alternatively, R^{M} and R^{M′} form a 3-8 membered ring together with the carbon atom connected to it, and the ring can also optionally contain 0, 1, 2 heteroatoms selected from N, O, S or oxo, —NRₐ group.

6. (canceled)

7. (canceled)

8. (canceled)

9. The compound or pharmaceutically acceptable salt, isotope derivative, stereoisomer thereof as claimed in claim 1, wherein R₁ represents hydrogen, C₁-C₆ alkyl, halogen, ORₐ, NRₐR_{a′}, cyano, —SO₂Rₐ, halogenated (C₁-C₆) alkyl, (C₃-C₆) cycloalkyl; preferably hydrogen, C₁-C₆ alkyl, halogen, halogenated (C₁-C₆) alkyl; more preferably hydrogen, C₁-C₆ alkyl.

10. The compound or pharmaceutically acceptable salt, isotope derivative, stereoisomer thereof as claimed in claim 1, wherein X₂ represents CR₇, wherein R₇ represents hydrogen, halogen, hydroxyl, cyano, (C₁-C₆)alkyl, (C₃-C₆)cycloalkyl, halo(C₁-C₆)alkyl.

11. A compound having the structure represented by formula (III) or a pharmaceutically acceptable salt, an isotopic derivative, a stereoisomer thereof (III): 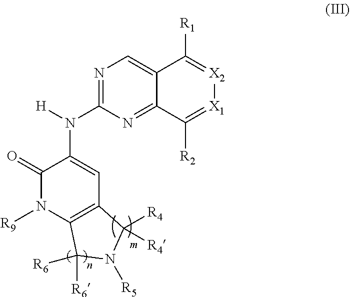
wherein R₁ represents hydrogen, halogen, hydroxyl, (C₁-C₆) alkyl, (C₂-C₆) alkenyl, (C₁-C₆) alkoxy;
wherein R₂ represents hydrogen, halogen, hydroxyl, (C₁-C₆) alkyl, (C₂-C₆) alkenyl, —(C₀-C₆ alkylene) (C₁-C₆) alkoxy, —(C₀-C₆ alkylene) (C₆-C₁₀) aryl, —(C₀-C₆ alkylene) (5-10) heteroaryl, —(C₀-C₆ alkylene) (4-10) heterocycloalkyl, —(C₀-C₆ alkylene) (C₃-C₈) cycloalkyl, —NR^{L}R^{L′}, —OR^{L}, —SR^{L};
wherein R₄ and R_{4′} independently represent hydrogen, (C₁-C₆) alkyl, (C₂-C₆) alkenyl, halogen;
alternatively, R₄ and R_{4′} form a 3-6 membered ring together with the carbon atom connected to it, and the ring can also optionally contain 0, 1, 2 heteroatoms selected from N, O, and S;
wherein R₅ represents hydrogen, C₁-C₆ alkyl, (C₃-C₆) alkenyl, (C₃-C₈) cycloalkyl, (4-8 membered) heterocycloalkyl;
wherein R₆ and R_{6′} independently represent hydrogen, C₁-C₆ alkyl, (C₂-C₆) alkenyl, halogen;
alternatively, R₆ and R_{6′} form a 3-6 membered ring together with the carbon atom connected to it, and the ring can also optionally contain 0, 1, 2 heteroatoms selected from N, O, and S;
wherein X₁ represents N or CH;
wherein X₂ represents N or CR₇;
wherein R₇ represents hydrogen, halogen, (C₁-C₆) alkyl, (C₂-C₆) alkenyl, (C₃-C₈) cycloalkyl, (C₁-C₆) alkoxy;
wherein R represents hydrogen, (C₁-C₆) alkyl, (C₃-C₆) alkenyl, —(C₀-C₆ alkylene) (C₃-C₈) cycloalkyl, —(C₀-C₆ alkylene) (4-10 membered) heterocycloalkyl, —(C₀-C₆ alkylene) (C₆-C₁₀) aryl, —(C₀-C₆ alkylene) (5-10) membered heteroaryl;
wherein R^{L} and R^{L′} independently represent hydrogen, (C₁-C₆) alkyl, (C₃-C₆) alkenyl, (C₃-C₆) cycloalkyl, (4-8) heterocycloalkyl, (C₀-C₆ alkylene) (C₆-C₁₀) aryl, —(C₀-C₆ alkylene) (5-10) member heteroaryl, —(C₀-C₆) alkylene-(CR^{M}R^{M′})—(C₀-C₆)alkyl, —(C₀-C₆)alkylene-(CR^{M}R^{M′})-halogen;
alternatively, R^{L} and R^{L′} form a 4-8 membered ring together with the nitrogen atom connected to it, and the ring may additionally contain 0, 1, 2 heteroatoms selected from nitrogen, oxygen, sulfur or oxo, —NRₐ groups, and the ring may also optionally be fused to another 5-6 membered carbocycle, 5-6 cycloheteroalkane, 3-4 membered carbocycle, 3-4 cycloheteroalkane, 5-6 membered aromatic heterocycle ring or benzene ring to form a fused ring bicyclic ring system;
or the ring can also be connected to another (4-6 membered) ring carbocycle or (4-6 membered) heterocycle through a spiro carbon atom to form a spirobicyclic ring system;
wherein the fused ring bicyclic system or spirocyclic bicyclic system can be optionally substituted by 0, 1, 2, 3 members selected from halogen, cyano, (C₁-C₆) alkyl, (C₂-C₆) alkenyl, —NR^{a}R^{a′}, —OR^{a}, —SR^{a}, —(C₁-C₆)alkylene-OR^{a}, —(C₁-C₆)alkylene-SR^{a}, —(C₁-C₆ alkylene)hydroxyl, —C(O)R^{a}, —N(R^{a})C(O)R^{a}, —N(R^{a})C(O)OR^{a}, —N(R^{a})SO₂R^{a}, —C(O)OR^{a}, —C(O)NR^{a}R^{a′}, —S(O)₂NR^{a}R^{a′}, —S(O)R^{a}, —S(O)₂R^{a};
wherein R^{M} and R^{M′} independently represent hydrogen, C₁-C₆ alkyl;
alternatively, R^{M} and R^{M′} form a 3-8 membered ring together with the carbon atom connected to it, and the ring can also optionally contain 0, 1, 2 heteroatoms selected from N, O, S or oxo, —NRₐ group;
For the above-mentioned defined alkyl, ring, cycloalkyl, heterocycloalkyl, aryl, heteroaryl, it can be optionally substituted by 0, 1, 2, 3 substituents selected from the following: (C₁-C₆) alkyl, (C₂-C₆) alkenyl, halo (C₁-C₆) alkyl, halo (C₁-C₆) alkoxy, (C₃-C₈) cycloalkyl, —(C₁-C₆ alkylene) —O—(C₁-C₆) alkyl, halogenated (C₃-C₈) cycloalkyl, halogen, —CN, oxo, —NRₐR_{a′}, —ORₐ, —SRₐ, —(C₁-C₆ alkyl) hydroxy, —C(O)Rₐ, —N(Rₐ)C(O)Rₐ, —NRₐC(O)ORₐ, —NRₐSO₂Rₐ, —C(O)ORₐ, —C(O)NRₐR_{a′}, —S(O)₂NRₐR_{a′}, —S(O)Rₐ, —S(O)₂Rₐ, —P(O)RₐR_{a′};
wherein Rₐ and R_{a′} each independently represent hydrogen, (C₁-C₆) alkyl, (C₂-C₆) alkenyl, (C₃-C₈) cycloalkyl, or Rₐ and R_{a′} can form a 4-7 membered cycloheteroalkanes together with the nitrogen atom connected to it;
wherein m and n represent 0, 1, 2, 3.

12. The compound or pharmaceutically acceptable salt, isotope derivative, stereoisomer thereof as claimed in claim 11, wherein, R^{L} and R^{L′} independently represent hydrogen, (C₁-C₆) alkyl, (C₃-C₆) alkenyl, (C₃-C₆) cycloalkyl, (4-8 members) heterocycloalkyl, (C₀-C₆ alkylene) (C₆-C₁₀) aryl, —(C₀-C₆ alkylene) (5-10) membered heteroaryl, —(C₀-C₆) alkylene-(CR^{M}R^{M′})—(C₀-C₆) alkyl, —(C₀-C₆) alkylene-(CR^{M}R^{M′})-halogen;
alternatively, R^{L} and R^{L′} form a 4-8 membered ring together with the nitrogen atom connected to it, and the ring may additionally contain 0, 1, 2 heteroatoms selected from nitrogen, oxygen, sulfur or oxo, —NRₐ groups and this ring may also optionally be fused to another 5-6 membered carbocycle, 5-6 cycloheteroalkane, 5-6 membered aromatic heterocycle or benzene ring to form a fused ring bicyclic system;
or the ring can also be connected to another (4-6 membered) ring carbocycle or (4-6 membered) heterocycle through a spiro carbon atom to form a spirobicyclic ring system;
wherein the fused ring bicyclic system or spirocyclic bicyclic system can be optionally substituted by 0, 1, 2, 3 members selected from halogen, cyano, (C₁-C₆) alkyl, (C₂-C₆) alkenyl, —NRₐR_{a′}, —ORₐ, —SRₐ, —(C₁-C₆ alkylene) hydroxyl, —C(O)Rₐ, —N(Rₐ)C(O)Rₐ, —N(Rₐ)C(O)ORₐ, —N(Rₐ)SO₂Rₐ, —C(O)ORₐ, —C(O)NRₐR_{a′}, —S(O)₂NRₐR_{a′}, —S(O)Rₐ, —S(O)₂Rₐ; or
R^{L} and R^{L′} independently represent hydrogen, (C₁-C₆) alkyl, (C₃-C₆) alkenyl, (C₃-C₆) cycloalkyl, (C₀-C₆ alkylene) (C₆-C₁₀) aryl, —(C₀-C₆ alkylene) (5-10) member heteroaryl, —(C₀-C₆)alkylene-(CR^{M}R^{M′})—(C₀-C6)alkyl, —(C₀-C6)alkylene-(CR^{M′}R^{M′})-halogen;
alternatively, R^{L} and R^{L′} form a 4-8 membered ring together with the nitrogen atom connected to it, and the ring may additionally contain 0, 1, 2 heteroatoms selected from nitrogen, oxygen, sulfur or oxo, —NRₐ groups and this ring may also optionally be fused to another 5-6 membered carbocycle, 5-6 cycloheteroalkane, 5-6 membered aromatic heterocycle or benzene ring to form a fused ring bicyclic system;
or the ring can also be connected to another (4-6 membered) ring carbocycle or (4-6 membered) heterocycle through a spiro carbon atom to form a spirobicyclic ring system;
wherein the fused ring bicyclic system or spirocyclic bicyclic system can be optionally substituted by 0, 1, 2, 3 members selected from halogen, cyano, (C₁-C₆) alkyl, (C₂-C₆) alkenyl, —NRₐR_{a′}, —ORₐ, —SRₐ, —(C₁-C₆ alkylene) hydroxyl, —C(O)Rₐ, —N(Rₐ)C(O)Rₐ, —N(Rₐ)C(O)ORₐ, —N(Rₐ)SO₂Rₐ, —C(O)ORₐ, —C(O)NRₐR_{a′}, —S(O)2NRₐR_{a′}, —S(O)Rₐ, —S(O)₂Rₐ; or
R^{L} and R^{L′} independently represent hydrogen, (C₁-C₆) alkyl, (C₃-C₆) alkenyl, (C₃-C₆) cycloalkyl, (C₀-C₆ alkylene) (C₆-C₁₀) aryl, —(C₀-C₆ alkylene) (5-10) member heteroaryl, —(C₀-C₆)alkylene-(CR^{M}R^{M′})—(C₀-C6)alkyl, —(C₀-C6)alkylene-(CR^{M′}R^{M′})-halogen;
alternatively, R^{L} and R^{L′} form a 4-8 membered ring together with the nitrogen atom connected to it, and the ring may additionally contain 0, 1, 2 heteroatoms selected from nitrogen, oxygen, sulfur or oxo, —NRₐ groups and this ring may also optionally be fused to another 5-6 membered carbocycle, 5-6 cycloheteroalkane, 5-6 membered aromatic heterocycle or benzene ring to form a fused ring bicyclic system;
alternatively, the ring may also be connected via a spiro carbon atom to another (4-6 membered) ring carbocycle or (4-6 membered) heterocycle to form a spirobicyclic ring system.

13. (canceled)

14. (canceled)

15. The compound or pharmaceutically acceptable salt, isotope derivative, stereoisomer thereof as claimed in claim 11 having the following formula (IV) structure: 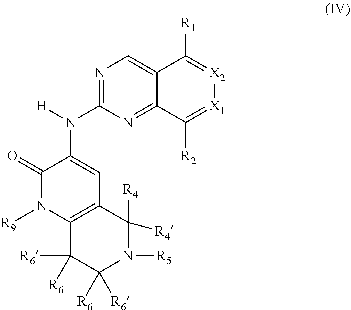
wherein R₁, R₂, R₃, R₄, R_{4′}, R₅, R_{5′}, R₆, R_{6′}, R₉, X₁, X₂ are as defined in claim 11.

16. The compound or pharmaceutically acceptable salt, isotope derivative, stereoisomer thereof as claimed in claim 11, wherein R represents (C₁-C₆) alkyl, —(C₀-C₆ alkylene) (C₆-C₁₀) aryl, —(C₀-C₆ alkylene) (5-10) membered heteroaryl, —(C₀-C₆ alkylene) (4-10 membered) heterocycloalkyl, —(C₀-C₆ alkylene) (C₃-C₈) cycloalkyl; wherein, said alkyl, aryl, heteroaryl, cycloalkyl, heterocycloalkyl can be optionally substituted by 0, 1, 2 members selected from halogen, C₁-C₆ alkyl, —ORₐ, —SRₐ, halogenated (C₁-C₆) alkyl, halogenated (C₁-C₆) alkoxy, —(C₁-C₆ alkylene) —O—(C₁-C6) alkyl, C₃-C₆ cycloalkyl, —NRₐR_{a′}, C(O)Rₐ, —N(Rₐ)C(O)Rₐ, —NRₐC(O)ORₐ, —NRₐSO₂Rₐ, —C(O)ORₐ, —C(O)NRₐR_{a′}, —S(O)₂NRₐR_{a′}, —S(O)Rₐ, —S(O)₂Rₐ, —P(O)RₐR_{a′}; or
R₂ represents NR^{L}R^{L′}, wherein R^{L} represents hydrogen or C₁-C₆ alkyl; R^{L′} represents C₁-C₆ alkyl, (C₃-C₆) cycloalkyl, (4-8 membered) heterocycloalkyl, (C₀-C₆ alkylene) (C₆-C₁₀) aryl, —(C₀-C₆ alkylene) (5-10) membered heteroaryl, wherein, said R^{L} and R^{L′} can be independently optionally substituted by 0, 1, 2 members selected from halogen, C₁-C₆ alkyl, halogenated (C₁-C₆) alkyl, ORₐ, cyano, wherein Rₐ represents hydrogen, (C₁-C₆) alkyl; or
R₂ represents NR^{L}R^{L′}, wherein said R^{L}, R^{L′} form a 4-8 membered ring together with the nitrogen atom connected to it which may additionally contain 0, 1, or 2 heteroatoms selected from nitrogen, oxygen, and sulfur or oxo, —NRₐ groups:
wherein the ring can also be optionally fused to another 5-6 membered carbocycle, 5-6 cycloheteroalkane, 3-4 membered carbocycle, 3-4 cycloheteroalkane, 5-6 membered aromatic heterocycle or benzene ring to form a fused ring bicyclic system;
or the ring can also be connected to another (4-6 membered) ring carbocycle or (4-6 membered) heterocycle through a spiro carbon atom to form a spirobicyclic ring system;
wherein the ring can be optionally substituted by 0, 1, 2, 3 members selected from halogen, cyano, (C₁-C₆) alkyl, oxo, —NRₐR_{a′}, —ORₐ, —SRₐ, —(C1-C6)alkylene-ORₐ, —(C₁-C₆)alkylene-SRₐ, —C(O)Rₐ, —N(Rₐ)C(O)Rₐ, —N(Rₐ)C(O)ORₐ, —N(Rₐ)SO₂Rₐ, —C(O)ORₐ, —C(O)NRₐR_{a′}, —S(O)₂NRₐR_{a′}, —S(O)Rₐ, —S(O)₂Rₐ, wherein Rₐ, R_{a′} each independently represent hydrogen, (C₁-C₆) alkyl; or
R₂ represents NR^{L}R^{L′}, wherein R^{L} represents hydrogen or C₁-C₆ alkyl; R^{L′} represents —(C₀-C₆ alkylene)-(CR^{M}R^{M′})—(C₀-C6)alkyl, —(C₀-C6 alkylene)-(CR^{M′}R^{M′})—(C₀-C6)alkyl, —(C₀-C6 alkylene)- (CR^{M}R^{M′})-halogen, wherein R^{M} and R^{M′} each independently represent hydrogen, C₁-C₆ alkyl;
alternatively, R^{M} and R^{M′} form a 3-8 membered ring together with the carbon atom connected to it, and the ring can also optionally contain 0, 1, 2 heteroatoms selected from N, O, S or oxo, —NRₐ group.

17. (canceled)

18. (canceled)

19. The compound or pharmaceutically acceptable salt, isotope derivative, stereoisomer thereof as claimed in claim 11, wherein R₂ represents —(C₀-C₆ alkylene) (C₆-C₁₀) aryl, —(C₀-C₆ alkylene) (5-10) member heteroaryl, —(C₀-C₆ alkylene) (4-10 membered) heterocycloalkyl, —(C₀-C₆ alkylene) (C₃-C₈) cycloalkyl, wherein said R₂ can be optionally substituted by 0, 1, 2 members selected from halogen, C₁-C₆ alkyl, —ORₐ, —SRₐ, —(C₁-C₆alkylene) hydroxyl, halogenated (C₁-C₆) alkyl, halogenated (C₁-C₆) alkoxy, —(C₁-C₆ alkylene)-O—(C1-C6) alkyl, C₃-C₆ cycloalkyl, —NRₐR_{a′}, —C(O)Rₐ, —N(Rₐ)C(O)Rₐ, —NRₐC(O)ORₐ, —NRₐSO2Rₐ, —C(O)ORₐ, —C(O)NRₐR_{a′}, —S(O)2NRₐR_{a′}, —S(O)Rₐ, —S(O)2Ra, —P(O)RₐR_{a′}, wherein Rₐ, R_{a′} each independently represent hydrogen, (C₁-C₆) alkyl; or
R₂ represents halogen, C₁-C₆ alkyl, —ORₐ, —C(O)ORₐ, —C(O)NRₐR_{a′}, —(C₁-C₆ alkylene)hydroxyl, (C₆-C₁₀)aryl substituted by halogenated (C₁-C₆)alkoxy, (5-10)membered heteroaryl; or
R₂ represents phenyl, pyridyl, pyrazolyl,
R₂ represents phenyl, pyridyl, pyrazolyl, 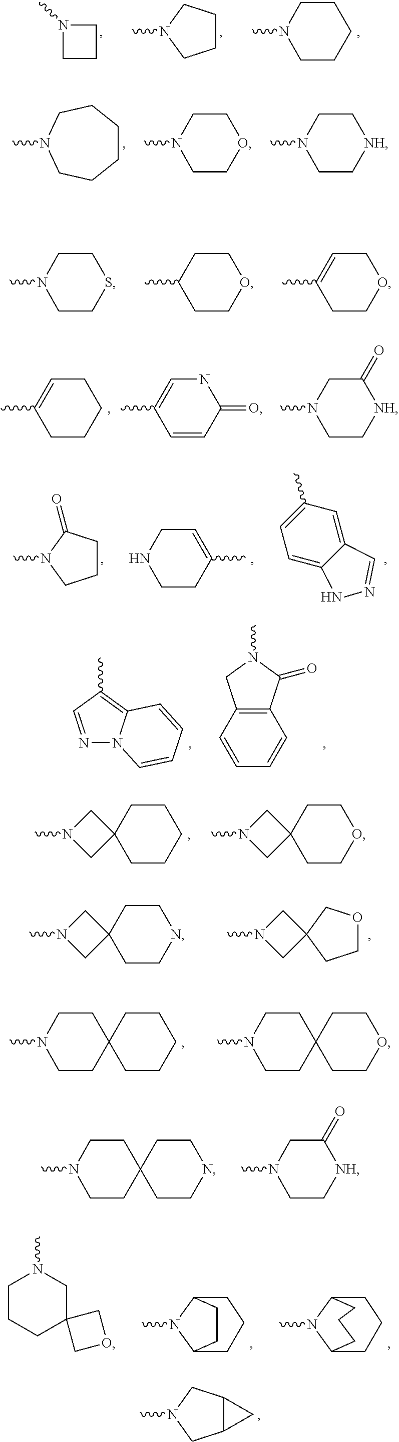
where the dotted line indicates the junction site,
wherein the R₂ can be optionally substituted by members selected from halogen, C₁-C₆ alkyl, ORₐ, SRₐ, C₁-C₆ alkylene hydroxyl, —(C₁-C₆ alkylene)-O—(C₁-C₆) alkane Group, —C(O)Rₐ, —C(O)ORₐ, —C(O)NRₐR_{a′}, —S(O)₂NRₐR_{a′}, —S(O)₂Rₐ, —S(O)Rₐ, halogenated (C₁-C₆) alkyl, halo (C₁-C₆) alkoxy.

20. (canceled)

21. (canceled)

22. (canceled)

23. The compound or pharmaceutically acceptable salt, isotope derivative, stereoisomer thereof as claimed in claim 1, wherein R represents hydrogen, C₁-C₆ alkyl, halogen, ORₐ, NRₐR_{a′}, cyano, —SO₂Rₐ, halogenated (C₁-C₆) alkyl, (C₃-C₆) cycloalkyl, wherein, Rₐ, R_{a′} each independently represent hydrogen, (C₁-C₆) alkyl; preferably hydrogen, C₁-C₆ alkyl, halogen, halo(C₁-C₆) alkyl; more preferably hydrogen, C₁-C₆ alkyl.

24. The compound or pharmaceutically acceptable salt, isotope derivative, stereoisomer thereof as claimed in claim 1, wherein X₂ represents CR₇, wherein R₇ represents hydrogen, halogen, hydroxyl, cyano, (C₁-C₆)alkyl, (C₃-C₆)cycloalkyl, halo(C₁-C₆)alkyl.

25. The compound or pharmaceutically acceptable salt, isotope derivative, stereoisomer thereof as claimed in claim 1, wherein R₄ and R_{4′} each independently represent hydrogen, C₁-C₆ alkyl, halogen.

26. The compound or pharmaceutically acceptable salt, isotope derivative, stereoisomer thereof as claimed in claim 1, wherein R₅ represents hydrogen, C₁-C₆ alkyl, (C₃-C₆) alkenyl, (C₃-C₈) cycloalkyl, preferably hydrogen, C₁-C₆ alkyl, (C₃-C₈) cycloalkyl.

27. The compound or pharmaceutically acceptable salt, isotope derivative, stereoisomer thereof as claimed in claim 1, wherein R₆ and R_{6′} each independently represent hydrogen, C₁-C₆ alkyl, (C₂-C₆)alkenyl, halogen, preferably hydrogen, C₁-C₆ alkyl, halogen.

28. The compound or pharmaceutically acceptable salt, isotope derivative, stereoisomer thereof as claimed in claim 1, wherein R₃ represents hydrogen, halogen, hydroxyl, (C₁-C₆) alkyl, —(C₀-C₆ alkylene) (C₃-C₈) cycloalkyl, —(C₀-C₆ alkylene) (4-8 membered) heterocycloalkyl, (C₁-C₆) alkoxy.

29. The compound or pharmaceutically acceptable salt, isotopic derivative, stereoisomer thereof as claimed in claim 1 having the following structure:

| serial | |
|---|---|
| number | Compound structure |
| 1 | 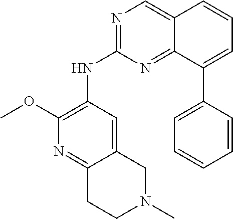 [SMILES: COc1nc2c(cc1Nc1ncc3cccc(-c4ccccc4)c3n1)CN(C)CC2] |
| 2 | 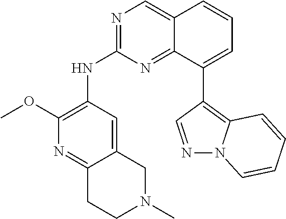 [SMILES: COc1nc2c(cc1Nc1ncc3cccc(-c4cnn5ccccc45)c3n1)CN(C)CC2] |
| 3 | 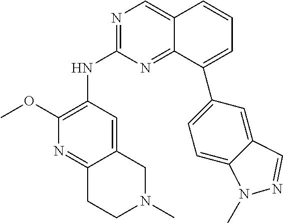 [SMILES: COc1nc2c(cc1Nc1ncc3cccc(-c4ccc5c(cnn5C)c4)c3n1)CN(C)CC2] |
| 4 | 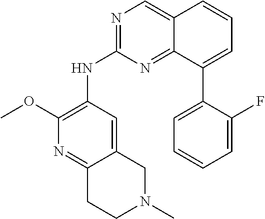 [SMILES: COc1nc2c(cc1Nc1ncc3cccc(-c4ccccc4F)c3n1)CN(C)CC2] |
| 5 | 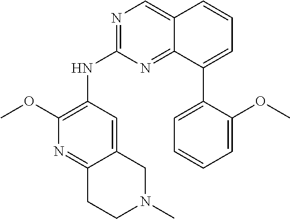 |
| 6 | 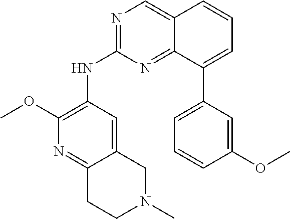 |
| 7 | 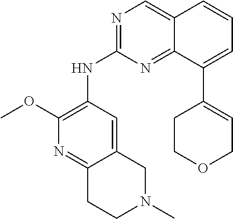 [SMILES: COc1nc2c(cc1Nc1ncc3cccc(C4=CCOCC4)c3n1)CN(C)CC2] |
| 8 | 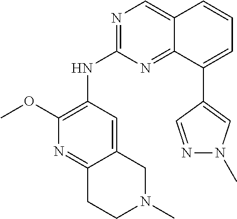 |
| 9 | 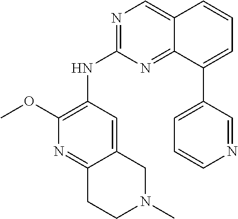 [SMILES: COc1nc2c(cc1Nc1ncc3cccc(-c4cccnc4)c3n1)CN(C)CC2] |
| 10 | 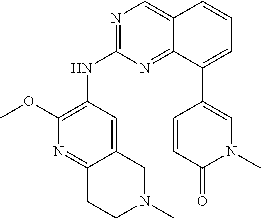 |
| 11 | 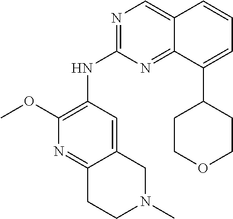 [SMILES: COc1nc2c(cc1Nc1ncc3cccc(C4CCOCC4)c3n1)CN(C)CC2] |
| 12 | 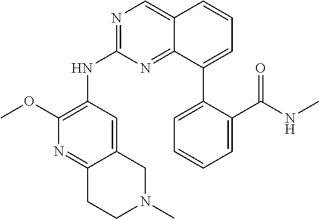 |
| 13 | 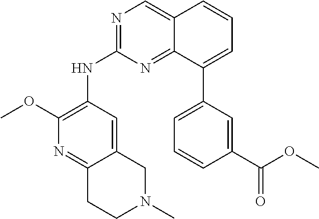 |
| 14 | 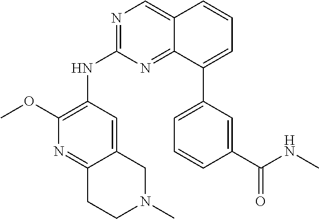 |
| 15 | 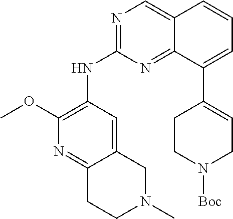 [SMILES: COc1nc2c(cc1Nc1ncc3cccc(C4=CCN(C(=O)OC(C)(C)C)CC4)c3n1)CN(C)CC2] |
| 16 | 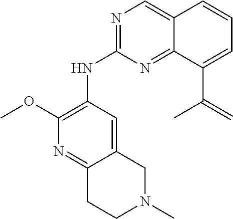 [SMILES: C=C(C)c1cccc2cnc(Nc3cc4c(nc3OC)CCN(C)C4)nc12] |
| 17 | 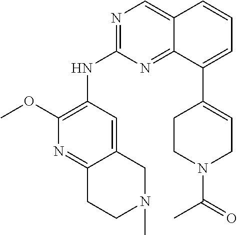 [SMILES: COc1nc2c(cc1Nc1ncc3cccc(C4=CCN(C(C)=O)CC4)c3n1)CN(C)CC2] |
| 18 | 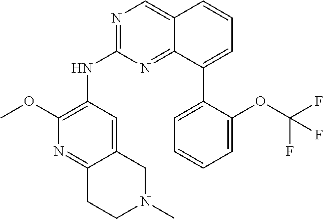 [SMILES: COc1nc2c(cc1Nc1ncc3cccc(-c4ccccc4OC(F)(F)F)c3n1)CN(C)CC2] |
| 19 | 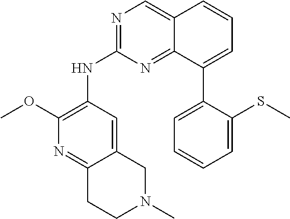 |
| 20 | 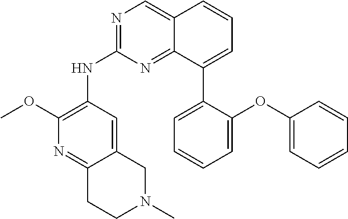 [SMILES: COc1nc2c(cc1Nc1ncc3cccc(-c4ccccc4Oc4ccccc4)c3n1)CN(C)CC2] |
| 21 | 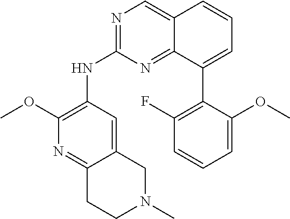 |
| 22 | 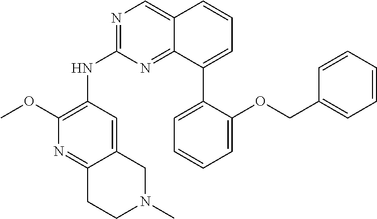 [SMILES: COc1nc2c(cc1Nc1ncc3cccc(-c4ccccc4OCc4ccccc4)c3n1)CN(C)CC2] |
| 23 | 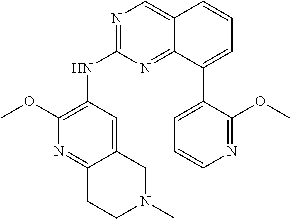 |
| 24 | 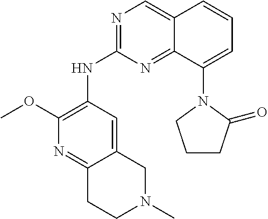 [SMILES: COc1nc2c(cc1Nc1ncc3cccc(N4CCCC4=O)c3n1)CN(C)CC2] |
| 25 | 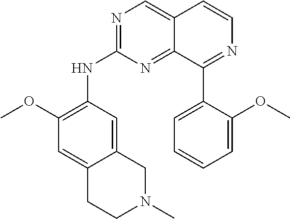 |
| 26 | 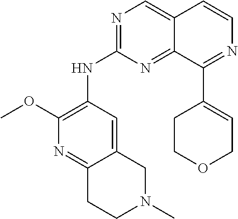 [SMILES: COc1nc2c(cc1Nc1ncc3ccnc(C4=CCOCC4)c3n1)CN(C)CC2] |
| 27 | 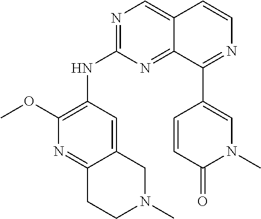 |
| 28 | 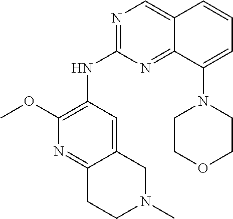 [SMILES: COc1nc2c(cc1Nc1ncc3cccc(N4CCOCC4)c3n1)CN(C)CC2] |
| 29 | 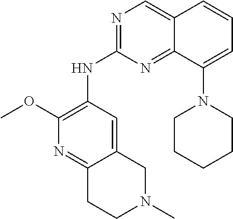 [SMILES: COc1nc2c(cc1Nc1ncc3cccc(N4CCCCC4)c3n1)CN(C)CC2] |
| 30 | 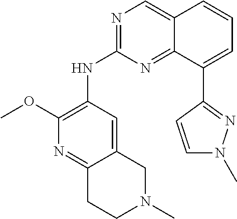 |
| 31 | 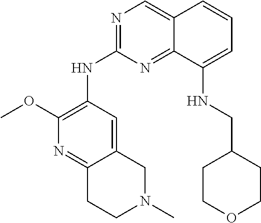 [SMILES: COc1nc2c(cc1Nc1ncc3cccc(NCC4CCOCC4)c3n1)CN(C)CC2] |
| 32 | 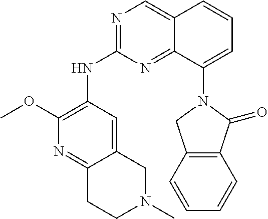 [SMILES: COc1nc2c(cc1Nc1ncc3cccc(N4Cc5ccccc5C4=O)c3n1)CN(C)CC2] |
| 33 | 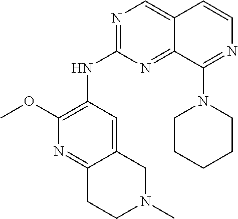 [SMILES: COc1nc2c(cc1Nc1ncc3ccnc(N4CCCCC4)c3n1)CN(C)CC2] |
| 34 | 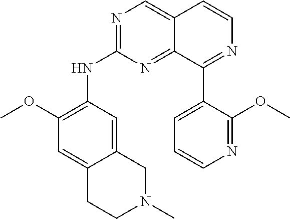 |
| 35 | 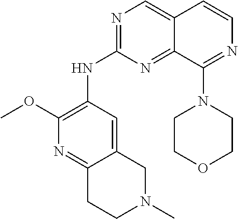 [SMILES: COc1nc2c(cc1Nc1ncc3ccnc(N4CCOCC4)c3n1)CN(C)CC2] |
| 36 | 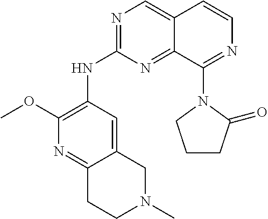 [SMILES: COc1nc2c(cc1Nc1ncc3ccnc(N4CCCC4=O)c3n1)CN(C)CC2] |
| 37 | 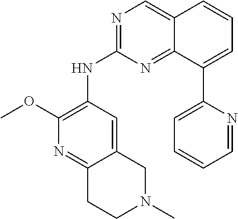 [SMILES: COc1nc2c(cc1Nc1ncc3cccc(-c4ccccn4)c3n1)CN(C)CC2] |
| 38 | 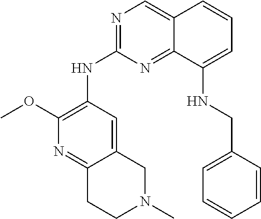 [SMILES: COc1nc2c(cc1Nc1ncc3cccc(NCc4ccccc4)c3n1)CN(C)CC2] |
| 39 | 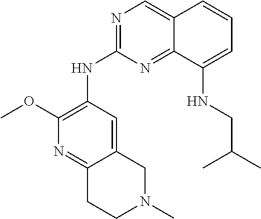 [SMILES: COc1nc2c(cc1Nc1ncc3cccc(NCC(C)C)c3n1)CN(C)CC2] |
| 40 | 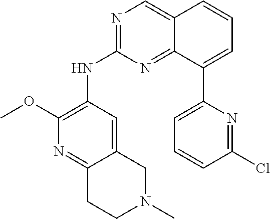 [SMILES: COc1nc2c(cc1Nc1ncc3cccc(-c4cccc(Cl)n4)c3n1)CN(C)CC2] |
| 41 | 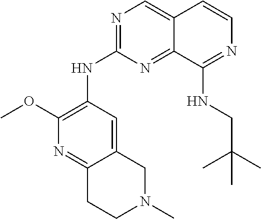 [SMILES: COc1nc2c(cc1Nc1ncc3ccnc(NCC(C)(C)C)c3n1)CN(C)CC2] |
| 42 | 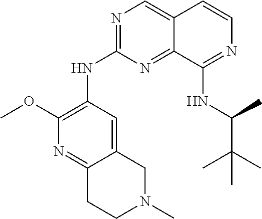 [SMILES: COc1nc2c(cc1Nc1ncc3ccnc(N[C@@H](C)C(C)(C)C)c3n1)CN(C)CC2] |
| 43 | 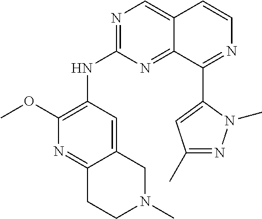 |
| 44 | 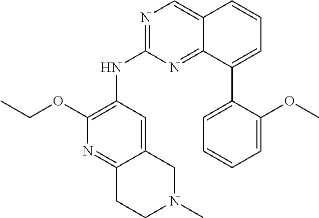 [SMILES: CCOc1nc2c(cc1Nc1ncc3cccc(-c4ccccc4OC)c3n1)CN(C)CC2] |
| 45 | 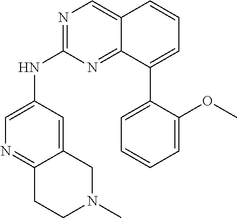 [SMILES: COc1ccccc1-c1cccc2cnc(Nc3cnc4c(c3)CN(C)CC4)nc12] |
| 46 | 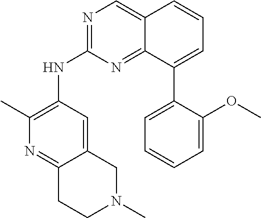 |
| 47 | 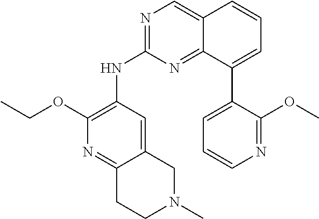 [SMILES: CCOc1nc2c(cc1Nc1ncc3cccc(-c4cccnc4OC)c3n1)CN(C)CC2] |
| 48 | 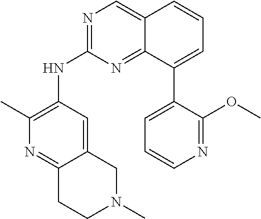 |
| 49 | 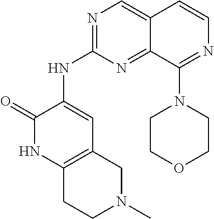 [SMILES: CN1CCc2[nH]c(=O)c(Nc3ncc4ccnc(N5CCOCC5)c4n3)cc2C1] |
| 50 | 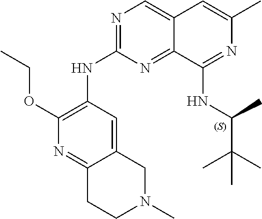 |
| 51 | 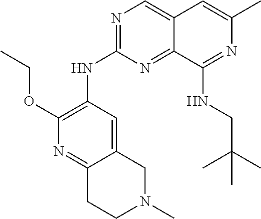 [SMILES: CCOc1nc2c(cc1Nc1ncc3cc(C)nc(NCC(C)(C)C)c3n1)CN(C)CC2] |
| 52 | 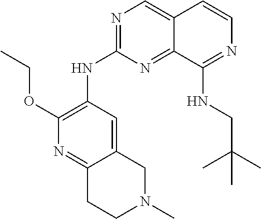 [SMILES: CCOc1nc2c(cc1Nc1ncc3ccnc(NCC(C)(C)C)c3n1)CN(C)CC2] |
| 53 | 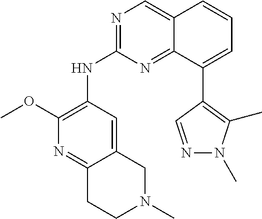 |
| 54 | 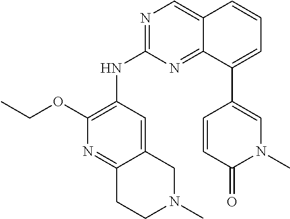 [SMILES: CCOc1nc2c(cc1Nc1ncc3cccc(-c4ccc(=O)n(C)c4)c3n1)CN(C)CC2] |
| 55 | 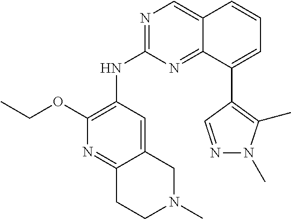 [SMILES: CCOc1nc2c(cc1Nc1ncc3cccc(-c4cnn(C)c4C)c3n1)CN(C)CC2] |
| 56 | 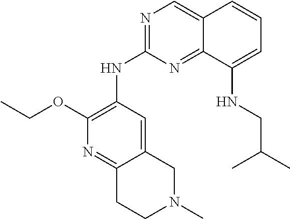 [SMILES: CCOc1nc2c(cc1Nc1ncc3cccc(NCC(C)C)c3n1)CN(C)CC2] |
| 57 | 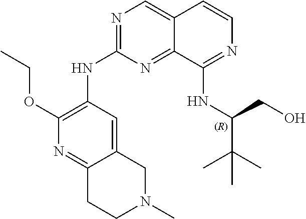 |
| 58 | 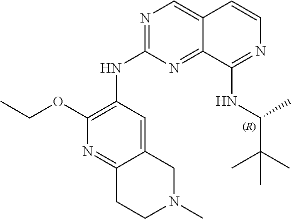 |
| 59 | 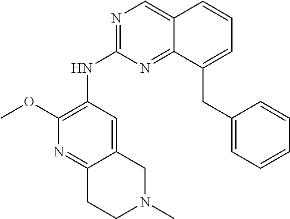 [SMILES: COc1nc2c(cc1Nc1ncc3cccc(Cc4ccccc4)c3n1)CN(C)CC2] |
| 60 | 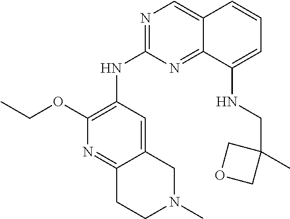 [SMILES: CCOc1nc2c(cc1Nc1ncc3cccc(NCC4(C)COC4)c3n1)CN(C)CC2] |
| 61 | 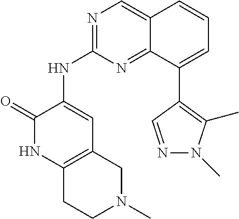 [SMILES: Cc1c(-c2cccc3cnc(Nc4cc5c([nH]c4=O)CCN(C)C5)nc23)cnn1C] |
| 62 | 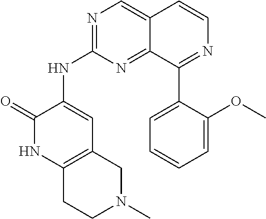 [SMILES: COc1ccccc1-c1nccc2cnc(Nc3cc4c([nH]c3=O)CCN(C)C4)nc12] |
| 63 | 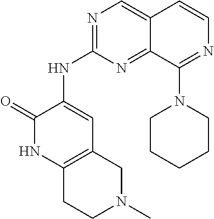 [SMILES: CN1CCc2[nH]c(=O)c(Nc3ncc4ccnc(N5CCCCC5)c4n3)cc2C1] |
| 64 | 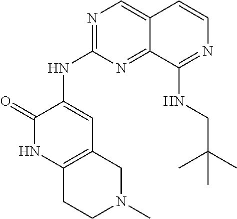 [SMILES: CN1CCc2[nH]c(=O)c(Nc3ncc4ccnc(NCC(C)(C)C)c4n3)cc2C1] |
| 65 | 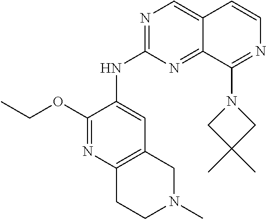 [SMILES: CCOc1nc2c(cc1Nc1ncc3ccnc(N4CC(C)(C)C4)c3n1)CN(C)CC2] |
| 66 | 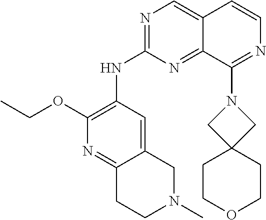 [SMILES: CCOc1nc2c(cc1Nc1ncc3ccnc(N4CC5(CCOCC5)C4)c3n1)CN(C)CC2] |
| 67 | 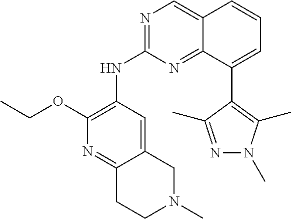 [SMILES: CCOc1nc2c(cc1Nc1ncc3cccc(-c4c(C)nn(C)c4C)c3n1)CN(C)CC2] |
| 68 | 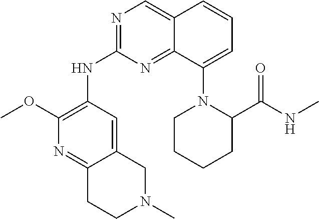 |
| 69 | 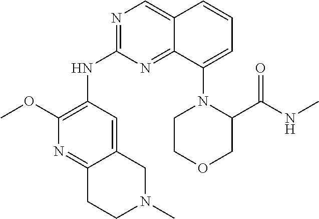 |
| 70 | 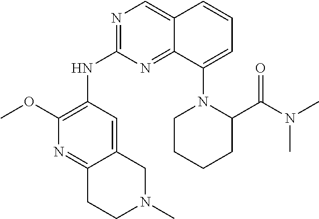 |
| 71 | 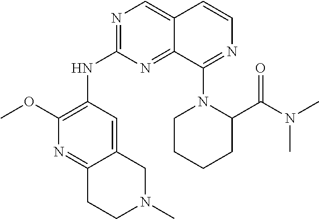 |
| 72 | 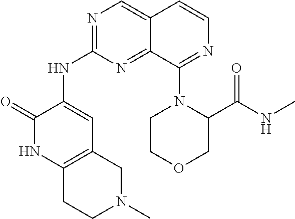 [SMILES: CNC(=O)C1COCCN1c1nccc2cnc(Nc3cc4c([nH]c3=O)CCN(C)C4)nc12] |
| 73 | 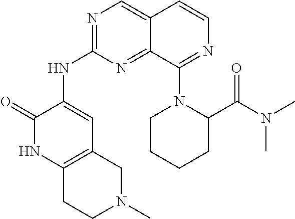 [SMILES: CN1CCc2[nH]c(=O)c(Nc3ncc4ccnc(N5CCCCC5C(=O)N(C)C)c4n3)cc2C1] |
| 74 | 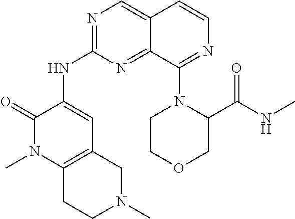 [SMILES: CNC(=O)C1COCCN1c1nccc2cnc(Nc3cc4c(n(C)c3=O)CCN(C)C4)nc12] |
| 75 | 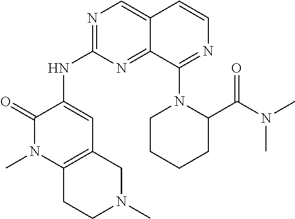 [SMILES: CN1CCc2c(cc(Nc3ncc4ccnc(N5CCCCC5C(=O)N(C)C)c4n3)c(=O)n2C)C1] |
| 76 | 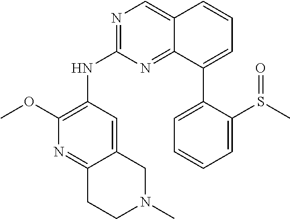 |
| 77 | 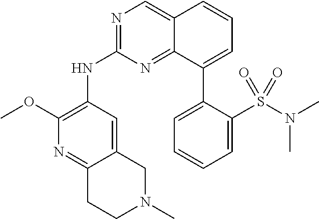 |
| 78 | 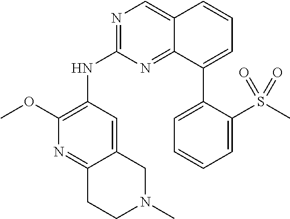 |
| 79 | 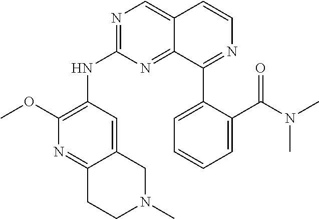 |
| 80 | 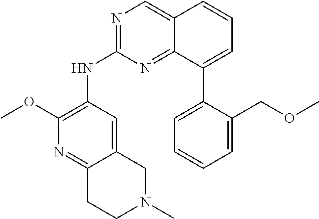 |
| 81 | 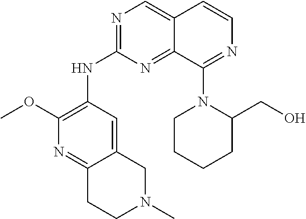 [SMILES: COc1nc2c(cc1Nc1ncc3ccnc(N4CCCCC4CO)c3n1)CN(C)CC2] |
| 82 | 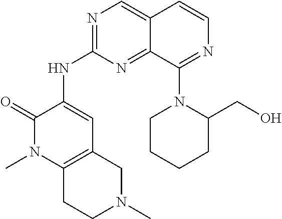 [SMILES: CN1CCc2c(cc(Nc3ncc4ccnc(N5CCCCC5CO)c4n3)c(=O)n2C)C1] |
| 83 | 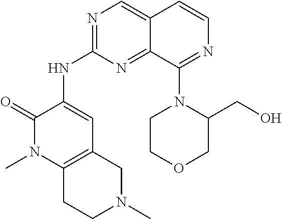 [SMILES: CN1CCc2c(cc(Nc3ncc4ccnc(N5CCOCC5CO)c4n3)c(=O)n2C)C1] |
| 84 | 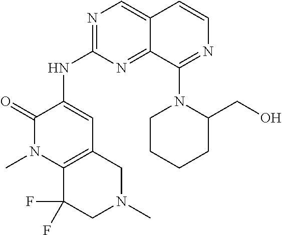 [SMILES: CN1Cc2cc(Nc3ncc4ccnc(N5CCCCC5CO)c4n3)c(=O)n(C)c2C(F)(F)C1] |
| 85 | 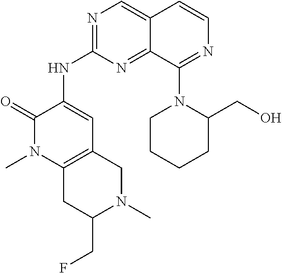 [SMILES: CN1Cc2cc(Nc3ncc4ccnc(N5CCCCC5CO)c4n3)c(=O)n(C)c2CC1CF] |
| 86 | 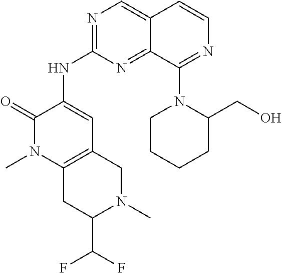 [SMILES: CN1Cc2cc(Nc3ncc4ccnc(N5CCCCC5CO)c4n3)c(=O)n(C)c2CC1C(F)F] |
| 87 | 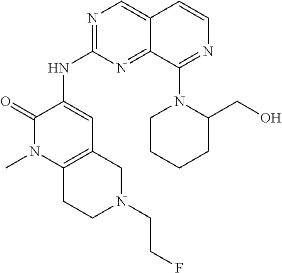 [SMILES: Cn1c2c(cc(Nc3ncc4ccnc(N5CCCCC5CO)c4n3)c1=O)CN(CCF)CC2] |
| 88 | 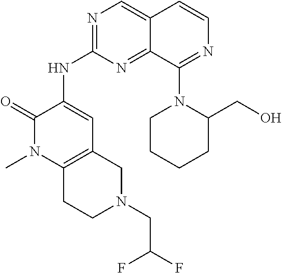 [SMILES: Cn1c2c(cc(Nc3ncc4ccnc(N5CCCCC5CO)c4n3)c1=O)CN(CC(F)F)CC2] |
| 89 | 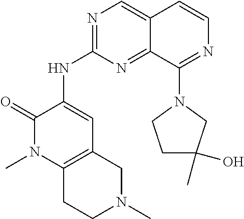 [SMILES: CN1CCc2c(cc(Nc3ncc4ccnc(N5CCC(C)(O)C5)c4n3)c(=O)n2C)C1] |
| 90 | 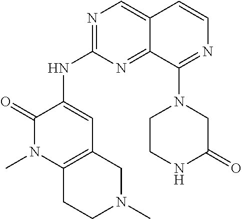 [SMILES: CN1CCc2c(cc(Nc3ncc4ccnc(N5CCNC(=O)C5)c4n3)c(=O)n2C)C1] |
| 91 | 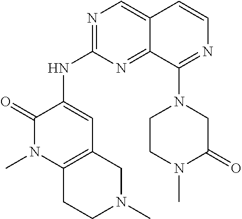 [SMILES: CN1CCc2c(cc(Nc3ncc4ccnc(N5CCN(C)C(=O)C5)c4n3)c(=O)n2C)C1] |
| 92 | 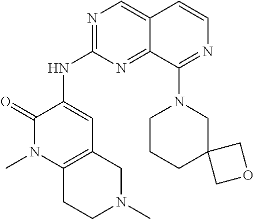 [SMILES: CN1CCc2c(cc(Nc3ncc4ccnc(N5CCCC6(COC6)C5)c4n3)c(=O)n2C)C1] |
| 93 | 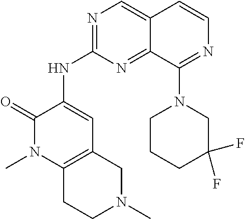 [SMILES: CN1CCc2c(cc(Nc3ncc4ccnc(N5CCCC(F)(F)C5)c4n3)c(=O)n2C)C1] |
| 94 | 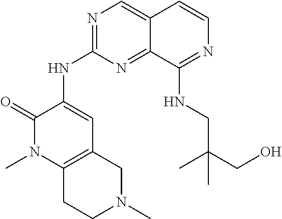 [SMILES: CN1CCc2c(cc(Nc3ncc4ccnc(NCC(C)(C)CO)c4n3)c(=O)n2C)C1] |
| 95 | 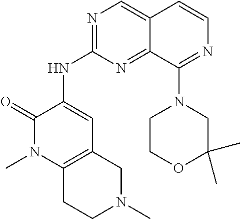 [SMILES: CN1CCc2c(cc(Nc3ncc4ccnc(N5CCOC(C)(C)C5)c4n3)c(=O)n2C)C1] |
| 96 | 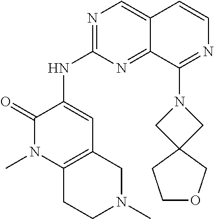 [SMILES: CN1CCc2c(cc(Nc3ncc4ccnc(N5CC6(CCOC6)C5)c4n3)c(=O)n2C)C1] |
| 97 | 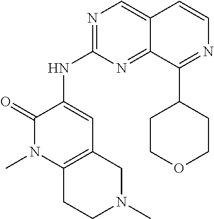 [SMILES: CN1CCc2c(cc(Nc3ncc4ccnc(C5CCOCC5)c4n3)c(=O)n2C)C1] |
| 98 | 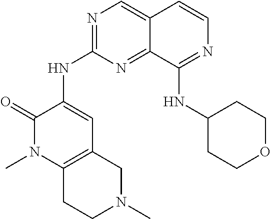 [SMILES: CN1CCc2c(cc(Nc3ncc4ccnc(NC5CCOCC5)c4n3)c(=O)n2C)C1] |
| 99 | 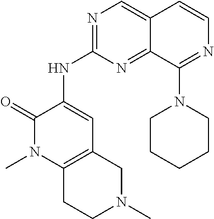 [SMILES: CN1CCc2c(cc(Nc3ncc4ccnc(N5CCCCC5)c4n3)c(=O)n2C)C1] |
| 100 | 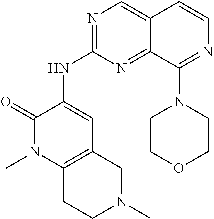 [SMILES: CN1CCc2c(cc(Nc3ncc4ccnc(N5CCOCC5)c4n3)c(=O)n2C)C1] |
| 101 | 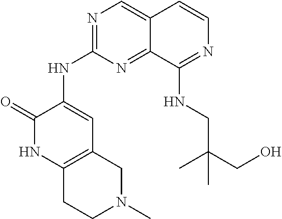 [SMILES: CN1CCc2[nH]c(=O)c(Nc3ncc4ccnc(NCC(C)(C)CO)c4n3)cc2C1] |
| 102 | 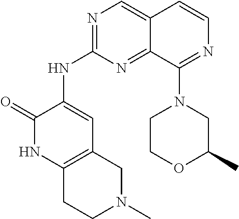 [SMILES: C[C@@H]1CN(c2nccc3cnc(Nc4cc5c([nH]c4=O)CCN(C)C5)nc23)CCO1] |
| 103 | 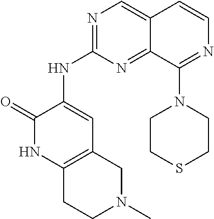 [SMILES: CN1CCc2[nH]c(=O)c(Nc3ncc4ccnc(N5CCSCC5)c4n3)cc2C1] |
| 104 | 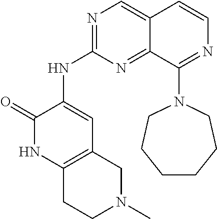 [SMILES: CN1CCc2[nH]c(=O)c(Nc3ncc4ccnc(N5CCCCCC5)c4n3)cc2C1] |
| 105 | 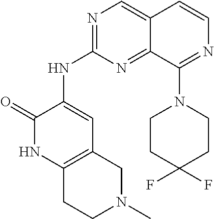 [SMILES: CN1CCc2[nH]c(=O)c(Nc3ncc4ccnc(N5CCC(F)(F)CC5)c4n3)cc2C1] |
| 106 | 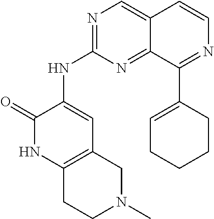 [SMILES: CN1CCc2[nH]c(=O)c(Nc3ncc4ccnc(C5=CCCCC5)c4n3)cc2C1] |
| 107 | 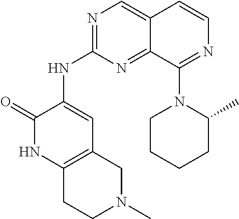 [SMILES: C[C@@H]1CCCCN1c1nccc2cnc(Nc3cc4c([nH]c3=O)CCN(C)C4)nc12] |
| 108 | 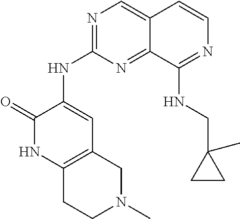 [SMILES: CN1CCc2[nH]c(=O)c(Nc3ncc4ccnc(NCC5(C)CC5)c4n3)cc2C1] |
| 109 | 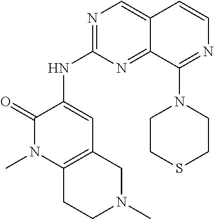 [SMILES: CN1CCc2c(cc(Nc3ncc4ccnc(N5CCSCC5)c4n3)c(=O)n2C)C1] |
| 110 | 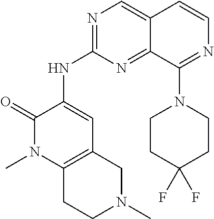 [SMILES: CN1CCc2c(cc(Nc3ncc4ccnc(N5CCC(F)(F)CC5)c4n3)c(=O)n2C)C1] |
| 111 | 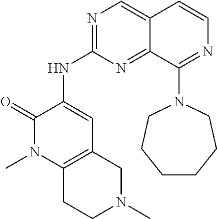 [SMILES: CN1CCc2c(cc(Nc3ncc4ccnc(N5CCCCCC5)c4n3)c(=O)n2C)C1] |
| 112 | 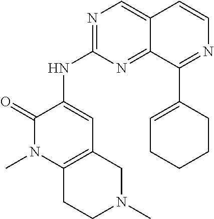 [SMILES: CN1CCc2c(cc(Nc3ncc4ccnc(C5=CCCCC5)c4n3)c(=O)n2C)C1] |
| 113 | 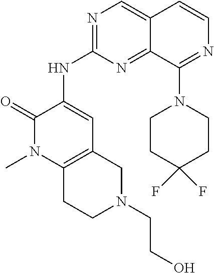 [SMILES: Cn1c2c(cc(Nc3ncc4ccnc(N5CCC(F)(F)CC5)c4n3)c1=O)CN(CCO)CC2] |
| 114 | 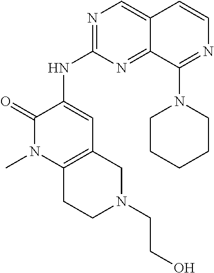 [SMILES: Cn1c2c(cc(Nc3ncc4ccnc(N5CCCCC5)c4n3)c1=O)CN(CCO)CC2] |
| 115 | 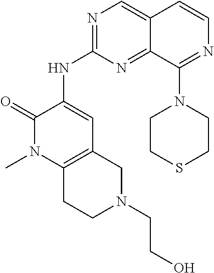 [SMILES: Cn1c2c(cc(Nc3ncc4ccnc(N5CCSCC5)c4n3)c1=O)CN(CCO)CC2] |
| 116 | 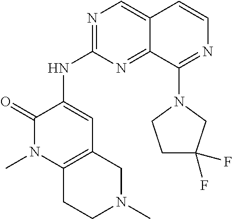 [SMILES: CN1CCc2c(cc(Nc3ncc4ccnc(N5CCC(F)(F)C5)c4n3)c(=O)n2C)C1] |
| 117 | 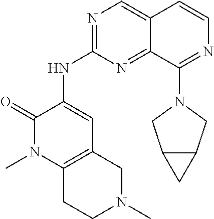 [SMILES: CN1CCc2c(cc(Nc3ncc4ccnc(N5CC6CC6C5)c4n3)c(=O)n2C)C1] |
| 118 | 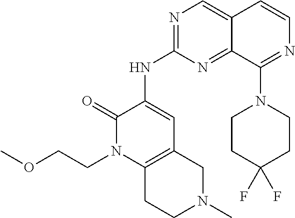 [SMILES: COCCn1c2c(cc(Nc3ncc4ccnc(N5CCC(F)(F)CC5)c4n3)c1=O)CN(C)CC2] |
| 119 | 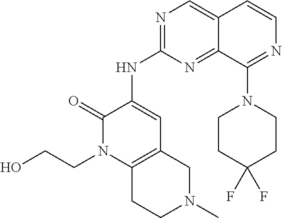 [SMILES: CN1CCc2c(cc(Nc3ncc4ccnc(N5CCC(F)(F)CC5)c4n3)c(=O)n2CCO)C1] |
| 120 | 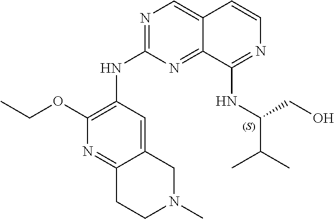 |
| 121 | 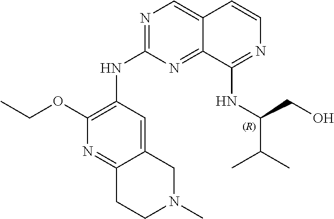 |
| 122 | 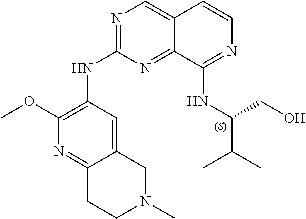 |
| 123 | 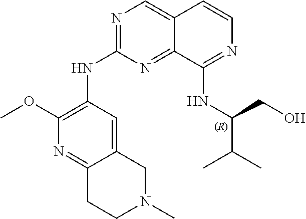 |
| 124 | 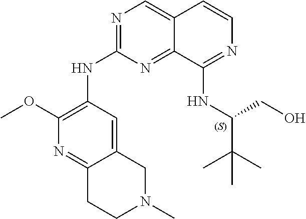 |
| 125 | 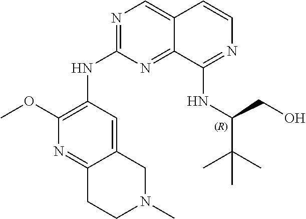 |
| 126 | 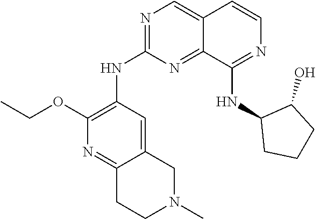 [SMILES: CCOc1nc2c(cc1Nc1ncc3ccnc(N[C@@H]4CCC[C@H]4O)c3n1)CN(C)CC2] |
| 127 | 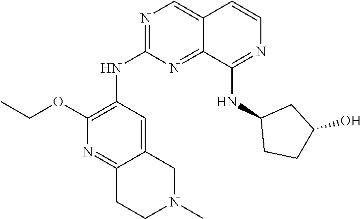 [SMILES: CCOc1nc2c(cc1Nc1ncc3ccnc(N[C@@H]4CC[C@@H](O)C4)c3n1)CN(C)CC2] |
| 128 | 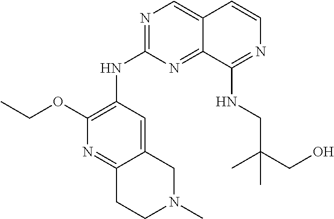 [SMILES: CCOc1nc2c(cc1Nc1ncc3ccnc(NCC(C)(C)CO)c3n1)CN(C)CC2] |
| 129 | 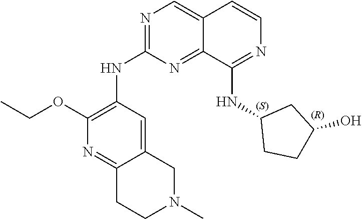 |
| 130 | 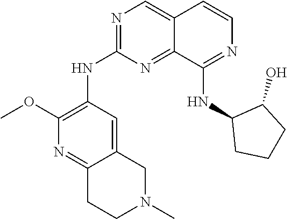 [SMILES: COc1nc2c(cc1Nc1ncc3ccnc(N[C@@H]4CCC[C@H]4O)c3n1)CN(C)CC2] |
| 131 | 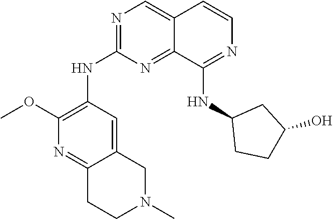 [SMILES: COc1nc2c(cc1Nc1ncc3ccnc(N[C@@H]4CC[C@@H](O)C4)c3n1)CN(C)CC2] |
| 132 | 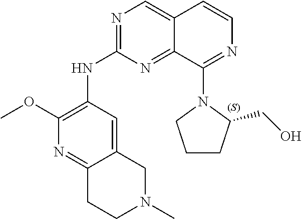 |
| 133 | 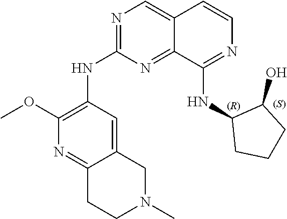 |
| 134 | 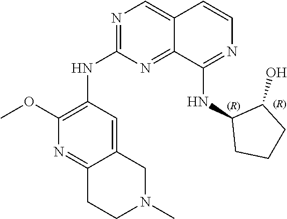 |
| 135 | 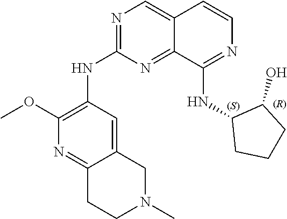 |
| 136 | 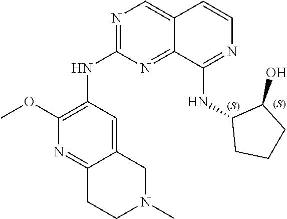 |
| 137 | 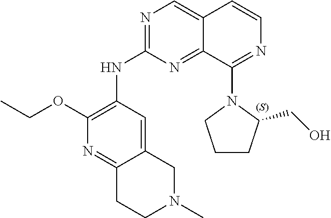 |
| 138 | 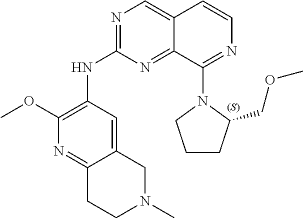 |
| 139 | 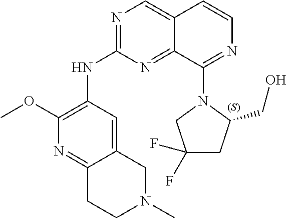 |
| 140 | 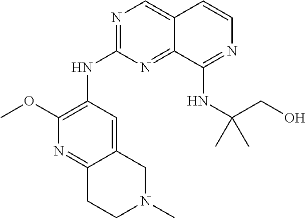 [SMILES: COc1nc2c(cc1Nc1ncc3ccnc(NC(C)(C)CO)c3n1)CN(C)CC2] |
| 141 | 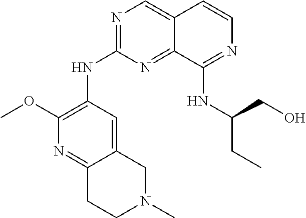 [SMILES: CC[C@H](CO)Nc1nccc2cnc(Nc3cc4c(nc3OC)CCN(C)C4)nc12] |
| 141 | 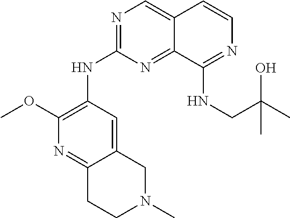 [SMILES: COc1nc2c(cc1Nc1ncc3ccnc(NCC(C)(C)O)c3n1)CN(C)CC2] |
| 143 | 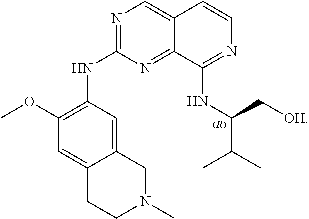 |

30. A pharmaceutical composition comprising the compound according to claim 1 and a pharmaceutically acceptable carrier.

31. A method for preventing and/or treating cancer, tumors, inflammatory diseases, autoimmune diseases or immune-mediated diseases, comprising administering the compound or pharmaceutically acceptable salt, isotope derivative, stereoisomer thereof described in claim 1 to a subject in need thereof.
